(19) United States
(12) Patent Application Publication
(10) Pub. No.: US 2023/0381231 A1
(43) Pub. Date: Nov. 30, 2023

(54) COMPOSITIONS FOR CANCER TREATMENT AND METHODS AND USES FOR CANCER TREATMENT AND PROGNOSIS

(71) Applicants: La Jolla Institute for Immunology, La Jolla, CA (US); University of Southampton, Hampshire (GB)

(72) Inventors: Pandurangan Vijayanand, La Jolla, CA (US); Christian Ottensmeier, Hampshire (GB); Anusha Preethi Ganesan, La Jolla, CA (US); James Clarke, Hampshire (GB); Tilman Sanchez-Elsner, Hampshire (GB)

(21) Appl. No.: 18/076,193

(22) Filed: Dec. 6, 2022

Related U.S. Application Data
(63) Continuation of application No. 16/465,983, filed on May 31, 2019, now abandoned.
(60) Provisional application No. 62/431,265, filed on Dec. 7, 2016.
(60) Provisional application No. 62/522,048, filed on Jun. 19, 2017.

(51) Int. Cl. A61K 35/17; A61P 35/00; C12Q 1/6886; C12N 5/0783; C12N 5/10

(52) U.S. Cl. CPC A61K 35/17; A61P 35/00; C12Q 1/6886; C12N 5/0636; C12N 5/10; A61K 45/06

(57) ABSTRACT

Global transcriptional profiling of CTLs in tumors and adjacent non-tumor tissue from treatment-naïve patients with early stage lung cancer revealed molecular features associated with robustness of anti-tumor immune responses. Major differences in the transcriptional program of tumor-infiltrating CTLs were observed that are shared across tumor subtypes. Pathway analysis revealed enrichment of genes in cell cycle, T cell receptor (TCR) activation and co-stimulation pathways, indicating tumor-driven expansion of presumed tumor antigen-specific CTLs. Marked heterogeneity in the expression of molecules associated with TCR activation and immune checkpoints such as 4-1BB, PD1, TIM3, was also observed and their expression was positively correlated with the density of tumor-infiltrating CTLs. Transcripts linked to tissue-resident memory cells (TRM), such as CD103, were enriched in tumors containing a high density of CTLs, and CTLs from CD103^{high} tumors displayed features of enhanced cytotoxicity, implying better anti-tumor activity. In an independent cohort of 689 lung cancer patients, patients with CD103^{high} (TRM rich) tumors survived significantly longer. In summary, the molecular fingerprint of tumor-infiltrating CTLs at the site of primary tumor was defined and a number of novel targets identified that appear to be important in modulating the magnitude and specificity of anti-tumor immune responses in lung cancer.

### CROSS REFERENCE TO RELATED APPLICATIONS

The present application claims priority under 35 U.S.C. § 119(e) to U.S. Provisional Application 62/431,265, filed on Dec. 7, 2016, and U.S. Provisional Application 62/522,048, filed on Jun. 19, 2017, the contents of which are hereby incorporated by reference in their entirety.

### BACKGROUND

Throughout and within this disclosure reference is made to patent and technical literature by reference to an identifying citation or an Arabic numeral, the complete bibliographic information for which is found immediately preceding the claims. These disclosure provide a background of the state of the art to which this disclosure pertains.

Immunotherapy is rapidly gaining its place as a standard treatment for solid tumors^{1, 2}, including lung cancer³. Nonetheless, only ˜30% of patients benefit from this approach⁴. Much remains to be learned about how immunotherapies work and how to choose the right treatment or combination for a particular patient. Understanding the mechanisms and molecular basis of effective anti-tumor immune responses will be essential to develop novel immunotherapeutic agents for those patients who do not respond to currently available immunotherapies.

Immunotherapies are thought to enhance the antitumor responses of cytotoxic T lymphocytes (CTLs) i.e., CD8⁺ T cells that infiltrate into the tumor⁵. Indeed, a high density of tumor-infiltrating lymphocytes (TIL) predicts good prognosis in a wide range of cancers, and in some, is the most important predictor of patient survival, surpassing standard pathological and clinical staging^{6,7}. However, it remains unclear why the degree of infiltration by TILs varies significantly even between individuals with the same cancer. It is also unknown whether there are merely quantitative differences in the number of TILs or whether qualitative differences also exist in TILs from tumors with high TIL density that may contribute to the superior outcome seen in these patients. An understanding of the TIL transcriptome and the molecular basis of TIL heterogeneity could lead not only to novel biomarkers for patient stratification for therapy but also identify novel immune pathways to be targeted by future immunotherapeutic strategies. This disclosure provides these benefits and provides related advantages as well.

### SUMMARY OF THE DISCLOSURE

Aspects of this disclosure relate to selecting and/or modifying cells for the treatment of cancer, as well as diagnosing and assessing cancer prognosis and/or survival.

Aspects of this disclosure relate to methods of treating cancer in a subject and/or eliciting an anti-tumor response comprising, or alternatively consisting essentially of, or yet further consisting of, administering to the subject and/or contacting the tumor or a tumor cell with, respectively, an effective amount of a population of T-cells that exhibit one or more of the following characteristics:
(i) higher than baseline expression of one or more genes set forth in Table 1, Table 4, Table 7 and/or Table 8;
(ii) lower than baseline expression of one or more genes set forth in Table 1, Table 4, Table 7 and/or Table 8;
(iii) higher than baseline expression of genes involved in one or more pathways set forth in Table 5 and/or Table 9;
(iv) lower than baseline expression of genes involved in one or more pathways set forth in Table 5 and/or Table 9;
(v) higher than baseline expression of one or more genes set forth in Table 12; and/or
(vi) lower than baseline expression of one or more genes set forth in Table 13.

In some embodiments, the T-cells are CD8+ and/or tumor infiltrating lymphocytes (TILs). Such embodiments include (i) to (iv) but are not limited to listed above. In some embodiments, the T-cells are tissue-resident memory cells (T_{RM}). Such embodiments include (v) and (vi) listed above. Similar aspects relate to methods of treating cancer in a subject and/or eliciting an anti-tumor response comprising, or alternatively consisting essentially of, or yet further consisting of, administering to the subject and/or contacting the tumor or a tumor cell with, respectively, an effective amount of one or more an active agent that induces in T-cells, one or more of:
(i) higher than baseline expression of one or more genes set forth in Table 1, Table 4, Table 7 and/or Table 8;
(ii) lower than baseline expression of one or more genes set forth in Table 1, Table 4, Table 7 and/or Table 8;
(iii) higher than baseline expression of genes involved in one or more pathways set forth in Table 5 and/or Table 9;
(iv) lower than baseline expression of genes involved in one or more pathways set forth in Table 5 and/or Table 9;
(v) higher than baseline expression of one or more genes set forth in Table 12; and/or
(vi) lower than baseline expression of one or more genes set forth in Table 13.

In some embodiments, the T-cells are CD8+ and/or tumor infiltrating lymphocytes (TILs). Such embodiments include but are not limited to (i) to (iv) listed above. In some embodiments, the T-cells are tissue-resident memory cells (T_{RM}). Such embodiments include (v) and (vi) listed above. In some embodiments, the active agent is an antibody, a small molecule, or a nucleic acid.

Additional aspects relate to methods of modulating protein expression in a subject or a sample comprising, or alternatively consisting essentially of, or yet further consisting of, administering an effective amount of one or more an active agent that induces in T-cells, higher or lower than baseline expression of one or more proteins encoded by the genes set forth in any one of Tables 1-13 to the subject or sample, optionally one or more of: (i) higher than baseline expression of one or more proteins encoded by genes set forth in Table 1, Table 4, Table 7 and/or Table 8;
(ii) lower than baseline expression of one or more proteins encoded by genes set forth in Table 1, Table 4, Table 7 and/or Table 8;
(iii) higher than baseline expression of proteins encoded by genes involved in one or more pathways set forth in Table 5 and/or Table 9;
(iv) lower than baseline expression of proteins encoded by genes involved in one or more pathways set forth in Table 5 and/or Table 9;
(v) higher than baseline expression of one or more proteins encoded by genes set forth in Table 12; and/or
(vi) lower than baseline expression of one or more proteins encoded by genes set forth in Table 13.

Additional aspects relate to methods of modulating protein activity in a subject or a sample comprising, or alternatively consisting essentially of, or yet further consisting of, administering an effective amount of one or more an active agent that modulates in T-cells, one or more proteins encoded by the genes set forth in any one of Tables 1-13 to the subject or sample, optionally one or more of:
(i) induce activity of one or more proteins encoded by genes set forth in Table 1, Table 4, Table 7 and/or Table 8;
(ii) inhibit activity of one or more proteins encoded by genes set forth in Table 1, Table 4, Table 7 and/or Table 8;
(iii) induce activity of one or more proteins encoded by genes involved in one or more pathways set forth in Table 5 and/or Table 9;
(iv) inhibit activity of one or more of proteins encoded by genes involved in one or more pathways set forth in Table 5 and/or Table 9;
(v) induce activity of one or more proteins encoded by genes set forth in Table 12; and/or
(vi) inhibit activity of one or more proteins encoded by genes set forth in Table 13.

In some embodiments, the method is effective for treating cancer in a subject and/or eliciting an anti-tumor response; thus, the method comprises, or alternatively consists essentially of, or yet further consists of, administering the agent to the subject and/or contacting the tumor or a tumor cell with the agent, respectively. In some embodiments, the T-cells are CD8+ and/or tumor infiltrating lymphocytes (TILs). Such embodiments include but are not limited to (i) to (iv) listed above. In some embodiments, the T-cells are tissue-resident memory cells (T_{RM}). Such embodiments include (v) and (vi) listed above. In some embodiments, the active agent is an antibody, a small molecule, or a nucleic acid.

Still further aspects relate to a modified T-cell, which is modified to exhibit one or more of:
(i) higher than baseline expression of one or more genes set forth in Table 1, Table 4, Table 7 and/or Table 8;
(ii) lower than baseline expression of one or more genes set forth in Table 1, Table 4, Table 7 and/or Table 8;
(iii) higher than baseline expression of genes involved in one or more pathways set forth in Table 5 and/or Table 9;
(iv) lower than baseline expression of genes involved in one or more pathways set forth in Table 5 and/or Table 9;
(v) higher than baseline expression of one or more genes set forth in Table 12; and/or
(vi) lower than baseline expression of one or more genes set forth in Table 13.

In some embodiments, the T-cells are CD8+. Such embodiments include but are not limited to (i) to (iv) listed above. In some embodiments, the T-cells are tissue-resident memory cells (T_{RM}). Such embodiments include (v) and (vi) listed above. In some embodiments, the T-cell is modified using techniques of genetic modification, such as but not limited to those techniques employing recombinant methods and/or CRISPR/Cas systems. In some embodiments, the T-cell is further modified to express a protein that binds to a cytokine, chemokine, lymphokine, or a receptor each thereof and/or CD19. In further embodiments, this protein comprises, or alternatively consists essentially of, or yet further consisting of, an antibody or antigen binding fragment thereof, optionally wherein the antibody is IgG, IgA, IgM, IgE or IgD, or a subclass thereof or the antigen binding fragment is an Fab, Fab′, F(ab′)2, Fv, Fd, single-chain Fvs (scFv), disulfide-linked Fvs (sdFv) or V_{L} or V_{H}. Regarding antibodies, non-limiting exemplary subclasses of IgG relevant to aspects disclosed herein include but are not limited to IgG₁, IgG₂, IgG₃ and IgG₄.

Further aspects relate to compositions comprising, or alternatively consisting essentially of, or yet further consisting of, the aforementioned modified T-cell. Still further aspects relate to treating cancer in a subject and/or eliciting an anti-tumor response with one or more of the modified T-cell and/or compositions disclosed herein.

Some aspects relate to diagnostic and prognostic methods utilizing the expression profiles disclosed herein above.

For example, aspects disclosed herein relate to a method of determining the density of tumor infiltrating lymphocytes (TILs), optionally T-cells, in a cancer, tumor, or sample thereof comprising, or alternatively consisting essentially of, or yet further consisting of, measuring expression of one or more gene selected from the group of 4-1BB, PD-1, or TIM3 in the cancer, tumor, or sample thereof, wherein higher than baseline expression indicates higher density of TILs in the cancer, tumor, or sample thereof. Additional aspects relate to a method to determine the density of tissue-resident memory cells (T_{RM}), optionally T-cells, in a cancer, tumor, or sample thereof comprising, or alternatively consisting essentially of, or yet further consisting of, measuring the level of CD103 in the cancer, tumor, or sample thereof, wherein higher than baseline levels of CD103 indicates a high density of T_{RM} in the cancer, tumor, or sample thereof. In some method aspects, prognosis of a subject having cancer is determined based on the density of TILs and/or T_{RM} in the cancer or a sample thereof, i.e. wherein a high density of TILs and/or T_{RM} indicates an increased probability and/or duration of survival. As disclosed herein, measuring CD103 levels can be used to determine density of T_{RM}. Thus, density or frequency of CD103 can serve as a prognostic indicator in the same manner as density of T_{RM}. Further, in embodiments relating to the density of TILs, these cells can be enriched for T_{RM}, for example by contacting the TILs with an effective amount of an active agent that induces higher than baseline expression of one or more genes set forth in Table 12 and/or an active agent that induces lower than base line expression of one or more genes set forth in Table 13 in TILs. As noted above, such an active agent can optionally be an antibody, a small molecule, or a nucleic acid. It is appreciated that in such an enriched population, in some embodiments, the TILs enriched for T_{RM} have enhanced cytotoxicity and proliferation.

Further aspects relate to a method of diagnosing, determining prognosis in a subject, and/or responsiveness to cancer therapy by detecting the presence of one or more of:
(i) one or more genes set forth in Table 1, Table 4, Table 7 and/or Table 8, wherein higher than baseline levels is diagnostic of cancer and/or indicates an increased probability and/or duration of survival and/or indicates that the subject is likely to respond to cancer therapy;
(ii) one or more genes set forth in Table 1, Table 4, Table 7 and/or Table 8, wherein lower than baseline levels is diagnostic of cancer and/or indicates an increased probability and/or duration of survival and/or indicates that the subject is likely to respond to cancer therapy;
(iii) one or more genes set forth in Table 12, wherein higher than baseline levels is diagnostic of cancer and/or indicates an increased probability and/or duration of survival and/or indicates that the subject is likely to respond to cancer therapy; and/or
(iv) one or more genes set forth in Table 13, wherein lower than baseline levels is diagnostic of cancer and/or indicates an increased probability and/or duration of survival and/or indicates that the subject is likely to respond to cancer therapy.

In some embodiments, the T-cells are CD8+ and/or tumor infiltrating lymphocytes (TILs). Such embodiments include but are not limited to (i) to (ii) listed above. In some embodiments, the T-cells are tissue-resident memory cells (T_{RM}). Such embodiments include (iii) and (iv) listed above. In further embodiments of these aspects, the detection is conducted by contacting the cancer, tumor, or sample (as relevant) with an agent, optionally including a detectable label or tag. The detectable label or tag can comprise a radioisotope, a metal, horseradish peroxidase, alkaline phosphatase, avidin or biotin. Further, the agent may comprise a polypeptide that binds to an expression product encoded by the gene, or a polynucleotide that hybridizes to a nucleic acid sequence encoding all or a portion of the gene or that binds to an expression product encoded by the gene, or a polynucleotide that hybridizes to a nucleic acid sequence encoding all or a portion of the gene. In some aspects, the polypeptide comprises, or alternatively consisting essentially of, or yet further consisting of, an antibody, an antigen binding fragment thereof, or a receptor that binds to the gene.

Further exemplary aspects are disclosed herein, including:
a method of determining prognosis of a subject having cancer, optionally lung cancer, comprising, or alternatively consisting essentially of, or yet further consisting of, contacting tumor infiltrating lymphocytes (TILs) of the cancer or a sample thereof with an antibody that recognizes and binds CD103 to determine the frequency of CD103+ TILs, wherein a high frequency of CD103+ TILs indicates an increased probability and/or duration of survival;
a method of determining the responsiveness of a subject having cancer to immunotherapy comprising, or alternatively consisting essentially of, or yet further consisting of, contacting tumor infiltrating lymphocytes (TILs) of the cancer or a sample thereof with an antibody that recognizes and binds CD8, and antibody that recognizes and binds PD-1, an antibody that recognizes and binds TIM3, an antibody that recognizes and binds LAG3, and an antibody that recognizes and binds CTLA4 to determine the frequency of CD8⁺PD1⁺, CD8⁺TIM3⁺, CD8⁺LAG3⁺, CD8⁺CTLA4⁺, CD8⁺PD1⁺TIM3⁺, CD8⁺PD1⁺LAG3⁺, CD8⁺PD1⁺CTLA4⁺, CD8⁺TIM3⁺LAG3⁺, CD8⁺TIM3⁺CTLA4⁺, CD8⁺LAG3⁺CTLA4⁺, CD8⁺PD1⁺TIM3⁺LAG3⁺, CD8⁺PD1⁺LAG3⁺CTLA4⁺, or CD8⁺PD1⁺TIM3⁺CTLA4⁺ TILs, wherein a high frequency of one or more of these TILs indicates responsiveness to immunotherapy
a method of determining the responsiveness of a subject having cancer to immunotherapy comprising, or alternatively consisting essentially of, or yet further consisting of, contacting tumor infiltrating lymphocytes (TILs) of the cancer or a sample thereof with an antibody that recognizes and binds CD8, and antibody that recognizes and binds SlPRl, and an antibody that recognizes and binds KLF2 to determine the frequency of CD8+S1PR1− or CD8+KLF2− TILs, wherein a high frequency of one or more of these TILs indicates an increased responsiveness to immunotherapy.

It is appreciated that in any such embodiment disclosed herein, such as the exemplary embodiments of the paragraph above, similar embodiments may include the use of antibodies or detection of expression of one or more proteins encoded by one or more genes or related genes in pathways disclosed in Tables 1-13. Non-limiting exemplary embodiments thereof are described in the claims below.

In aspects where responsiveness to therapy for example, cancer therapy or immunotherapy, is assessed further embodiments may include the administration of the therapy to the subject being assessed. Non-limiting examples of cancer therapies include but are not limited to chemotherapy, immunotherapy, and/or radiation therapy.

It is understood that, in the aforementioned aspects and embodiments, baseline expression refers to normalized mean gene expression. Thus, in further embodiments, higher than baseline expression refers to at least about a 2-fold increase in expression relative to baseline expression and/or lower than baseline expression is at least about a 2-fold decrease in expression relative to baseline expression.

More generally, the term “baseline” is employed to refer to the condition of the cells absent exposure to a tumor or cancer. And, unless explicitly stated otherwise, terms of degree such as “higher” and “lower” are used in reference to a “baseline” value calculated thusly.

It is also understood in aspects relating to the use of an antibody or antigen binding fragment thereof, the full scope of these terms are intended. For examples, antibodies may be of any class and/or subclass, including but not limited to IgG, IgA, IgM, IgE or IgD, or a subclass thereof. Exemplary subclasses of IgG are provided herein and include IgG₁, IgG₂, IgG₃ and IgG₄. Antigen binding fragments may comprise a variety of antibody components, e.g. the antigen binding fragment may be a Fab, Fab′, F(ab′)2, Fv, Fd, single-chain Fvs (scFv), disulfide-linked Fvs (sdFv) or V_{L} or V_{H}.

In general, it is noted that agents or antibodies disclosed herein can be contacted with the cancer, tumor, or sample in conditions under which it can bind to the gene or protein it targets to assess expression and/or presence of the aforementioned genes or proteins.

Analytic techniques useful for the purposes of detection required by some method aspects include but are not limited to immunohistochemistry (IHC), in-situ hybridization (ISH), ELISA, immunoprecipitation, immunofluorescence, chemiluminescence, radioactivity, X-ray, nucleic acid hybridization, protein-protein interaction, immunoprecipitation, flow cytometry, Western blotting, polymerase chain reaction, DNA transcription, Northern blotting, and Southern blotting.

To the extent that samples are required in the method aspects disclosed herein they can optionally comprise comprises cells, tissue, or an organ biopsy; be an epithelial sample; originate from lung, respiratory or airway tissue or organ, a circulatory tissue or organ, a skin tissue, bone tissue, or muscle tissue; and/or originate from head, neck, brain, skin, bone, or blood. Likewise, the term cancer or tumor may refer to a cancer or tumor in the head, neck, lung, lung, prostate, colon, pancreas, esophagus, liver, skin, kidney, adrenal gland, brain, or comprises a lymphoma, breast, endometrium, uterus, ovary, testes, lung, prostate, colon, pancreas, esophagus, liver, skin, kidney, adrenal gland, or brain; and can include a metastasis from the primary cancer or a recurring tumor, cancer or neoplasia; and/or comprising a non-small cell lung cancer (NSCLC) or head and neck squamous cell cancer (HNSCC).

### BRIEF DESCRIPTION OF DRAWINGS

FIG. 1. Core CD8+ TIL transcriptional profile. FIG. 1: GSEA of various gene sets in the transcriptome of CD8+ TILs versus that of CD8+ N-TILs from donors with NSCLC, presented as the running enrichment score (RES) for the gene set as the analysis ‘walks down’ the ranked list of genes (reflective of the degree to which the gene set is over-represented at the top or bottom of the ranked list of genes) (top), the position of the gene-set members (vertical lines) in the ranked list of genes (middle), and the value of the ranking metric (bottom). P values, Kolmogorov-Smirnov test. Data are from one experiment with n=32 donors (lung N-TILs), n=36 donors (NSCLC TILs) and n=41 donors (HNSCC TILs).

FIGS. 2A-2F. Pathways for which CD8+ TILs show enrichment. FIG. 2A: Analysis of canonical pathways from the Ingenuity pathway analysis database (horizontal axis; bars in plot) for which CD8+ TILs show enrichment, presented as the frequency of differentially expressed genes encoding components of each pathway that are upregulated or downregulated (key) in CD8+ TILs relative to their expression in CD8+ N-TILs (left vertical axis), and adjusted P values (right vertical axis; line; Fisher's exact test); numbers above bars indicate total genes in each pathway. HBCS, hereditary breast cancer signaling; BRCA, tumor suppressor; RA, rheumatoid arthritis; CHK, checkpoint kinase; APRIL, proliferation-inducing ligand; dTMP, deoxythymidine monophosphate; NF-κB, transcription factor; iNOS, inducible nitric oxide synthase. FIG. 2B: Overlap of genes encoding components of the cell-cycle and proliferation pathways in CD8+ TILs and in CD8+ N-TILs: numbers in parentheses indicate total genes in each pathway; numbers along lines indicate total genes shared by the pathways connected by the line. FIG. 2C: RNA-Seq analysis of PLK1 (encoding the serine-threonine kinase PLK1), CCNB1 (encoding cyclin B1), 4-1BB, CD27 and JUN (encoding the transcription factor c-Jun) in lung N-TILs and NSCLC TILs (key in FIG. 2F). Each symbol represents an individual sample. FIG. 2D: Ingenuity pathway analysis of genes upregulated in CD8+ TILs relative to their expression in N-TILs (yellow), encoding components of the canonical 4-1BB and CD27 signaling pathways (shape indicates function (key)) in lymphocytes. FIG. 2E: Flow-cytometry analysis of the surface expression of 4-1BB and CD8 on live and singlet-gated CD45+CD3+ T cells obtained from peripheral blood mononuclear cells (PBMC), lung N-TILs and NSCLC TILs (above plots) from the same patient. Numbers in quadrants indicate percent cells in each throughout; red indicates percent cells among TILs throughout. FIG. 2F: Quantification of clonotypes (average values) among CD8+ N-TILs and NSCLC CD8+ TILs (key) according to their frequency in each donor (horizontal axis), derived from RNA-Seq analysis of genes encoding TCR β-chains. Small horizontal lines indicate the mean (±s.e.m.). *P<0.05 (unpaired Student's two-tailed t-test).

FIG. 3. Heterogeneity in the expression of immunotherapy target molecules. FIG. 3 shows RNA-Seq analysis of PDCD1, 4-1BB, HAVCR2, LAG3 and TIGIT in N-TILS and TILs from TIL^{high} or TIL^{low} tumors (key).

FIG. 4A-4F. Tissue residency features in TIL^{high} tumors. FIG. 4A: RNA-Seq analysis of ITGAE, CXCR6, S1PR1, KLF2 and STK38. Each symbol (bottom) represents an individual sample; small horizontal lines indicate the mean (s.e.m.). FIG. 4B: Immunobistochemistry microscopy of CD8α, PD-1 and CD103 (above images) in TIL^{low} and TIL^{hgh}NSCLC tumors (left margin). Scale bars, 100 μm. FIG. 4C: Flow-cytometry analysis of the surface expression of CD8 and CD103 (top), PD-1 and CD103 (middle) and 4-1BB and CD103 (bottom) on live and singlet-gated CD45+CD3+ T cells obtained from peripheral blood mononuclear cells, lung N-TILs and NSCLC TILs (above plots) from the same patient. FIG. 4D: Flow-cytometry analysis of the expression of CD69 or CD49a versus that of CD103 (top row, left and middle), and of KLRG1, CD62L or CCR7 versus that of CD103 (bottom row) in live and singlet-gated CD45+CD3+CD8+ T cells; top right, overlay of CD103+CD8+ TILs with CD103-CD8+ TILs. FIG. 4E: GSEA of T_{RM} cell signature genes upregulated (top) or downregulated (bottom) in the transcriptome of CD8+ TILs from NSCLC TIL^{high} tumors relative to their expression in other TILs and N-TILs. FIG. 4F: Ingenuity pathway analysis of upregulated transcripts (perimeter) in NSCLC TIL^{high} tumors that are regulated by interferon-γ (arrows) and encode products with various functions (key); an arrow indicates an unpredicted effect of IFN-γ.

FIG. 5A-5G. CD103 density predicts survival in lung cancer. FIG. 5A: RNA-Seq analysis of DLGAP5, CDC20, AURKB, CCNB2A and BIRC5, all encoding products linked to cell cycle and proliferation. Each symbol (bottom) represents an individual sample; small horizontal lines indicate the mean (±s.e.m.). FIG. 5B: Flow-cytometry analysis of the expression of Ki67 and CD103 in live and singlet-gated CD45+CD3+CD8+ T cells obtained from peripheral blood mononuclear cells, lung N-TILs and NSCLC TILs (above plots) from the same patient. FIG. 5C: Expression of GZMB, GZA1 and 1FNG transcripts (log 2 normalized counts) in cells as in 5A (key). FIG. 5D: Expression of granzyme B (mean fluorescence intensity (MFI)) in CD8+ TILs from CD103^{low} tumors (n=5) or CD103^{high} tumors (n=7) (top left), and flow-cytometry analysis of the expression of granzyme B, granzyme A, perforin, CD107a (LAMP-1) or IFN-γ versus that of CD103 in live and singlet-gated CD45+CD3+CD8+ T cells obtained from NSCLC TILs. *P=0.0025 (Mann-Whitney test). FIGS. 5E and 5F: Survival of patients (n=689) with lung cancer, with a low density (CD8^{low}) or high density (CD8^{high}) of CD8+ cells (key) in tumors (FIG. 5E) or a low density (CD103^{low}) or high density (CD103^{high}) of CD103+ cells (key) in tumors (FIG. 5F), presented as Kaplan-Meier curves. NS, P:=0.086 (FIG. 5G), and *P=0.043 (FIG. 5F) (log-rank test). FIG. 5G: Survival of patients with lung cancer with CD3 tumors sub-classified according to the density of CD103-expressing cells (key) (right), presented as Kaplan-Meier curves. *P=0.036 (log-rank test). Each symbol (FIGS. 5C/5D) represents an individual sample (FIG. 5C) or patient (FIG. 5D); small horizontal lines indicate the mean (±s.e.m.).

FIGS. 6A-6B. FIG. 6A: Expression of gene transcripts (log 2 normalized counts) in N-TILs or in NSCLC CD8+ TILs from CD103^{high} or CD103^{low} tumors (key). FIG. 6B: Flow-cytometry analysis of the expression of KIR2D14, CD38 or CD39 versus that of CD103 in live and singlet-gated CD45+CD3+CD8+ T cells obtained from NSCLC TILs (left), and frequency of CD38+ cells or CD39+ cells among CD8+CD103− TILs or CD8+CD103+ TILs (key). *P=0.0006, CD38+ cells, or P<0.0001, CD39+ cells (paired Student's two-tailed t-test). Each symbol (FIGS. 6A-6B) represents an individual patient or sample; small horizontal lines (FIG. 6A) indicate the mean (±s.e.m.); diagonal lines (FIG. 6B) connect data from the same patient.

FIGS. 7A-7C. FIG. 7A Ingenuity pathway analysis of genes downregulated in CD8+ TLs from NSCLC TIL^{high} tumors relative to their expression in TIL^{low} tumors, encoding molecules associated with tissue egress (shape indicates function (key)). FIG. 7B Flow-cytometry analysis of the expression of CD69, CD49a, KLRG1, CD62L or CCR7 versus that of CD103 in live and singlet-gated CD45+CD3+CD8+ T cells obtained from NSCLC TILs (left); frequency of CD103+CD8+ or CD103−CD8+ TILs (n=6) that express the indicated surface marker (right). * P=0.0025 (CD69), P=0.0025 (CD49a), P=0.0016 (KLRG1), P=0.0021 (CD62L) (paired Student's two-tailed t-test). FIG. 7C Analysis of canonical pathways from the Ingenuity pathway analysis database (horizontal axis; bars in plot) for which CD8+ TILs from NSCLC TIL^{high} tumors show enrichment (presented as in FIG. 2A) relative to their expression in TIL^{low} tumors (P values as in FIG. 2A). Each symbol (FIG. 7B) represents an individual sample; small horizontal lines indicate the mean (+, s.e.m.). Data are from one experiment (FIG. 7A, 7C) or from six experiments (FIG. 7B).

FIGS. 8A-8C show RNA-Seq analysis of NSCLC CD103+CD8+(TRMs, right most; tumor+) and CD103-CD8+ (non-TRMs, second from right; tumor−) TILs and CD103+CD8+ (TRMs, second from left; non-tumor+) and CD103-CD8+ (non-TRMs, left most; non-tumor−) NTILs from lung cancer patients (n>20). The expression of the indicated transcripts is represented as bar graphs (Transcript per million (TPM) counts; error bars are mean±SEM); each dot represents data from a single patient.

### DETAILED DESCRIPTION OF THE DISCLOSURE

It is to be understood that the present disclosure is not limited to particular aspects described, as such may, of course, vary. It is also to be understood that the terminology used herein is for the purpose of describing particular aspects only, and is not intended to be limiting, since the scope of the present disclosure will be limited only by the appended claims.

Unless defined otherwise, all technical and scientific terms used herein have the same meanings as commonly understood by one of ordinary skill in the art to which this technology belongs. Although any methods and materials similar or equivalent to those described herein can be used in the practice or testing of the present technology, the preferred methods, devices and materials are now described. All technical and patent publications cited herein are incorporated herein by reference in their entirety. Nothing herein is to be construed as an admission that the present technology is not entitled to antedate such disclosure by virtue of prior invention.

The practice of the present technology will employ, unless otherwise indicated, conventional techniques of tissue culture, immunology, molecular biology, microbiology, cell biology, and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

It is to be inferred without explicit recitation and unless otherwise intended, that when the present technology relates to a polypeptide, protein, polynucleotide or antibody, an equivalent or a biologically equivalent of such is intended within the scope of the present technology.

### Definitions

As used in the specification and claims, the singular form “a”, “an”, and “the” include plural references unless the context clearly dictates otherwise. For example, the term “a cell” includes a plurality of cells, including mixtures thereof.

As used herein, the term “animal” refers to living multi-cellular vertebrate organisms, a category that includes, for example, mammals and birds. The term “mammal” includes both human and non-human mammals.

The terms “subject,” “host,” “individual,” and “patient” are as used interchangeably herein to refer to human and veterinary subjects, for example, humans, animals, non-human primates, dogs, cats, sheep, mice, horses, and cows. In some embodiments, the subject is a human.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 103 M−1 greater, at least 104 M−1 greater or at least 105 M−1 greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3rd Ed., W.H. Freeman & Co., New York, 1997. An “antigen binding fragment” of an antibody is a portion of an antibody that retains the ability to specifically bind to the target antigen of the antibody.

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies and human antibodies.

In terms of antibody structure, an immunoglobulin has heavy (H) chains and light (L) chains interconnected by disulfide bonds. There are two types of light chain, lambda (k) and kappa (x). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopts a p-sheet conformation and the CDRs form loops which connect, and in some cases form part of, the 3-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found. An antibody that binds DCLK1 will have a specific VH region and the VL region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

As used herein, the term “antigen binding domain” refers to any protein or polypeptide domain that can specifically bind to an antigen target.

A “composition” typically intends a combination of the active agent, e.g., an immune cell, an antibody, a compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term “consensus sequence” as used herein refers to an amino acid or nucleic acid sequence that is determined by aligning a series of multiple sequences and that defines an idealized sequence that represents the predominant choice of amino acid or base at each corresponding position of the multiple sequences. Depending on the sequences of the series of multiple sequences, the consensus sequence for the series can differ from each of the sequences by zero, one, a few, or more substitutions. Also, depending on the sequences of the series of multiple sequences, more than one consensus sequence may be determined for the series. The generation of consensus sequences has been subjected to intensive mathematical analysis. Various software programs can be used to determine a consensus sequence.

As used herein, the term “B cell,” refers to a type of lymphocyte in the humoral immunity of the adaptive immune system. B cells principally function to make antibodies, serve as antigen presenting cells, release cytokines, and develop memory B cells after activation by antigen interaction. B cells are distinguished from other lymphocytes, such as T cells, by the presence of a B-cell receptor on the cell surface. B cells may either be isolated or obtained from a commercially available source. Non-limiting examples of commercially available B cell lines include lines AHH-1 (ATCC® CRL-8146™), BC-1 (ATCC® CRL-2230™), BC-2 (ATCC® CRL-2231™), BC-3 (ATCC® CRL-2277™), CA46 (ATCC® CRL-1648™), DG-75 [D.G.-75] (ATCC® CRL-2625™), DS-1 (ATCC® CRL-11102™) EB-3 [EB3] (ATCC® CCL-85™), Z-138 (ATCC #CRL-3001), DB (ATCC CRL-2289), Toledo (ATCC CRL-2631), Pfiffer (ATCC CRL-2632), SR (ATCC CRL-2262), JM-1 (ATCC CRL-10421), NFS-5 C-1 (ATCC CRL-1693); NFS-70 C10 (ATCC CRL-1694), NFS-25 C-3 (ATCC CRL-1695), AND SUP-B15 (ATCC CRL-1929). Further examples include but are not limited to cell lines derived from anaplastic and large cell lymphomas, e.g., DEL, DL-40, FE-PD, JB6, Karpas 299, Ki-JK, Mac-2A Ply1, SR-786, SU-DHL-1, -2, -4, -5, -6, -7, -8, -9, -10, and -16, DOHH-2, NU-DHL-1, U-937, Granda 519, USC-DHL-1, RL; Hodgkin's lymphomas, e.g., DEV, HD-70, HDLM-2, HD-MyZ, HKB-1, KM-H2, L 428, L 540, L1236, SBH-1, SUP-HD1, SU/RH-HD-1. Non-limiting exemplary sources for such commercially available cell lines include the American Type Culture Collection, or ATCC, (www.atcc.org/) and the German Collection of Microorganisms and Cell Cultures (https.//www.dsmz.de/).

As used herein, the term “T-cell,” refers to a type of lymphocyte that matures in the thymus. T cells play an important role in cell-mediated immunity and are distinguished from other lymphocytes, such as B cells, by the presence of a T-cell receptor (TCR) on the cell surface. T-cells may either be isolated or obtained from a commercially available source. “T-cell” includes all types of immune cells expressing CD3. Non-limiting examples of T-cells and markers for isolation thereof including naïve T cells (CCR7+, CD45RA+), double-negative T-cells (CD3+, CD4−, CD8−), CD4+ T-cells (such as but not limited to T-helper (“Th”) cells such as: T-regulatory cells, Tregs (CD25+), Th1 cells (CDCR3+, CCR5+), Th2 cells (CXCR4+, CCR3+, CCR4+, CCR5+, CCR7+, CD30+), Th17 cells (CD4+, IL-17A+) and näive CD4+ T-cells (CD4+, CD45RA+, CD62L+)), CD8+ T-cells, natural killer T-cells, central memory T-cells (CCR7+, CD45RA−), effector memory T-cells (CCR7−, CD45RA−), and gamma-delta T cells. Natural killer T cells (NKT) co-express NK cell markers and a semi-invariant T cell receptor (TCR). They are implicated in the regulation of immune responses associated with a broad range of diseases. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PERO117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4; 11 (ATCC CRL-1873), CCRF-CEM (ATCC CRM-CCL-119); and cutaneous T-cell lymphoma lines, e.g., HuT78 (ATCC CRM-TIB-161), MJ[G11] (ATCC CRL-8294), HuT102 (ATCC TIB-162). Null leukemia cell lines, including but not limited to REH, NALL-1, KM-3, L92-221, are another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein, the term “NK cell,” also known as natural killer cell, refers to a type of lymphocyte that originates in the bone marrow and play a critical role in the innate immune system. NK cells provide rapid immune responses against viral-infected cells, tumor cells or other stressed cell, even in the absence of antibodies and major histocompatibility complex on the cell surfaces. NK cells may either be isolated or obtained from a commercially available source. Non-limiting examples of commercial NK cell lines include lines NK-92 (ATCC® CRL-2407™), NK-92MI (ATCC® CRL-2408™). Further examples include but are not limited to NK lines HANK1, KHYG-1, NKL, NK-YS, NOI-90, and YT. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term signal peptide or signal polypeptide intends an amino acid sequence usually present at the N-terminal end of newly synthesized secretory or membrane polypeptides or proteins. It acts to direct the polypeptide across or into a cell membrane and is then subsequently removed. Examples of such are well known in the art. Non-limiting examples are those described in U.S. Pat. Nos. 8,853,381 and 5,958,736.

As used herein, the term “vector” refers to a nucleic acid construct deigned for transfer between different hosts, including but not limited to a plasmid, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

As used herein, the term “isolated cell” generally refers to a cell that is substantially separated from other cells of a tissue. “Immune cells” includes, e.g., white blood cells (leukocytes) which are derived from hematopoietic stem cells (HSC) produced in the bone marrow, lymphocytes (T cells, B cells, natural killer (NK) cells), myeloid-derived cells (neutrophil, eosinophil, basophil, monocyte, macrophage, dendritic cells), as well as precursors thereof committed to immune lineages. Precursors of T-cells are lineage restricted stem and progenitor cells capable of differentiating to produce a mature T-cell. Precursors of T-cells include HSCs, long term HSCs, short term HSCs, multipotent progenitor cells (MPPs), lymphoid primed multipotent progenitor cells (LMPPs), early lymphoid progenitor cells (ELPs), common lymphoid progenitor cells (CLPs), Pro-T-cells (ProT), early T-lineage progenitors/double negative 1 cells (ETPs/DN1), double negative (DN) 2a, DN2b, DN3a, DN3b, DN4, and double positive (DP) cells. Markers of such T-cell precursors in humans include but are not limited to: HSCs: CD34+ and, optionally, CD38−; long term HSCs: CD34+CD38− and lineage negative, wherein lineage negative means negative for one or more lineage specific markers selected from the group of TER119, Mac1, Gr1, CD45R/B220, CD3, CD4, and CD8; MPPs: CD34+ CD38− CD45RA− CD90− and, optionally, lineage negative; CLP: CD34+ CD38+ CD10+ and, optionally, lineage negative; LMPP/ELP: CD45RA+ CD62L+ CD38− and, optionally, lineage negative; DN1: CD117− CD34+ CD38− CD1a−; DN2: CD117+ CD34+ CD38+ CD1a−; DN3: CD34+ CD38+ CD1a+; DN4: CD4+ CD3−; DP: CD4+ CD8+ and, optionally, CD3+. Precursors of NK cells are lineage restricted stem and progenitor cells capable of differentiating to produce a mature NK cell. NK precursors include HSCs, long term HSCs, short term HSCs, multipotent progenitor cells (MPPs), common myeloid progenitors (CMP), granulocyte-macrophage progenitors (GMP), pro-NK, pre-NK, and immature NK (iNK). Markers of such NK precursors include but are not limited to: CMP: CD56− CD36− CD33+ CD34+ NKG2D− NKp46−; GMP: CD56− CD36− CD33+ CD34+ NKG2D− NKp46−; pro-NK: CD34+ CD45RA+ CD10+ CD117− CD161−; pre-NK: CD34+ CD45RA+ CD10− CD117+ CD161+/−; and iNK: CD34− CD117+ CD161+ NKp46− CD94/NKG2A−. In some aspects, markers of NK cell precursors include but are not limited to CD117+ CD161+ CD244+ CD33+ CD56− NCR− CD94/NKG2A− and LFA-1−. Phenotyping reagents to detect precursor cell surface markers are available from, for example, BD Biosciences (San Jose, CA) and BioLegend (San Diego, CA). “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses.

Certain terms are used herein to describe subsets of immune cells categorized based on location and/or function. The term “tumor infiltrating lymphocytes” or “TILs” as used herein describes immune cells which have left the bloodstream and migrated into a tumor.

The term “tissue resident memory cells” or “TRM” or “T_{RM}” refers to cells that retain immune memory and reside in tissue without recirculating in the peripheral blood.

The term “transduce” or “transduction” as it is applied to the production of chimeric antigen receptor cells refers to the process whereby a foreign nucleotide sequence is introduced into a cell. In some embodiments, this transduction is done via a vector.

As used herein, the term “CRISPR” refers to a technique of sequence specific genetic manipulation relying on the clustered regularly interspaced short palindromic repeats pathway (CRISPR). CRISPR can be used to perform gene editing and/or gene regulation, as well as to simply target proteins to a specific genomic location. Gene editing refers to a type of genetic engineering in which the nucleotide sequence of a target polynucleotide is changed through introduction of deletions, insertions, or base substitutions to the polynucleotide sequence. In some aspects, CRISPR-mediated gene editing utilizes the pathways of nonhomologous end-joining (NHEJ) or homologous recombination to perform the edits. Gene regulation refers to increasing or decreasing the production of specific gene products such as protein or RNA.

The term “guide RNA” or “gRNA” as used herein refers to the guide RNA sequences used to target the CRISPR complex to a specific nucleotide sequence such as a specific region of a cell's genome. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, M. et al. (2016) J of Biotechnology 233 (2016) 74-83).

As used herein, the term “autologous,” in reference to cells refers to cells that are isolated and infused back into the same subject (recipient or host). “Allogeneic” refers to non-autologous cells.

An “effective amount” or “efficacious amount” refers to the amount of an agent, or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated.

As used herein, the term “cancer” refers to a disease characterized by the abnormal growth of cells caused by uncontrolled cell division. These cells may be malignant. A “neoplasia” is a new, abnormal growth of cells. A “tumor” is an abnormal mass of tissue that usually does not contain cysts or liquid areas. Tumors can be benign or malignant. Different types of tumors are named for the type of cells that form them. Examples of tumors include sarcomas, carcinomas, and lymphomas. The term “tumor” may optionally refer to a solid tumor. Malignant tumors may often shed “circulating tumor cells” or “CTCs” which are tumor cells that have shed into the vasculature or lymphatic system from a primary tumor and carried through these systems throughout the body. These CTCs may settle in another part of the body to generate additional tumors known as “metastases.” In some embodiments disclosed herein, the term cancer or tumor may refer to a cancer or tumor in the head, neck, lung, lung, prostate, colon, pancreas, esophagus, liver, skin, kidney, adrenal gland, brain, or comprises a lymphoma, breast, endometrium, uterus, ovary, testes, lung, prostate, colon, pancreas, esophagus, liver, skin, kidney, adrenal gland, or brain; comprising a metastasis or recurring tumor, cancer or neoplasia; and/or comprising a non-small cell lung cancer (NSCLC) or head and neck squamous cell cancer (HNSCC).

As used herein, the term “comprising” is intended to mean that the compositions and methods include the recited elements, but do not exclude others. “Consisting essentially of” when used to define compositions and methods, shall mean excluding other elements of any essential significance to the combination for the intended use. For example, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions disclosed herein. Aspects defined by each of these transition terms are within the scope of the present disclosure.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I.

As used herein, the term “purification marker” or “label” intends a directly or indirectly detectable compound or composition that is conjugated directly or indirectly to the composition to be detected or isolated, e.g., N-terminal histidine tags (N-His), HA tag, FLAG tag, 6XHis tag, magnetically active isotopes, e.g., ¹¹⁵Sn, 117Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6th ed). Examples of luminescent probes include, but are not limited to, aequorin and luciferases. Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th ed.). In another aspect, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, self-antigens, protozoa and other parasitic antigens, tumor/cancer antigens, antigens involved in autoimmune disease, allergy and graft rejection, toxins, and other miscellaneous antigens.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical”, percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6× SSC to about 10× SSC; formamide concentrations of about 0% to about 25%; and wash solutions from about 4× SSC to about 8× SSC. Examples of moderate hybridization conditions include: incubation temperatures of about 40° C. to about 50° C.; buffer concentrations of about 9× SSC to about 2×SSC; formamide concentrations of about 30% to about 50%; and wash solutions of about 5× SSC to about 2× SSC. Examples of high stringency conditions include: incubation temperatures of about 55° C. to about 68° C.; buffer concentrations of about 1× SSC to about 0.1× SSC; formamide concentrations of about 55% to about 75%; and wash solutions of about 1× SSC, 0.1× SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein refers to molecules or biologicals or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double and single stranded molecules. Unless otherwise specified or required, any aspect of this technology that is a polynucleotide encompasses both the double stranded form and each of two complementary single stranded forms known or predicted to make up the double stranded form.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, the term “specific binding” means the contact between an antibody and an antigen with a binding affinity of at least 10⁻⁶ M. In certain aspects, antibodies bind with affinities of at least about 10⁻⁷ M, and preferably 10⁻⁸ M, 10⁻⁹ M, 10⁻¹⁰ M, 10⁻¹¹ M, or 10⁻¹² M.

As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. The term “therapy” as used herein refers to the application of one or more treatments protocols to a disease in a subject.

“Cytoreductive therapy,” as used herein, refers to cancer therapy aimed at debulking a cancerous tumor. Such therapy includes but is not limited to chemotherapy, cryotherapy, and radiation therapy. Agents that act to reduce cellular proliferation are known in the art and widely used. Chemotherapy drugs that kill cancer cells only when they are dividing are termed cell-cycle specific. These drugs include agents that act in S-phase, including topoisomerase inhibitors and anti-metabolites. Cryotherapy also includes, but is not limited to, therapies involving decreasing the temperature, for example, hypothermic therapy.

Toposiomerase inhibitors are drugs that interfere with the action of topoisomerase enzymes (topoisomerase I and II). During the process of chemo treatments, topoisomerase enzymes control the manipulation of the structure of DNA necessary for replication, and are thus cell cycle specific. Examples of topoisomerase I inhibitors include the camptothecan analogs listed above, irinotecan and topotecan. Examples of topoisomerase II inhibitors include amsacrine, etoposide, etoposide phosphate, and teniposide.

Antimetabolites are usually analogs of normal metabolic substrates, often interfering with processes involved in chromosomal replication. They attack cells at very specific phases in the cycle. Antimetabolites include folic acid antagonists, e.g., methotrexate; pyrimidine antagonist, e.g., 5-fluorouracil, foxuridine, cytarabine, capecitabine, and gemcitabine; purine antagonist, e.g., 6-mercaptopurine and 6-thioguanine; adenosine deaminase inhibitor, e.g., cladribine, fludarabine, nelarabine and pentostatin; and the like.

Plant alkaloids are derived from certain types of plants. The vinca alkaloids are made from the periwinkle plant (Catharanthus rosea). The taxanes are made from the bark of the Pacific Yew tree (taxus). The vinca alkaloids and taxanes are also known as antimicrotubule agents. The podophyllotoxins are derived from the May apple plant. Camptothecan analogs are derived from the Asian “Happy Tree” (Camptotheca acuminata). Podophyllotoxins and camptothecan analogs are also classified as topoisomerase inhibitors. The plant alkaloids are generally cell-cycle specific.

Examples of these agents include vinca alkaloids, e.g., vincristine, vinblastine and vinorelbine; taxanes, e.g., paclitaxel and docetaxel; podophyllotoxins, e.g., etoposide and tenisopide; and camptothecan analogs, e.g., irinotecan and topotecan.

Radiation therapy includes, but is not limited to, exposure to radiation, e.g., ionizing radiation, UV radiation, as known in the art. Exemplary dosages include, but are not limited to, a dose of ionizing radiation at a range from at least about 2 Gy to not more than about 10 Gy and/or a dose of ultraviolet radiation at a range from at least about 5 J/m2 to not more than about 50 J/m2, usually about 10 J/m2.

“Immunotherapy,” as used herein, refers to cancer therapies that enhance the immune response to a tumor or cancer. Such therapy includes but is not limited to adoptive cell therapies, such as those utilizing chimeric antigen receptor expressing (“CAR”) cells, CD8+ cytotoxic cells, natural killer cells, or equivalents thereof; monoclonal antibodies and immunoconjugate based therapies designed to target and destroy tumors and/or cancer cells; cytokine, chemokine, or lymphokine therapy, such as interferon gamma (“IFNγ”) treatment; and vaccination.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited Nov. 15, 2017. Typically, a patient is given a subsequent chemotherapy regimen because the patient did not show a positive clinical or sub-clinical response to the first line therapy or the first line therapy has stopped.

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell.

As used herein, the term “enhancer”, as used herein, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

Disclosed herein are a plurality of genes of interest whose expression or presence is quantified and assessed in comparison to a baseline. As disclosed above, the term “baseline” is employed to refer to the condition of the cells absent exposure to a tumor or cancer. And, unless explicitly stated otherwise, terms of degree such as “higher” and “lower” are used in reference to a “baseline” value calculated thusly.

Further, in regard to the various genes, it is appreciated that the sequences of each of these genes and the resulting proteins are known in the art; thus, probes for detecting the genes, transcripts, and the resulting proteins as well are those other genes along the pathway may be readily determined based on the information disclosed herein. For example, in addition to the listing of the genes, Tables 1, 12, and 13 provide the Gene Cards database identification number for each of the listed genes. An ordinary skilled artisan may access the Gene Cards database at genecards.org (last accessed Dec. 5, 2017) to locate the sequence of each of these genes by searching the name or by utilizing the readily available Gene Cards identification number. Furthermore, using this identifier, an ordinary skilled artisan is able to access information on homologs, orthologs, and other gene sequences. In addition, the Gene Cards identification number provide the chromosome (first to numbers), position (plus (P) or minus (M)) strand), an kilboase number (last numbers) for the location of the gene of interest. Thus, demonstrating the availability of the sequences for the purposes of making and/or using the claimed invention. To provide further clarity as to this process, provided below is a summary of the Gene Cards reference information for non-limiting exemplary genes disclosed herein:

CD8, GCID: GC02M086784 is an alternate name for the CD8 protein, which is a cell surface glycoprotein found on most cytotoxic T lymphocytes that mediates efficient cell-cell interactions within the immune system. A non-limiting exemplary sequence of the human protein provided below may be found under UniProtKB Ref. No. P01732, accessible through the Gene Cards database (SEQ ID NO: 1):

MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV

CD103, GCID: GC17M003722 is an alternate name for ITGAE, which is the alpha subunit of a heterodimeric integral membrane protein and may have a role in adhesion and as an accessory moledule for IEL activation. A non-limiting exemplary sequence of the human protein provided below may be found under UniProtKB Ref No. P38570, accessible through the Gene Cards database (SEQ ID NO: 2):

MWLFHTLLCIASLALLAAFNVDVARPWLTPKGGAPFVLSSLLHQDPSTNQ
TWLLVTSPRTKRTPGPLHRCSLVQDEILCHPVEHVPIPKGRHRGVTVVRS
HHGVLICIQVLVRRPHSLSSELTGTCSLLGPDLRPQAQANFFDLENLLDP
DARVDTGDCYSNKEGGGEDDVNTARQRRALEKEEEEDKEEEEDEEEEEAG
TEIAIILDGSGSIDPPDFQRAKDFISNMMRNFYEKCFECNFALVQYGGVI
QTEFDLRDSQDVMASLARVQNITQVGSVTKTASAMQHVLDSIFTSSHGSR
RKASKVMVVLTDGGIFEDPLNLTTVINSPKMQGVERFAIGVGEEFKSART
ARELNLIASDPDETHAFKVTNYMALDGLLSKLRYNIISMEGTVGDALHYQ
LAQIGFSAQILDERQVLLGAVGAFDWSGGALLYDTRSRRGRFLNQTAAAA
ADAEAAQYSYLGYAVAVLHKTCSLSYIAGAPRYKHHGAVFELQKEGREAS
FLPVLEGEQMGSYFGSELCPVDIDMDGSTDFLLVAAPFYHVHGEEGRVYV
YRLSEQDGSFSLARILSGHPGFTNARFGFAMAAMGDLSQDKLTDVAIGAP
LEGFGADDGASFGSVYIYNGHWDGLSASPSQRIRASTVAPGLQYFGMSMA
GGFDISGDGLADITVGTLGQAVVFRSRPVVRLKVSMAFTPSALPIGFNGV
VNVRLCFEISSVTTASESGLREALLNFTLDVDVGKQRRRLQCSDVRSCLG
CLREWSSGSQLCEDLLLMPTEGELCEEDCFSNASVKVSYQLQTPEGQTDH
PQPILDRYTEPFAIFQLPYEKACKNKLFCVAELQLATTVSQQELVVGLTK
ELTLNINLTNSGEDSYMTSMALNYPRNLQLKRMQKPPSPNIQCDDPQPVA
SVLIMNCRIGHPVLKRSSAHVSVVWQLEENAFPNRTADITVTVTNSNERR
SLANETHTLQFRHGFVAVLSKPSIMYVNTGQGLSHHKEFLFHVHGENLFG
AEYQLQICVPTKLRGLQVVAVKKLTRTQASTVCTWSQERACAYSSVQHVE
EWHSVSCVIASDKENVTVAAEISWDHSEELLKDVTELQILGEISFNKSLY
EGLNAENHRTKITVVFLKDEKYHSLPIIIKGSVGGLLVLIVILVILFKCG
FFKRKYQQLNLESIRKAQLKSENLLEEEN

PD-1, GCID: GC02M241849 is an alternate name for PDCD1, which is a cell surface membrane protein of the immunoglobulin superfamily expressed in pro-B-cells and believe to play a role in their differentiation as well as be important to T-cell function. A non-limiting exemplary sequence of the human protein provided below may be found under UniProtKB Ref No. Q15116, accessible through the Gene Cards database (SEQ ID NO: 3):

MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL

TIM3, GCID: GC05M157063 is an alternate name for HAVCR2, which is a Th1-specific cell surface protein that regulates macrophage activation, and inhibits Th1-mediated auto- and alloimmune responses, and promotes immunological tolerance. A non-limiting exemplary sequence of the human protein provided below may be found under UniProtKB Ref No. Q8TDQ0, accessible through the Gene Cards database (SEQ ID NO: 4):

MFSHLPFDCVLLLLLLLLTRSSEVEYRAEVGQNAYLPCFYTPAAPGNLVP
VCWGKGACPVFECGNVVLRTDERDVNYWTSRYWLNGDFRKGDVSLTIENV
TLADSGIYCCRIQIPGIMNDEKFNLKLVIKPAKVTPAPTRQRDFTAAFPR
MLTTRGHGPAETQTLGSLPDINLTQISTLANELRDSRLANDLRDSGATIR
IGIYIGAGICAGLALALIFGALIFKWYSHSKEKIQNLSLISLANLPPSGL
ANAVAEGIRSEENIYTIEENVYEVEEPNEYYCYVSSRQQPSQPLGCRFAM
P

LAG3, GCID: GC12P006774 refers to a member of the Ig superfamily and contains 4 extracellular Ig-like domains. A non-limiting exemplary sequence of the human protein provided below may be found under UniProtKB Ref. No. P18627, accessible through the Gene Cards database (SEQ ID NO: 5):

MWEAQFLGLLFLQPLWVAPVKPLQPGAEVPVVWAQEGAPAQLPCSPTIPL
QDLSLLRRAGVTWQHQPDSGPPAAAPGHPLAPGPHPAAPSSWGPRPRRYT
VLSVGPGGLRSGRLPLQPRVQLDERGRQRGDFSLWLRPARRADAGEYRAA
VHLRDRALSCRLRLRLGQASMTASPPGSLRASDWVILNCSFSRPDRPASV
HWFRNRGQGRVPVRESPHHHLAESFLFLPQVSPMDSGPWGCILTYRDGFN
VSIMYNLTVLGLEPPTPLTVYAGAGSRVGLPCRLPAGVGTRSFLTAKWTP
PGGGPDLLVTGDNGDFTLRLEDVSQAQAGTYTCHIHLQEQQLNATVTLAI
ITVTPKSFGSPGSLGKLLCEVTPVSGQERFVWSSLDTPSQRSFSGPWLEA
QEAQLLSQPWQCQLYQGERLLGAAVYFTELSSPGAQRSGRAPGALPAGHL
LLFLILGVLSLLLLVTGAFGFHLWRRQWRPRRFSALEQGIHPPQAQSKIE
ELEQEPEPEPEPEPEPEPEPEPEQL

CTLA4, GCID: GC02P203867 refers to a member of the immunoglobulin superfamily and encodes a protein which transmits an inhibitory signal to T cells. A non-limiting exemplary sequence of the human protein provided below may be found under UniProtKB Ref No. P16410, accessible through the Gene Cards database (SEQ ID NO: 6):

MACLGFQRHKAQLNLATRTWPCTLLFFLLFIPVFCKAMHVAQPAVVLASS
RGIASFVCEYASPGKATEVRVTVLRQADSQVTEVCAATYMMGNELTFLDD
SICTGTSSGNQVNLTIQGLRAMDTGLYICKVELMYPPPYYLGIGNGTQIY
VIDPEPCPDSDFLLWILAAVSSGLFFYSFLLTAVSLSKMLKKRSPLTTGV
YVKMPPTEPECEKQFQPYFIPIN

S1PR5, GCID: GC19M010512 refers to a gene that regulates cell proliferation, apoptosis, motility, and neurite retraction. A non-limiting exemplary sequence of the human protein provided below may be found under UniProtKB Ref No. Q9H228, accessible through the Gene Cards database (SEQ ID NO: 7):

MESGLLRPAPVSEVIVLHYNYTGKLRGARYQPGAGLRADAVVCLAVCAFI
VLENLAVLLVLGRHPRFHAPMFLLLGSLTLSDLLAGAAYAANILLSGPLT
LKLSPALWFAREGGVFVALTASVLSLLAIALERSLTMARRGPAPVSSRGR
TLAMAAAAWGVSLLLGLLPALGWNCLGRLDACSTVLPLYAKAYVLFCVLA
FVGILAAICALYARIYCQVRANARRLPARPGTAGTTSTRARRKPRSLALL
RTLSVVLLAFVACWGPLFLLLLLDVACPARTCPVLLQADPFLGLAMANSL
LNPIIYTLTNRDLRHALLRLVCCGRHSCGRDPSGSQQSASAAEASGGLRR
CLPPGLDGSFSGSERSSPQRDGLDTSGSTGSPGAPTAARTLVSEPAAD

STK38, GCID: GC06M036493 refers to a member of the AGC serine/threonine kinase family of proteins. A non-limiting exemplary sequence of the human protein provided below may be found under UniProtKB Ref No. Q15208, accessible through the Gene Cards database (SEQ ID NO: 8):

MAMTGSTPCSSMSNHTKERVTMTKVTLENFYSNLIAQHEEREMRQKKLEK
VMEEEGLKDEEKRLRRSAHARKETEFLRLKRTRLGLEDFESLKVIGRGAF
GEVRLVQKKDTGHVYAMKILRKADMLEKEQVGHIRAERDILVEADSLWVV
KMFYSFQDKLNLYLIMEFLPGGDMMTLLMKKDTLTEEETQFYIAETVLAI
DSIHQLGFIHRDIKPDNLLLDSKGHVKLSDFGLCTGLKKAHRTEFYRNLN
HSLPSDFTFQNMNSKRKAETWKRNRRQLAFSTVGTPDYIAPEVFMQTGYN
KLCDWWSLGVIMYEMLIGYPPFCSETPQETYKKVMNWKETLTFPPEVPIS
EKAKDLILRFCCEWEHRIGAPGVEEIKSNSFFEGVDWEHIRERPAAISIE
IKSIDDTSNFDEFPESDILKPTVATSNHPETDYKNKDWVFINYTYKRFEG
LTARGAIPSYMKAAK

FAM65B, GCID: GC06M024805 is an alternate name for RIPOR2, which is an atypical inhibitor of the small G protein RhoA. A non-limiting exemplary sequence of the human protein provided below may be found under UniProtKB Ref. No. Q9Y4F9, accessible through the Gene Cards database (SEQ ID NO: 9):

MLVGSQSFSPGGPNGIIRSQSFAGFSGLQERRSRCNSFIENSSALKKPQA
KLKKMHNLGHKNNNPPKEPQPKRVEEVYRALKNGLDEYLEVHQTELDKLT
AQLKDMKRNSRLGVLYDLDKQIKTIERYMRRLEFHISKVDELYEAYCIQR
RLQDGASKMKQAFATSPASKAARESLTEINRSFKEYTENMCTIEVELENL
LGEFSIKMKGLAGFARLCPGDQYEIFMKYGRQRWKLKGKIEVNGKQSWDG
EETVFLPLIVGFISIKVTELKGLATHILVGSVTCETKELFAARPQVVAVD
INDLGTIKLNLEITWYPFDVEDMTASSGAGNKAAALQRRMSMYSQGTPET
PTFKDHSFFRWLHPSPDKPRRLSVLSALQDTFFAKLHRSRSFSDLPSLRP
SPKAVLELYSNLPDDIFENGKAAEEKMPLSLSFSDLPNGDCALTSHSTGS
PSNSTNPEITITPAEFNLSSLASQNEGMDDTSSASSRNSLGEGQEPKSHL
KEEDPEEPRKPASAPSEACRRQSSGAGAEHLFLENDVAEALLQESEEASE
LKPVELDTSEGNITKQLVKRLTSAEVPMATDRLLSEGSVGGESEGCRSFL
DGSLEDAFNGLLLALEPHKEQYKEFQDLNQEVMNLDDILKCKPAVSRSRS
SSLSLTVESALESFDFLNTSDFDEEEDGDEVCNVGGGADSVFSDTETEKH
SYRSVHPEARGHLSEALTEDTGVGTSVAGSPLPLTTGNESLDITIVRHLQ
YCTQLVQQIVFSSKTPFVARSLLEKLSRQIQVMEKLAAVSDENIGNISSV
VEAIPEFHKKLSLLSFWTKCCSPVGVYHSPADRVMKQLEASFARTVNKEY
PGLADPVFRTLVSQILDRAEPLLSSSLSSEVVTVFQYYSYFTSHGVSDLE
SYLSQLARQVSMVQTLQSLRDEKLLQTMSDLAPSNLLAQQEVLRTLALLL
TREDNEVSEAVTLYLAAASKNQHFREKALLYYCEALTKTNLQLQKAACLA
LKILEATESIKMLVTLCQSDTEEIRNVASETLLSLGEDGRLAYEQLDKFP
RDCVKVGGRHGTEVATAF

S1PR1, GCID: GC01P101236 refers to a protein structurally similar to G protein-coupled receptors that is highly expressed in endothelial cells. It binds the ligand sphingosine-1-phosphate with high affinity and high specificity. A non-limiting exemplary sequence of the human protein provided below may be found under UniProtKB Ref No. P21453, accessible through the Gene Cards database (SEQ ID NO: 10):

MGPTSVPLVKAHRSSVSDYVNYDIIVRHYNYTGKLNISADKENSIKLTSV
VFILICCFIILENIFVLLTIWKTKKFHRPMYYFIGNLALSDLLAGVAYTA
NLLLSGATTYKLTPAQWFLREGSMFVALSASVFSLLAIAIERYITMLKMK
LHNGSNNFRLFLLISACWVISLILGGLPIMGWNCISALSSCSTVLPLYHK
HYILFCTTVFTLLLLSIVILYCRIYSLVRTRSRRLTFRKNISKASRSSEK
SLALLKTVIIVLSVFIACWAPLFILLLLDVGCKVKTCDILFRAEYFLVLA
VLNSGTNPIIYTLTNKEMRRAFIRIMSCCKCPSGDSAGKFKRPIIAGMEF
SRSKSDNSSHPQKDEGDNPETIMSSGNVNSSS

KLF2, GCID: GC19P019293 refers to a protein that belongs to the Kruppel family of transcription factors. A non-limiting exemplary sequence of the human protein provided below may be found under UniProtKB Ref. No. Q9Y5W3, accessible through the Gene Cards database (SEQ ID NO: 11):

MALSEPILPSFSTFASPCRERGLQERWPRAEPESGGTDDDLNSVLDFILS
MGLDGLGAEAAPEPPPPPPPPAFYYPEPGAPPPYSAPAGGLVSELLRPEL
DAPLGPALHGRFLLAPPGRLVKAEPPEADGGGGYGCAPGLTRGPRGLKRE
GAPGPAASCMRGPGGRPPPPPDTPPLSPDGPARLPAPGPRASFPPPFGGP
GFGAPGPGLHYAPPAPPAFGLFDDAAAAAAALGLAPPAARGLLTPPASPL
ELLEAKPKRGRRSWPRKRTATHTCSYAGCGKTYTKSSHLKAHLRTHTGEK
PYHCNWDGCGWKFARSDELTRHYRKHTGHRPFQCHLCDRAFSRSDHLALH
MKRHM

MYO7A, GCID: GC11P077128 refers to an unconventional myosin with a very short tail. A non-limiting exemplary sequence of the human protein provided below may be found under UniProtKB Ref No. Q13402, accessible through the Gene Cards database (SEQ ID NO: 12):

MVILQQGDHVWMDLRLGQEFDVPIGAVVKLCDSGQVQVVDDEDNEHWISP
QNATHIKPMHPTSVHGVEDMIRLGDLNEAGILRNLLIRYRDHLIYTYTGS
ILVAVNPYQLLSIYSPEHIRQYTNKKIGEMPPHIFAIADNCYFNMKRNSR
DQCCIISGESGAGKTESTKLILQFLAAISGQHSWIEQQVLEATPILEAFG
NAKTIRNDNSSRFGKYIDIHFNKRGAIEGAKIEQYLLEKSRVCRQALDER
NYHVFYCMLEGMSEDQKKKLGLGQASDYNYLAMGNCITCEGRVDSQEYAN
IRSAMKVLMFTDTENWEISKLLAAILHLGNLQYEARTFENLDACEVLFSP
SLATAASLLEVNPPDLMSCLTSRTLITRGETVSTPLSREQALDVRDAFVK
GIYGRLFVWIVDKINAAIYKPPSQDVKNSRRSIGLLDIFGFENFAVNSFE
QLCINFANEHLQQFFVRHVFKLEQEEYDLESIDWLHIEFTDNQDALDMIA
NKPMNIISLIDEESKFPKGTDTTMLHKLNSQHKLNANYIPPKNNHETQFG
INHFAGIVYYETQGFLEKNRDTLHGDIIQLVHSSRNKFIKQIFQADVAMG
AETRKRSPTLSSQFKRSLELLMRTLGACQPFFVRCIKPNEFKKPMLFDRH
LCVRQLRYSGMMETIRIRRAGYPIRYSFVEFVERYRVLLPGVKPAYKQGD
LRGTCQRMAEAVLGTHDDWQIGKTKIFLKDHHDMLLEVERDKAITDRVIL
LQKVIRGFKDRSNFLKLKNAATLIQRHWRGHNCRKNYGLMRLGFLRLQAL
HRSRKLHQQYRLARQRIIQFQARCRAYLVRKAFRHRLWAVLTVQAYARGM
IARRLHQRLRAEYLWRLEAEKMRLAEEEKLRKEMSAKKAKEEAERKHQER
LAQLAREDAERELKEKEAARRKKELLEQMERARHEPVNHSDMVDKMFGFL
GTSGGLPGQEGQAPSGFEDLERGRREMVEEDLDAALPLPDEDEEDLSEYK
FAKFAATYFQGTTTHSYTRRPLKQPLLYHDDEGDQLAALAVWITILRFMG
DLPEPKYHTAMSDGSEKIPVMTKIYETLGKKTYKRELQALQGEGEAQLPE
GQKKSSVRHKLVHLTLKKKSKLTEEVTKRLHDGESTVQGNSMLEDRPTSN
LEKLHFIIGNGILRPALRDEIYCQISKQLTHNPSKSSYARGWILVSLCVG
CFAPSEKFVKYLRNFIHGGPPGYAPYCEERLRRTFVNGTRTQPPSWLELQ
ATKSKKPIMLPVTFMDGTTKTLLTDSATTAKELCNALADKISLKDRFGFS
LYIALFDKVSSLGSGSDHVMDAISQCEQYAKEQGAQERNAPWRLFFRKEV
FTPWHSPSEDNVATNLIYQQVVRGVKFGEYRCEKEDDLAELASQQYFVDY
GSEMILERLLNLVPTYIPDREITPLKTLEKWAQLAIAAHKKGIYAQRRTD
AQKVKEDVVSYARFKWPLLFSRFYEAYKFSGPSLPKNDVIVAVNWTGVYF
VDEQEQVLLELSFPEIMAVSSSRECRVWLSLGCSDLGCAAPHSGWAGLTP
AGPCSPCWSCRGAKTTAPSFTLATIKGDEYTFTSSNAEDIRDLVVTFLEG
LRKRSKYVVALQDNPNPAGEESGFLSFAKGDLIILDHDTGEQVMNSGWAN
GINERTKQRGDFPTDSVYVMPTVTMPPREIVALVTMTPDQRQDVVRLLQL
RTAEPEVRAKPYTLEEFSYDYFRPPPKHTLSRVMVSKARGKDRLWSHTRE
PLKQALLKKLLGSEELSQEACLAFIAVLKYMGDYPSKRTRSVNELTDQIF
EGPLKAEPLKDEAYVQILKQLTDNHIRYSEERGWELLWLCTGLFPPSNIL
LPHVQRFLQSRKHCPLAIDCLQRLQKALRNGSRKYPPHLVEVEAIQHKTT
QIFHKVYFPDDTDEAFEVESSTKAKDFCQNIATRLLLKSSEGFSLFVKIA
DKVLSVPENDFFFDFVRHLTDWIKKARPIKDGIVPSLTYQVFFMKKLWTT
TVPGKDPMADSIFHYYQELPKYLRGYHKCTREEVLQLGALIYRVKFEEDK
SYFPSIPKLLRELVPQDLIRQVSPDDWKRSIVAYFNKHAGKSKEEAKLAF
LKLIFKWPTFGSAFFEVKQTTEPNFPEILLIAINKYGVSLIDPKTKDILT
THPFTKISNWSSGNTYFHITIGNLVRGSKLLCETSLGYKMDDLLTSYISQ
MLTAMSKQRGSRSGKG

GPR25, GCID: GC01P200872 refers to a member of the G-protein coupled receptor 1 family, which generally activate signaling cascades as a response to extracellular stress. A non-limiting exemplary sequence of the human protein provided below may be found under UniProtKB Ref No. 000155, accessible through the Gene Cards database (SEQ ID NO: 13):

MAPTEPWSPSPGSAPWDYSGLDGLEELELCPAGDLPYGYVYIPALYLAAF
AVGLLGNAFVVWLLAGRRGPRRLVDTFVLHLAAADLGFVLTLPLWAAAAA
LGGRWPFGDGLCKLSSFALAGTRCAGALLLAGMSVDRYLAVVKLLEARPL
RTPRCALASCCGVWAVALLAGLPSLVYRGLQPLPGGQDSQCGEEPSHAFQ
GLSLLLLLLTFVLPLVVTLFCYCRISRRLRRPPHVGRARRNSLRIIFAIE
STFVGSWLPFSALRAVFHLARLGALPLPCPLLLALRWGLTIATCLAFVNS
CANPLIYLLLDRSFRARALDGACGRTGRLARRISSASSLSRDDSSVFRCR
AQAANTASASW

CLNK, GCID: GC04M010491 refers to a member of the SLP76 family of adaptors that plays a role in signalling. A non-limiting exemplary sequence of the human protein provided below may be found under UniProtKB Ref. No. Q7Z7G1, accessible through the Gene Cards database (SEQ ID NO: 14):

MNRQGNRKTTKEGSNDLKFQNFSLPKNRSWPRINSATGQYQRMNKPLLDW
ERNFAAVLDGAKGHSDDDYDDPELRMEETWQSIKILPARPIKESEYADTH
YFKVAMDTPLPLDTRTSISIGQPTWNTQTRLERVDKPISKDVRSQNIKGD
ASVRKNKIPLPPPRPLITLPKKYQPLPPEPESSRPPLSQRHTFPEVQRMP
SQISLRDLSEVLEAEKVPHNQRKPESTHLLENQNTQEIPLAISSSSFTTS
NHSVQNRDHRGGMQPCSPQRCQPPASCSPHENILPYKYTSWRPPFPKRSD
RKDVQHNEWYIGEYSRQAVEEAFMKENKDGSFLVRDCSTKSKEEPYVLAV
FYENKVYNVKIRFLERNQQFALGTGLRGDEKFDSVEDIIEHYKNFPIILI
DGKDKTGVHRKQCHLTQPLPLTRHLLPL

SRGAP3, GCID: GC03M008998 refers to a protein associated with the G-protein signaling pathway. A non-limiting exemplary sequence of the human protein provided below may be found under UniProtKB Ref No. 043295, accessible through the Gene Cards database (SEQ ID NO: 15):

MSSQTKFKKDKEIIAEYEAQIKEIRTQLVEQFKCLEQQSESRLQLLQDLQ
EFFRRKAEIELEYSRSLEKLAERFSSKIRSSREHQFKKDQYLLSPVNCWY
LVLHQTRRESRDHATLNDIFMNNVIVRLSQISEDVIRLFKKSKEIGLQMH
EELLKVTNELYTVMKTYHMYHAESISAESKLKEAEKQEEKQFNKSGDLSM
NLLRHEDRPQRRSSVKKIEKMKEKRQAKYSENKLKCTKARNDYLLNLAAT
NAAISKYYIHDVSDLIDCCDLGFHASLARTFRTYLSAEYNLETSRHEGLD
VIENAVDNLDSRSDKHTVMDMCNQVFCPPLKFEFQPHMGDEVCQVSAQQP
VQTELLMRYHQLQSRLATLKIENEEVRKTLDATMQTLQDMLTVEDFDVSD
AFQHSRSTESVKSAASETYMSKINIAKRRANQQETEMFYFTKFKEYVNGS
NLITKLQAKHDLLKQTLGEGERAECGTTRPPCLPPKPQKMRRPRPLSVYS
HKLFNGSMEAFIKDSGQAIPLVVESCIRYINLYGLQQQGIFRVPGSQVEV
NDIKNSFERGEDPLVDDQNERDINSVAGVLKLYFRGLENPLFPKERFQDL
ISTIKLENPAERVHQIQQILVTLPRVVIVVMRYLFAFLNHLSQYSDENMM
DPYNLAICFGPTLMHIPDGQDPVSCQAHINEVIKTIIIHHEAIFPSPREL
EGPVYEKCMAGGEEYCDSPHSEPGAIDEVDHDNGTEPHTSDEEVEQIEAI
AKFDYMGRSPRELSFKKGASLLLYHRASEDWWEGRHNGVDGLIPHQYIVV
QDMDDAFSDSLSQKADSEASSGPLLDDKASSKNDLQSPTEHISDYGFGGV
MGRVRLRSDGAAIPRRRSGGDTHSPPRGLGPSIDTPPRAAACPSSPHKIP
LTRGRIESPEKRRMATFGSAGSINYPDKKALSEGHSMRSTCGSTRHSSLG
DHKSLEAEALAEDIEKTMSTALHELRELERQNTVKQAPDVVLDTLEPLKN
PPGPVSSEPASPLHTIVIRDPDAAMRRSSSSSTEMMTTFKPALSARLAGA
QLRPPPMRPVRPVVQHRSSSSSSSGVGSPAVTPTEKMFPNSSADKSGTM

ATP8B4, GCID: GC15M049858 refers to a member of the cation transport ATPase (P-type) family and type IV subfamily. A non-limiting exemplary sequence of the human protein provided below may be found under UniProtKB Ref No. Q8TF62, accessible through the Gene Cards database (SEQ ID NO: 16):

MFCSEKKLREVERIVKANDREYNEKFQYADNRIHTSKYNILTFLPINLFE
QFQRVANAYFLCLLILQLIPEISSLTWFTTIVPLVLVITMTAVKDATDDY
FRHKSDNQVNNRQSEVLINSKLQNEKWMNVKVGDIIKLENNQFVAADLLL
LSSSEPHGLCYVETAELDGETNLKVRHALSVTSELGADISRLAGFDGIVV
CEVPNNKLDKFMGILSWKDSKHSLNNEKIILRGCILRNTSWCFGMVIFAG
PDTKLMQNSGKTKFKRTSIDRLMNTLVLWIFGFLICLGIILAIGNSIWES
QTGDQFRTFLFWNEGEKSSVFSGFLTFWSYIIILNTVVPISLYVSVEVIR
LGHSYFINWDRKMYYSRKAIPAVARTTTLNEELGQIEYIFSDKTGTLTQN
IMTFKRCSINGRIYGEVHDDLDQKTEITQEKEPVDFSVKSQADREFQFFD
HHLMESIKMGDPKVHEFLRLLALCHTVMSEENSAGELIYQVQSPDEGALV
TAARNFGFIFKSRTPETITIEELGTLVTYQLLAFLDFNNTRKRMSVIVRN
PEGQIKLYSKGADTILFEKLHPSNEVLLSLTSDHLSEFAGEGLRTLAIAY
RDLDDKYFKEWHKMLEDANAATEERDERIAGLYEEIERDLMLLGATAVED
KLQEGVIETVTSLSLANIKIWVLTGDKQETAINIGYACNMLTDDMNDVFV
IAGNNAVEVREELRKAKQNLFGQNRNFSNGHVVCEKKQQLELDSIVEETI
TGDYALIINGHSLAHALESDVKNDLLELACMCKTVICCRVTPLQKAQVVE
LVKKYRNAVTLAIGDGANDVSMIKSAHIGVGISGQEGLQAVLASDYSFAQ
FRYLQRLLLVHGRWSYFRMCKFLCYFFYKNFAFTLVHFWFGFFCGFSAQT
VYDQWFITLFNIVYTSLPVLAMGIFDQDVSDQNSVDCPQLYKPGQLNLLF
NKRKFFICVLHGIYTSLVLFFIPYGAFYNVAGEDGQHIADYQSFAVTMAT
SLVIVVSVQIALDTSYWTFINHVFIWGSIAIYFSILFTMHSNGIFGIFPN
QFPFVGNARHSLTQKCIWLVILLTTVASVMPVVAFRFLKVDLYPTLSDQI
RRWQKAQKKARPPSSRRPRTRRSSSRRSGYAFAHQEGYGELITSGKNMRA
KNPPPTSGLEKTHYNSTSWIENLCKKTTDTVSSFSQDKTVKL

AFAP1L2, GCID: GC10M114281 refers to a protein associated with Sh3 domain binding and protein tyrosine kinase activator activity. A non-limiting exemplary sequence of the human protein provided below may be found under UniProtKB Ref. No. Q8N4X5, accessible through the Gene Cards database (SEQ ID NO: 17):

MERYKALEQLLTELDDFLKILDQENLSSTALVKKSCLAELLRLYTKSSSS
DEEYIYMNKVTINKQQNAESQGKAPEEQGLLPNGEPSQHSSAPQKSLPDL
PPPKMIPERKQLAIPKTESPEGYYEEAEPYDTSLNEDGEAVSSSYESYDE
EDGSKGKSAPYQWPSPEAGIELMRDARICAFLWRKKWLGQWAKQLCVIKD
NRLLCYKSSKDHSPQLDVNLLGSSVIHKEKQVRKKEHKLKITPMNADVIV
LGLQSKDQAEQWLRVIQEVSGLPSEGASEGNQYTPDAQRFNCQKPDIAEK
YLSASEYGSSVDGHPEVPETKDVKKKCSAGLKLSNLMNLGRKKSTSLEPV
ERSLETSSYLNVLVNSQWKSRWCSVRDNHLHFYQDRNRSKVAQQPLSLVG
CEVVPDPSPDHLYSFRILHKGEELAKLEAKSSEEMGHWLGLLLSESGSKT
DPEEFTYDYVDADRVSCIVSAAKNSLLLMQRKFSEPNTYIDGLPSQDRQE
ELYDDVDLSELTAAVEPTEEATPVADDPNERESDRVYLDLTPVKSFLHGP
SSAQAQASSPTLSCLDNATEALPADSGPGPTPDEPCIKCPENLGEQQLES
LEPEDPSLRITTVKIQTEQQRISFPPSCPDAVVATPPGASPPVKDRLRVT
SAEIKLGKNRTEAEVKRYTEEKERLEKKKEEIRGHLAQLRKEKRELKETL
LKCTDKEVLASLEQKLKEIDEECRGEESRRVDLELSIMEVKDNLKKAEAG
PVTLGTTVDTTHLENVSPRPKAVTPASAPDCTPVNSATTLKNRPLSVVVT
GKGTVLQKAKEWEKKGAS

DAPK2, GCID: GC15M063907 refers to a protein that belongs to the serine/threonine protein kinase family. A non-limiting exemplary sequence of the human protein provided below may be found under UniProtKB Ref No. Q9UIK4, accessible through the Gene Cards database (SEQ ID NO: 18):

MFQASMRSPNMEPFKQQKVEDFYDIGEELGSGQFAIVKKCREKSTGLEYA
AKFIKKRQSRASRRGVSREEIEREVSILRQVLHHNVITLHDVYENRTDVV
LILELVSGGELFDFLAQKESLSEEEATSFIKQILDGVNYLHTKKIAHFDL
KPENIMLLDKNIPIPHIKLIDFGLAHEIEDGVEFKNIFGTPEFVAPEIVN
YEPLGLEADMWSIGVITYILLSGASPFLGDTKQETLANITAVSYDFDEEF
FSQTSELAKDFIRKLLVKETRKRLTIQEALRHPWITPVDNQQAMVRRESV
VNLENFRKQYVRRRWKLSFSIVSLCNHLTRSLMKKVHLRPDEDLRNCESD
TEEDIARRKALHPRRRSSTS

PTMS, GCID: GC12P006765 refers to a protein hypothesized to mediate immune function by blocking the effect of prothymosin alpha which confers resistance to certain opportunistic infections. A non-limiting exemplary sequence of the human protein provided below may be found under UniProtKB Ref No. P20962, accessible through the Gene Cards database (SEQ ID NO: 19):

MSEKSVEAAAELSAKDLKEKKEKVEEKASRKERKKEVVEEEENGAEEEEE
ETAEDGEEEDEGEEEDEEEEEEDDEGPALKRAAEEEDEADPKRQKTENGA
SA

ATP10D, GCID: GC04P047487 refers to a catalytic component of a P4-ATPase flippase complex which catalyzes the hydrolysis of ATP coupled to the transport of aminophospholipids from the outer to the inner leaflet of various membranes and ensures the maintenance of asymmetric distribution of phospholipids. A non-limiting exemplary sequence of the human protein provided below may be found under UniProtKB Ref No. Q9P241, accessible through the Gene Cards database (SEQ ID NO: 20):

MTEALQWARYHWRRLIRGATRDDDSGPYNYSSLLACGRKSSQTPKLSGRH
RIVVPHIQPFKDEYEKFSGAYVNNRIRTTKYTLLNFVPRNLFEQFHRAAN
LYFLFLVVLNWVPLVEAFQKEITMLPLVVVLTIIAIKDGLEDYRKYKIDK
QINNLITKVYSRKEKKYIDRCWKDVTVGDFIRLSCNEVIPADMVLLFSTD
PDGICHIETSGLDGESNLKQRQVVRGYAEQDSEVDPEKFSSRIECESPNN
DLSRFRGFLEHSNKERVGLSKENLLLRGCTIRNTEAVVGIVVYAGHETKA
MLNNSGPRYKRSKLERRANTDVLWCVMLLVIMCLTGAVGHGIWLSRYEKM
HFFNVPEPDGHIISPLLAGFYMFWTMIILLQVLIPISLYVSIEIVKLGQI
YFIQSDVDFYNEKMDSIVQCRALNIAEDLGQIQYLFSDKTGTLTENKMVF
RRCSVAGFDYCHEENARRLESYQEAVSEDEDFIDTVSGSLSNMAKPRAPS
CRTVHNGPLGNKPSNHLAGSSFTLGSGEGASEVPHSRQAAFSSPIETDVV
PDTRLLDKFSQITPRLFMPLDETIQNPPMETLYIIDFFIALAICNTVVVS
APNQPRQKIRHPSLGGLPIKSLEEIKSLFQRWSVRRSSSPSLNSGKEPSS
GVPNAFVSRLPLFSRMKPASPVEEEVSQVCESPQCSSSSACCTETEKQHG
DAGLLNGKAESLPGQPLACNLCYEAESPDEAALVYAARAYQCTLRSRTPE
QVMVDFAALGPLTFQLLHILPFDSVRKRMSVVVRHPLSNQVVVYTKGADS
VIMELLSVASPDGASLEKQQMIVREKTQKHLDDYAKQGLRTLCIAKKVMS
DTEYAEWLRNHFLAETSIDNREELLLESAMRLENKLTLLGATGIEDRLQE
GVPESIEALHKAGIKIWMLTGDKQETAVNIAYACKLLEPDDKLFILNTQS
KDACGMLMSTILKELQKKTQALPEQVSLSEDLLQPPVPRDSGLRAGLIIT
GKTLEFALQESLQKQFLELTSWCQAVVCCRATPLQKSEVVKLVRSHLQVM
TLAIGDGANDVSMIQVADIGIGVSGQEGMQAVMASDFAVSQFKHLSKLLL
VHGHWCYTRLSNMILYFFYKNVAYVNLLFWYQFFCGFSGTSMTDYWVLIF
FNLLFTSAPPVIYGVLEKDVSAETLMQLPELYRSGQKSEAYLPHTFWITL
LDAFYQSLVCFFVPYFTYQGSDTDIFAFGNPLNTAALFIVLLHLVIESKS
LTWIHLLVIIGSILSYFLFAIVFGAMCVTCNPPSNPYWIMQEHMLDPVFY
LVCILTTSIALLPRFVYRVLQGSLFPSPILRAKHFDRLTPEERTKALKKW
RGAGKMNQVTSKYANQSAGKSGRRPMPGPSAVFAMKSASSCAIEQGNLSL
CETALDQGYSETKAFEMAGPSKGKES

SLC7A2, GCID: GC08P017497 refers to a cationic amino acid transporter and a member of the APC (amino acid-polyamine-organocation) family of transporters. A non-limiting exemplary sequence of the human protein provided below may be found under UniProKB Ref No. P52569, accessible through the Gene Cards database (SEQ ID NO: 21):

MIPCRAALTFARCLIRRKIVTLDSLEDTKLCRCLSTMDLIALGVGSTLGA
GVYVLAGEVAKADSGPSIVVSFLIAALASVMAGLCYAEFGARVPKTGSAY
LYTYVTVGELWAFITGWNLILSYVIGTSSVARAWSGTFDELLSKQIGQFL
RTYFRMNYTGLAEYPDFFAVCLILLLAGLLSFGVKESAWVNKVFTAVNIL
VLLFVMVAGFVKGNVANWKISEEFLKNISASAREPPSENGTSIYGAGGFM
PYGFTGTLAGAATCFYAFVGFDCIATTGEEVRNPQKAIPIGIVTSLLVCF
MAYFGVSAALTLMMPYYLLDEKSPLPVAFEYVGWGPAKYVVAAGSLCALS
TSLLGSIFPMPRVIYAMAEDGLLFKCLAQINSKTKTPIIATLSSGAVAAL
MAFLFDLKALVDMMSIGTLMAYSLVAACVLILRYQPGLSYDQPKCSPEKD
GLGSSPRVTSKSESQVTMLQRQGFSMRTLFCPSLLPTQQSASLVSFLVGF
LAFLVLGLSVLTTYGVHAITRLEAWSLALLALFLVLFVAIVLTIWRQPQN
QQKVAFMVPFLPFLPAFSILVNIYLMVQLSADTWVRFSIWMAIGFLIYFS
YGIRHSLEGHLRDENNEEDAYPDNVHAAAEEKSAIQANDHHPRNLSSPFI
FHEKTSEF

LAYN, GCID: GC11P111541 refers to a putative hyaluronate receptor. A non-limiting exemplary sequence of the human protein provided below may be found under UniProtKB Ref. No. Q6UX15, accessible through the Gene Cards database (SEQ ID NO: 22):

MRPGTALQAVLLAVLLVGLRAATGRLLSASDLDLRGGQPVCRGGTQRPCY
KVIYFHDTSRRLNFEEAKEACRRDGGQLVSIESEDEQKLIEKFIENLLPS
DGDFWIGLRRREEKQSNSTACQDLYAWTDGSISQFRNWYVDEPSCGSEVC
VVMYHQPSAPAGIGGPYMFQWNDDRCNMKNNFICKYSDEKPAVPSREAEG
EETELTTPVLPEETQEEDAKKTFKESREAALNLAYILIPSIPLLLLLVVT
TVVCWVWICRKRKREQPDPSTKKQHTIWPSPHQGNSPDLEVYNVIRKQSE
ADLAETRPDLKNISFRVCSGEATPDDMSCDYDNMAVNPSESGFVTLVSVE
SGFVTNDIYEFSPDQMGRSKESGWVENEIYGY

TNS3, GCID: GC07M047281 refers to a protein believed to be involved in actin remodeling, e.g. the dissociation of the integrin-tensin-actin complex. A non-limiting exemplary sequence of the human protein provided below may be found under UniProtKB Ref No. Q68CZ2, accessible through the Gene Cards database (SEQ ID NO: 23):

MEEGHGLDLTYITERIIAVSFPAGCSEESYLHNLQEVTRMLKSKHGDNYL
VLNLSEKRYDLTKLNPKIMDVGWPELHAPPLDKMCTICKAQESWLNSNLQ
HVVVIHCRGGKGRIGVVISSYMHFTNVSASADQALDRFAMKKFYDDKVSA
LMQPSQKRYVQFLSGLLSGSVKMNASPLFLHFVILHGTPNFDTGGVCRPF
LKLYQAMQPVYTSGIYNVGPENPSRICIVIEPAQLLKGDVMVKCYHKKYR
SATRDVIFRLQFHTGAVQGYGLVFGKEDLDNASKDDRFPDYGKVELVFSA
TPEKIQGSEHLYNDHGVIVDYNTTDPLIRWDSYENLSADGEVLHTQGPVD
GSLYAKVRKKSSSDPGIPGGPQAIPATNSPDHSDHTLSVSSDSGHSTASA
RTDKTEERLAPGTRRGLSAQEKAELDQLLSGFGLEDPGSSLKEMTDARSK
YSGTRHVVPAQVHVNGDAALKDRETDILDDEMPHHDLHSVDSLGTLSSSE
GPQSAHLGPFTCHKSSQNSLLSDGFGSNVGEDPQGTLVPDLGLGMDGPYE
RERTFGSREPKQPQPLLRKPSVSAQMQAYGQSSYSTQTWVRQQQMVVAHQ
YSFAPDGEARLVSRCPADNPGLVQAQPRVPLTPTRGTSSRVAVQRGVGSG
PHPPDTQQPSPSKAFKPRFPGDQVVNGAGPELSTGPSPGSPTLDIDQSIE
QLNRLILELDPTFEPIPTHMNALGSQANGSVSPDSVGGGLRASSRLPDTG
EGPSRATGRQGSSAEQPLGGRLRKLSLGQYDNDAGGQLPFSKCAWGKAGV
DYAPNLPPFPSPADVKETMTPGYPQDLDIIDGRILSSKESMCSTPAFPVS
PETPYVKTALRHPPFSPPEPPLSSPASQHKGGREPRSCPETLTHAVGMSE
SPIGPKSTMLRADASSTPSFQQAFASSCTISSNGPGQRRESSSSAERQWV
ESSPKPMVSLLGSGRPTGSPLSAEFSGTRKDSPVLSCFPPSELQAPFHSH
ELSLAEPPDSLAPPSSQAFLGFGTAPVGSGLPPEEDLGALLANSHGASPT
PSIPLTATGAADNGFLSHNFLTVAPGHSSHHSPGLQGQGVTLPGQPPLPE
KKRASEGDRSLGSVSPSSSGFSSPHSGSTISIPFPNVLPDFSKASEAASP
LPDSPGDKLVIVKFVQDTSKFWYKADISREQAIAMLKDKEPGSFIVRDSH
SFRGAYGLAMKVATPPPSVLQLNKKAGDLANELVRHFLIECTPKGVRLKG
CSNEPYFGSLTALVCQHSITPLALPCKLLIPERDPLEEIAESSPQTAANS
AAELLKQGAACNVWYLNSVEMESLTGHQAIQKALSITLVQEPPPVSTVVH
FKVSAQGITLTDNQRKLFFRRHYPVNSVIFCALDPQDRKWIKDGPSSKVF
GFVARKQGSATDNVCHLFAEHDPEQPASAIVNFVSKVMIGSPKKV

KIR2DL4, GCID: GC19P054994 refers to a transmembrane glycoprotein expressed by natural killer cells and subsets of T cells. A non-limiting exemplary sequence of the human protein provided below may be found under UniProtKB Ref. No. Q99706, accessible through the Gene Cards database (SEQ ID NO: 24):

MSMSPTVIILACLGFFLDQSVWAHVGGQDKPFCSAWPSAVVPQGGHVTLR
CHYRRGFNIFTLYKKDGVPVPELYNRIFWNSFLISPVTPAHAGTYRCRGF
HPHSPTEWSAPSNPLVIMVTGLYEKPSLTARPGPTVRAGENVTLSCSSQS
SFDIYHLSREGEAHELRLPAVPSINGTFQADFPLGPATHGETYRCFGSFH
GSPYEWSDPSDPLPVSVTGNPSSSWPSPTEPSFKTGIARHLHAVIRYSVA
IILFTILPFFLLHRWCSKKKDAAVMNQEPAGHRTVNREDSDEQDPQEVTY
AQLDHCIFTQRKITGPSQRSKRPSTDTSVCIELPNAEPRALSPAHEHHSQ
ALMGSSRETTALSQTQLASSNVPAAGI

ENTPD1, GCID: GC10P095711 refers to a plasma membrane protein that hydrolyzes extracellular ATP and ADP to AMP. A non-limiting exemplary sequence of the human protein provided below may be found under UniProtKB Ref. No. P49961, accessible through the Gene Cards database (SEQ ID NO: 25):

MEDTKESNVKTFCSKNILAILGFSSIIAVIALLAVGLTQNKALPENVKYG
IVLDAGSSHTSLYIYKWPAEKENDTGVVHQVEECRVKGPGISKFVQKVNE
IGIYLTDCMERAREVIPRSQHQETPVYLGATAGMRLLRMESEELADRVLD
VVERSLSNYPFDFQGARIITGQEEGAYGWITINYLLGKFSQKTRWFSIVP
YETNNQETFGALDLGGASTQVTFVPQNQTIESPDNALQFRLYGKDYNVYT
HSFLCYGKDQALWQKLAKDIQVASNEILRDPCFHPGYKKVVNVSDLYKTP
CTKRFEMTLPFQQFEIQGIGNYQQCHQSILELFNTSYCPYSQCAFNGIFL
PPLQGDFGAFSAFYFVMKFLNLTSEKVSQEKVTEMMKKFCAQPWEEIKTS
YAGVKEKYLSEYCFSGTYILSLLLQGYHFTADSWEHIHFIGKIQGSDAGW
TLGYMLNLTNMIPAEQPLSTPLSHSTYVFLMVLFSLVLFTVAIIGLLIFH
KPSYFWKDMV

AKAP5, GCID: GC14P064465 refers to a member of the AKAP family of proteins, which are capable of binding to the regulatory subunit of protein kinase A (PKA) and confining the holoenzyme to discrete locations within the cell. A non-limiting exemplary sequence of the human protein provided below may be found under UniProtKB Ref No. P24588, accessible through the Gene Cards database (SEQ ID NO: 26):

METTISEIHVENKDEKRSAEGSPGAERQKEKASMLCFKRRKKAAKALKPK
AGSEAADVARKCPQEAGASDQPEPTRGAWASLKRLVTRRKRSESSKQQKP
LEGEMQPAINAEDADLSKKKAKSRLKIPCIKFPRGPKRSNHSKIIEDSDC
SIKVQEEAEILDIQTQTPLNDQATKAKSTQDLSEGISRKDGDEVCESNVS
NSTTSGEKVISVELGLDNGHSAIQTGTLILEEIETIKEKQDVQPQQASPL
ETSETDHQQPVLSDVPPLPAIPDQQIVEEASNSTLESAPNGKDYESTEIV
AEETKPKDTELSQESDFKENGITEEKSKSEESKRMEPIAIIITDTEISEF
DVTKSKNVPKQFLISAENEQVGVFANDNGFEDRTSEQYETLLIETASSLV
KNAIQLSIEQLVNEMASDDNKINNLLQ

TTYH3, GCID: GC07P002638 refers to a member of the tweety family of proteins, which function as chloride anion channels. A non-limiting exemplary sequence of the human protein provided below may be found under UniProtKB Ref No. Q9C0H2, accessible through the Gene Cards database (SEQ ID NO: 27):

MAGVSYAAPWWVSLLHRLPHFDLSWEATSSQFRPEDTDYQQALLLLGAAA
LACLALDLLFLLFYSFWLCCRRRKSEEHLDADCCCTAWCVIIATLVCSAG
IAVGFYGNGETSDGIHRATYSLRHANRTVAGVQDRVWDTAVGLNHTAEPS
LQTLERQLAGRPEPLRAVQRLQGLLETLLGYTAAIPFWRNTAVSLEVLAE
QVDLYDWYRWLGYLGLLLLDVIICLLVLVGLIRSSKGILVGVCLLGVLAL
VISWGALGLELAVSVGSSDFCVDPDAYVTKMVEEYSVLSGDILQYYLACS
PRAANPFQQKLSGSHKALVEMQDVVAELLRTVPWEQPATKDPLLRVQEVL
NGTEVNLQHLTALVDCRSLHLDYVQALTGFCYDGVEGLIYLALFSFVTAL
MFSSIVCSVPHTWQQKRGPDEDGEEEAAPGPRQAHDSLYRVHMPSLYSCG
SSYGSETSIPAAAHTVSNAPVTEYMSQNANFQNPRCENTPLIGRESPPPS
YTSSMRAKYLATSQPRPDSSGSH

ASB2, GCID: GC14M093934 refers to a member of the ankyrin repeat and SOCS box-containing (ASB) protein family, which play a role in protein degradation by coupling suppressor of cytokine signalling (SOCS) proteins with the elongin BC complex. A non-limiting exemplary sequence of the human protein provided below may be found under UniProtKB Ref. No. Q96Q27, accessible through the Gene Cards database (SEQ ID NO: 28):

MTRFSYAEYFSLFHSCSAPSRSTAPPESSPARAPMGLFQGVMQKYSSSLF
KTSQLAPADPLIKAIKDGDEEALKTMIKEGKNLAEPNKEGWLPLHEAAYY
GQVGCLKVLQRAYPGTIDQRTLQEETAVYLATCRGHLDCLLSLLQAGAEP
DISNKSRETPLYKACERKNAEAVKILVQHNADTNHRCNRGWTALHESVSR
NDLEVMQILVSGGAKVESKNAYGITPLFVAAQSGQLEALRFLAKYGADIN
TQASDNASALYEACKNEHEEVVEFLLSQGADANKTNKDGLLPLHIASKKG
NYRIVQMLLPVTSRTRIRRSGVSPLHLAAERNHDEVLEALLSARFDVNTP
LAPERARLYEDRRSSALYFAVVNNNVYATELLLQHGADPNRDVISPLLVA
IRHGCLRTMQLLLDHGANIDAYIATHPTAFPATIMFAMKCLSLLKFLMDL
GCDGEPCFSCLYGNGPHPPAPQPSSRFNDAPAADKEPSVVQFCEFVSAPE
VSRWAGPIIDVLLDYVGNVQLCSRLKEHIDSFEDWAVIKEKAEPPRPLAH
LCRLRVRKAIGKYRIKLLDTLPLPGRLIRYLKYENTQ

DBN1, GCID: GC05M177456 refers to a cytoplasmic actin-binding protein thought to play a role in the process of neuronal growth. A non-limiting exemplary sequence of the human protein provided below may be found under UniProtKB Ref. No. Q16643, accessible through the Gene Cards database (SEQ ID NO: 29):

MAGVSFSGHRLELLAAYEEVIREESAADWALYTYEDGSDDLKLAASGEGG
LQELSGHFENQKVMYGFCSVKDSQAALPKYVLINWVGEDVPDARKCACAS
HVAKVAEFFQGVDVIVNASSVEDIDAGAIGQRLSNGLARLSSPVLHRLRL
REDENAEPVGTTYQKTDAAVEMKRINREQFWEQAKKEEELRKEEERKKAL
DERLRFEQERMEQERQEQEERERRYREREQQIEEHRRKQQTLEAEEAKRR
LKEQSIFGDHRDEEEETHMKKSESEVEEAAAIIAQRPDNPREFFKQQERV
ASASAGSCDVPSPFNHRPGSHLDSHRRMAPTPIPTRSPSDSSTASTPVAE
QIERALDEVTSSQPPPLPPPPPPAQETQEPSPILDSEETRAAAPQAWAGP
MEEPPQAQAPPRGPGSPAEDLMFMESAEQAVLAAPVEPATADATEIHDAA
DTIETDTATADTTVANNVPPAATSLIDLWPGNGEGASTLQGEPRAPTPPS
GTEVTLAEVPLLDEVAPEPLLPAGEGCATLLNFDELPEPPATFCDPEEVE
GESLAAPQTPTLPSALEELEQEQEPEPHLLTNGETTQKEGTQASEGYFSQ
SQEEEFAQSEELCAKAPPPVFYNKPPEIDITCWDADPVPEEEEGFEGGD

ACP5, GCID: GC19M011574 refers to an iron containing glycoprotein which catalyzes the conversion of orthophosphoric monoester to alcohol and orthophosphate. A non-limiting exemplary sequence of the human protein provided below may be found under UniProtKB Ref No. P13686, accessible through the Gene Cards database (SEQ ID NO: 30):

MDMWTALLILQALLLPSLADGATPALRFVAVGDWGGVPNAPFHTAREMAN
AKEIARTVQILGADFILSLGDNFYFTGVQDINDKRFQETFEDVFSDRSLR
KVPWYVLAGNHDHLGNVSAQIAYSKISKRWNFPSPFYRLHFKIPQTNVSV
AIFMLDTVTLCGNSDDFLSQQPERPRDVKLARTQLSWLKKQLAAAREDYV
LVAGHYPVWSIAEHGPTHCLVKQLRPLLATYGVTAYLCGHDHNLQYLQDE
NGVGYVLSGAGNFMDPSKRHQRKVPNGYLRFHYGTEDSLGGFAYVEISSK
EMTVTYIEASGKSLFKTRLPRRARP

ABCB1, GCID: GC07M087504 refers to a member of the superfamily of ATP-binding cassette (ABC) transporters, which transport various molecules across the extra- and/or intra-cellular membranes. A non-limiting exemplary sequence of the human protein provided below may be found under UniProtKB Ref. No. P08183, accessible through the Gene Cards database (SEQ ID NO: 31):

MDLEGDRNGGAKKKNFFKLNNKSEKDKKEKKPTVSVFSMFRYSNWLDKLY
MVVGTLAAIIHGAGLPLMMLVFGEMTDIFANAGNLEDLMSNITNRSDIND
TGFFMNLEEDMTRYAYYYSGIGAGVLVAAYIQVSFWCLAAGRQIHKIRKQ
FFHAIMRQEIGWFDVHDVGELNTRLTDDVSKINEGIGDKIGMFFQSMATF
FTGFIVGFTRGWKLTLVILAISPVLGLSAAVWAKILSSFTDKELLAYAKA
GAVAEEVLAAIRTVIAFGGQKKELERYNKNLEEAKRIGIKKAITANISIG
AAFLLIYASYALAFWYGTTLVLSGEYSIGQVLTVFFSVLIGAFSVGQASP
SIEAFANARGAAYEIFKIIDNKPSIDSYSKSGHKPDNIKGNLEFRNVHFS
YPSRKEVKILKGLNLKVQSGQTVALVGNSGCGKSTTVQLMQRLYDPTEGM
VSVDGQDIRTINVRFLREIIGVVSQEPVLFATTIAENIRYGRENVTMDEI
EKAVKEANAYDFIMKLPHKFDTLVGERGAQLSGGQKQRIAIARALVRNPK
ILLLDEATSALDTESEAVVQVALDKARKGRTTIVIAHRLSTVRNADVIAG
FDDGVIVEKGNHDELMKEKGIYFKLVTMQTAGNEVELENAADESKSEIDA
LEMSSNDSRSSLIRKRSTRRSVRGSQAQDRKLSTKEALDESIPPVSFWRI
MKLNLTEWPYFVVGVFCAIINGGLQPAFAIIFSKIIGVFTRIDDPETKRQ
NSNLFSLLFLALGIISFITFFLQGFTFGKAGEILTKRLRYMVFRSMLRQD
VSWFDDPKNTTGALTTRLANDAAQVKGAIGSRLAVITQNIANLGTGIIIS
FIYGWQLTLLLLAIVPIIAIAGVVEMKMLSGQALKDKKELEGSGKIATEA
IENFRTVVSLTQEQKFEHMYAQSLQVPYRNSLRKAHIFGITFSFTQAMMY
FSYAGCFRFGAYLVAHKLMSFEDVLLVFSAVVFGAMAVGQVSSFAPDYAK
AKISAAHIIMIIEKTPLIDSYSTEGLMPNTLEGNVTFGEVVFNYPTRPDI
PVLQGLSLEVKKGQTLALVGSSGCGKSTVVQLLERFYDPLAGKVLLDGKE
IKRLNVQWLRAHLGIVSQEPILFDCSIAENIAYGDNSRVVSQEEIVRAAK
EANIHAFIESLPNKYSTKVGDKGTQLSGGQKQRIAIARALVRQPHILLLD
EATSALDTESEKVVQEALDKAREGRTCIVIAHRLSTIQNADLIVVFQNGR
VKEHGTHQQLLAQKGIYFSMVSVQAGTKRQ

KLRB1, GCID: GC12M011717 refers to a protein that an extracellular domain with several motifs characteristic of C-type lectins, a transmembrane domain, and a cytoplasmic domain. The KLRB1 protein is classified as a type II membrane protein because it has an external C terminus and may be involved with the regulation of NK cell function. A non-limiting exemplary sequence of the human protein provided below may be found under UniProtKB Ref No. Q12918, accessible through the Gene Cards database (SEQ ID NO: 32):

MDQQAIYAELNLPTDSGPESSSPSSLPRDVCQGSPWHQFALKLSCAGIIL
LVLVVTGLSVSVTSLIQKSSIEKCSVDIQQSRNKTTERPGLLNCPIYWQQ
LREKCLLFSHTVNPWNNSLADCSTKESSLLLIRDKDELIHTQNLIRDKAI
LFWIGLNFSLSEKNWKWINGSFLNSNDLEIRGDAKENSCISISQTSVYSE
YCSTEIRWICQKELTPVRNKVYPDS

ALOX5AP, GCID: GC13P030713 refers to a protein which, with 5-lipoxygenase, is required for leukotriene synthesis. A non-limiting exemplary sequence of the human protein provided below may be found under UniProtKB Ref. No. P20292, accessible through the Gene Cards database (SEQ ID NO: 33):

MDQETVGNVVLLAIVTLISVVQNGFFAHKVEHESRTQNGRSFQRTGTLAF
ERVYTANQNCVDAYPTFLAVLWSAGLLCSQVPAAFAGLMYLFVRQKYFVG
YLGERTQSTPGYIFGKRIILFLFLMSVAGIFNYYLIFFFGSDFENYIKTI
STTISPLLLIP

GALNT2, GCID: GC01P230057 refers to a member of the glycosyltransferase 2 protein family, which are known to initiate mucin-type O-glycosylation of peptides in the Goldi apparatus. A non-limiting exemplary sequence of the human protein provided below may be found under UniProtKB Ref No. Q10471, accessible through the Gene Cards database (SEQ ID NO: 34):

MRRRSRMLLCFAFLWVLGIAYYMYSGGGSALAGGAGGGAGRKEDWNEIDP
IKKKDLHHSNGEEKAQSMETLPPGKVRWPDFNQEAYVGGTMVRSGQDPYA
RNKFNQVESDKLRMDRAIPDTRHDQCQRKQWRVDLPATSVVITFHNEARS
ALLRTVVSVLKKSPPHLIKEIILVDDYSNDPEDGALLGKIEKVRVLRNDR
REGLMRSRVRGADAAQAKVLTFLDSHCECNEHWLEPLLERVAEDRTRVVS
PIIDVINMDNFQYVGASADLKGGFDWNLVFKWDYMTPEQRRSRQGNPVAP
IKTPMIAGGLFVMDKFYFEELGKYDMMMDVWGGENLEISFRVWQCGGSLE
IIPCSRVGHVFRKQHPYTFPGGSGTVFARNTRRAAEVWMDEYKNFYYAAV
PSARNVPYGNIQSRLELRKKLSCKPFKWYLENVYPELRVPDHQDIAFGAL
QQGTNCLDTLGHFADGVVGVYECHNAGGNQEWALTKEKSVKHMDLCLTVV
DRAPGSLIKLQGCRENDSRQKWEQIEGNSKLRHVGSNLCLDSRTAKSGGL
SVEVCGPALSQQWKFTLNLQQ

SIRPG, GCID: GC20M001628 refers to a member of the signal-regulatory protein (SRP) family, which receptor-type transmembrane glycoproteins known to be involved in the negative regulation of receptor tyrosine kinase-coupled signaling processes. A non-limiting exemplary sequence of the human protein provided below may be found under UniProtKB Ref No. Q9P1W8, accessible through the Gene Cards database (SEQ ID NO: 35):

MPVPASWPHPPGPFLLLTLLLGLTEVAGEEELQMIQPEKLLLVTVGKTAT
LHCTVTSLLPVGPVLWFRGVGPGRELIYNQKEGHFPRVTTVSDLTKRNNM
DFSIRISSITPADVGTYYCVKFRKGSPENVEFKSGPGTEMALGAKPSAPV
VLGPAARTTPEHTVSFTCESHGFSPRDITLKWFKNGNELSDFQTNVDPTG
QSVAYSIRSTARVVLDPWDVRSQVICEVAHVTLQGDPLRGTANLSEAIRV
PPTLEVTQQPMRVGNQVNVTCQVRKFYPQSLQLTWSENGNVCQRETASTL
TENKDGTYNWTSWFLVNISDQRDDVVLTCQVKHDGQLAVSKRLALEVTVH
QKDQSSDATPGPASSLTALLLIAVLLGPIYVPWKQKT

NDFIP2, GCID: GC13P079481 refers to a protein associated with signal transduced activity and WW domain binding which is a paralog of NDFIP1. A non-limiting exemplary sequence of the human protein provided below may be found under UniProtKB Ref No. Q9NV92, accessible through the Gene Cards database (SEQ ID NO: 36):

MARRRSQRVCASGPSMLNSARGAPELLRGTATNAEVSAAAAGATGSEELP
PGDRGCRNGGGRGPAATTSSTGVAVGAEHGEDSLSRKPDPEPGRMDHHQP
GTGRYQVLLNEEDNSESSAIEQPPTSNPAPQIVQAASSAPALETDSSPPP
YSSITVEVPTTSDTEVYGEFYPVPPPYSVATSLPTYDEAEKAKAAAMAAA
AAETSQRIQEEECPPRDDFSDADQLRVGNDGIFMLAFFMAFIFNWLGFCL
SFCITNTIAGRYGAICGFGLSLIKWILIVRFSDYFTGYFNGQYWLWWIFL
VLGLLLFFRGFVNYLKVRNMSESMAAAHRTRYFFLL

SNAP47, GCID: GC01P227730 refers to a protein that plays a role in intra-cellular membrane fusion. A non-limiting exemplary sequence of the human protein provided below may be found under UniProtKB Ref No. Q5SQN1, accessible through the Gene Cards database (SEQ ID NO: 37):

MRAARRGLHCAGAERPRRRGRLWDSSGVPQRQKRPGPWRTQTQEQMSRDV
CIHTWPCTYYLEPKRRWVTGQLSLTSLSLRFMTDSTGEILVSFPLSSIVE
IKKEASHFIFSSITILEKGHAKHWFSSLRPSRNVVFSIIEHFWRELLLSQ
PGAVADASVPRTRGEELTGLMAGSQKRLEDTARVLHHQGQQLDSVMRGLD
KMESDLEVADRLLTELESPAWWPFSSKLWKTPPETKPREDVSMTSCEPFG
KEGILIKIPAVISHRTESHVKPGRLTVLVSGLEIHDSSSLLMHRFEREDV
DDIKVHSPYEISIRQRFIGKPDMAYRLISAKMPEVIPILEVQFSKKMELL
EDALVLRSARTSSPAEKSCSVWHAASGLMGRTLHREPPAGDQEGTALHLQ
TSLPALSEADTQELTQILRRMKGLALEAESELERQDEALDGVAAAVDRAT
LTIDKHNRRMKRLT

CD200R1, GCID: GC03M112921 refers to a receptor for the OX-2 membrane glycoprotein. A non-limiting exemplary sequence of the human protein provided below may be found under UniProtKB Ref. No. Q8TD46, accessible through the Gene Cards database (SEQ ID NO: 38):

MLCPWRTANLGLLLILTIFLVAASSSLCMDEKQITQNYSKVLAEVNTSWP
VKMATNAVLCCPPIALRNLIIITWEIILRGQPSCTKAYRKETNETKETNC
TDERITWVSRPDQNSDLQIRPVAITHDGYYRCIMVTPDGNFHRGYHLQVL
VTPEVTLFQNRNRTAVCKAVAGKPAAQISWIPEGDCATKQEYWSNGTVTV
KSTCHWEVHNVSTVTCHVSHLTGNKSLYIELLPVPGAKKSAKLYIPYIIL
TIIILTIVGFIWLLKVNGCRKYKLNKTESTPVVEEDEMQPYASYTEKNNP
LYDTTNKVKASEALQSEVDTDLHTL

PATL2, GCID: GC15M044665 refers to an RNA-binding protein that acts as a translational repressor. A non-limiting exemplary sequence of the human protein provided below may be found under UniProtKB Ref No. C9JE40, accessible through the Gene Cards database (SEQ ID NO: 39):

MNCLEGPGKTCGPLASEEELVSACQLEKEEENEGEEEEEEEDEEDLDPDL
DPDLEEEENDLGDPAVLGAVHNTQRALLSSPGVKAPGMLGMSLASLHFLW
QTLDYLSPIPFWPTFPSTSSPAQHFGPRLPSPDPTLFCSLLTSWPPRFSH
LTQLHPRHQRILQQQQHSQTPSPPAKKPWSQQPDPYANLMTRKEKDWVIK
VQMVQLQSAKPRLDDYYYQEYYQKLEKKQADEELLGRRNRVESLKLVTPY
IPKAEAYESVVRIEGSLGQVAVSTCFSPRRAIDAVPHGTQEQDIEAASSQ
RLRVLYRIEKMFLQLLEIEEGWKYRPPPPCFSEQQSNQVEKLFQTLKTQE
QNNLEEAADGFLQVLSVRKGKALVARLLPFLPQDQAVTILLAITHHLPLL
VRRDVADQALQMLFKPLGKCISHLTLHELLQGLQGLTLLPPGSSERPVTV
VLQNQFGISLLYALLSHGEQLVSLHSSLEEPNSDHTAWTDMVVLIAWEIA
QMPTASLAEPLAFPSNLLPLFCHHVDKQLVQQLEARMEFAWIY

ADRB2, GCID: GC05P148825 refers to a beta-2-adrenergic receptor which is a member of the G protein-coupled receptor superfamily. A non-limiting exemplary sequence of the human protein provided below may be found under UniProtKB Ref. No. P07550, accessible through the Gene Cards database (SEQ ID NO: 40):

MGQPGNGSAFLLAPNGSHAPDHDVTQERDEVWVVGMGIVMSLIVLAIVFG
NVLVITAIAKFERLQTVTNYFITSLACADLVMGLAVVPFGAAHILMKMWT
FGNFWCEFWTSIDVLCVTASIETLCVIAVDRYFAITSPFKYQSLLTKNKA
RVIILMVWIVSGLTSFLPIQMHWYRATHQEAINCYANETCCDFFTNQAYA
IASSIVSFYVPLVIMVFVYSRVFQEAKRQLQKIDKSEGRFHVQNLSQVEQ
DGRTGHGLRRSSKFCLKEHKALKTLGIIMGTFTLCWLPFFIVNIVHVIQD
NLIRKEVYILLNWIGYVNSGFNPLIYCRSPDFRIAFQELLCLRRSSLKAY
GNGYSSNGNTGEQSGYHVEQEKENKLLCEDLPGTEDFVGHQGTVPSDNID
SQGRNCSTNDSLL

SORL1, GCID: GC11P121452 refers to a mosaic protein that belongs to at least two families: the vacuolar protein sorting 10 (VPS10) domain-containing receptor family, and the low-density lipoprotein receptor (LDLR) family. A non-limiting exemplary sequence of the human protein provided below may be found under UniProtKB Ref. No. Q92673, accessible through the Gene Cards database (SEQ ID NO: 41):

MATRSSRRESRLPFLFTLVALLPPGALCEVWTQRLHGGSAPLPQDRGFLV
VQGDPRELRLWARGDARGASRADEKPLRRKRSAALQPEPIKVYGQVSLND
SHNQMVVHWAGEKSNVIVALARDSLALARPKSSDVYVSYDYGKSFKKISD
KLNFGLGNRSEAVIAQFYHSPADNKRYIFADAYAQYLWITFDFCNTLQGF
SIPFRAADLLLHSKASNLLLGFDRSHPNKQLWKSDDFGQTWIMIQEHVKS
FSWGIDPYDKPNTIYIERHEPSGYSTVFRSTDFFQSRENQEVILEEVRDF
QLRDKYMFATKVVHLLGSEQQSSVQLWVSFGRKPMRAAQFVTRHPINEYY
IADASEDQVFVCVSHSNNRTNLYISEAEGLKFSLSLENVLYYSPGGAGSD
TLVRYFANEPFADFHRVEGLQGVYIATLINGSMNEENMRSVITFDKGGTW
EFLQAPAFTGYGEKINCELSQGCSLHLAQRLSQLLNLQLRRMPILSKESA
PGLIIATGSVGKNLASKTNVYISSSAGARWREALPGPHYYTWGDHGGIIT
AIAQGMETNELKYSTNEGETWKTFIFSEKPVFVYGLLTEPGEKSTVFTIF
GSNKENVHSWLILQVNATDALGVPCTENDYKLWSPSDERGNECLLGHKTV
FKRRTPHATCFNGEDFDRPVVVSNCSCTREDYECDFGFKMSEDLSLEVCV
PDPEFSGKSYSPPVPCPVGSTYRRTRGYRKISGDTCSGGDVEARLEGELV
PCPLAEENEFILYAVRKSIYRYDLASGATEQLPLTGLRAAVALDFDYEHN
CLYWSDLALDVIQRLCLNGSTGQEVIINSGLETVEALAFEPLSQLLYWVD
AGFKKIEVANPDGDFRLTIVNSSVLDRPRALVLVPQEGVMFWTDWGDLKP
GIYRSNMDGSAAYHLVSEDVKWPNGISVDDQWIYWTDAYLECIERITFSG
QQRSVILDNLPHPYAIAVFKNEIYWDDWSQLSIFRASKYSGSQMEILANQ
LTGLMDMKIFYKGKNTGSNACVPRPCSLLCLPKANNSRSCRCPEDVSSSV
LPSGDLMCDCPQGYQLKNNTCVKQENTCLRNQYRCSNGNCINSIWWCDFD
NDCGDMSDERNCPTTICDLDTQFRCQESGTCIPLSYKCDLEDDCGDNSDE
SHCEMHQCRSDEYNCSSGMCIRSSWVCDGDNDCRDWSDEANCTAIYHTCE
ASNFQCRNGHCIPQRWACDGDTDCQDGSDEDPVNCEKKCNGFRCPNGTCI
PSSKHCDGLRDCSDGSDEQHCEPLCTHFMDFVCKNRQQCLFHSMVCDGII
QCRDGSDEDAAFAGCSQDPEFHKVCDEFGFQCQNGVCISLIWKCDGMDDC
GDYSDEANCENPTEAPNCSRYFQFRCENGHCIPNRWKCDRENDCGDWSDE
KDCGDSHILPFSTPGPSTCLPNYYRCSSGTCVMDTWVCDGYRDCADGSDE
EACPLLANVTAASTPTQLGRCDRFEFECHQPKTCIPNWKRCDGHQDCQDG
RDEANCPTHSTLTCMSREFQCEDGEACIVLSERCDGFLDCSDESDEKACS
DELTVYKVQNLQWTADFSGDVTLTWMRPKKMPSASCVYNVYYRVVGESIW
KTLETHSNKTNTVLKVLKPDTTYQVKVQVQCLSKAHNTNDFVTLRTPEGL
PDAPRNLQLSLPREAEGVIVGHWAPPIHTHGLIREYIVEYSRSGSKMWAS
QRAASNFTEIKNLLVNTLYTVRVAAVTSRGIGNWSDSKSITTIKGKVIPP
PDIHIDSYGENYLSFTLTMESDIKVNGYVVNLFWAFDTHKQERRTLNFRG
SILSHKVGNLTAHTSYEISAWAKTDLGDSPLAFEHVMTRGVRPPAPSLKA
KAINQTAVECTWTGPRNVVYGIFYATSFLDLYRNPKSLTTSLHNKTVIVS
KDEQYLFLVRVVVPYQGPSSDYVVVKMIPDSRLPPRHLHVVHTGKTSVVI
KWESPYDSPDQDLLYAVAVKDLIRKTDRSYKVKSRNSTVEYTLNKLEPGG
KYHIIVQLGNMSKDSSIKITTVSLSAPDALKIITENDHVLLFWKSLALKE
KHFNESRGYEIHMFDSAMNITAYLGNTTDNFFKISNLKMGHNYTFTVQAR
CLFGNQICGEPAILLYDELGSGADASATQAARSTDVAAVVVPILFLILLS
LGVGFAILYTKHRRLQSSFTAFANSHYSSRLGSAIFSSGDDLGEDDEDAP
MITGFSDDVPMVIA

CD300A, GCID: GC17P074466 refers to a member of the CD300 glycoprotein family of cell surface proteins found on leukocytes involved in immune response signaling pathways. A non-limiting exemplary sequence of the human protein provided below may be found under UniProtKB Ref. No. Q9UGN4, accessible through the Gene Cards database (SEQ ID NO: 42):

MWLPWALLLLWVPGCFALSKCRTVAGPVGGSLSVQCPYEKEHRTLNKYWC
RPPQIFLCDKIVETKGSAGKRNGRVSIRDSPANLSFTVTLENLTEEDAGT
YWCGVDTPWLRDFHDPVVEVEVSVFPASTSMTPASITAAKTSTITTAFPP
VSSTTLFAVGATHSASIQEETEEVVNSQLPLLLSLLALLLLLLVGASLLA
WRMFQKWIKAGDHSELSQNPKQAATQSELHYANLELLMWPLQEKPAPPRE
VEVEYSTVASPREELHYASVVFDSNTNRIAAQRPREEEPDSDYSVIRKT

Clorf12, GCID: GC01M231363 is an alternate name for EGLN1, which is a catalyzes the post-translational formation of 4-hydroxyproline in hypoxia-inducible factor (HIF) alpha proteins. A non-limiting exemplary sequence of the human protein provided below may be found under UniProtKB Ref. No. Q9GZT9, accessible through the Gene Cards database (SEQ ID NO: 43):

MANDSGGPGGPSPSERDRQYCELCGKMENLLRCSRCRSSFYCCKEHQRQD
WKKHKLVCQGSEGALGHGVGPHQHSGPAPPAAVPPPRAGAREPRKAAARR
DNASGDAAKGKVKAKPPADPAAAASPCRAAAGGQGSAVAAEAEPGKEEPP
ARSSLFQEKANLYPPSNTPGDALSPGGGLRPNGQTKPLPALKLALEYIVP
CMNKHGICVVDDFLGKETGQQIGDEVRALHDTGKFTDGQLVSQKSDSSKD
IRGDKITWIEGKEPGCETIGLLMSSMDDLIRHCNGKLGSYKINGRTKAMV
ACYPGNGTGYVRHVDNPNGDGRCVTCIYYLNKDWDAKVSGGILRIFPEGK
AQFADIEPKFDRLLFFWSDRRNPHEVQPAYATRYAITVWYFDADERARAK
VKYLTGEKGVRVELNKPSDSVGKDVF

PLEK, GCID: GC02P068365 refers to a protein associated with protein homodimerization activity and phosphatidylinositol-3.4-biphosphate binding. A non-limiting exemplary sequence of the human protein provided below may be found under UniProtKB Ref No. P08567, accessible through the Gene Cards database (SEQ ID NO: 44):

MEPKRIREGYLVKKGSVFNTWKPMWVVLLEDGIEFYKKKSDNSPKGMIPL
KGSTLTSPCQDFGKRMFVFKITTTKQQDHFFQAAFLEERDAWVRDIKKAI
KCIEGGQKFARKSTRRSIRLPETIDLGALYLSMKDTEKGIKELNLEKDKK
IFNHCFTGNCVIDWLVSNQSVRNRQEGLMIASSLLNEGYLQPAGDMSKSA
VDGTAENPFLDNPDAFYYFPDSGFFCEENSSDDDVILKEEFRGVIIKQGC
LLKQGHRRKNWKVRKFILREDPAYLHYYDPAGAEDPLGAIHLRGCVVTSV
ESNSNGRKSEEENLFEIITADEVHYFLQAATPKERTEWIRAIQMASRTGK

PLAC8, GCID: GC04M083090 refers to a protein associated with metabolism, the immune system, and chromatin binding. A non-limiting exemplary sequence of the human protein provided below may be found under UniProtKB Ref No. Q9NZF1, accessible through the Gene Cards database (SEQ ID NO: 45):

MQAQAPVVVVTQPGVGPGPAPQNSNWQTGMCDCFSDCGVCLCGTFCFPCL
GCQVAADMNECCLCGTSVAMRTLYRTRYGIPGSICDDYMATLCCPHCTLC
QIKRDINRRRAMRTF

ATM, GCID: GC11P108127 refers to a protein closely related to kinase ATR, which belongs to the PI3/PI4 kinase family and functions as a regulator of a wide variety of downstream proteins, including tumor suppressor proteins p53 and BRCA1, checkpoint kinase CHK2, checkpoint proteins RAD17 and RAD9, and DNA repair protein NBS1. A non-limiting exemplary sequence of the human protein provided below may be found under UniProtKB Ref No. Q13315, accessible through the Gene Cards database (SEQ ID NO: 46):

MSLVLNDLLICCRQLEHDRATERKKEVEKFKRLIRDPETIKHLDRHSDSK
QGKYLNWDAVFRFLQKYIQKETECLRIAKPNVSASTQASRQKKMQEISSL
VKYFIKCANRRAPRLKCQELLNYIMDTVKDSSNGAIYGADCSNILLKDIL
SVRKYWCEISQQQWLELFSVYFRLYLKPSQDVHRVLVARIIHAVTKGCCS
QTDGLNSKFLDFFSKAIQCARQEKSSSGLNHILAALTIFLKTLAVNFRIR
VCELGDEILPTLLYIWTQHRLNDSLKEVIIELFQLQIYIHHPKGAKTQEK
GAYESTKWRSILYNLYDLLVNEISHIGSRGKYSSGFRNIAVKENLIELMA
DICHQVFNEDTRSLEISQSYTTTQRESSDYSVPCKRKKIELGWEVIKDHL
QKSQNDFDLVPWLQIATQLISKYPASLPNCELSPLLMILSQLLPQQRHGE
RTPYVLRCLTEVALCQDKRSNLESSQKSDLLKLWNKIWCITFRGISSEQI
QAENFGLLGAIIQGSLVEVDREFWKLFTGSACRPSCPAVCCLTLALTTSI
VPGTVKMGIEQNMCEVNRSFSLKESIMKWLLFYQLEGDLENSTEVPPILH
SNFPHLVLEKILVSLTMKNCKAAMNFFQSVPECEHHQKDKEELSFSEVEE
LFLQTTFDKMDFLTIVRECGIEKHQSSIGFSVHQNLKESLDRCLLGLSEQ
LLNNYSSEITNSETLVRCSRLLVGVLGCYCYMGVIAEEEAYKSELFQKAK
SLMQCAGESITLFKNKTNEEFRIGSLRNMMQLCTRCLSNCTKKSPNKIAS
GFFLRLLTSKLMNDIADICKSLASFIKKPFDRGEVESMEDDTNGNLMEVE
DQSSMNLFNDYPDSSVSDANEPGESQSTIGAINPLAEEYLSKQDLLFLDM
LKFLCLCVTTAQTNTVSFRAADIRRKLLMLIDSSTLEPTKSLHLHMYLML
LKELPGEEYPLPMEDVLELLKPLSNVCSLYRRDQDVCKTILNHVLHVVKN
LGQSNMDSENTRDAQGQFLTVIGAFWHLTKERKYIFSVRMALVNCLKTLL
EADPYSKWAILNVMGKDFPVNEVFTQFLADNHHQVRMLAAESINRLFQDT
KGDSSRLLKALPLKLQQTAFENAYLKAQEGMREMSHSAENPETLDEIYNR
KSVLLTLIAVVLSCSPICEKQALFALCKSVKENGLEPHLVKKVLEKVSET
FGYRRLEDFMASHLDYLVLEWLNLQDTEYNLSSFPFILLNYTNIEDFYRS
CYKVLIPHLVIRSHFDEVKSIANQIQEDWKSLLTDCFPKILVNILPYFAY
EGTRDSGMAQQRETATKVYDMLKSENLLGKQIDHLFISNLPEIVVELLMT
LHEPANSSASQSTDLCDFSGDLDPAPNPPHFPSHVIKATFAYISNCHKTK
LKSILEILSKSPDSYQKILLAICEQAAETNNVYKKHRILKIYHLFVSLLL
KDIKSGLGGAWAFVLRDVIYTLIHYINQRPSCIMDVSLRSFSLCCDLLSQ
VCQTAVTYCKDALENHLHVIVGTLIPLVYEQVEVQKQVLDLLKYLVIDNK
DNENLYITIKLLDPFPDHVVFKDLRITQQKIKYSRGPFSLLEEINHFLSV
SVYDALPLTRLEGLKDLRRQLELHKDQMVDIMRASQDNPQDGIMVKLVVN
LLQLSKMAINHTGEKEVLEAVGSCLGEVGPIDFSTIAIQHSKDASYTKAL
KLFEDKELQWTFIMLTYLNNTLVEDCVKVRSAAVTCLKNILATKTGHSFW
EIYKMTTDPMLAYLQPFRTSRKKFLEVPRFDKENPFEGLDDINLWIPLSE
NHDIWIKTLTCAFLDSGGTKCEILQLLKPMCEVKTDFCQTVLPYLIHDIL
LQDTNESWRNLLSTHVQGFFTSCLRHFSQTSRSTTPANLDSESEHFFRCC
LDKKSQRTMLAVVDYMRRQKRPSSGTIFNDAFWLDLNYLEVAKVAQSCAA
HFTALLYAEIYADKKSMDDQEKRSLAFEEGSQSTTISSLSEKSKEETGIS
LQDLLLEIYRSIGEPDSLYGCGGGKMLQPITRLRTYEHEAMWGKALVTYD
LETAIPSSTRQAGIIQALQNLGLCHILSVYLKGLDYENKDWCPELEELHY
QAAWRNMQWDHCTSVSKEVEGTSYHESLYNALQSLRDREFSTFYESLKYA
RVKEVEEMCKRSLESVYSLYPTLSRLQAIGELESIGELFSRSVTHRQLSE
VYIKWQKHSQLLKDSDFSFQEPIMALRTVILEILMEKEMDNSQRECIKDI
LTKHLVELSILARTFKNTQLPERAIFQIKQYNSVSCGVSEWQLEEAQVFW
AKKEQSLALSILKQMIKKLDASCAANNPSLKLTYTECLRVCGNWLAETCL
ENPAVIMQTYLEKAVEVAGNYDGESSDELRNGKMKAFLSLARFSDTQYQR
IENYMKSSEFENKQALLKRAKEEVGLLREHKIQTNRYTVKVQRELELDEL
ALRALKEDRKRFLCKAVENYINCLLSGEEHDMWVFRLCSLWLENSGVSEV
NGMMKRDGMKIPTYKFLPLMYQLAARMGTKMMGGLGFHEVLNNLISRISM
DHPHHTLFIILALANANRDEFLTKPEVARRSRITKNVPKQSSQLDEDRTE
AANRIICTIRSRRPQMVRSVEALCDAYIILANLDATQWKTQRKGINIPAD
QPITKLKNLEDVVVPTMEIKVDHTGEYGNLVTIQSFKAEFRLAGGVNLPK
IIDCVGSDGKERRQLVKGRDDLRQDAVMQQVFQMCNTLLQRNTETRKRKL
TICTYKVVPLSQRSGVLEWCTGTVPIGEFLVNNEDGAHKRYRPNDFSAFQ
CQKKMMEVQKKSFEEKYEVFMDVCQNFQPVFRYFCMEKFLDPAIWFEKRL
AYTRSVATSSIVGYILGLGDRHVQNILINEQSAELVHIDLGVAFEQGKIL
PTPETVPFRLTRDIVDGMGITGVEGVFRRCCEKTMEVMRNSQETLLTIVE
VLLYDPLFDWTMNPLKALYLQQRPEDETELHPTLNADDQECKRNLSDIDQ
SFNKVAERVLMRLQEKLKGVEEGTVLSVGGQVNLLIQQAIDPKNLSRLFP
GWKAWV

PTGDR, GCID: GC14P052267 refers to a member of the guanine nucleotide-binding protein (G protein)-coupled receptor (GPCR) superfamily, which are seven-pass transmembrane proteins that respond to extracellular cues and activate intracellular signal transduction pathways. A non-limiting exemplary sequence of the human protein provided below may be found under UniProtKB Ref No. Q13258, accessible through the Gene Cards database (SEQ ID NO: 47):

MKSPFYRCQNTTSVEKGNSAVMGGVLFSTGLLGNLLALGLLARSGLGWCS
RRPLRPLPSVFYMLVCGLTVTDLLGKCLLSPVVLAAYAQNRSLRVLAPAL
DNSLCQAFAFFMSFFGLSSTLQLLAMALECWLSLGHPFFYRRHITLRLGA
LVAPVVSAFSLAFCALPFMGFGKFVQYCPGTWCFIQMVHEEGSLSVLGYS
VLYSSLMALLVLATVLCNLGAMRNLYAMHRRLQRHPRSCTRDCAEPRADG
REASPQPLEELDHLLLLALMTVLFTMCSLPVIYRAYYGAFKDVKEKNRTS
EEAEDLRALRFLSVISIVDPWIFIIFRSPVFRIFFHKIFIRPLRYRSRCS
NSTNMESSL

PXN, GCID: GC12M120210 refers to a cytoskeletal protein involved in actin-membrane attachment at sites of cell adhesion to the extracellular matrix (focal adhesion). A non-limiting exemplary sequence of the human protein provided below may be found under UniProtKB Ref No. P49023, accessible through the Gene Cards database (SEQ ID NO: 48):

MDDLDALLADLESTTSHISKRPVFLSEETPYSYPTGNHTYQEIAVPPPVP
PPPSSEALNGTILDPLDQWQPSSSRFIHQQPQSSSPVYGSSAKTSSVSNP
QDSVGSPCSRVGEEEHVYSFPNKQKSAEPSPTVMSTSLGSNLSELDRLLL
ELNAVQHNPPGFPADEANSSPPLPGALSPLYGVPETNSPLGGKAGPLTKE
KPKRNGGRGLEDVRPSVESLLDELESSVPSPVPAITVNQGEMSSPQRVTS
TQQQTRISASSATRELDELMASLSDFKIQGLEQRADGERCWAAGWPRDGG
RSSPGGQDEGGFMAQGKTGSSSPPGGPPKPGSQLDSMLGSLQSDLNKLGV
ATVAKGVCGACKKPIAGQVVTAMGKTWHPEHFVCTHCQEEIGSRNFFERD
GQPYCEKDYHNLFSPRCYYCNGPILDKVVTALDRTWHPEHFFCAQCGAFF
GPEGFHEKDGKAYCRKDYFDMFAPKCGGCARAILENYISALNTLWHPECF
VCRECFTPFVNGSFFEHDGQPYCEVHYHERRGSLCSGCQKPITGRCITAM
AKKFHPEHFVCAFCLKQLNKGTFKEQNDKPYCQNCFLKLFC

DHRS3, GCID: GC01M012567 refers to a short-chain dehydrogenase/reductase (SDR) that catalyzes the oxidation/reduction of a wide range of substrates, including retinoids and steroids. A non-limiting exemplary sequence of the human protein provided below may be found under UniProtKB Ref No. 075911, accessible through the Gene Cards database (SEQ ID NO: 49):

MVWKRLGALVMFPLQMIYLVVKAAVGLVLPAKLRDLSRENVLITGGGRGI
GRQLAREFAERGARKIVLWGRTEKCLKETTEEIRQMGTECHYFICDVGNR
EEVYQTAKAVREKVGDITILVNNAAVVHGKSLMDSDDDALLKSQHINTLG
QFWTTKAFLPRMLELQNGHIVCLNSVLALSAIPGAIDYCTSKASAFAFME
SLTLGLLDCPGVSATTVLPFHTSTEMFQGMRVRFPNLFPPLKPETVARRT
VEAVQLNQALLLLPWTMHALVILKSILPQAALEEIHKFSGTYTCMNTFKG
RT

It is appreciated that for all the proteins disclosed herein, the short hand term may also refer to isoforms, orthologs, variants, and equivalents thereof, as well as the gene encoding the protein—whose sequence can be readily determined through reverse transcription of the exemplary protein sequence and/or by accessing the gene sequence provided in the Gene Cards database.

### MODES OF CARRYING OUT THE DISCLOSURE

To date, transcriptional studies of CD8⁺ T cells from cancer patients have analyzed cells in peripheral blood or metastatic sites^{8,9,10,11}. The precise state of CD8⁺ T cell activation, differentiation and function within primary tumors, where they are persistently challenged with tumor antigens, is poorly understood; however, this must be a key reference point from which to begin unraveling the biology of immune attack at the time of diagnosis, tumor progression and after intervention with immunotherapies. In order to fully characterize the molecular nature of immune responses at the tumor site, an unbiased approach was taken to define the global transcriptional profile of purified CD8⁺ TILs from well-characterized cohorts of patients with two epithelial cancers, non-small cell lung cancer (NSCLC) and head and neck squamous cell cancer (HNSCC).

The global gene expression profile of tumor-infiltrating CTLs (CD8⁺ TILs) in human cancers has not been fully characterized^{8,9,10,11}. To identify the core transcriptional signature of CD8+ TILs, RNA sequencing (RNA-Seq) of purified populations of CD8⁺ T cells present in tumor samples (CD8⁺ TILs) from human patients was performed. Disclosed herein are expression profiles, as set forth in Tables 1-13 herein, which characterize CD8+ TILs and their association with disease prognosis. Based on this information, Applicants arrived at the cells, compositions, and methods disclosed herein.

### Cells of Interest

Aspects of this disclosure relate to a cell that exhibits or is modified to exhibit one or more of the following characteristics:
(i) higher than baseline expression of one or more genes set forth in Table 1, Table 4, Table 7 and/or Table 8;
(ii) lower than baseline expression of one or more genes set forth in Table 1, Table 4, Table 7 and/or Table 8;
(iii) higher than baseline expression of genes involved in one or more pathways set forth in Table 5 and/or Table 9;
(iv) lower than baseline expression of genes involved in one or more pathways set forth in Table 5 and/or Table 9;
(v) higher than baseline expression of one or more genes set forth in Table 12; and/or
(vi) lower than baseline expression of one or more genes set forth in Table 13.

In some aspects the cell is an immune cell, such as but not limited to a tumor infiltrating lymphocyte (TILs), a tissue resident memory cell (T_{RM}), and/or a CD8+ T-cell.

It is understood that, in the aforementioned aspects and embodiments, baseline expression refers to normalized mean gene expression. Thus, in further embodiments, higher than baseline expression refers to at least about a 2-fold increase in expression relative to baseline expression and/or lower than baseline expression is at least about a 2-fold decrease in expression relative to baseline expression.

More generally, the term “baseline” is employed to refer to the condition of the cells absent exposure to a tumor or cancer. And, unless explicitly stated otherwise, terms of degree such as “higher” and “lower” are used in reference to a “baseline” value calculated thusly.

### Methods of Detection and Isolation

In aspects relating to cells aforementioned cells without further modification, detection of presence or absence of these cells may be used for diagnosis of, prognosis of, or determining suitable therapy for a cancer, tumor, or neoplasia in a subject.

For example, aspects disclosed herein relate to a method of determining the density of tumor infiltrating lymphocytes (TILs), optionally T-cells, in a cancer, tumor, or sample thereof comprising measuring expression of one or more gene selected from the group of 4-1BB, PD-1, or TIM3, or one or more genes selected from Table 12 in the cancer, tumor, or sample thereof, wherein higher than baseline expression indicates higher density of TILs in the cancer, tumor, or sample thereof, or one or more genes selected from Table 13 in the cancer, tumor, or sample thereof, wherein lower than baseline expression indicates higher density of TILs in the cancer, tumor, or sample thereof. Additional aspects relate to a method to determine the density of tissue-resident memory cells (T_{RM}), optionally T-cells, in a cancer, tumor, or sample thereof comprising measuring the level of CD103 or one or more genes selected from Table 12 in the cancer, tumor, or sample thereof, wherein higher than baseline levels of CD103 indicates a high density of T_{RM} in the cancer, tumor, or sample thereof, or one or more genes selected from Table 13 in the cancer, tumor, or sample thereof, wherein lower than baseline levels of CD103 indicates a high density of T_{RM} in the cancer, tumor, or sample thereof. In some method aspects, prognosis of a subject having cancer is determined based on the density of TILs and/or T_{RM} in the cancer or a sample thereof, i.e. wherein a high density of TILs and/or T_{RM} indicates an increased probability and/or duration of survival. As disclosed herein, measuring CD103 levels may be used to determine density of T_{RM}. Thus, density or frequency of CD103 may likewise serve as a prognostic indicator in the same manner as density of T_{RM}. Further, in embodiments relating to the density of TILs, these cells may be enriched for T_{RM}, for example by contacting the TILs with an effective amount of an active agent that induces higher than baseline expression of one or more genes set forth in Table 12 and/or an active agent that induces lower than base line expression of one or more genes set forth in Table 13 in TILs. As noted above, such an active agent may optionally be an antibody, protein, peptide, a small molecule, or a nucleic acid. It is appreciated that in such an enriched population, in some embodiments, the TILs enriched for T_{RM} have enhanced cytotoxicity and proliferation.

Further aspects relate to a method of diagnosing, determining prognosis in a subject, and/or responsiveness to cancer therapy by detecting the presence of one or more of:
(i) one or more genes set forth in Table 1, Table 4, Table 7 and/or Table 8, wherein higher than baseline levels is diagnostic of cancer and/or indicates an increased probability and/or duration of survival and/or indicates that the subject is likely to respond to cancer therapy;
(ii) one or more genes set forth in Table 1, Table 4, Table 7 and/or Table 8, wherein lower than baseline levels is diagnostic of cancer and/or indicates an increased probability and/or duration of survival and/or indicates that the subject is likely to respond to cancer therapy;
(iii) one or more genes set forth in Table 12, wherein higher than baseline levels is diagnostic of cancer and/or indicates an increased probability and/or duration of survival and/or indicates that the subject is likely to respond to cancer therapy; and/or
(iv) one or more genes set forth in Table 13, wherein lower than baseline levels is diagnostic of cancer and/or indicates an increased probability and/or duration of survival and/or indicates that the subject is likely to respond to cancer therapy.

In some embodiments, the T-cells are CD8+ and/or tumor infiltrating lymphocytes (TILs). Such embodiments include but are not limited to (i) to (ii) listed above. In some embodiments, the T-cells are tissue-resident memory cells (T_{RM}). Such embodiments include (iii) and (iv) listed above. In further embodiments of these aspects, the detection is conducted by contacting the cancer, tumor, or sample (as relevant) with an agent, optionally including a detectable label or tag. The detectable label or tag may comprise a radioisotope, a metal, horseradish peroxidase, alkaline phosphatase, avidin or biotin. Further, the agent may comprise a polypeptide that binds to an expression product encoded by the gene, or a polynucleotide that hybridizes to a nucleic acid sequence encoding all or a portion of the gene or that binds to an expression product encoded by the gene, or a polynucleotide that hybridizes to a nucleic acid sequence encoding all or a portion of the gene. In some aspects, the polypeptide comprises an antibody, an antigen binding fragment thereof, or a receptor that binds to the gene.

Further exemplary aspects are disclosed herein, including:
a method of determining prognosis of a subject having cancer, optionally lung cancer, comprising, or alternatively consisting essentially of, or yet further consisting of, contacting tumor infiltrating lymphocytes (TILs) of the cancer or a sample thereof with an antibody that recognizes and binds CD103 to determine the frequency of CD103+ TILs, or an antibody that recognizes and binds a protein encoded by a gene listed in Table 12 or Table 13, wherein a high frequency of CD103+ TILs or TILs expressing proteins encoded by a gene listed in Table 12 indicates an increased probability and/or duration of survival and low frequency of or TILs expressing proteins encoded by a gene listed in Table 13 indicates an increased probability and/or duration of survival;
a method of determining the responsiveness of a subject having cancer to immunotherapy comprising, or alternatively consisting essentially of, or yet further consisting of, contacting tumor infiltrating lymphocytes (TILs) of the cancer or a sample thereof with an antibody that recognizes and binds a protein encoded by a gene listed in Table 12 or Table 13, wherein a high frequency of TILs expressing proteins encoded by a gene listed in Table 12 indicates responsiveness to immunotherapy and low frequency of or TILs expressing proteins encoded by a gene listed in Table 13 indicates responsiveness to immunotherapy; a method of determining the responsiveness of a subject having cancer to immunotherapy comprising, or alternatively consisting essentially of, or yet further consisting of, contacting tumor infiltrating lymphocytes (TILs) of the cancer or a sample thereof with an antibody that recognizes and binds CD8, and antibody that recognizes and binds PD-1, an antibody that recognizes and binds TIM3, an antibody that recognizes and binds LAG3, and an antibody that recognizes and binds CTLA4 to determine the frequency of CD8⁺PD1⁺, CD8⁺TIM3⁺, CD8⁺LAG3⁺, CD8⁺CTLA4⁺CD8⁺PD1⁺TIM3⁺, CD8⁺PD1⁺LAG3⁺, CD8⁺PD1⁺CTLA4⁺, CD8⁺TIM3⁺LAG3⁺, CD8⁺TIM3⁺CTLA4⁺, CD8⁺LAG3⁺CTLA4⁺, CD8⁺PD1⁺TIM3⁺LAG3⁺, CD8⁺PD1⁺LAG3⁺CTLA4⁺, or CD8⁺PD1⁺TIM3⁺CTLA4⁺ TILs, wherein a high frequency of one or more of these TILs indicates responsiveness to immunotherapy;
a method of determining the responsiveness of a subject having cancer to immunotherapy comprising, or alternatively consisting essentially of, or yet further consisting of, contacting tumor infiltrating lymphocytes (TILs) of the cancer or a sample thereof with an antibody that recognizes and binds a protein encoded by a gene listed in Table 12 or Table 13, wherein a high frequency of TILs expressing proteins encoded by a gene listed in Table 12 indicates responsiveness to immunotherapy and low frequency of or TILs expressing proteins encoded by a gene listed in Table 13 indicates responsiveness to immunotherapy; and/or
a method of determining the responsiveness of a subject having cancer to immunotherapy comprising, or alternatively consisting essentially of, or yet further consisting of, contacting tumor infiltrating lymphocytes (TILs) of the cancer or a sample thereof with an antibody that recognizes and binds CD8, and antibody that recognizes and binds S1PR1, and an antibody that recognizes and binds KLF2 to determine the frequency of CD8+S1PR1− or CD8+KLF2− TILs, wherein a high frequency of one or more of these TILs indicates an increased responsiveness to immunotherapy.

It is appreciated that in any such embodiment disclosed herein, such as the exemplary embodiments of the paragraph above, similar embodiments may include the use of antibodies or detection of expression of one or more proteins encoded by one or more genes or related genes in pathways disclosed in Tables 1-13. Non-limiting exemplary embodiments thereof are described in the claims below.

In aspects where responsiveness to therapy—e.g. cancer therapy or immunotherapy—is assessed further embodiments may include the administration of the therapy to the subject being assessed. Non-limiting examples of cancer therapies include but are not limited to chemotherapy, immunotherapy, and/or radiation therapy.

Methods of detecting gene expression are well known in the art and can be readily adapted to the present disclosure. Such methods include but are not limited to Northern, Southern, and Western blotting, ISH, ELISA, X-ray, IHC, FISH, immunoprecipitation, immunofluorescence, chemiluminescence, radioactivity, X-ray, nucleic acid hybridization, protein-protein interaction, immunoprecipitation, flow cytometry, PCR, RT-PCR, qRT-PCR, SAGE, DNA microarray, DNA transcription, RNA Seq, and tiling arrays. Kits are available for carrying out such assays, such as but not limited to those produced by Thermo Fisher Scientific, Illumina®, QIAGEN, Life Technologies™, and other commercial vendors. In some embodiments, the gene expression may be detected at the transcriptional or translational level, i.e. either based on levels of mRNA transcribed or by levels of actual protein produced.

In general it is noted that agents or antibodies disclosed herein may be contacted with the cancer, tumor, or sample in conditions under which it can bind to the gene it targets to assess expression and/or presence of the aforementioned genes.

Methods of isolating relevant cells are well known in the art and can be readily adapted to the present disclosure. Isolation methods for use in relation to this disclosure include, but are not limited to Life Technologies Dynabeads® system; STEMcell Technologies EasySep™, RoboSep™, RosetteSep™, SepMate™; Miltenyi Biotec MACS™ cell separation kits, fluorescence activated cell sorting (FACS), and other commercially available cell separation and isolation kits. Particular subpopulations of immune cells may be isolated through the use of beads or other binding agents available in such kits specific to unique cell surface markers. For example, MACS™ CD4+ and CD8+ MicroBeads or complement depletion may be used to isolate CD4+ and CD8+ T-cells.

To the extent that samples are required in the method aspects disclosed herein they may optionally comprise comprises cells, tissue, or an organ biopsy; be an epithelial sample; originate from lung, respiratory or airway tissue or organ, a circulatory tissue or organ, a skin tissue, bone tissue, or muscle tissue; and/or originate from head, neck, brain, skin, bone, or blood.

### Methods of Modification

In aspects relating to cells that are modified to exhibit or isolated as exhibiting the traits disclosed herein, administration of these cells can be useful in the treatment of a cancer, tumor, or neoplasia in a subject. In some embodiments, the cells to be modified are isolated from the subject, and, thus, are autologous to the subject. In some embodiments, the cells to be modified are obtained from a source other than the subject (e.g. another subject, a cell line, or an “off-the-shelf” source of cells).

Some aspects relate to a modified T-cell, which is modified to exhibit one or more of:
(i) higher than baseline expression of one or more genes set forth in Table 1, Table 4, Table 7 and/or Table 8;
(ii) lower than baseline expression of one or more genes set forth in Table 1, Table 4, Table 7 and/or Table 8;
(iii) higher than baseline expression of genes involved in one or more pathways set forth in Table 5 and/or Table 9;
(iv) lower than baseline expression of genes involved in one or more pathways set forth in Table 5 and/or Table 9;
(v) higher than baseline expression of one or more genes set forth in Table 12; and/or
(vi) lower than baseline expression of one or more genes set forth in Table 13.

In some embodiments, the T-cells are CD8+. Such embodiments include but are not limited to (i) to (iv) listed above. In some embodiments, the T-cells are tissue-resident memory cells (T_{RM}). Such embodiments include (v) and (vi) listed above.

Methods of modifying gene expression are well known in the art and can be readily adapted to the present disclosure. For example, genes of interest may be packaged using a packaging vector and cell lines and introduced via a traditional recombinant methods. Alternatively or in addition, gene expression may be modified using a CRISPR/Cas9 system.

In some embodiments, the packaging vector may include, but is not limited to retroviral vector, lentiviral vector, adenoviral vector, and adeno-associated viral vector. The packaging vector contains elements and sequences that facilitate the delivery of genetic materials into cells. For example, the retroviral constructs are packaging plasmids comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer and/or promoter of the viral 5′ LTR of the virus, and lacks both the psi function sequence responsible for packaging helper genome and the 3′ LTR, but encodes a foreign polyadenylation site, for example the SV40 polyadenylation site, and a foreign enhancer and/or promoter which directs efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter.

The retroviral packaging plasmid may consist of two retroviral helper DNA sequences encoded by plasmid based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T cell leukemia (HTLV) type I and II env gene products, chimeric envelope gene derived from combinations of one or more of the aforementioned env genes or chimeric envelope genes encoding the cytoplasmic and transmembrane of the aforementioned env gene products and a monoclonal antibody directed against a specific surface molecule on a desired target cell. Similar vector based systems may employ other vectors such as sleeping beauty vectors or transposon elements.

Additional modifications can be made to the cell to render it more suitable for use in treatment. For example, the cells may be further modified to express or not express one or more antibodies, signaling molecules, receptors, or other immune effector in order to enhance their anti-cancer effect.

In some embodiments, the T-cell is further modified to express a protein that binds to a cytokine, chemokine, lymphokine, or a receptor each thereof and/or CD19. In further embodiments, this protein comprises an antibody or antigen binding fragment thereof, optionally wherein the antibody is IgG, IgA, IgM, IgE or IgD, or a subclass thereof or the antigen binding fragment is an Fab, Fab′, F(ab′)2, Fv, Fd, single-chain Fvs (scFv), disulfide-linked Fvs (sdFv) or V_{L} or V_{H}. Regarding antibodies, non-limiting exemplary subclasses of IgG relevant to aspects disclosed herein include but are not limited to IgG₁, IgG₂, IgG₃ and IgG₄.

### Compositions

Further aspects of the disclosure relate to a composition comprising one or more of the cells disclosed herein.

Briefly, pharmaceutical compositions of the present disclosure including but not limited to any one of the claimed compositions may comprise a target cell population as described herein, in combination with one or more pharmaceutically or physiologically acceptable carriers, diluents or excipients.

Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the disclosure. Those skilled in the art will know of other suitable carriers for binding antibodies, or will be able to ascertain such, using routine experimentation.

Such compositions may also comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for oral, intravenous, topical, enteral, and/or parenteral administration. In certain embodiments, the compositions of the present disclosure are formulated for intravenous administration.

Administration of the cells or compositions can be effected in one dose, continuously or intermittently throughout the course of treatment. Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. Suitable dosage formulations and methods of administering the agents are known in the art. In a further aspect, the cells and composition of the disclosure can be administered in combination with other treatments.

The cells and populations of cell are administered to the host using methods known in the art. This administration of the cells or compositions of the disclosure can be done to generate an animal model of the desired disease, disorder, or condition for experimental and screening assays.

Briefly, pharmaceutical compositions of the present disclosure including but not limited to any one of the claimed compositions may comprise a cell or population of cells as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for oral, intravenous, topical, enteral, and/or parenteral administration. In certain embodiments, the compositions of the present disclosure are formulated for intravenous administration.

Briefly, pharmaceutical compositions of the present disclosure including but not limited to any one of the claimed compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

Pharmaceutical compositions of the present disclosure may be administered in a manner appropriate to the disease to be treated or prevented. The quantity and frequency of administration will be determined by such factors as the condition of the patient, and the type and severity of the patient's disease, although appropriate dosages may be determined by clinical trials.

### Methods of Treatment

As disclosed hereinabove, the cells of the present disclosure may be used to treat cancer, tumor, and neoplasia. These cells may be administered either alone or in combination with diluents, known anti-cancer therapeutics, and/or with other components such as cytokines or other cell populations that are immunostimulatory.

Aspects of this disclosure relate to methods of treating cancer in a subject and/or eliciting an anti-tumor response comprising, or alternatively consisting essentially of, or yet further consisting of, administering to the subject and/or contacting the tumor or a tumor cell with, respectively, an effective amount of a population of T-cells that exhibit one or more of the following characteristics:
(i) higher than baseline expression of one or more genes set forth in Table 1, Table 4, Table 7 and/or Table 8;
(ii) lower than baseline expression of one or more genes set forth in Table 1, Table 4, Table 7 and/or Table 8;
(iii) higher than baseline expression of genes involved in one or more pathways set forth in Table 5 and/or Table 9;
(iv) lower than baseline expression of genes involved in one or more pathways set forth in Table 5 and/or Table 9;
(v) higher than baseline expression of one or more genes set forth in Table 12; and/or lower than baseline expression of one or more genes set forth in Table 13.

In some embodiments, the T-cells are CD8+ and/or tumor infiltrating lymphocytes (TILs). Such embodiments include (i) to (iv) but are not limited to listed above. In some embodiments, the T-cells are tissue-resident memory cells (T_{RM}). Such embodiments include (v) and (vi) listed above. Similar aspects relate to methods of treating cancer in a subject and/or eliciting an anti-tumor response comprising, or alternatively consisting essentially of, or yet further consisting of, administering to the subject and/or contacting the tumor to a tumor cell with, respectively, an effective amount of one or more an active agent that induces in T-cells:
(i) higher than baseline expression of one or more genes set forth in Table 1, Table 4, Table 7 and/or Table 8;
(ii) lower than baseline expression of one or more genes set forth in Table 1, Table 4, Table 7 and/or Table 8;
(iii) higher than baseline expression of genes involved in one or more pathways set forth in Table 5 and/or Table 9;
(iv) lower than baseline expression of genes involved in one or more pathways set forth in Table 5 and/or Table 9;
(v) higher than baseline expression of one or more genes set forth in Table 12; and/or
(vi) lower than baseline expression of one or more genes set forth in Table 13.

In some embodiments, the T-cells are CD8+ and/or tumor infiltrating lymphocytes (TILs). Such embodiments include but are not limited to (i) to (iv) listed above. In some embodiments, the T-cells are tissue-resident memory cells (T_{RM}). Such embodiments include (v) and (vi) listed above. In some embodiments, the active agent is an antibody, a small molecule, or a nucleic acid.

Additional aspects relate to methods of modulating protein expression in a subject or sample comprising, or alternatively consisting essentially of, or yet further consisting of, administering an effective amount of one or more an active agent that induces in T-cells, higher or lower than baseline expression of one or more proteins encoded by the genes set forth in any one of Tables 1-13 to the subject or sample, optionally one or more of:
(i) higher than baseline expression of one or more proteins encoded by genes set forth in Table 1, Table 4, Table 7 and/or Table 8;
(ii) lower than baseline expression of one or more proteins encoded by genes set forth in Table 1, Table 4, Table 7 and/or Table 8;
(iii) higher than baseline expression of proteins encoded by genes involved in one or more pathways set forth in Table 5 and/or Table 9;
(iv) lower than baseline expression of proteins encoded by genes involved in one or more pathways set forth in Table 5 and/or Table 9;
(v) higher than baseline expression of one or more proteins encoded by genes set forth in Table 12; and/or
(vi) lower than baseline expression of one or more proteins encoded by genes set forth in Table 13.

Additional aspects relate to methods of modulating protein activity in a subject or a sample comprising, or alternatively consisting essentially of, or yet further consisting of, administering an effective amount of one or more an active agent that modulates in T-cells, one or more proteins encoded by the genes set forth in any one of Tables 1-13 to the subject or sample, optionally one or more of:
(i) induce activity of one or more proteins encoded by genes set forth in Table 1, Table 4, Table 7 and/or Table 8;
(ii) inhibit activity of one or more proteins encoded by genes set forth in Table 1, Table 4, Table 7 and/or Table 8;
(iii) induce activity of one or more proteins encoded by genes involved in one or more pathways set forth in Table 5 and/or Table 9;
(iv) inhibit activity of one or more of proteins encoded by genes involved in one or more pathways set forth in Table 5 and/or Table 9;
(v) induce activity of one or more proteins encoded by genes set forth in Table 12; and/or
(vi) inhibit activity of one or more proteins encoded by genes set forth in Table 13.

In some embodiments, the method is effective for treating cancer in a subject and/or eliciting an anti-tumor response; thus, the method comprises, or alternatively consists essentially of, or yet further consists of, administering the agent to the subject and/or contacting the tumor or a tumor cell with the agent, respectively. In some embodiments, the T-cells are CD8+ and/or tumor infiltrating lymphocytes (TILs). Such embodiments include but are not limited to (i) to (iv) listed above. In some embodiments, the T-cells are tissue-resident memory cells (T_{RM}). Such embodiments include (v) and (vi) listed above. In some embodiments, the active agent is an antibody, a small molecule, or a nucleic acid.

Methods of modulating gene expression and/or protein expression are well known in the art. With regard to gene expression, agents can be used to silence genes through affecting gene regulation and/or methylation. The recombinant methods and CRISPR/Cas systems disclosed hereinabove may be useful in such methods. With regard to protein expression, agents can be used to affect protein expression at either the transcriptional level or the translational level (protein). Non-limiting examples of modulation at the transcriptional level include the use of interfering RNA molecules which disrupt transcription of the mRNA encoding the protein (to reduce expression) and/or the introduction of additional mRNA transcripts of the protein to increase production of the protein (to increase expression). Non-limiting examples of modulation at the translational level include the use of an agent that renders the protein unstable or otherwise non-functional for its putative function (to reduce expression) or the introduction of additional protein to increase the quantity of protein performing the putative function (to increase expression). Further methods of modulation include the use of active agents that affect downstream and/or upstream elements of the pathway in which the protein is involved.

Methods of assessing protein activity according the aspects disclosed herein are well understood in the art and include any protocol and/or assay designed to determine whether there has been an increase or decrease in the activity of a protein from the baseline of normal protein activity. Non-limiting examples of assays that are suitable are those that assess enzyme activity and/or catalysis; assess co-association and/or precipitation, assess phylphorylation/glycosylation/amidation/ubiquitination as a result of the protein, and/or any other appropriate mechanism related to the protein, e.g., where a protein functions along a specified pathway, assays analyzing levels of the relevant upstream pathway functions. In some embodiments, the change in activity is at least 0.1X, at least 0.2X, at least 0.3X, at least 0.4X, at least 0.5X, at least 1.0X, at least 1.25X, at least 1.5X, at least 2.0X, at least 2.5X, at least 3.0X, at least 3.5X, at least 4.0X, at least 4.5X, at least 5.0X, at least 5.5X, at least 6.0X, at least 6.5X, at least 7.0X, at least 7.5X, at least 8.0X, at least 8.5X, at least 9.0X, at least 9.5X, at least 10X fold.

The cells as disclosed herein may be administered either alone or in combination with diluents, known anti-cancer therapeutics, and/or with other components such as cytokines, chemokines, lymphokines, antibodies, or other cell populations that are immunostimulatory. They may be administered as a first line therapy, a second line therapy, a third line therapy, or further therapy. As such, the disclosed cells may be combined with other therapies (e.g., chemotherapy, radiation, etc.). Non-limiting examples of additional therapies include chemotherapeutics or biologics. Appropriate treatment regimens will be determined by the treating physician or veterinarian.

In some embodiments, the disclosed cells can be delivered or administered into a cavity formed by the resection of tumor tissue (i.e. intracavity delivery) or directly into a tumor prior to resection (i.e. intratumoral delivery). In some embodiments, the disclosed cells can be administered intravenously, intrathecally, intraperitoneally, intramuscularly, subcutaneously, or by other suitable means of administration.

Pharmaceutical compositions of the present disclosure can be administered in a manner appropriate to the disease to be treated or prevented. The quantity and frequency of administration will be determined by such factors as the condition of the patient, and the type and severity of the patient's disease, although appropriate dosages may be determined by clinical trials.

Kits

In one particular aspect, the present disclosure provides kits for performing any of the methods disclosed herein as well as instructions for carrying out the methods of the present disclosure such as detecting, isolating, or modifying cells and/or analyzing the results or administering the cells.

The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components can be packaged in a manner customary for use by those of skill in the art. For example, these suggested kit components may be provided in solution or as a liquid dispersion or the like.

The following examples are illustrative of procedures which can be used in various instances in carrying the disclosure into effect.

### EXAMPLES

Example 1—Immune Profiling of CD8+ Tumor Infiltrating Lymphocytes

### Results

### Major Transcriptional Changes Characterize Tumor-Infiltrating CTLs

To identify the core transcriptional signature of tumor infiltrating CTL's (CD8+ TILs), the inventors performed RNA sequencing (RNA-Seq) of purified populations of CD8⁺ T cells present in tumor samples (CD8+ TILs) from 36 patients with treatment-naïve early stage non-small cell lung cancer (NSCLC), categorized based on their histological subtype into adenocarcinoma and squamous cell carcinoma (Table 2). Matched transcriptional profiles of CD8⁺ T cells isolated from the adjacent non-tumor lung tissue (CD8⁺ N-TILs) were matched to discriminate features linked to lung tissue residence from those related to tumor infiltration. To assess the conservation of the transcriptional program of CD8⁺ TILs in a related solid tumor of epithelial-origin, a similar data set generated in 41 patients with head and neck squamous cell carcinoma (HNSCC) from both human papilloma virus (HPV)-positive (virally-driven) and HPV-negative subtypes was utilized (Table 2 and Table 3).

A large number of transcripts (n=1403) were identified that were differentially expressed by CD8⁺ TILs when compared to CD8⁺ N-TILs (Benjamini-Hochberg adjusted P<0.05 and 1.5-fold change (Table 4); indicating major changes in the transcriptional landscape of CD8⁺ TILs in lung tumor tissue. This set of ‘CD8⁺ TIL-associated transcripts’ reflects tumor-specific transcriptional programming as they were revealed by comparison with CD8⁺ N-TILs from uninvolved lung tissue; such a comparison excludes confounding factors introduced by lung tissue residence-related gene expression.

The expression of lung cancer ‘CD8⁺ TIL-associated transcripts’ did not differ according to histological subtype (adenocarcinoma versus squamous cell carcinoma). Principal component analysis (PCA) and hierarchical clustering also showed that CD8⁺ TILs from both subtypes of lung cancer mostly clustered together, distinct from the CD8⁺ N-TILs. Interestingly, this set of lung cancer ‘CD8⁺ TIL-associated transcripts’ were similarly expressed in CD8⁺ TILs in both subtypes of HNSCC, which also clustered together with CD8⁺ TILs from lung cancer, indicating a conserved TIL transcriptome for these two tumor types.

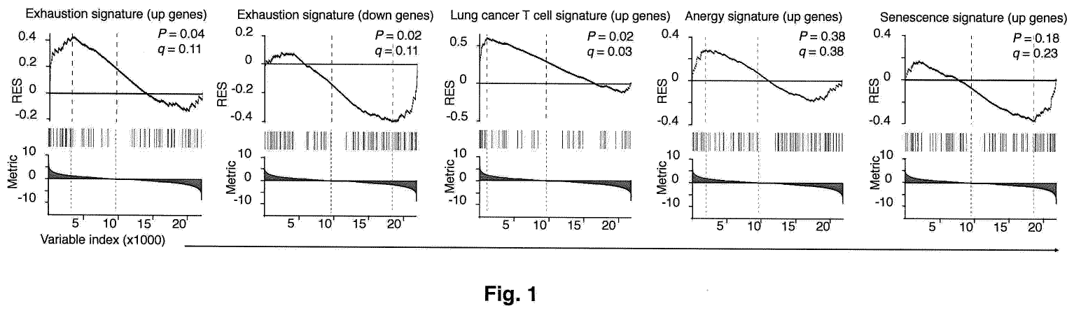

Features associated with inhibited T cell function, anergy and senescence have been described in TILs^{12, 13, 14}. Gene set enrichment analysis (GSEA) revealed significant enrichment of genes linked to the so-called exhaustion stage, such as PDCD1 (which encodes for PD1), CTLA4, HAVCR2 (which encodes for TIM3) and KLRG1, although some of these are also associated with activation, while genes associated with T cell anergy and senescence were not enriched FIG. 1). T cell-associated genes derived from The Cancer Genome Atlas (TCGA) of lung cancer¹⁵ were also enriched (FIG. 1). Together these findings suggest the strategy disclosed herein for micro-scaled RNA-Seq analysis of freshly purified ex vivo CD8⁺ TILs and CD8⁺ N-TILs reliably identifies transcripts previously linked to TILs.

### Cell Proliferation- and TCR Activation-Related Genes in CD8+ TILs

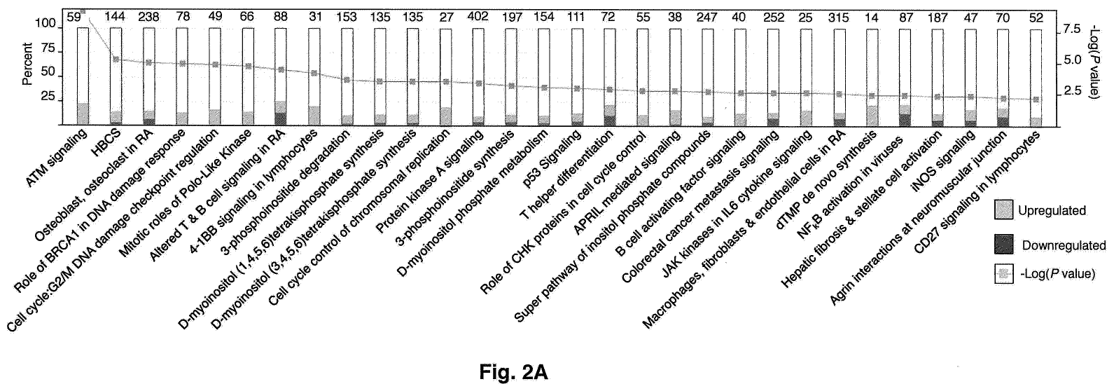

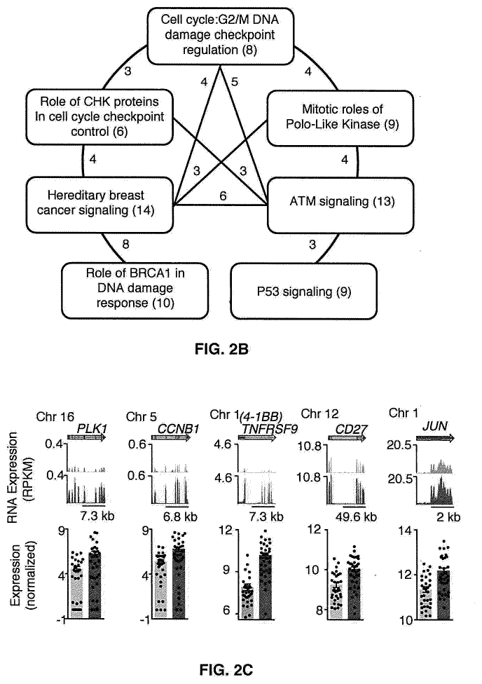

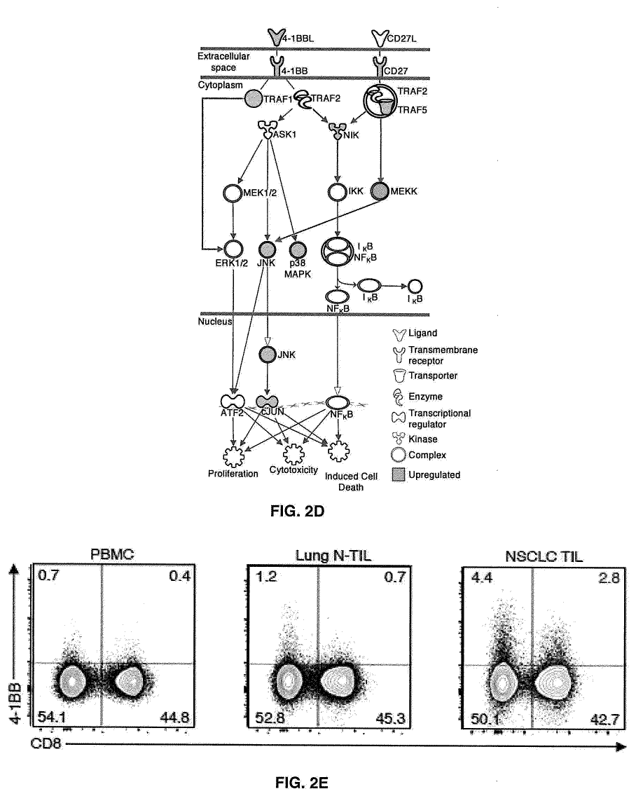

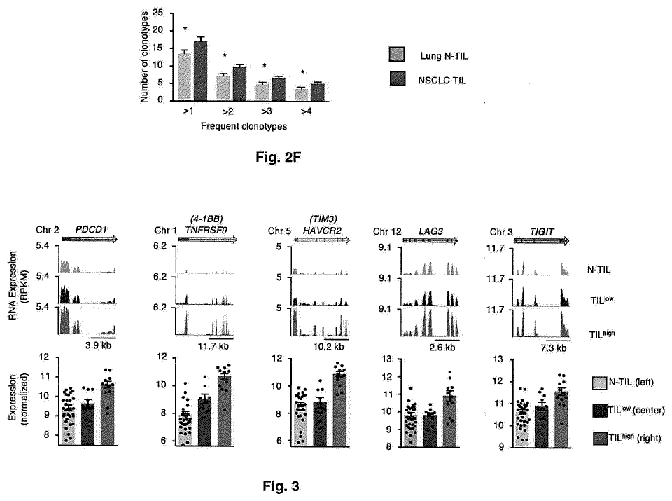

To gain broad insight into the functional relevance of the CD8⁺ TIL transcriptional program, gene pathway analysis was performed. Interestingly, in TILs, there was observed significant enrichment of transcripts encoding overlapping sets of genes involved in cell cycle control, mitosis, DNA replication and signaling via the tumor suppressor p53, ataxia telangiectasia mutated (ATM) and polo-like kinase (PLK) pathways (FIG. 2A-C and Table 5), indicating that proliferating CD8⁺ T cells are enriched in TILs (tumors) when compared to N-TILs (adjacent uninvolved lung tissue). Furthermore, the inventors observed enrichment of canonical pathways involved in antigen-specific T cell activation, especially the 4-1BB (tumor necrosis factor receptor superfamily member 9, TNFRSF9)-mediated and CD27 co-stimulatory pathways that are activated following T cell receptor (TCR) engagement and co-stimulation by antigen-presenting cells (APC), respectively^{16,17} (FIGS. 2A, 2D). The increased expression of 4-1BB in CD8⁺ TILs was confirmed at the protein level by flow cytometry (FIG. 2E). Together these data suggest that TCR engagement and co-stimulation, presumably provided by APCs expressing tumor-associated antigens (TAA), are likely to be involved in antigen-specific activation and proliferation of CD8⁺ TILs, implying that the tumor milieu sustains clonal expansion of presumed TAA-specific CD8⁺ T cells. This suggestion was further supported by analysis of the TCR repertoire, which indicated significantly greater clonal expansion of CD8⁺ TTLs compared to N-TILs (FIG. 2F, Table 6).

### Heterogeneity in the Expression of Immunotherapy Target Molecules

Immune checkpoint blockers such as anti-PD1 and anti-CTLA4 agents in humans and in model organisms^{4, 18} suggests that CD8⁺ TILs with features of TCR engagement and strong co-stimulation are likely to mount robust anti-tumor immune responses. However, the response to such treatments is highly variable and limited to a minority of patients. Although not wishing to be bound by theory, it was hypothesized that such inter-individual variability in response may be dictated by the underlying molecular profile of CD8⁺ TILs, which may also reveal other immune evasion mechanisms besides PD1 and CTLA-4-based pathways. Therefore, expression of a spectrum of potential immunotherapy target molecules was examined to uncover the extent of molecular heterogeneity in CD8⁺ TILs. Substantial variability was observed in the expression of transcripts encoding PD-1 and other potential targets of immunotherapy by CD8+ TILs from patients with lung cancer or HNSCC. The inventors confirmed PD-1 expression at the protein level and showed that the abundance of PDCD1 transcripts correlated with the average number of PD-1-expressing cells in the tumors. Varying combinations of expression of co-inhibitory molecules were also found: for example, CD8+ TILs from some patients with lung cancer had upregulation of transcripts encoding four targets of immunotherapy (PD-1, TIM-3, LAG-3 and CTLA-4) relative to the expression of those transcripts by other patients, while some patients showed upregulation of expression of three or two molecules or even a single molecule. The high molecular resolution and breadth of the data suggests that baseline transcriptional profiling of tumor-infiltrating CD8+ T cells might guide the selection of appropriate immunotherapies for each patient and the development of biomarkers that can be used to predict the clinical response to checkpoint blockade with monotherapy or combination therapies.

PDCD1 Expression Correlates with TIL Density

The marked heterogeneity observed in PDCD1 transcript levels led the inventors to investigate factors linked to PDCD1 expression in CD8⁺ TILs. Despite the perceived negative regulatory role of PD1 as an immune checkpoint, it serves as a marker for clonally expanded, antigen-specific T cells capable of lysing autologous tumor cells^{19, 20}. Furthermore, the inventors found a strong positive correlation between the expression of PDCD1 and 4-JB, a molecule expressed following TCR engagement and thus a marker of antigen-specific T cells^{16, 17, 21} The heterogeneity in the expression of these surrogate markers for antigen specificity suggests that not all tumors contain similar numbers of tumor-reactive CD8⁺ TILs. Hence, the inventors asked what factors might influence the enrichment of PDCD1- and 4-1BB-expressing CD8⁺ TILs, i.e. TAA-specific cells, in some patients. The inventors found no correlation of PDCD1 or 4-1BB transcript levels with clinical or pathological characteristics such as patient age, gender, histological subtype, stage of disease, performance status or smoking status. However, there was a positive correlation between the abundance of each of those transcripts and the average number of CD8+ TILs that infiltrated each tumor sample. A similar correlation was also observed between the abundance of each of those transcripts and CD8A transcripts (encoding the co-receptor CD8a) in lung-tumor samples from the TCGA RNA-Seq data set. In addition to their higher expression of PDCD1 and 4-1BB, tumors with a high density of TILs (‘TIL^{high}’, tumors; tumors were classified as TIL^{high}, TIL^{int} and TIL_{low} on the basis of the average number of CD8+ T cells that infiltrated the tumors; also had higher expression of transcripts encoding several other targets of immunotherapy, such as TIM-3, LAG-3 or TIGIT, than that of TIL_{low} tumors. Published studies have linked PD-1 and 4-1BB to both exhaustion²² and antigen-specific TCR activation^{19,20} but the positive correlation of their expression with TIL density indicated that their higher expression reflects enrichment for activated TAA-specific CD8+ T cells.

CD8⁺ T_{RM} Cells are Enriched in TIL^{high} Tumors

Patients with a high density of TILs in tumors have a better survival outcome than that of patients with low TIL density⁶. Besides the numerical changes in T cells, it is not known if there are qualitative differences in tumor-infiltrating CD8⁺ T cells between these groups, i.e. whether any molecular features in CD8⁺ TILs are unique to tumors with high TIL density. Defining such features provides insight into the mechanisms that govern the magnitude and specificity of anti-tumor CD8⁺ T cells responses.

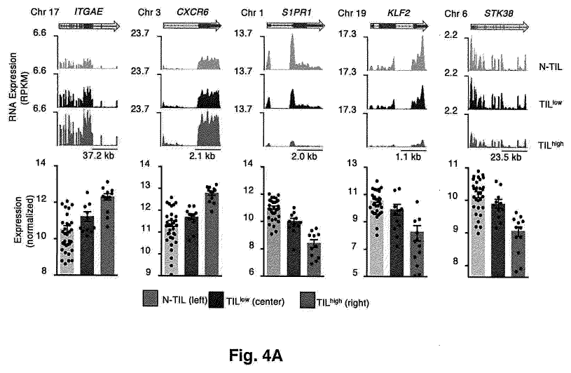

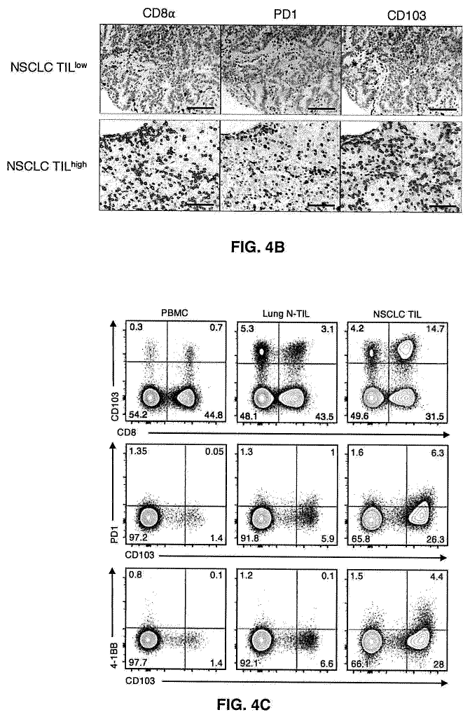

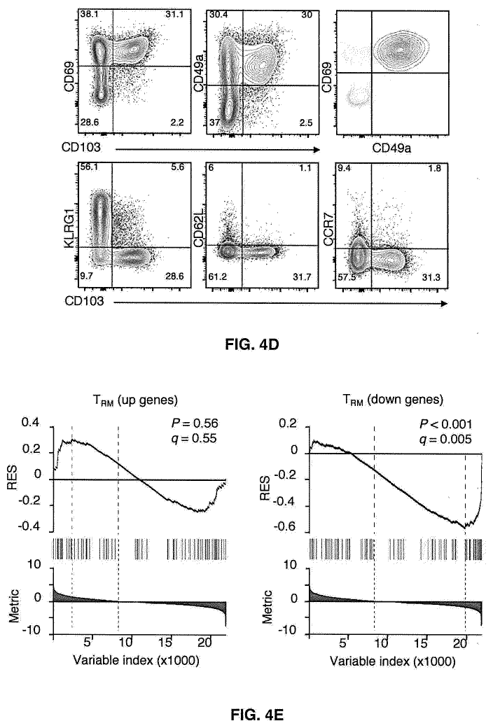

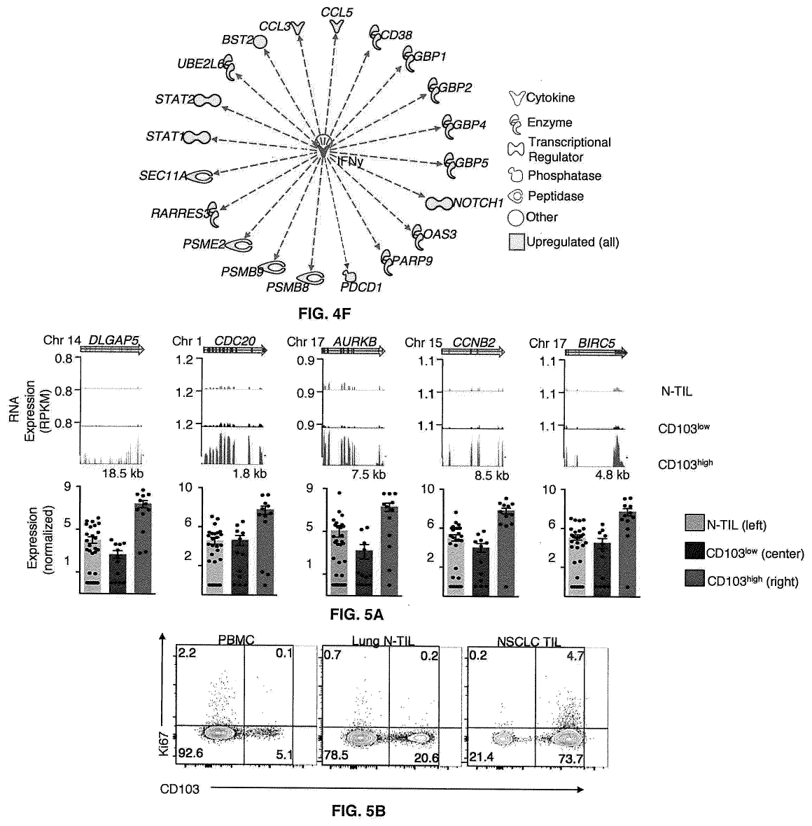

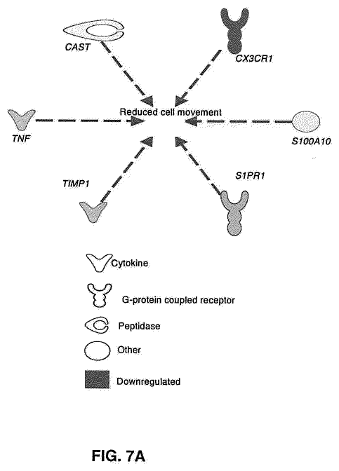

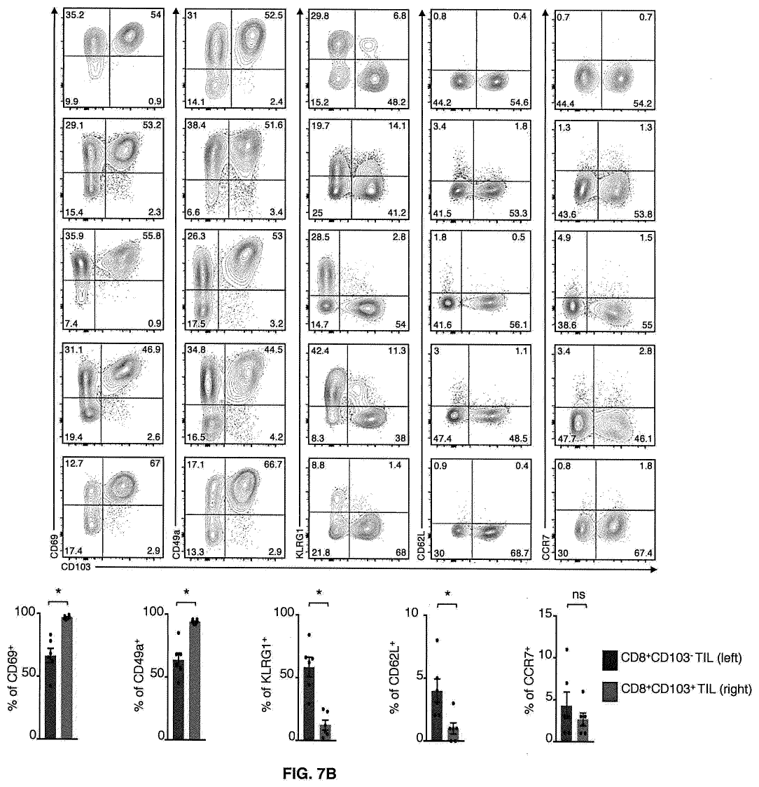

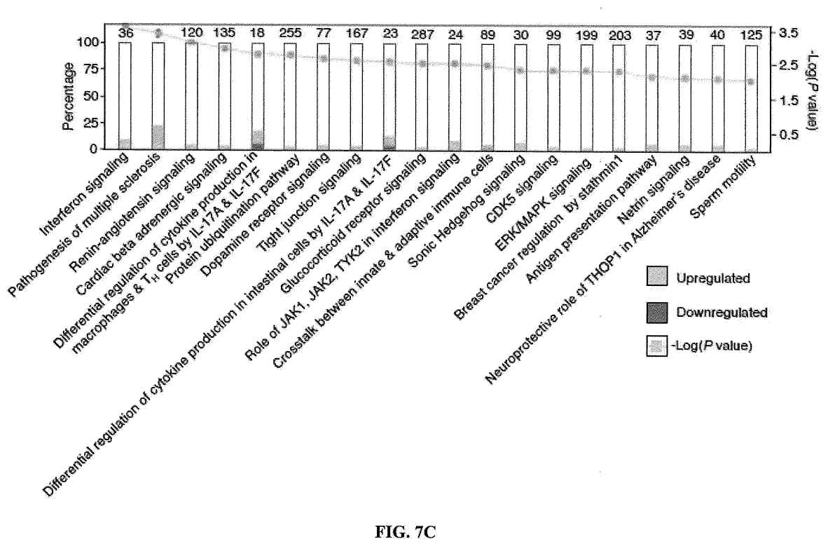

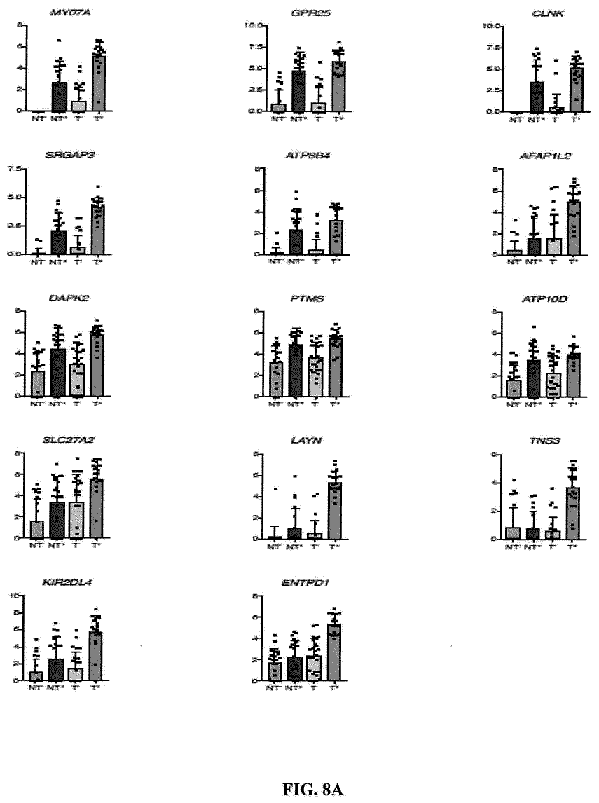

109 transcripts were found for which expression differed significantly between TIL^{high} versus TIL^{low} tumors (Benjamini-Hochberg adjusted P<0.05, Table 7). As expected, transcripts involved in TCR activation (4-1BB, PDCD1) were upregulated in TIL^{high} tumors, consistent with the enrichment of presumed TAA-specific CD8⁺ T cells. Several other transcripts associated with tissue retention of lymphocytes and tissue-resident memory T cells (T_{RM}) were differentially expressed in TIL^{high} tumors (Table 7). For example, ITGAE (CD103) encodes the α-subunit of the integrin molecule α_{E}β₇ (human mucosal lymphocyte-1 antigen), which binds the adhesion molecule E-cadherin expressed by epithelial cells in barrier tissues^{22,23}. Expression of this marker of T_{RM} cells was enriched in TIL^{high} tumors (FIG. 4A) and positively correlated with the average number of CD8⁺ cells within tumors in the patient cohort. This finding was also validated in the TCGA lung cancer data set. The inventors confirmed CD103 expression in CD8⁺ TILs at the protein level by immunohistochemistry and flow cytometry (FIGS. 4B, 4C). Surface molecules linked to T_{RM} cells^{25,26} such as CD69 and CD49a (ITGA1), were co-expressed with CD103, and surface molecules linked to effector memory cells (KLRG1) and central memory cells (CCR7 and CD62L) had lower expression on CD103+CD8+ TILs than on CD103−CD8+ TILs (FIGS. 4D and 7B), which suggested that the former population represented TRM cells. The inventors also observed co-expression of PD-1 and 4-1BB in 6% of CD103+CD8+ TILs and 4% of CD103+CD8+ TILs, respectively, in a representative patient sample (FIG. 4C).

Another transcript enriched in TIL^{high} tumors was CXCR6 (FIG. 4A), whose expression is not only linked to T_{RM} cells²⁴, but is also important for the localization and function of tissue-residing T cells^{25,26}. S1PR1 and KLF2 transcripts, known to be downregulated in T_{RM} cells²³, were also diminished in TIL^{high} tumors (FIG. 4A). Downregulation of S1PR1, which encodes sphingosine 1-phosphate receptor 1 (S1P1), is necessary for the egress of T cells from the lymph nodes and subsequent retention in tissues, as T cells expressing high levels of S1P1 are retained in the lymph nodes and also easily exit from tissues due to the higher levels of its ligand, sphingosine-1 phosphate (S1P) in the lymph nodes and blood. S1PR1 is a target gene of KLF2, a transcription factor; its downregulation has been shown to result in reduced S1PR1 expression, and both of these genes together play an important role in the establishment and retention of T_{RM} cells in tissues²⁷. Gene set enrichment analysis (GSEA) also revealed that TIL^{high} tumors express low levels of genes that are typically downregulated in a core set of T_{RM} signature genes, such as SIPR5, STK38, FAM65B^{23, 25} (FIG. 4E). Pathway analysis of the genes enriched in TIL^{high} tumors revealed a significant overrepresentation of genes involved in the canonical interferon (IFN) pathway (FIG. 7C), which was also predicted to be an upstream regulator by IPA upstream regulator analysis (FIG. 4F). Because IFN-γ produced by T_{RM} cells has been shown to recruit circulating T cells to potentiate robust immune responses in tissues^{28, 29}, the inventors, without being bound to any particular theory, infer that the IFN response signature seen in TIL^{high} tumors may be the result of T_{RM} activation by TAA (tumor-specific T_{RM} activity). Overall, these results demonstrate that T_{RM} cells are enriched in TIL^{high} tumors.

### CD103 Density Predicts Survival in Lung Cancer

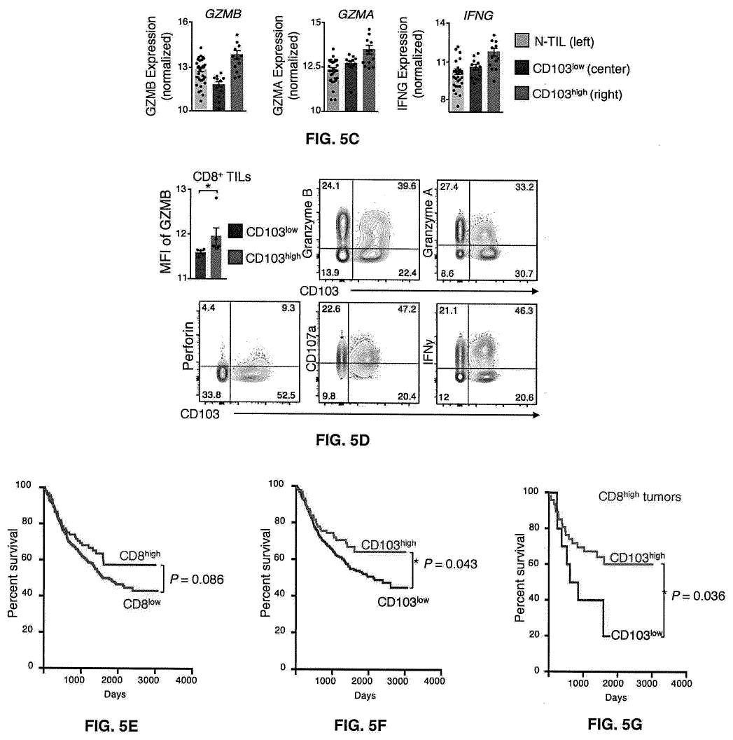

CD8⁺ TILs from tumors enriched for T_{RM} cells (CD103^{high}) were next examined for features that would support a robust (clinically-relevant) anti-tumor immune response. Ingenuity pathway analysis of the genes differentially expressed in CD103^{high} versus CD103^{low} TILs (classified based on the expression of ITGAE (CD103) transcripts in CD8⁺TILs, Table 8) pointed to cell proliferation and cytotoxicity as the key activated functions (Table 9). Consistent with this analysis, several transcripts linked to cell cycle and proliferation³⁰ were markedly upregulated in CD103^{high}CD8⁺ TILs. The inventors confirmed by flow cytometry that CD103+CD8+ TILs express the cell proliferation marker Ki67. Several transcripts linked to cytotoxic function of CD8⁺ T cells (IFNG, GZMA, GZMB, SEMA7A, KLRB1, CCL3, STAT1, RAB27A, IL21R, FKBP1A³¹) were also significantly upregulated in CD103^{high} tumors (FIG. 5C). The inventors confirmed at the protein level that CD103+CD8+ TILs expressed molecules linked to cytotoxicity, such as granzyme B, granzyme A, perforin and CD107a, and produced IFN-γ (FIG. 5D), and demonstrated that CD103+CD8+ TILs were the main producers, among CD8+ TILs, of both granzyme A and granzyme B. To address the question of whether CD8+ TILs from CD103^{high} tumors (Table 8) had greater effector potential, the mean fluorescence intensity of those molecules were compared against the frequency of cells expressing them in CD103^{high} tumors relative to that in CD103^{low} tumors (FIG. 5D). Notably, the inventors found that CD8+ TILs from CD103^{high} tumors had significantly higher expression of granzyme B than that of CD103^{low} tumors (FIG. 5D). These results suggested that tumors rich in TRM cells (CD103^{high} tumors) harbored CD8+ T cells that actively proliferated in the tumor milieu and displayed enhanced production of cytotoxic molecules, all hallmarks of robust anti-tumor immunity.

Based on this finding, but without wishing to be bound by any particular theory, it was hypothesized that a high density of CD103 in tumors (T_{RM}-enriched tumors) also confers a survival advantage beyond that previously found to be associated with CD8⁺ TIL density^{6,7}. In an independent large cohort of predominantly early stage lung cancer patients (n=689; 83% Stage I to IIIA, Table 10) followed up from 2007 to 2016, the inventors assessed retrospectively the survival outcome for patients whose tumors were classified based on the density of cells expressing CD8a or CD103 (Table 10). A higher density of CD8⁺ TILs was associated with a 28% reduction in mortality, although this did not reach statistical significance (Cox proportional hazards model, P=0.077; Kaplan-Meier plot with log-rank test P value is shown in FIG. 5E). Importantly, lung cancer patients with CD103^{high} tumors had significantly reduced mortality compared to those with CD103^{low} tumors (34% reduced risk of mortality, Cox proportional hazards model, P=0.045; Kaplan-Meier plot with log-rank test P value is shown in FIG. 5F). This finding was also observed in the TCGA data set for lung cancer. To better understand the dependence of CD103 and CD8 density in tumors, the inventors determined the status of CD103 density (CD10 3^{high}, CD103^{int}, CD103^{low}) in tumors pre-classified based on CD8 density. As expected, the proportion of CD103^{high} tumors was higher in CD8^{high} compared to CD8^{low} tumors; however, there is some discordance as tumors with CD103^{low} or CD103^{int} status were also observed in CD8^{high} tumors (FIG. 5G). Notably, even in the subgroup of lung cancer patients with high CD8⁺ TIL density (CD8^{high} tumors), patients with higher CD103 density had significantly reduced mortality (60% reduced risk of mortality, Cox proportional hazards model, P=0.043) and survived significantly longer compared to patients with CD103^{low} tumors (Kaplan-Meier plot with log-rank test P=0.036, FIG. 5G). These results suggest that patients with a robust intra-tumoral T_{RM} response have better long-term survival outcomes, and this effect is over and above that conferred by density of CD8⁺ TILs.

### New Molecules Linked to Tumor Immune Response

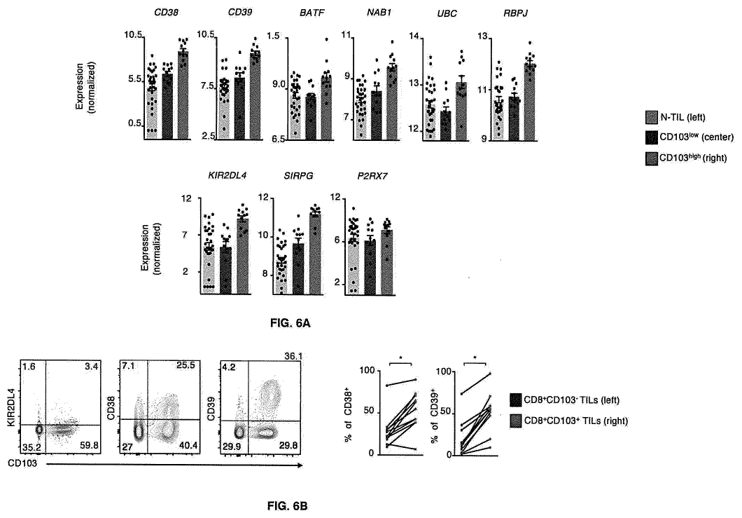

Transcripts for molecules that have been shown to be effective immunotherapy targets, such as PDCD1, TIM3 and LAG3, were among the most enriched in tumors with CD8^{high} and CD103^{high} TIL status, which were both independently linked to better anti-tumor immunity and survival outcomes. Therefore, the inventors have discovered that other molecules in the list of genes upregulated in tumors with CD8^{high} and CD103^{high} TIL status play an important functional role in modulating the magnitude and specificity of anti-tumor immune responses (Table 8). Some examples include CD39 (encoded by ENTPD1), a cell-surface ectonucleotidase that dephosphorylates ATP to AMP (FIG. 6A). The inventors found that the expression of CD39 protein was much higher in CD103+CD8+ TILs than in CD103−CD8+ TILs (FIG. 68). High concentrations of ATP in the tumor microenvironment can have toxic effects on cells via signaling through the purinergic receptor P2RX7^{33,34}. Given that CD8+ TILs from CD103^{high} tumors and those from CD103^{low} tumors exhibited similar expression of transcripts encoding P2RX7 (FIG. 6A), the inventors, without being bound to any particular theory, speculated that the greater abundance of CD39 ‘preferentially’ protects TRM cells (CD103+CD8+ TILs) from ATP-induced cell death. Notably, however, adenosine produced by CD39 might also suppress the function of natural killer T cells, natural killer cells and CD8+ T cells^{35,36}. CD38 is another ectonucleotidase and type II trans-membrane glycoprotein with various functions, including regulation of adenosine signaling, adhesion and transduction of activation and proliferation signals^{37,38}. Expression of CD38 protein was also higher in CD103+CD8+ TILs than in CD103-CD8+ TILs (FIG. 6B). Given that purinergic receptors can be targeted therapeutically, it might be pertinent to determine how CD39 and CD38 modulate ATP and purinergic signaling path-ways to influence the development and function of anti-tumor TRM cells (CD103+CD8+ TILs).

CD8+ TILs from CD10 3^{high} tumors had higher expression of several transcripts encoding components of the Notch signaling pathway (NOTCH, RBPJ, DTX2, UBC and UBB), relative to their expression in CD8+ TILs from CD103^{low} tumors (FIG. 6A), suggestive of an important role for this pathway in boosting TRM cell responses in lung cancer; this speculation is supported by a report showing that the Notch pathway supports the development of TRM cells in the lungs³⁹. CD8+ TILs from CD103^{high} tumors had higher expression of transcripts encoding two transcription factors (BATF and NAB1) potentially linked to CD4+ T cell-mediated help of CD8+ T cells, relative to their expression in CD8+ TfLs from CD103^{low} tumors (FIG. 6A).

Other examples of transcripts upregulated in CD103^{high} CD8⁺ TILs include KIR2DL4, which encodes a killer cell immunoglobulin-like receptor KIR2DL4 with activating and inhibitory functions³¹; expression of KIR2DL4 protein was confirmed in CD103+CD8+ TILs (FIG. 6D). HLA-G, a non-classical MHC class I molecule, has been shown to engage KIR2DL4 and increase cytokine and chemokine production by NK cells³² Though the expression of HLA-G is highly restricted, several reports have shown its increased expression in tumor tissue, especially in lung cancer³³, and therefore, without being bound to a particular theory, the inventors hypothesize that HLA-G expressed in tumors conveys activation signals via the KIR2DL4 receptor to CTLs and thus enhance their anti-tumor activities. SIRPG encodes for SIRPG, a member of the immunoglobulin superfamily of signal-regulatory proteins (SIRPs) that interact with the ubiquitously expressed CD47 molecule³⁴. Interestingly, SIRPG is the only member of the SIPR family that is expressed on T cells, and its interaction with CD47 expressed on APCs was shown to enhance T cell proliferation and IFN-γ production³⁵. Based on the increased expression of SIRPG transcripts in CD103^{high}CD8⁺ TILs (FIG. 6A), SIRPG serves as an important co-stimulatory molecule and its function could be exploited to enhance anti-tumor function of CTLs. Overall, these examples highlight the value of this large data set of CD8⁺ TIL transcriptional maps.

### DISCUSSION

An unbiased discovery-based approach was undertaken to identify transcripts that are enriched in CD8⁺ TILs and those that are linked to robust anti-tumor immune responses and good outcomes. Prior transcriptional studies of anti-tumor CD8⁺ T cells from patients with cancer have been largely restricted to analysis of whole tumor tissue or CD8⁺ T cells in peripheral blood or metastatic sites^{8, 9, 10, 11}. Further, most of those patients had advanced disease and were heavily pre-treated with chemotherapy or immunotherapies. Thus, these studies may not fully capture the molecular program of CD8⁺ T cells generated de novo at the primary tumor site, which is the focal point for immunotherapies. Further, studies that compare the transcriptional profile of tumor-infiltrating CD8⁺ T cells with their circulating counterparts are most likely to capture features linked to tissue residency rather than those linked to tumor infiltration (anti-tumor function/response). This study design avoided these confounding factors by using ‘micro-scaled’ RNA-Seq assays to generate transcriptomic maps of purified populations of CD8⁺ TILs and CD8⁺ T cells from adjacent non-involved lung tissue (N-TILs) from treatment-naïve patients with well-characterized early stage lung cancer. Bioinformatic analysis of these data sets revealed a core CD8⁺ TIL transcriptional profile comprising of ˜1400 genes that is shared across different tumor subtypes and is distinct from N-TILs, i.e. excluding differences that arise merely from lung tissue residency. This profile suggests extensive molecular reprogramming within the tumor microenvironment and the enrichment of presumably TAA-specific cells that are actively proliferating following TCR engagement and co-stimulation, all hallmarks of effective anti-tumor immunity.

In purified CD8⁺ TIL populations for the analyses, there was significant heterogeneity in the expression of cell cycle, TCR activation, co-stimulation and inhibitory genes across patients. This underlying molecular heterogeneity in anti-tumor CTL response addresses the variability in clinical responses to currently available immune checkpoint blockers. As set forth herein, baseline transcriptional profiling of purified tumor-infiltrating CTLs is a means of rationally selecting immunotherapies. The strategy disclosed herein of purifying relevant immune-cell populations from relatively small tumor samples and performing ‘micro-scaled’ RNA-Seq assays to generate high-resolution genome-wide data can be readily applied to any accessible tumor type. This approach can thus be used to develop biomarkers of the response to immunotherapy and to discover novel targets for immunotherapy. Another unique aspect of the present disclosed study is the inventor's evaluation of CD8+αTIL transcriptomes relative to TIL density (a feature linked to outcome). This analysis revealed various features linked to robust anti-tumor immune responses, such as TIL density; the most striking of these was tissue residence. CD8+ TILs with enrichment for TRM cells (CD103^{high}) had features of enhanced cytotoxicity and proliferation, which suggested that patients whose tumors had a high density of TRM cell markers, such as CD103, had a more-robust anti-tumor immune response and that this feature in the tumor might independently influence clinical outcome. In a large, independent cohort of patients with lung cancer, the inventors showed that a higher density of cells expressing CD103 was predictive of a better survival outcome. Most notably, the inventors confirmed that this effect was independent of that conferred by the density of CD8+ TILs; this finding was biologically relevant and has not been addressed by published studies-. Thus, the present disclosure has not only revealed a close link among TIL density, TRM cell features and enhanced survival but has also shed light on the global molecular features that endow CD8+ TILs from TRM cell-rich tumors with robust anti-tumor properties. Accordingly, the generation of a robust anti-tumor TRM cell response is an important goal of vaccination approaches targeting neo antigens or shared tumor antigens.

Since patients with lung cancer who had a high density of CD8+ or CD103+ TILs had a better survival outcome, the comparison of the transcriptional profiles of CD 8+ TILs from tumors with either a high density or a low density of cells expressing CD)8 or CD103 highlights features linked to the generation of robust anti-tumor immunity. The list of transcripts expressed differentially included those encoding molecules such as PD-1, TIM-3, CTLA-4, LAG-3, CD27, CD8 and OX40, which are effective targets of cancer immunotherapy in humans or in model organisms. Other molecules in that list might also have an important role in modulating the magnitude and specificity of anti-tumor immune response. For example, several promising molecules that were identified, such as CD38, CD39, BATF, NAB1, KIR2DL4, SIPRG and components of Notch signaling, are promising as immunotherapeutic targets in cancer. BATF has been shown to regulate the metabolism and survival of CD8+ T cells and to diminish the inhibited phenotype of CD8+ T cells^{48,49}. In a model of infection with lymphocytic choriomeningitis virus, the expression of BATF in CD8+ T cells, induced by the cytokine IL-21 derived from CD4⁺ T cells, was shown to be essential for maintaining the effector response of CTLs, and overexpression of BATF restored the effector function of CD8+ T cells that had not received help from CD4+ T cells49. NAB1 is a transcription factor whose mouse homolog (NAB2) is induced in CD8+ T cells that have received help from CD4+ T cells and is needed to prevent activation-induced cell death of those ‘helped’ CD8+ T cells50. Thus, without being bound to a particular theory, NAB1, which has high sequence homology to NAB2, has a similar role in preventing the apoptosis of tumor-infiltrating CTLs and that its increased expression might identify tumors in which CD8+ TILs have received help from CD4+ T cells.

The present disclosure reveals the transcriptional program of CD8+ TILs at the tumor site and has identified the inter-patient heterogeneity that presumably underlies the variability in clinical responses to checkpoint blockade. It has provided insight into the molecular mechanisms that govern robust anti-tumor CTL responses and lends support to the proposal that anti-tumor vaccines should be designed to enable the generation of CD8+ TRM cells for durable immunity. The ability to perform ‘micro-scaled’ RNA-Seq analysis of purified CD8+ TILs from patients' tumors allowed the inventors to identify gene-expression programs that might inform personalized immunotherapeutic treatment strategies and thereby provide a useful tool for translational application.

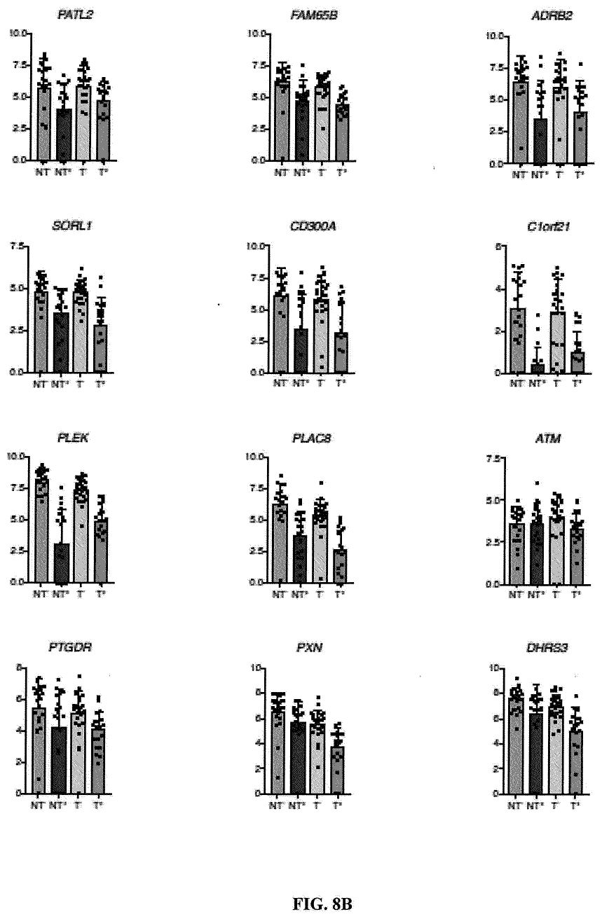

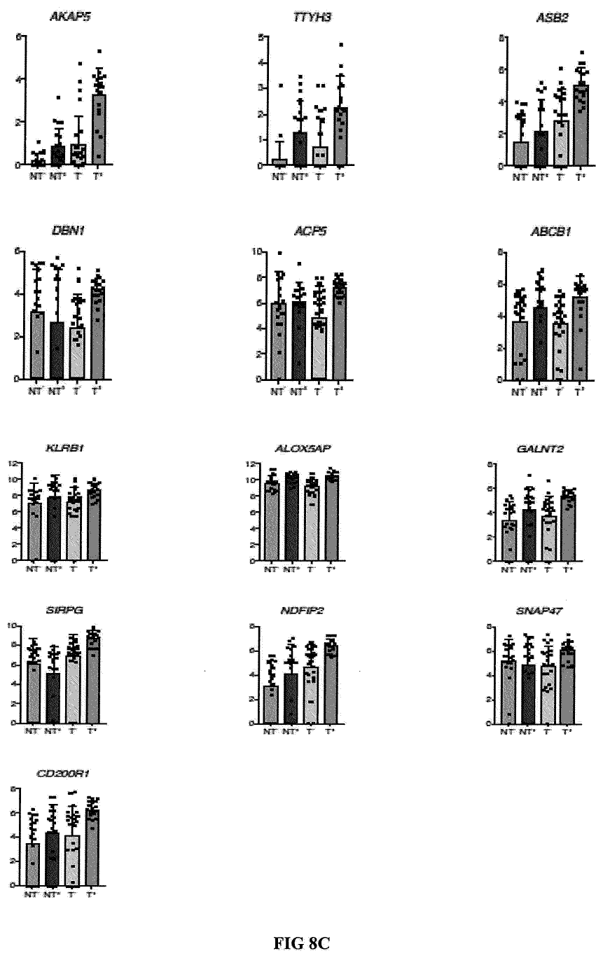

Further characterization was performed to determine differentially expressed genes in T_{RM} cells. RNA-seq analysis in a purified population of T_{RM} cells (CD8+C103+) and non-T_{RM} cells (CD8+C103−) from lung tumor and adjacent uninvolved lung (n>20). A total of 27 genes showed increased expression in T_{RM} cells and 12 genes showed reduced expression in T_{RM} cells (Table 12, Table 13). Based on this unique expression pattern, these molecules are deemed important in T_{RM} cells (FIGS. 8A-C, mean and individual expression levels (dots) from each patient).

Materials and Methods

### Patient Characteristics and Sample Processing.

Written informed consent was obtained from all subjects. Newly diagnosed, untreated patients with NSCLC and HNSCC (Table 2) referred to Southampton University Hospitals NHS Foundation Trust and Poole Hospital NHS Foundation trust, UK between 2014 and 2016 were prospectively recruited. Freshly resected tumor tissue and matched adjacent non-tumor lung tissue (in the case of patients with NSCLC) was obtained following surgical resection. T cells were isolated from tumor (TILs) or adjacent uninvolved lung (N-TILs) using a combination of mechanical and enzymatic dissociation. In brief, tumor or lung tissue was cut into small fragments and incubated at 37° C. for 15 min in an orbital shaker with 2 ml RPMI-1640 medium (Fisher Scientific) containing 0.15 WU/ml Liberase DL (Roche) and 800 units/ml DNase I (Sigma-Aldrich). Dispersed cells were then passed through a 70-μm filter and centrifuged and were re-suspended in MACS buffer (phosphate-buffered saline containing 2 mM EDTA and 0.5% bovine serum albumin) for sorting or analysis by flow cytometry. For isolating and phenotyping of CD8+ T cells from tumor or lung tissue, dispersed cells were first incubated with FcR block (Miltenyi Biotec), then were stained with a mixture of the following fluorescence-conjugated antibodies (each at the concentration recommended by the manufacturer): anti-CD45-FITC (H130; BioLegend), anti-CD4-PE (RPA-T4; BD Biosciences), anti-CD3-PE-Cy7 (SK7; BioLegend), anti-CD8α-PerCP-Cy5.5 (cSK1; BD Biosciences), anti-HLA-DR-APC (1243; BD Biosciences), anti-CD14-APC-H7 (MϕP9; BD Biosciences), anti-CD19-PerCP-Cy5.5 (clone HIB19; BioLegend) and anti-CD20-PerCP-Cy5.5 (clone 2H7; BioLegend). Stained samples were analyzed with a BD FACSAria (BD Biosciences) and FlowJo software (Treestar), and CD8+ T cells were sorted into ice-cold TRIzol LS reagent (Ambion)51,52. Phenotypic analysis of CD8+ TILs for T_{RM} markers was performed by staining with anti-CD69-BV605 (FN50; BioLegend), anti-CD49a-PE (TS2/7; BioLegend), anti-KLRG1-APC (SA231A2; BioLegend), anti-CD62L-BV510 (DREG-56; BioLegend), anti-CCR7-AF700 (TS2/7; BioLegend) (each at the concentration recommended by the manufacturer). Flow-cytometry analysis of CD8+CD103+ T cells and intra-cellular assessment of Ki67 were carried out with the following antibodies (each at the concentration recommended by the manufacturer): anti-CD45-FITC (HI30; BioLegend), anti-Ki67-PE (Ki67; BioLegend), anti-CD3-APC-Cy7 (SK7; BioLegend), anti-CD8α-PerCP-Cy5.5 (SK1; BD Biosciences), anti-CD103-APC (Ber-ACT8; BioLegend), anti-PD-1-PE-Cy7 (eBioJ105; eBioscience), anti-4-1BB-Pacific blue (4B4-1; BioLegend). The True-Nuclear Transcription Factor Buffer set (BioLegend) was used for the intracellular staining of Ki67. Flow-cytometry analysis of novel molecules and intracellular assessment of cytotoxic molecules were performed using the following antibodies (each at the concentration recommended by the manufacturer): anti-granzyme A-APC (CB9; BioLegend), anti-granzyme B-PE (REA226; Miltenyi Biotec), anti-Perforin-PE or -BV421 (B-D48; BioLegend), anti-KIR2DL4-PE (mAb33; BD BioLegend), anti-CD38-APC-Cy7 (1HB-7; BioLegend), anti-CD39-PE (A1; BioLegend). For cytokine and CD107a assays, CD8+ TILs were stimulated ex vivo with 20 nM PMA (phorbol 12-myristate 13-acetate) and 1 μM ionomycin for 4 b, and 5 μg/ml brefeldin was added during the final 2 h of stimulation. Anti-CD107a-PE (H4A3; BioLegend; at the concentration recommended by the manufacturer) was added to the PMA-and-ionomycin stimulation mixture for the final 2 h. Intracellular assessment of interferon-γ was performed using anti-IFNG-BV-421 (4S.B3; BioLegend; at the concentration recommended by the manufacturer) at the end of stimulation. Assays were performed in at least six patients and representative plots are presented. Stained samples were analyzed using a BD FACSCanto II (BD Biosciences). Dead cells were excluded using a LIVE/DEAD Fixable Aqua dead cell stain kit (Life Technologies) or DAPI (4,6-diamidino-2-phenylindole).

### Histology and Immunohistochemistry

Immunohistochemistry (IHC) was performed on FFPE tumor sections against CD8a (clone: C8/144B, Dako), CD103 (clone: ab129202, Abcam) and PD1 (clone: ab52587. Abcam). TILs were quantified using a Zeiss AxioCam MRc5 microscope (Zeiss, Cambridge, UK) and Zeiss Axiovision software (version 4.8.1.0; Zeiss). An average of 10 high-power (×400) fields across representative areas of each tumor was counted to account for intratumoral heterogeneity; these were averaged to generate an intratumoral TIL score. Tumors with an average CD8 count in the top ⅓ or bottom ⅓ percentile were classified as TIL^{high} or TIL^{low}, respectively; the lowest CD8 count in the TIL^{high} tumors was at least 2-fold greater than the highest CD8 count in the TIL^{low} tumors. For overall survival analyses (FIGS. 5C-5E), tumor tissue microarrays from NSCLC patients were stained with anti-CD8a (clone: C8/144B, Dako) and anti-CD103 (clone: ab129202, Abcam) antibodies and viewed under low-power magnification (×2.5 objective) to determine CD8 and CD103 density, as described previously⁵⁰.

### Survival Data and Analysis

In an independent large cohort of predominantly early stage NSCLC patients (n=689, Table 10) followed up from January 2007 to June 2016 (minimum follow up 3.4 years) the inventors retrospectively analyzed survival according to CD8 and CD103 TIL density. The primary endpoint was overall survival, and survival time was measured from the date of diagnosis until date of death or date last seen alive. Kaplan-Meier plots (with log-rank tests to determine significance of overall survival, P values shown in FIGS. 5C-5G) and unadjusted Cox proportional hazards model (to determine relative risk of death) were used to analyze the survival data, as described previously⁵¹. Patients were excluded from analysis if survival was <30 days to exclude possibility of surgery-related mortality. Survival analysis based on the expression of ITGAE (CD103) transcripts in tumor samples from lung adenocarcinoma patients in the TCGA was derived from http://www.oncolnc.org.

RNA sequencing. Total RNA was purified using a miRNAeasy micro kit (Qiagen, USA) and quantified as described previously⁵². Purified total RNA (5 ng) was amplified following the smart-seq2 protocol⁵². cDNA was purified using AMPure XP beads (1:1.1 ratio, Beckman Coulter). From this step, 1 ng of cDNA was used to prepare a standard Nextera XT sequencing library (Nextera XT DNA sample preparation kit and index kit, Illumina). Samples were sequenced using HiSeq2500 (Illumina) to obtain 50-bp single-end reads. Quality control steps were included to determine total RNA quality and quantity, optimal number of PCR pre-amplification cycles, and cDNA fragment size. Samples that failed quality control were eliminated from further downstream steps.

### RNA-Seq Analysis.

RNA-Seq data was mapped against the hg19 reference using TopHat⁵³ (v1.4.1., —library-type fr-secondstrand -C) and the RefSeq gene annotation downloaded from the UCSC Genome Bioinformatics site. Sequencing read coverage per gene was counted using HTSeq-count (-m union-s yes-t exon-i gene_id, http://www-huber.embl.de/users/anders/HTSeq/). To identify genes differentially expressed between patient groups, the inventors performed negative binomial tests for paired and unpaired comparisons by employing the Bioconductor package DESeq2 disabling the default options for independent filtering and Cooks cutoff⁵⁴. The inventors considered genes differentially expressed between any pairwise comparison when the DESeq2 analysis resulted in a Benjamini-Hochberg-adjusted P value <0.05. The Qlucore Omics Explorer 3.2 software package was used for visualization and representation (heat maps, principal component analysis) of RNA-Seq data⁴⁹. Unsupervised hierarchical clustering of samples based on the expression of genes (n=1,000) with the highest variance, which accounted for 20% of the total variance, was performed using DESeq package functions and custom scripts on R. T cell receptor (TCR) sequences were retrieved from CD8⁺ T cell RNA-Seq data sets and the frequency of TCR beta chain clonotypes were determined using default parameters of the MiXCR package⁵⁵ (Table 6). The CD103 status of TILs was determined based on the transcript levels of ITGAE (CD103) in CD8⁺ TILs. Tumors with CD8⁺ TILs expression of ITGAE transcripts in the top ⅓ or bottom ⅓ percentile were classified as CD103^{high} or CD103^{low}, respectively.

### Knowledge-Based Network Generation and Pathway Analysis.

The biological relevance of differentially expressed genes identified by DESeq2 analysis was further investigated using the Ingenuity Pathways Analysis platform. The enrichment of canonical pathways (pre-defined, well-described metabolic and signaling pathways curated from literature reviews) amongst differentially expressed genes was assessed, with significance determined by right-tailed Fisher's exact test, P<0.05. For network analysis, differentially expressed genes were progressively linked together based on a measure of their interconnection, which is derived from previously characterized functional interactions.

### Gene Set Enrichment Analysis (GSEA).

The Qlucore Omics Explorer 3.2 software package was used for GSEA analysis. GSEA was used to further assess whether specific biological pathways or signatures were significantly enriched between two groups. GSEA determines whether an apriori defined ‘set’ of genes (such as a signature) show statistically significant cumulative changes in gene expression between phenotypic subgroups⁵⁶. In brief, all genes are ranked based on their differential expression between two groups. Next, a running enrichment score (RES) is calculated for a given gene set based on how often its members appear at the top or bottom of the ranked differential list. 1000 random permutations of the phenotypic subgroups are used to establish a null distribution of RES against which a normalized running enrichment score (NES) and FDR-corrected q values are calculated using Kolmogorov-Smirnov statistic. GSEA was run with a focused group of gene signatures, namely exhaustion²², lung cancer associated T cell signature¹⁵, anergy⁵⁷, senescence⁵⁸, tissue residency²⁵. These gene signatures (FIGS. 1, 4E and Table 11) were selected to test the null hypothesis that different CD8 T cell phenotypes were not significantly enriched in CD8+ T cell groups.

### Statistical Analysis.

Comparison between two groups was assessed with two-tailed unpaired or paired Student's t-test (FIGS. 2F, 6D, 7B) or Mann-Whitney test (FIG. 5D) or Kolmogorov-Smirnov test using GraphPad Prism 6. Spearman correlation coefficient (r value) was calculated to assess the significance of correlation of the expression of any two transcripts of interest.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were

TABLE 1
Gene List

| OFFICIAL_GENE_SYMBOL | Name | GENE CARDS ID |
|---|---|---|
| ACTN4 | actinin, alpha 4 | GC19P038647 |
| ADD3 | adducin 3 (gamma) | GC10P109996 |
| ADRB2 | adrenergic, beta-2-, receptor, surface | GC05P148825 |
| AHCTF1 | AT hook containing transcription factor 1; AT hook | GC01M246840 |
| | containing transcription factor 1 pseudogene | |
| AKAP5 | A kinase (PRKA) anchor protein 5 | GC14P064465 |
| ANP32E | acidic (leucine-rich) nuclear phosphoprotein 32 family, | GC01M150190 |
| | member E | |
| ANTXR2 | anthrax toxin receptor 2 | GC04M079901 |
| ARL6IP6 | ADP-ribosylation-like factor 6 interacting protein 6 | GC02P152717 |
| ASB2 | ankyrin repeat and SOCS box-containing 2 | GC14M093934 |
| ATP1B1 | ATPase, Na+/K+ transporting, beta 1 polypeptide | GC01P169105 |
| ATP5G2 | ATP synthase, H+ transporting, mitochondrial F0 | GC12M053648 |
| | complex, subunit C2 (subunit 9) | |
| BCAS4 | breast carcinoma amplified sequence 4 | GC20P050794 |
| BST2 | NPC-A-7; bone marrow stromal cell antigen 2 | GC19M017403 |
| C6orf108 | chromosome 6 open reading frame 108 | GC06M043193 |
| CA5B | inactivation escape 2 (non-protein coding); carbonic | GC0XP015706 |
| | anhydrase VB, mitochondrial | |
| CAST | Calpastatin | GC05P096525 |
| CCL3 | chemokine (C-C motif) ligand 3 | GC17M036088 |
| CCL5 | chemokine (C-C motif) ligand 5 | GC17M035871 |
| CD200R1 | CD200 receptor 1 | GC03M112921 |
| CD38 | CD38 molecule | GC04P015779 |
| CD8A (also | CD8a molecule | GC02M086784 |
| known as CD8) | | |
| CD39 | CD39 molecule | GC10P095711 |
| CTLA4 | Cytotoxic T-Lymphocyte Associated Protein 4 | GC02P203867 |
| COTL1 | coactosin-like 1 (Dictyostelium) | GC16M084599 |
| CX3CR1 | chemokine (C—X3—C motif) receptor 1 | GC03M039279 |
| CXCR6 | chemokine (C—X—C motif) receptor 6 | GC03P045982 |
| DSTN | destrin (actin depolymerizing factor) | GC20P017550 |
| DUSP6 | dual specificity phosphatase 6 | GC12M089347 |
| EPSTI1 | epithelial stromal interaction 1 (breast) | GC13M042886 |
| FAM113B | family with sequence similarity 113, member B | GC12P047079 |
| FCGR3A | Fc fragment of IgG, low affinity IIIa, receptor (CD16a) | GC01M161541 |
| FGFBP2 | fibroblast growth factor binding protein 2 | GC04M015961 |
| FUT8 | fucosyltransferase 8 (alpha (1,6) fucosyltransferase) | GC14P065411 |
| GBP1 | guanylate binding protein 1, interferon-inducible, 67 kDa | GC01M089052 |
| GBP2 | guanylate binding protein 2, interferon-inducible | GC01M089106 |
| GBP4 | guanylate binding protein 4 | GC01M089181 |
| GBP5 | guanylate binding protein 5 | GC01M089259 |
| GMPS | guanine monphosphate synthetase | GC03P155870 |
| GNL3L | guanine nucleotide binding protein-like 3 (nucleolar)- | GC0XP054573 |
| | like | |
| GPI | glucose phosphate isomerase | GC19P034359 |
| GZMA | granzyme A (granzyme 1, cytotoxic T-lymphocyte- | GC05P055102 |
| | associated serine esterase 3) | |
| HAVCR2 | hepatitis A virus cellular receptor 2 | GC05M157063 |
| (also known as | | |
| TIM3) | | |
| HNRNPK | heterogeneous nuclear ribonucleoprotein K; similar to | GC09M083969 |
| | heterogeneous nuclear ribonucleoprotein K | |
| HNRPLL | heterogeneous nuclear ribonucleoprotein L-like | GC02M038561 |
| IGFLR1 | IGF Like Family Receptor 1 | GC19M038029 |
| IL21R | interleukin 21 receptor | GC16P027413 |
| ITGAE | integrin, alpha E (antigen CD103, human mucosal | GC17M003722 |
| (also known as | lymphocyte antigen 1; alpha polypeptide) | |
| CD103) | | |
| KLF2 | Kruppel-like factor 2 (lung) | GC19P019293 |
| KIR2DL4 | Killer cell immunoglobin like receptor | GC19P054994 |
| LAG3 | Lymphocyte Activating 3 | GC12P006774 |
| LDHB | lactate dehydrogenase B | GC12M021635 |
| LPAR6 | purinergic receptor P2Y, G-protein coupled, 5 | GC13M048389 |
| MCM4 | minichromosome maintenance complex component 4 | GC08P047965 |
| MLLT10 | myeloid/lymphoid or mixed-lineage leukemia (trithorax | GC10P021534 |
| | homolog, Drosophila); translocated to, 10 | |
| MRPL37 | mitochondrial ribosomal protein L37 | GC01P054185 |
| NAB1 | NGFI-A binding protein 1 (EGR1 binding protein 1) | GC02P190646 |
| NDUFS8 | NADH dehydrogenase (ubiquinone) Fe—S protein 8, | GC11P068030 |
| | 23 kDa (NADH-coenzyme Q reductase) | |
| NECAP1 | NECAP endocytosis associated 1 | GC12P008082 |
| NOTCH1 | Notch homolog 1, translocation-associated (Drosophila) | GC09M136505 |
| NPC2 | Niemann-Pick disease, type C2 | GC14M074476 |
| OAS3 | 2′-5′-oligoadenylate synthetase 3, 100 kDa | GC12P112938 |
| PAG1 | phosphoprotein associated with glycosphingolipid | GC08M080967 |
| | microdomains 1 | |
| PARP9 | poly (ADP-ribose) polymerase family, member 9 | GC03M122527 |
| PCMTD2 | protein-L-isoaspartate (D-aspartate) O- | GC20P064255 |
| | methyltransferase domain containing 2 | |
| PCNT | Pericentrin | GC21P046324 |
| PDCD1 (also | programmed cell death 1 | GC02M241849 |
| known as PD-1) | | |
| PLAC8 | placenta-specific 8 | GC04M083090 |
| POLR1D | polymerase (RNA) I polypeptide D, 16 kDa | GC13P027620 |
| PPM1M | protein phosphatase 1M (PP2C domain containing) | GC03P052245 |
| PPP2R4 | protein phosphatase 2A activator, regulatory subunit 4 | GC09P129111 |
| PRDM2 | PR domain containing 2, with ZNF domain | GC01P013776 |
| PRKAG1 | protein kinase, AMP-activated, gamma 1 non-catalytic | GC12M049002 |
| | subunit | |
| PRKAR1A | protein kinase, cAMP-dependent, regulatory, type I, | GC17P068414 |
| | alpha (tissue specific extinguisher 1) | |
| PSMB8 | proteasome (prosome, macropain) subunit, beta type, 8 | GC06M032840 |
| | (large multifunctional peptidase 7) | |
| PSMB9 | proteasome (prosome, macropain) subunit, beta type, 9 | GC06P032825 |
| | (large multifunctional peptidase 2) | |
| PSMD8 | proteasome (prosome, macropain) 26S subunit, non- | GC19P038374 |
| | ATPase, 8 | |
| PSME2 | proteasome (prosome, macropain) activator subunit 2 | GC14M024143 |
| | (PA28 beta) | |
| PTTG1 | pituitary tumor-transforming 1; pituitary tumor- | GC05P160422 |
| | transforming 2 | |
| PURA | purine-rich element binding protein A | GC05P140076 |
| R3HDM1 | R3H domain containing 1 | GC02P135531 |
| RAB3GAP1 | RAB3 GTPase activating protein subunit 1 (catalytic) | GC02P135052 |
| RABAC1 | Rab acceptor 1 (prenylated) | GC19M041956 |
| RARRES3 | retinoic acid receptor responder (tazarotene induced) 3 | GC11P063536 |
| RBBP4 | hypothetical LOC642954; retinoblastoma binding | GC01P032651 |
| | protein 4 | |
| S100A10 | S100 calcium binding protein A10 | GC01M151955 |
| S1PR1 | sphingosine-1-phosphate receptor 1 | GC01P101236 |
| SEC11A | SEC11 homolog A (S. cerevisiae) | GC15M084669 |
| SF3B3 | splicing factor 3b, subunit 3, 130 kDa | GC16P070523 |
| SIRPG | signal-regulatory protein gamma | GC20M001628 |
| SLC27A2 | solute carrier family 27 (fatty acid transporter), member 2 | GC15P050182 |
| SNX17 | sorting nexin 17 | GC02P027370 |
| SRA1 | steroid receptor RNA activator 1 | GC05M140537 |
| STAT1 | signal transducer and activator of transcription 1, 91 kDa | GC02M190964 |
| STAT2 | signal transducer and activator of transcription 2, | GC12M056341 |
| | 113 kDa | |
| STK38 | serine/threonine kinase 38 | GC06M036493 |
| STMN1 | stathmin 1 | GC01M025884 |
| SYT11 | synaptotagmin XI | GC01P155829 |
| TAZ | Tafazzin | GC0XP154411 |
| TGFBR3 | transforming growth factor, beta receptor III | GC01M091619 |
| TIAM1 | T-cell lymphoma invasion and metastasis 1 | GC21M031118 |
| TIMP1 | TIMP metallopeptidase inhibitor 1 | GC0XP047583 |
| TMEM140 | transmembrane protein 140 | GC07P135148 |
| TNF | tumor necrosis factor (TNF superfamily, member 2) | GC06P031673 |
| TNFRSF9 (also | tumor necrosis factor receptor superfamily, member 9 | GC01M007915 |
| known as 41BB) | | |
| TNFSF4 (also | tumor necrosis factor (ligand) superfamily, member 4 | GC01M173152 |
| known as OX40- | | |
| Ligand) | | |
| TNRC6C | trinucleotide repeat containing 6C | GC17P077959 |
| TOP2A | topoisomerase (DNA) II alpha 170 kDa | GC17M040388 |
| TP53BP2 | tumor protein p53 binding protein, 2 | GC01M223779 |
| TRAPPC10 | trafficking protein particle complex 10 | GC21P044012 |
| TUG1 | taurine upregulated 1 (non-protein coding) | GC22P030969 |
| UBE2L6 | ubiquitin-conjugating enzyme E2L 6 | GC11M057571 |
| UBE2Q2 | ubiquitin-conjugating enzyme E2Q family member 2 | GC15P075843 |
| ZFYVE26 | zinc finger, FYVE domain containing 26 | GC14M067727 |

TABLE 2
Demographic, clinical and histopathological characteristics of cancer patients.
A. Non-small cell lung cancer

| | | | | | | | | | | | | Number |
|---|---|---|---|---|---|---|---|---|---|---|---|---|
| | | | | | Metas- | | | | ALK | | | of CD8a+ |
| | | | | Tumor | tasis | Perfor- | Smok- | | trans- | EGFR | | cells |
| | Age | | | status | status | mance | ing | Asbestos | location | mutation | Tumor | (average |
| Patient ID | (years) | Gender | Stage | (T) | (M) | status | status | exposure | status | status | histology | per HPF) |
| NSCLC_01 | 87 | M | IA | 1A | 0 | 0 | Ex | No | Negative | Negative | adenocarcinoma | 32.7 |
| NSCLC_02 | 74 | M | IIB | 2B | 0 | 0 | Ex | No | Negative | Negative | squamous | 8.6 |
| | | | | | | | | | | | carcinoma | |
| NSCLC_03 | 77 | M | IA | 2B | 0 | 0 | Ex | Yes | Negative | Negative | adenocarcinoma | 28.2 |
| NSCLC_04 | 67 | M | IB | 2A | 0 | 0 | Ex | Yes | Negative | Negative | squamous | 14.7 |
| | | | | | | | | | | | carcinoma | |
| NSCLC_05 | 84 | F | IIA | 1B | 0 | 0 | Ex | No | Negative | Negative | adenocarcinoma | 11.4 |
| NSCLC_06 | 72 | M | IA | 1B | 0 | 1 | Ex | No | Negative | Negative | adenocarcinoma | 15.6 |
| NSCLC_07 | 74 | M | IIB | 3 | 0 | 0 | Ex | No | N/A | N/A | adenocarcinoma | 80.3 |
| NSCLC_08 | 63 | M | IB | 2A | 0 | 0 | Ex | No | Negative | Negative | adenocarcinoma | 21.2 |
| NSCLC_09 | 83 | M | IIA | 2B | 0 | 0 | Ex | Yes | Negative | Negative | squamous | 11.4 |
| | | | | | | | | | | | carcinoma | |
| NSCLC_10 | 64 | M | IB | 1A | 0 | 0 | Ex | Yes | Negative | Negative | adenocarcinoma | 23.2 |
| NSCLC_11 | 72 | F | IIIA | 4 | 0 | 0 | Current | No | Negative | Negative | adenocarcinoma | 9.9 |
| NSCLC_12 | 72 | F | IIA | 2B | 0 | 0 | Never | No | Negative | Negative | adenocarcinoma | 28.1 |
| NSCLC_13 | 68 | F | IIA | 2A | 0 | 1 | Ex | No | Negative | Negative | adenocarcinoma | 9.2 |
| NSCLC_14 | 50 | M | IB | 2B | 0 | 0 | Current | No | N/A | Negative | adenocarcinoma | 7.1 |
| NSCLC_15 | 74 | M | IB | 2A | 0 | 1 | Ex | No | Negative | Negative | adenocarcinoma | 8.3 |
| NSCLC_16 | 65 | F | IA | 1A | 0 | 1 | Ex | No | Negative | Negative | adenocarcinoma | 3.0 |
| NSCLC_17 | 68 | M | IIA | 2B | 0 | 0 | Ex | No | Negative | Negative | squamous | 17.5 |
| | | | | | | | | | | | carcinoma | |
| NSCLC_18 | 71 | F | IIIA | 3 | 0 | 0 | Current | No | Negative | Negative | squamous | 15.0 |
| | | | | | | | | | | | carcinoma | |
| NSCLC_19 | 68 | F | IA | 1A | 0 | 0 | Ex | No | N/A | N/A | adenocarcinoma | 6.5 |
| NSCLC_20 | 72 | F | IB | 2A | 0 | 0 | Ex | No | Negative | Negative | adenocarcinoma | 38.7 |
| NSCLC_21 | 72 | M | IV | 1A | 1B | 1 | Ex | No | Negative | Negative | adenocarcinoma | 10.3 |
| NSCLC_22 | 70 | M | IIIA | 3 | 0 | 1 | Ex | Yes | Negative | Negative | Adenocarcinoma | 4.1 |
| NSCLC_23 | 51 | F | IB | 2A | 0 | 0 | Never | No | Negative | Positive | adenocarcinoma | 9.6 |
| NSCLC_24 | 77 | F | IB | 2A | 0 | 1 | Ex | No | Negative | Negative | adenocarcinoma | 10.8 |
| NSCLC_25 | 60 | F | IA | 1B | 0 | 1 | Ex | No | Negative | Negative | adenocarcinoma | 10.7 |
| NSCLC_26 | 77 | F | IIA | 2A | 0 | 0 | Ex | No | Negative | Positive | adenocarcinoma | 6.3 |
| NSCLC_27 | 81 | F | IIB | 3 | 0 | 0 | Ex | No | N/A | N/A | squamous | 10.8 |
| | | | | | | | | | | | carcinoma | |
| NSCLC_28 | 69 | F | IB | 2A | 0 | 0 | Ex | No | Negative | Negative | adenocarcinoma | 6.8 |
| NSCLC_29 | 73 | M | IB | 2A | 0 | 0 | Ex | No | Negative | Negative | adenocarcinoma | 3.7 |
| NSCLC_30 | 81 | F | IIIB | 4 | 0 | 1 | Never | No | Negative | Negative | adenocarcinoma | 4.3 |
| NSCLC_31 | 76 | M | IA | 1B | 0 | 0 | Current | No | Negative | Negative | squamous | 2.7 |
| | | | | | | | | | | | carcinoma | |
| NSCLC_32 | 77 | F | IIIA | 2A | 0 | 1 | Never | No | Negative | Negative | adenocarcinoma | 4.8 |
| NSCLC_33 | 67 | M | IIB | 3 | 0 | 1 | Current | Yes | Negative | Negative | squamous | 4.4 |
| | | | | | | | | | | | carcinoma | |
| NSCLC_34 | 70 | F | IA | 1B | 0 | 1 | Ex | No | Negative | Negative | adenocarcinoma | 12.6 |
| NSCLC_35 | 66 | M | IA | 1A | 0 | 0 | Ex | No | Negative | Negative | adenocarcinoma | 10.1 |
| NSCLC_36 | 80 | M | IB | 2A | 0 | 1 | Ex | No | Negative | Negative | squamous | 18.6 |
| | | | | | | | | | | | carcinoma | |
| NSCLC_37 | 81 | M | IA | 1A | 0 | 1 | Ex | Yes | N/A | N/A | squamous | N/A |
| | | | | | | | | | | | carcinoma | |
| NSCLC_38 | 69 | M | IB | 1B | 0 | 0 | Ex | No | N/A | N/A | adenocarcinoma | N/A |
| NSCLC_39 | 75 | M | IIIA | 3 | 0 | 0 | Current | Yes | Negative | Negative | squamous | N/A |
| | | | | | | | | | | | carcinoma | |
| NSCLC_40 | 58 | F | IA | 1A | 0 | 0 | Current | No | Negative | Negative | adenocarcinoma | N/A |
| NSCLC_41 | 76 | M | IB | 2A | 0 | 0 | Ex | No | Negative | Negative | adenocarcinoma | N/A |
| NSCLC_42 | 74 | M | IA | 1A | 0 | 0 | Ex | No | N/A | N/A | adenocarcinoma | N/A |
| NSCLC_43 | 79 | M | IIB | 3 | 0 | 0 | Ex | No | Negative | Negative | squamous | N/A |
| | | | | | | | | | | | carcinoma | |

Data not available is indicated by ‘N/A’.
“ALK translocation status” negative indicates the absence of a translocation involving anaplastic lymphoma kinase gene (ALK)
“EGFR mutation status” positive indicates presence of activating mutations in epidermal growth factor receptor gene (EGFR)
B. Head & neck squamous cell cancer

| | | | | Tumor | Nodal | Metastasis | | | Number of | | QC passed |
|---|---|---|---|---|---|---|---|---|---|---|---|
| | Age | | | status | status | status | Smoking | HPV | CD8+ cells | | TIL |
| Patient ID | (years) | Gender | Stage | (T) | (N) | (M) | status | Status | (average per HPF) | TIL status | RNA-Seq |
| HNSCC_01 | 82 | M | III | 2 | 1 | 0 | Ex | Negative | 25 | Intermediate | Yes |
| HNSCC_02 | 55 | F | IVA | 4 | 1 | 0 | Ex | Negative | 12.5 | Intermediate | Yes |
| HNSCC_03 | 94 | F | IVA | 3 | 0 | 0 | N/A | Negative | 3.1 | Low | Yes |
| HNSCC_04 | 69 | M | III | 2 | 1 | 0 | N/A | Positive | 26.1 | Intermediate | Yes |
| HNSCC_05 | 57 | M | IVA | 1 | 2B | 0 | Smoker | Negative | 35.7 | High | Yes |
| HNSCC_06 | 66 | M | IVA | 4 | 2B | 0 | Never | Positive | 23.9 | Intermediate | Yes |
| HNSCC_07 | 64 | F | I | 1 | 0 | 0 | Ex | Negative | 35.5 | High | Yes |
| HNSCC_08 | 63 | M | IVA | 4 | 2C | 0 | Current | Negative | 29.3 | High | Yes |
| HNSCC_09 | 66 | F | IVA | 2 | 2B | 0 | N/A | Negative | 28.5 | High | Yes |
| HNSCC_10 | 86 | F | IVA | 4A | 0 | 0 | Never | Positive | 24.1 | Intermediate | Yes |
| HNSCC_11 | 70 | M | IVA | 4B | 2B | 0 | N/A | Negative | 10.2 | Low | Yes |
| HNSCC_12 | 56 | M | IVA | 3 | 2B | 0 | Never | Negative | 32 | High | Yes |
| HNSCC_13 | 47 | M | IVA | 3 | 2A | 0 | Current | Positive | N/A | N/A | Yes |
| HNSCC_14 | 67 | M | IVA | 4A | 2B | 0 | Never | Positive | 24.8 | Intermediate | Yes |
| HNSCC_15 | 74 | M | III | 2 | 1 | 0 | N/A | Negative | 11 | Low | Yes |
| HNSCC_16 | 57 | M | IVC | 4 | 3 | 1 | Current | Negative | 37.2 | High | Yes |
| HNSCC_17 | 60 | F | IVA | 4A | 2B | 0 | Ex | Negative | 1.6 | Low | Yes |
| HNSCC_18 | 48 | M | IVA | 2 | 2A | 0 | Ex | Positive | 32.5 | High | Yes |
| HNSCC_19 | 60 | M | III | 3 | 1 | 0 | Current | Positive | 26.8 | Intermediate | Yes |
| HNSCC_20 | 51 | M | IVA | 4 | 0 | 0 | Ex | Positive | 25.5 | Intermediate | Yes |
| HNSCC_21 | 62 | M | II | 2 | 0 | 0 | Never | Negative | 28.4 | High | Yes |
| HNSCC_22 | 55 | M | IVA | 2 | 2C | 0 | Current | Negative | 31.1 | High | Yes |
| HNSCC_23 | 68 | M | IVA | 2 | 2C | 0 | Ex | Positive | 24.4 | Intermediate | Yes |
| HNSCC_24 | 75 | M | III | 2 | 1 | 0 | Ex | Negative | N/A | N/A | Yes |
| HNSCC_25 | 50 | M | III | 1 | 1 | 0 | Never | Positive | N/A | N/A | Yes |
| HNSCC_26 | 68 | M | IVA | 3 | 2B | 0 | Never | Positive | 2 | Low | Yes |
| HNSCC_27 | 62 | F | IVA | 4 | 1 | 0 | Current | Negative | 2.4 | Low | Yes |
| HNSCC_28 | 29 | F | II | 2 | 0 | 0 | Ex | Positive | 27.4 | Intermediate | Yes |
| HNSCC_29 | 61 | F | IVA | 2 | 2C | 0 | Current | Negative | 20.5 | Intermediate | Yes |
| HNSCC_30 | 52 | M | IVA | 4 | 0 | 0 | Current | Negative | 1.5 | Low | Yes |
| HNSCC_31 | 70 | F | II | 2 | 0 | 0 | N/A | Negative | 11.2 | Low | Yes |
| HNSCC_32 | 67 | F | II | 2 | 0 | 0 | Ex | Negative | 2.2 | Low | Yes |
| HNSCC_33 | 60 | M | IVA | 2 | 2C | 0 | Never | Positive | 45 | High | Yes |
| HNSCC_34 | 57 | M | IVA | 1 | 2B | 0 | Smoker | Negative | 47.7 | High | Yes |
| HNSCC_35 | 51 | M | IVB | 2 | 3 | 0 | Never | Positive | 24.4 | Intermediate | Yes |
| HNSCC_36 | 71 | F | III | 3 | 0 | 0 | Never | Positive | 41.4 | High | Yes |
| HNSCC_37 | 63 | M | IVA | 3 | 2B | 0 | Ex | Negative | 24.1 | Intermediate | Yes |
| HNSCC_38 | 61 | M | IVA | 1 | 2B | 0 | N/A | Negative | N/A | N/A | Yes |
| HNSCC_39 | 63 | M | IVA | 4 | 2B | 0 | Current | Negative | 6.4 | Low | Yes |
| HNSCC_40 | 38 | M | IVA | 3 | 2B | 0 | Current | Positive | 5 | Low | Yes |
| HNSCC_41 | 62 | M | II | 2 | 0 | 0 | Ex | Negative | 2.5 | Low | Yes |

Data not available is indicated by ‘N/A’.
“HPV status” positive indicates presence of human papilloma virus (HPV) infection in tumors as determined by over expression of p16
in tumor samples”

TABLE 3
Details of libraries run for RNA sequencing.

| | | | |
|---|---|---|---|
| | | | Total number of |
| | | | uniquely mapped |
| | | | reads (excluding |
| Sample ID | Patient ID | Cell type | mitochondrial reads) |

A. Non small cell lung cancer
For each RNA-Seq assay, the table lists sample ID, patient ID, cell type and total number of uniquely
mapped reads excluding mitochondrial reads.

| | | | |
|---|---|---|---|
| NSCLC_01_TIL | NSCLC_01 | FACS-sorted CD8+ TILs from NSCLC | 13,020,371 |
| NSCLC_02_TIL | NSCLC_02 | FACS-sorted CD8+ TILs from NSCLC | 11,542,850 |
| NSCLC_03_TIL | NSCLC_03 | FACS-sorted CD8+ TILs from NSCLC | 12,216,079 |
| NSCLC_04_TIL | NSCLC_04 | FACS-sorted CD8+ TILs from NSCLC | 14,162,563 |
| NSCLC_05_TIL | NSCLC_05 | FACS-sorted CD8+ TILs from NSCLC | 10,909,550 |
| NSCLC_06_TIL | NSCLC_06 | FACS-sorted CD8+ TILs from NSCLC | 16,098,077 |
| NSCLC_07_TIL | NSCLC_07 | FACS-sorted CD8+ TILs from NSCLC | 12,350,892 |
| NSCLC_08_TIL | NSCLC_08 | FACS-sorted CD8+ TILs from NSCLC | 16,349,349 |
| NSCLC_09_TIL | NSCLC_09 | FACS-sorted CD8+ TILs from NSCLC | 12,273,924 |
| NSCLC_10_TIL | NSCLC_10 | FACS-sorted CD8+ TILs from NSCLC | 12,699,628 |
| NSCLC_11_TIL | NSCLC_11 | FACS-sorted CD8+ TILs from NSCLC | 11,436,022 |
| NSCLC_12_TIL | NSCLC_12 | FACS-sorted CD8+ TILs from NSCLC | 13,757,125 |
| NSCLC_13_TIL | NSCLC_13 | FACS-sorted CD8+ TILs from NSCLC | 12,440,359 |
| NSCLC_14_TIL | NSCLC_14 | FACS-sorted CD8+ TILs from NSCLC | 19,173,715 |
| NSCLC_15_TIL | NSCLC_15 | FACS-sorted CD8+ TILs from NSCLC | 17,406,814 |
| NSCLC_16_TIL | NSCLC_16 | FACS-sorted CD8+ TILs from NSCLC | 11,122,554 |
| NSCLC_17_TIL | NSCLC_17 | FACS-sorted CD8+ TILs from NSCLC | 13,645,925 |
| NSCLC_18_TIL | NSCLC_18 | FACS-sorted CD8+ TILs from NSCLC | 15,697,087 |
| NSCLC_19_TIL | NSCLC_19 | FACS-sorted CD8+ TILs from NSCLC | 11,938,530 |
| NSCLC_20_TIL | NSCLC_20 | FACS-sorted CD8+ TILs from NSCLC | 14,223,418 |
| NSCLC_21_TIL | NSCLC_21 | FACS-sorted CD8+ TILs from NSCLC | 13,413,370 |
| NSCLC_22_TIL | NSCLC_22 | FACS-sorted CD8+ TILs from NSCLC | 12,971,479 |
| NSCLC_23_TIL | NSCLC_23 | FACS-sorted CD8+ TILs from NSCLC | 11,719,664 |
| NSCLC_24_TIL | NSCLC_24 | FACS-sorted CD8+ TILs from NSCLC | 13,589,132 |
| NSCLC_25_TIL | NSCLC_25 | FACS-sorted CD8+ TILs from NSCLC | 13,509,805 |
| NSCLC_26_TIL | NSCLC_26 | FACS-sorted CD8+ TILs from NSCLC | 12,019,742 |
| NSCLC_27_TIL | NSCLC_27 | FACS-sorted CD8+ TILs from NSCLC | 13,984,367 |
| NSCLC_28_TIL | NSCLC_28 | FACS-sorted CD8+ TILs from NSCLC | 11,155,688 |
| NSCLC_29_TIL | NSCLC_29 | FACS-sorted CD8+ TILs from NSCLC | 12,834,065 |
| NSCLC_30_TIL | NSCLC_30 | FACS-sorted CD8+ TILs from NSCLC | 14,242,019 |
| NSCLC_31_TIL | NSCLC_31 | FACS-sorted CD8+ TILs from NSCLC | 11,305,292 |
| NSCLC_32_TIL | NSCLC_32 | FACS-sorted CD8+ TILs from NSCLC | 12,714,146 |
| NSCLC_33_TIL | NSCLC_33 | FACS-sorted CD8+ TILs from NSCLC | 13,242,761 |
| NSCLC_34_TIL | NSCLC_34 | FACS-sorted CD8+ TILs from NSCLC | 11,853,168 |
| NSCLC_35_TIL | NSCLC_35 | FACS-sorted CD8+ TILs from NSCLC | 13,833,776 |
| NSCLC_36_TIL | NSCLC_36 | FACS-sorted CD8+ TILs from NSCLC | 12,798,616 |
| NSCLC_01_N-TIL | NSCLC_01 | FACS-sorted CD8+ N-TILs from uninvolved lung | 10,724,899 |
| NSCLC_02_N-TIL | NSCLC_02 | FACS-sorted CD8+ N-TILs from uninvolved lung | 15,708,837 |
| NSCLC_03_N-TIL | NSCLC_03 | FACS-sorted CD8+ N-TILs from uninvolved lung | 11,576,281 |
| NSCLC_05_N-TIL | NSCLC_05 | FACS-sorted CD8+ N-TILs from uninvolved lung | 15,739,299 |
| NSCLC_08_N-TIL | NSCLC_08 | FACS-sorted CD8+ N-TILs from uninvolved lung | 23,744,700 |
| NSCLC_10_N-TIL | NSCLC_10 | FACS-sorted CD8+ N-TILs from uninvolved lung | 12,566,143 |
| NSCLC_11_N-TIL | NSCLC_11 | FACS-sorted CD8+ N-TILs from uninvolved lung | 12,482,491 |
| NSCLC_12_N-TIL | NSCLC_12 | FACS-sorted CD8+ N-TILs from uninvolved lung | 12,919,370 |
| NSCLC_14_N-TIL | NSCLC_14 | FACS-sorted CD8+ N-TILs from uninvolved lung | 11,586,753 |
| NSCLC_16_N-TIL | NSCLC_16 | FACS-sorted CD8+ N-TILs from uninvolved lung | 10,708,372 |
| NSCLC_17_N-TIL | NSCLC_17 | FACS-sorted CD8+ N-TILs from uninvolved lung | 13,056,386 |
| NSCLC_19_N-TIL | NSCLC_19 | FACS-sorted CD8+ N-TILs from uninvolved lung | 11,417,787 |
| NSCLC_22_N-TIL | NSCLC_22 | FACS-sorted CD8+ N-TILs from uninvolved lung | 13,100,404 |
| NSCLC_23_N-TIL | NSCLC_23 | FACS-sorted CD8+ N-TILs from uninvolved lung | 10,835,392 |
| NSCLC_25_N-TIL | NSCLC_25 | FACS-sorted CD8+ N-TILs from uninvolved lung | 13,287,194 |
| NSCLC_26_N-TIL | NSCLC_26 | FACS-sorted CD8+ N-TILs from uninvolved lung | 12,874,088 |
| NSCLC_27_N-TIL | NSCLC_27 | FACS-sorted CD8+ N-TILs from uninvolved lung | 12,510,907 |
| NSCLC_28_N-TIL | NSCLC_28 | FACS-sorted CD8+ N-TILs from uninvolved lung | 12,639,045 |
| NSCLC_29_N-TIL | NSCLC_29 | FACS-sorted CD8+ N-TILs from uninvolved lung | 11,857,037 |
| NSCLC_30_N-TIL | NSCLC_30 | FACS-sorted CD8+ N-TILs from uninvolved lung | 14,246,557 |
| NSCLC_32_N-TIL | NSCLC_32 | FACS-sorted CD8+ N-TILs from uninvolved lung | 12,696,885 |
| NSCLC_33_N-TIL | NSCLC_33 | FACS-sorted CD8+ N-TILs from uninvolved lung | 12,242,225 |
| NSCLC_34_N-TIL | NSCLC_34 | FACS-sorted CD8+ N-TILs from uninvolved lung | 12,334,230 |
| NSCLC_35_N-TIL | NSCLC_35 | FACS-sorted CD8+ N-TILs from uninvolved lung | 12,993,603 |
| NSCLC_36_N-TIL | NSCLC_36 | FACS-sorted CD8+ N-TILs from uninvolved lung | 13,434,111 |
| NSCLC_37_N-TIL | NSCLC_37 | FACS-sorted CD8+ N-TILs from uninvolved lung | 12,773,058 |
| NSCLC_38_N-TIL | NSCLC_38 | FACS-sorted CD8+ N-TILs from uninvolved lung | 14,484,549 |
| NSCLC_39_N-TIL | NSCLC_39 | FACS-sorted CD8+ N-TILs from uninvolved lung | 14,472,842 |
| NSCLC_40_N-TIL | NSCLC_40 | FACS-sorted CD8+ N-TILs from uninvolved lung | 11,720,532 |
| NSCLC_41_N-TIL | NSCLC_41 | FACS-sorted CD8+ N-TILs from uninvolved lung | 11,337,189 |
| NSCLC_42_N-TIL | NSCLC_42 | FACS-sorted CD8+ N-TILs from uninvolved lung | 12,460,707 |
| NSCLC_43_N-TIL | NSCLC_43 | FACS-sorted CD8+ N-TILs from uninvolved lung | 12,509,756 |

B. Head & neck squamous cell cancer
For each RNA-Seq assay, the table lists sample ID, patient ID, cell type and total number of uniquely
mapped reads excluding mitochondrial reads.

| | | | |
|---|---|---|---|
| HNSCC_01_TIL | HNSCC_01 | FACS-sorted CD8+ TILs from HNSCC | 6,057,956 |
| HNSCC_02_TIL | HNSCC_02 | FACS-sorted CD8+ TILs from HNSCC | 8,160,090 |
| HNSCC_03_TIL | HNSCC_03 | FACS-sorted CD8+ TILs from HNSCC | 5,089,047 |
| HNSCC_04_TIL | HNSCC_04 | FACS-sorted CD8+ TILs from HNSCC | 5,442,594 |
| HNSCC_05_TIL | HNSCC_05 | FACS-sorted CD8+ TILs from HNSCC | 9,503,393 |
| HNSCC_06_TIL | HNSCC_06 | FACS-sorted CD8+ TILs from HNSCC | 11,726,291 |
| HNSCC_07_TIL | HNSCC_07 | FACS-sorted CD8+ TILs from HNSCC | 15,579,048 |
| HNSCC_08_TIL | HNSCC_08 | FACS-sorted CD8+ TILs from HNSCC | 9,280,208 |
| HNSCC_09_TIL | HNSCC_09 | FACS-sorted CD8+ TILs from HNSCC | 10,429,966 |
| HNSCC_10_TIL | HNSCC_10 | FACS-sorted CD8+ TILs from HNSCC | 11,292,924 |
| HNSCC_11_TIL | HNSCC_11 | FACS-sorted CD8+ TILs from HNSCC | 15,327,902 |
| HNSCC_12_TIL | HNSCC_12 | FACS-sorted CD8+ TILs from HNSCC | 10,115,277 |
| HNSCC_13_TIL | HNSCC_13 | FACS-sorted CD8+ TILs from HNSCC | 17,982,291 |
| HNSCC_14_TIL | HNSCC_14 | FACS-sorted CD8+ TILs from HNSCC | 10,281,548 |
| HNSCC_15_TIL | HNSCC_15 | FACS-sorted CD8+ TILs from HNSCC | 14,985,658 |
| HNSCC_16_TIL | HNSCC_16 | FACS-sorted CD8+ TILs from HNSCC | 7,541,577 |
| HNSCC_17_TIL | HNSCC_17 | FACS-sorted CD8+ TILs from HNSCC | 10,282,472 |
| HNSCC_18_TIL | HNSCC_18 | FACS-sorted CD8+ TILs from HNSCC | 10,332,233 |
| HNSCC_19_TIL | HNSCC_19 | FACS-sorted CD8+ TILs from HNSCC | 14,519,215 |
| HNSCC_20_TIL | HNSCC_20 | FACS-sorted CD8+ TILs from HNSCC | 10,025,567 |
| HNSCC_21_TIL | HNSCC_21 | FACS-sorted CD8+ TILs from HNSCC | 13,350,981 |
| HNSCC_22_TIL | HNSCC_22 | FACS-sorted CD8+ TILs from HNSCC | 2,239,887 |
| HNSCC_23_TIL | HNSCC_23 | FACS-sorted CD8+ TILs from HNSCC | 14,813,440 |
| HNSCC_24_TIL | HNSCC_24 | FACS-sorted CD8+ TILs from HNSCC | 11,763,101 |
| HNSCC_25_TIL | HNSCC_25 | FACS-sorted CD8+ TILs from HNSCC | 15,701,995 |
| HNSCC_26_TIL | HNSCC_26 | FACS-sorted CD8+ TILs from HNSCC | 10,522,801 |
| HNSCC_27_TIL | HNSCC_27 | FACS-sorted CD8+ TILs from HNSCC | 15,878,485 |
| HNSCC_28_TIL | HNSCC_28 | FACS-sorted CD8+ TILs from HNSCC | 11,362,339 |
| HNSCC_29_TIL | HNSCC_29 | FACS-sorted CD8+ TILs from HNSCC | 7,039,987 |
| HNSCC_30_TIL | HNSCC_30 | FACS-sorted CD8+ TILs from HNSCC | 13,324,623 |
| HNSCC_31_TIL | HNSCC_31 | FACS-sorted CD8+ TILs from HNSCC | 12,943,157 |
| HNSCC_32_TIL | HNSCC_32 | FACS-sorted CD8+ TILs from HNSCC | 11,820,527 |
| HNSCC_33_TIL | HNSCC_33 | FACS-sorted CD8+ TILs from HNSCC | 6,562,823 |
| HNSCC_34_TIL | HNSCC_34 | FACS-sorted CD8+ TILs from HNSCC | 12,331,493 |
| HNSCC_35_TIL | HNSCC_35 | FACS-sorted CD8+ TILs from HNSCC | 15,876,608 |
| HNSCC_36_TIL | HNSCC_36 | FACS-sorted CD8+ TILs from HNSCC | 12,755,890 |
| HNSCC_37_TIL | HNSCC_37 | FACS-sorted CD8+ TILs from HNSCC | 12,197,671 |
| HNSCC_38_TIL | HNSCC_38 | FACS-sorted CD8+ TILs from HNSCC | 9,774,851 |
| HNSCC_39_TIL | HNSCC_39 | FACS-sorted CD8+ TILs from HNSCC | 7,740,986 |
| HNSCC_40_TIL | HNSCC_40 | FACS-sorted CD8+ TILs from HNSCC | 15,133,640 |
| HNSCC_41_TIL | HNSCC_41 | FACS-sorted CD8+ TILs from HNSCC | 9,704,871 |

TABLE 4
List of differentially expressed genes in CD8+ TILs from NSCLC

| | Normalized mean counts | | DE-Seq statistics | | |
|---|---|---|---|---|---|
| Gene symbol | CD8+ N-TILs | CD8+ TILs | Fold Change | P value | P adj |
| ABAT | 53.63 | 98.54 | 1.84 | 0.0069 | 0.039 |
| ABCD2 | 361.15 | 603.45 | 1.67 | 1.30E−08 | 6.80E−07 |
| ABL2 | 143.76 | 91.59 | 0.64 | 0.00092 | 0.0084 |
| ABTB2 | 12.22 | 44.61 | 3.65 | 0.00021 | 0.0026 |
| ACAP3 | 119.89 | 69.02 | 0.58 | 0.0013 | 0.011 |
| ACOT1 | 18.94 | 6.88 | 0.36 | 0.00022 | 0.0027 |
| ACOT4 | 160.61 | 79.96 | 0.50 | 0.00077 | 0.0072 |
| ACSS1 | 405.34 | 262.36 | 0.65 | 0.00035 | 0.0039 |
| ACSS2 | 69.24 | 27.87 | 0.40 | 0.0022 | 0.016 |
| ACTG2 | 1.77 | 48.59 | 27.45 | 0.00057 | 0.0058 |
| ACTN1 | 370.81 | 127.43 | 0.34 | 4.40E−09 | 2.50E−07 |
| ACVR2B | 29 | 8.49 | 0.29 | 0.00093 | 0.0085 |
| ACVRL1 | 68.81 | 10.65 | 0.15 | 4.70E−07 | 1.60E−05 |
| ACYP1 | 75.89 | 114.88 | 1.51 | 0.0032 | 0.022 |
| ADAM28 | 17.64 | 112.69 | 6.39 | 1.20E−11 | 1.40E−09 |
| ADAMTS1 | 57.97 | 10.35 | 0.18 | 1.30E−05 | 0.00025 |
| ADAMTSL4 | 204.57 | 46.68 | 0.23 | 1.40E−09 | 8.80E−08 |
| ADARB2 | 19.22 | 48.59 | 2.53 | 4.10E−05 | 0.00067 |
| ADAT2 | 49.38 | 79.41 | 1.61 | 0.0081 | 0.044 |
| ADRB2 | 1682.72 | 1038.05 | 0.62 | 0.0014 | 0.012 |
| ADTRP | 58.53 | 12.59 | 0.22 | 5.60E−06 | 0.00013 |
| AES | 1829.67 | 993.81 | 0.54 | 2.20E−15 | 5.70E−13 |
| AFAP1 | 123.88 | 196.75 | 1.59 | 0.0083 | 0.045 |
| AFAP1L2 | 85.58 | 318.53 | 3.72 | 2.70E−07 | 9.90E−06 |
| AGER | 72.45 | 20.02 | 0.28 | 0.00033 | 0.0037 |
| AGMAT | 35.93 | 59.92 | 1.67 | 0.0068 | 0.039 |
| AGPAT4 | 204.21 | 72.75 | 0.36 | 9.30E−09 | 5.00E−07 |
| AGPAT4-IT1 | 48.69 | 14.25 | 0.29 | 0.00054 | 0.0055 |
| AGRP | 17.3 | 7.21 | 0.42 | 0.0078 | 0.043 |
| AHI1 | 235.37 | 396.99 | 1.69 | 0.00014 | 0.0019 |
| AIF1 | 284.23 | 127.58 | 0.45 | 2.70E−05 | 0.00047 |
| AIM2 | 60.38 | 181.05 | 3.00 | 1.00E−06 | 3.10E−05 |
| AK4 | 19.4 | 90.97 | 4.69 | 5.40E−07 | 1.80E−05 |
| AK5 | 19.03 | 4.99 | 0.26 | 0.0018 | 0.014 |
| AKAP5 | 114.91 | 369.89 | 3.22 | 4.60E−10 | 3.30E−08 |
| AKR1B10 | 5.93 | 18.09 | 3.05 | 0.0044 | 0.028 |
| AKR1C3 | 151 | 55.59 | 0.37 | 0.00017 | 0.0022 |
| ALDH1A1 | 288.71 | 86.34 | 0.30 | 2.10E−05 | 0.00038 |
| ALDH2 | 2024.53 | 226.94 | 0.11 | 2.50E−19 | 1.70E−16 |
| ALDH3B1 | 126.29 | 31.88 | 0.25 | 3.90E−08 | 1.70E−06 |
| ALDH7A1 | 17.4 | 8.06 | 0.46 | 0.0018 | 0.014 |
| ALG10 | 34.07 | 69.51 | 2.04 | 0.00054 | 0.0055 |
| ALG3 | 602.89 | 401.53 | 0.67 | 0.0023 | 0.017 |
| ALOX15 | 23.2 | 3.04 | 0.13 | 2.10E−07 | 7.80E−06 |
| ALOX5 | 692.43 | 133.98 | 0.19 | 4.80E−24 | 1.10E−20 |
| AMOTL1 | 27.27 | 6.61 | 0.24 | 0.00087 | 0.008 |
| AMZ1 | 20.3 | 56.94 | 2.80 | 0.0074 | 0.041 |
| ANKLE1 | 7.07 | 20.24 | 2.86 | 0.0082 | 0.045 |
| ANKRD30BL | 46.82 | 28.63 | 0.61 | 0.00073 | 0.0069 |
| ANKRD32 | 570.38 | 1047.89 | 1.84 | 6.60E−11 | 5.90E−09 |
| ANKRD35 | 30.44 | 87.93 | 2.89 | 0.0039 | 0.025 |
| ANKRD9 | 21.15 | 6.53 | 0.31 | 0.006 | 0.036 |
| ANKS1B | 6.58 | 41.75 | 6.34 | 6.70E−05 | 0.001 |
| ANKS4B | 6.34 | 10.41 | 1.64 | 0.0084 | 0.045 |
| ANPEP | 223.38 | 29.87 | 0.13 | 1.40E−10 | 1.10E−08 |
| ANTXRL | 8.82 | 13.8 | 1.56 | 0.0093 | 0.049 |
| ANXA1 | 10537.02 | 6033.29 | 0.57 | 2.40E−13 | 3.60E−11 |
| ANXA2 | 3611.39 | 2122.75 | 0.59 | 1.70E−14 | 3.40E−12 |
| ANXA2P2 | 14.68 | 8.04 | 0.55 | 0.00024 | 0.0028 |
| ANXA4 | 474.5 | 286.66 | 0.60 | 0.0011 | 0.0094 |
| AOC3 | 93.24 | 7.88 | 0.08 | 9.20E−08 | 3.70E−06 |
| AP5B1 | 259.93 | 152.31 | 0.59 | 0.00061 | 0.006 |
| APLP2 | 1344.84 | 700.79 | 0.52 | 1.10E−10 | 9.10E−09 |
| APOBEC3D | 308.49 | 471.97 | 1.53 | 8.80E−09 | 4.70E−07 |
| APOC1 | 1105.55 | 325.48 | 0.29 | 7.40E−10 | 5.10E−08 |
| APOE | 1090.42 | 563.57 | 0.52 | 0.00011 | 0.0015 |
| APOL4 | 77.65 | 17.36 | 0.22 | 9.90E−07 | 3.00E−05 |
| APOLD1 | 29.67 | 58.56 | 1.97 | 8.90E−05 | 0.0013 |
| AQP9 | 120.64 | 36.76 | 0.30 | 0.00092 | 0.0084 |
| ARHGAP1 | 1038.19 | 674.81 | 0.65 | 4.30E−07 | 1.50E−05 |
| ARHGAP10 | 131.02 | 83.87 | 0.64 | 0.0048 | 0.03 |
| ARHGAP11A | 112.75 | 182.17 | 1.62 | 0.0064 | 0.037 |
| ARHGEF10L | 55.98 | 12.17 | 0.22 | 2.30E−06 | 5.90E−05 |
| ARHGEF26- | 8.81 | 14.06 | 1.60 | 0.0027 | 0.019 |
| AS1 | | | | | |
| ARL11 | 48.85 | 28.15 | 0.58 | 0.0093 | 0.049 |
| ARL3 | 134.08 | 250.31 | 1.87 | 0.00014 | 0.0018 |
| ARPM1 | 10.61 | 56.39 | 5.31 | 0.0065 | 0.038 |
| ARRB1 | 251.87 | 93.17 | 0.37 | 3.00E−06 | 7.60E−05 |
| ARRB2 | 749.61 | 480.04 | 0.64 | 1.40E−09 | 9.10E−08 |
| ARRDC2 | 433.21 | 281.53 | 0.65 | 0.00038 | 0.0041 |
| ARRDC4 | 149.11 | 25.18 | 0.17 | 2.70E−11 | 2.70E−09 |
| ARVCF | 14.67 | 4.76 | 0.32 | 0.0091 | 0.048 |
| ASAH1 | 1356.57 | 724.42 | 0.53 | 8.30E−11 | 7.30E−09 |
| ASB13 | 65.46 | 23.05 | 0.35 | 0.00037 | 0.0041 |
| ASB2 | 113.58 | 505.83 | 4.45 | 2.50E−11 | 2.60E−09 |
| ASCL2 | 69.43 | 17.47 | 0.25 | 3.30E−07 | 1.20E−05 |
| ASGR1 | 24.94 | 2.5 | 0.10 | 2.60E−11 | 2.70E−09 |
| ASPM | 94.64 | 357.68 | 3.78 | 5.60E−06 | 0.00013 |
| ATAD2 | 249.67 | 459.52 | 1.84 | 5.50E−05 | 0.00087 |
| ATF3 | 218.88 | 628.23 | 2.87 | 9.30E−05 | 0.0013 |
| ATF7IP2 | 109.83 | 189.16 | 1.72 | 0.00044 | 0.0047 |
| ATG4D | 284.32 | 183.84 | 0.65 | 0.0019 | 0.015 |
| ATP13A2 | 273.5 | 157.24 | 0.57 | 0.0011 | 0.0094 |
| ATP1B1 | 410.59 | 258.4 | 0.63 | 0.0063 | 0.037 |
| ATP2B1 | 1721.7 | 827.76 | 0.48 | 8.20E−09 | 4.40E−07 |
| ATP6AP1 | 1876.39 | 1233.41 | 0.66 | 9.90E−11 | 8.50E−09 |
| ATP6V0D1 | 1480.25 | 930.53 | 0.63 | 5.00E−07 | 1.60E−05 |
| ATP8B4 | 86.94 | 243.66 | 2.80 | 6.10E−07 | 2.00E−05 |
| ATP9A | 18.39 | 68.38 | 3.72 | 0.00016 | 0.0021 |
| AXIN1 | 264.98 | 149.26 | 0.56 | 0.00011 | 0.0016 |
| AXL | 509.67 | 137.04 | 0.27 | 1.80E−07 | 6.70E−06 |
| B3GNT7 | 162.71 | 64.93 | 0.40 | 2.60E−05 | 0.00046 |
| BANK1 | 8.22 | 31.84 | 3.87 | 0.0094 | 0.049 |
| BARD1 | 163.25 | 262.33 | 1.61 | 0.00051 | 0.0052 |
| BBC3 | 42.39 | 23.53 | 0.56 | 0.0013 | 0.011 |
| BBS1 | 139.13 | 69.69 | 0.50 | 3.60E−05 | 6.00E−04 |
| BCAR3 | 25.31 | 5.39 | 0.21 | 0.00047 | 0.005 |
| BCL9 | 116.84 | 77.23 | 0.66 | 0.0087 | 0.047 |
| BCORL1 | 96.43 | 58.03 | 0.60 | 0.0085 | 0.046 |
| BEND4 | 4.04 | 41.14 | 10.18 | 0.00011 | 0.0015 |
| BFSP1 | 44.04 | 12.54 | 0.28 | 0.0015 | 0.012 |
| BHLHE41 | 86.92 | 21.41 | 0.25 | 8.70E−08 | 3.50E−06 |
| BLM | 121.66 | 184.86 | 1.52 | 0.0062 | 0.036 |
| BLOC1S3 | 249.13 | 152.5 | 0.61 | 0.0013 | 0.011 |
| BLZF1 | 197.94 | 298.32 | 1.51 | 3.20E−08 | 1.50E−06 |
| BMF | 42.87 | 78.35 | 1.83 | 0.0022 | 0.017 |
| BMP8A | 23.62 | 46.78 | 1.98 | 0.0079 | 0.043 |
| BMP8B | 13.74 | 8.14 | 0.59 | 0.0042 | 0.027 |
| BNC2 | 23.84 | 6.2 | 0.26 | 0.001 | 0.0092 |
| BPIFB1 | 276.03 | 37.37 | 0.14 | 8.10E−05 | 0.0012 |
| BRI3 | 336.65 | 197.88 | 0.59 | 1.00E−05 | 0.00022 |
| BST1 | 44.45 | 10.43 | 0.23 | 0.00014 | 0.0019 |
| BTBD16 | 1.47 | 19.46 | 13.24 | 0.0026 | 0.019 |
| BTBD6 | 115.68 | 68.62 | 0.59 | 0.00095 | 0.0086 |
| BTG3 | 307 | 469.89 | 1.53 | 0.0039 | 0.025 |
| BTK | 85.38 | 21.01 | 0.25 | 4.80E−08 | 2.10E−06 |
| BUB1 | 69.94 | 292.41 | 4.18 | 1.50E−06 | 4.20E−05 |
| BUB1B | 35.04 | 123.63 | 3.53 | 0.0094 | 0.049 |
| C10orf116 | 13.66 | 2.8 | 0.20 | 0.0011 | 0.0098 |
| C10orf54 | 2282.17 | 1480.44 | 0.65 | 7.80E−07 | 2.40E−05 |
| C11orf74 | 14.99 | 4.1 | 0.27 | 0.0016 | 0.013 |
| C15orf38 | 15.17 | 4.03 | 0.27 | 0.0013 | 0.011 |
| C16orf54 | 2615.69 | 1537.25 | 0.59 | 4.40E−13 | 6.50E−11 |
| C17orf51 | 24.97 | 9.07 | 0.36 | 0.00026 | 0.003 |
| C17orf72 | 16.75 | 7.92 | 0.47 | 0.00074 | 0.007 |
| C18orf1 | 403.83 | 889.77 | 2.20 | 1.30E−11 | 1.50E−09 |
| C19orf35 | 60.12 | 18.99 | 0.32 | 1.70E−06 | 4.60E−05 |
| C19orf59 | 779.36 | 56.56 | 0.07 | 9.60E−17 | 3.50E−14 |
| C1orf106 | 13.37 | 46.42 | 3.47 | 0.00026 | 0.0031 |
| C1orf162 | 591.44 | 230.41 | 0.39 | 6.40E−35 | 8.60E−31 |
| C1orf173 | 28.04 | 3.25 | 0.12 | 0.0048 | 0.03 |
| C1orf177 | 28.02 | 5.04 | 0.18 | 0.00011 | 0.0015 |
| C1orf186 | 5 | 11.43 | 2.29 | 0.0026 | 0.019 |
| C1orf187 | 68.1 | 33.19 | 0.49 | 0.0018 | 0.014 |
| C1orf21 | 520.55 | 246.69 | 0.47 | 2.90E−05 | 5.00E−04 |
| C1orf38 | 899.8 | 334.6 | 0.37 | 2.60E−11 | 2.70E−09 |
| C1orf63 | 582.43 | 880.04 | 1.51 | 5.90E−08 | 2.50E−06 |
| C1QA | 2050.2 | 412.99 | 0.20 | 5.80E−13 | 8.30E−11 |
| C1QB | 3288.56 | 592.68 | 0.18 | 2.50E−14 | 4.80E−12 |
| C1QC | 1780.2 | 418.5 | 0.24 | 5.60E−11 | 5.20E−09 |
| C2 | 264.75 | 66.81 | 0.25 | 0.00012 | 0.0016 |
| C20orf197 | 12.85 | 3.37 | 0.26 | 0.00098 | 0.0088 |
| C20orf27 | 286.57 | 160.31 | 0.56 | 0.00074 | 0.007 |
| C20orf85 | 33.13 | 3.57 | 0.11 | 2.20E−07 | 8.30E−06 |
| C20orf96 | 17.76 | 5.08 | 0.29 | 0.0012 | 0.01 |
| C21orf63 | 181.64 | 97.26 | 0.54 | 1.00E−05 | 0.00021 |
| C2orf18 | 1341.87 | 779.84 | 0.58 | 2.50E−07 | 9.00E−06 |
| C2orf89 | 96.82 | 40.73 | 0.42 | 4.50E−05 | 0.00074 |
| C3orf14 | 26.5 | 60.85 | 2.30 | 0.0087 | 0.047 |
| C4orf34 | 300.8 | 150.34 | 0.50 | 2.20E−05 | 0.00039 |
| C5 | 7.73 | 20.43 | 2.64 | 0.0079 | 0.043 |
| C5AR1 | 270.31 | 77.7 | 0.29 | 1.20E−08 | 6.30E−07 |
| C5orf25 | 45.44 | 103.99 | 2.29 | 0.00096 | 0.0087 |
| C6orf108 | 152.88 | 336.63 | 2.20 | 1.60E−05 | 0.00031 |
| C6orf211 | 215.34 | 327.39 | 1.52 | 0.0039 | 0.026 |
| C6orf225 | 30.51 | 12.6 | 0.41 | 3.00E−04 | 0.0034 |
| C7orf58 | 49.38 | 15.04 | 0.30 | 0.00053 | 0.0054 |
| C8orf45 | 10.04 | 21.26 | 2.12 | 0.0074 | 0.041 |
| C8orf82 | 57.61 | 32.3 | 0.56 | 3.60E−05 | 0.00061 |
| C9orf167 | 39.77 | 19.7 | 0.50 | 0.0035 | 0.024 |
| C9orf21 | 287.66 | 166.62 | 0.58 | 7.60E−05 | 0.0011 |
| C9orf24 | 36.42 | 9.47 | 0.26 | 0.00053 | 0.0054 |
| C9orf3 | 52.88 | 91.75 | 1.74 | 0.0048 | 0.03 |
| C9orf9 | 17.64 | 7.66 | 0.43 | 6.00E−04 | 0.006 |
| CACNA2D2 | 111.26 | 45.19 | 0.41 | 1.10E−05 | 0.00022 |
| CAMK1 | 54.84 | 182.01 | 3.32 | 1.40E−06 | 3.90E−05 |
| CAMK2N1 | 18.35 | 7.96 | 0.43 | 0.0019 | 0.015 |
| CAMK4 | 784.42 | 1183.1 | 1.51 | 2.60E−05 | 0.00046 |
| CAPS | 126.23 | 53.98 | 0.43 | 2.50E−05 | 0.00045 |
| CARD17 | 4.53 | 17.88 | 3.95 | 7.00E−04 | 0.0067 |
| CARD6 | 71.58 | 39.34 | 0.55 | 0.0075 | 0.042 |
| CARD9 | 31.12 | 8.52 | 0.27 | 0.007 | 0.04 |
| CASC5 | 50.01 | 163.86 | 3.28 | 8.50E−06 | 0.00018 |
| CASP10 | 186.48 | 116.92 | 0.63 | 0.0031 | 0.021 |
| CBR3 | 13.61 | 31.87 | 2.34 | 0.0061 | 0.036 |
| CBX3P2 | 17 | 8.51 | 0.50 | 0.0013 | 0.011 |
| CBX5 | 735.16 | 1138.79 | 1.55 | 1.30E−05 | 0.00026 |
| CBX6 | 231.58 | 142.68 | 0.62 | 3.00E−05 | 0.00051 |
| CCDC141 | 283.55 | 684.56 | 2.41 | 2.00E−15 | 5.20E−13 |
| CCDC28B | 83.1 | 51.2 | 0.62 | 0.0012 | 0.01 |
| CCDC65 | 143.23 | 73.76 | 0.51 | 0.00061 | 0.006 |
| CCL18 | 1685.27 | 403.43 | 0.24 | 3.70E−08 | 1.70E−06 |
| CCL23 | 18.65 | 6.91 | 0.37 | 0.0037 | 0.025 |
| CCNA2 | 49.46 | 203.23 | 4.11 | 5.70E−05 | 0.00089 |
| CCNB1 | 47.48 | 126.75 | 2.67 | 0.0043 | 0.027 |
| CCNB2 | 36.22 | 115.3 | 3.18 | 0.0074 | 0.041 |
| CCND3 | 5289.58 | 3170.53 | 0.60 | 1.90E−16 | 6.20E−14 |
| CCR5 | 1089.5 | 1691.96 | 1.55 | 7.50E−11 | 6.70E−09 |
| CD109 | 59.46 | 112.82 | 1.90 | 0.0025 | 0.018 |
| CD163 | 464.15 | 179.04 | 0.39 | 0.0021 | 0.016 |
| CD200 | 6.87 | 81.47 | 11.86 | 2.40E−07 | 8.80E−06 |
| CD200R1 | 366.89 | 685.98 | 1.87 | 1.20E−06 | 3.50E−05 |
| CD27 | 684.59 | 1196.19 | 1.75 | 5.20E−06 | 0.00012 |
| CD276 | 91.56 | 25.41 | 0.28 | 0.0012 | 0.01 |
| CD300A | 663.38 | 249.36 | 0.38 | 1.90E−11 | 2.10E−09 |
| CD300C | 81.9 | 11.32 | 0.14 | 2.70E−09 | 1.60E−07 |
| CD300LF | 118.42 | 23.68 | 0.20 | 2.80E−07 | 9.90E−06 |
| CD320 | 369.9 | 236.91 | 0.64 | 0.0079 | 0.044 |
| CD33 | 65.48 | 20.02 | 0.31 | 6.50E−05 | 0.00098 |
| CD36 | 119.8 | 53.56 | 0.45 | 0.00067 | 0.0065 |
| CD38 | 70.51 | 350.75 | 4.97 | 3.60E−08 | 1.60E−06 |
| CD4 | 549.92 | 184 | 0.33 | 5.10E−06 | 0.00012 |
| CD55 | 1651.23 | 1025.6 | 0.62 | 3.40E−05 | 0.00057 |
| CD59 | 691.75 | 423.87 | 0.61 | 7.20E−05 | 0.0011 |
| CD68 | 2154.58 | 503.18 | 0.23 | 2.40E−15 | 6.10E−13 |
| CD79A | 28.16 | 63.91 | 2.27 | 0.0059 | 0.035 |
| CD82 | 909.63 | 1573.58 | 1.73 | 1.50E−08 | 7.60E−07 |
| CD96 | 4917.4 | 7608.02 | 1.55 | 1.20E−10 | 9.90E−09 |
| CD97 | 6854.66 | 3986.74 | 0.58 | 1.40E−12 | 1.80E−10 |
| CDC20 | 26.82 | 113.07 | 4.22 | 0.0038 | 0.025 |
| CDC25C | 2.39 | 21.79 | 9.12 | 0.0016 | 0.013 |
| CDC42BPB | 52.16 | 10.2 | 0.20 | 2.30E−06 | 5.90E−05 |
| CDC45 | 17 | 65.69 | 3.86 | 0.00049 | 0.0051 |
| CDC6 | 22.78 | 91.64 | 4.02 | 2.40E−06 | 6.20E−05 |
| CDCA2 | 19.56 | 84.53 | 4.32 | 0.0011 | 0.0096 |
| CDCA3 | 12.48 | 43.58 | 3.49 | 0.0045 | 0.029 |
| CDCA7 | 150.38 | 327.37 | 2.18 | 0.0018 | 0.014 |
| CDCP1 | 81.08 | 37.28 | 0.46 | 0.009 | 0.048 |
| CDH17 | 0.33 | 15.38 | 46.61 | 0.00024 | 0.0029 |
| CDK1 | 84.88 | 245.7 | 2.89 | 2.10E−05 | 0.00038 |
| CDKN2D | 295.68 | 182.57 | 0.62 | 4.40E−05 | 0.00072 |
| CDKN3 | 56.42 | 130.57 | 2.31 | 0.0074 | 0.041 |
| CDT1 | 28.78 | 65.83 | 2.29 | 0.0094 | 0.049 |
| CEACAM5 | 11.23 | 76.01 | 6.77 | 3.10E−06 | 7.90E−05 |
| CEACAM6 | 96.15 | 457.13 | 4.75 | 0.00061 | 0.006 |
| CEBPA | 119.96 | 39.96 | 0.33 | 3.30E−05 | 0.00056 |
| CEBPB | 398.93 | 202.12 | 0.51 | 1.20E−08 | 6.30E−07 |
| CEND1 | 4.5 | 17.98 | 4.00 | 0.0026 | 0.019 |
| CENPBD1 | 66.43 | 38.18 | 0.57 | 0.0029 | 0.021 |
| CENPE | 97.02 | 235.23 | 2.42 | 8.20E−06 | 0.00018 |
| CENPF | 162.99 | 346.48 | 2.13 | 5.20E−05 | 0.00083 |
| CENPM | 87.67 | 159.14 | 1.82 | 0.0088 | 0.047 |
| CEP78 | 450.54 | 282.73 | 0.63 | 0.0025 | 0.018 |
| CES1 | 651.9 | 37.53 | 0.06 | 3.20E−29 | 2.20E−25 |
| CES4A | 37.89 | 15.19 | 0.40 | 0.0066 | 0.038 |
| CFD | 277.22 | 94.37 | 0.34 | 1.50E−09 | 9.60E−08 |
| CFH | 271.56 | 84.07 | 0.31 | 3.90E−10 | 2.90E−08 |
| CFP | 37.14 | 9.59 | 0.26 | 0.003 | 0.021 |
| CHDH | 22.3 | 12.4 | 0.56 | 0.0035 | 0.024 |
| CHEK1 | 67 | 117.91 | 1.76 | 0.0011 | 0.0096 |
| CHML | 67.9 | 107.76 | 1.59 | 0.0011 | 0.0098 |
| CHN1 | 94.15 | 345.78 | 3.67 | 1.00E−09 | 6.80E−08 |
| CHST14 | 44.67 | 23.02 | 0.52 | 0.00051 | 0.0053 |
| CISH | 2226.51 | 1438.93 | 0.65 | 0.0019 | 0.015 |
| CKAP2 | 235.94 | 669.65 | 2.84 | 6.50E−14 | 1.10E−11 |
| CKAP2L | 33.07 | 104.83 | 3.17 | 0.0066 | 0.038 |
| CKLF | 202.53 | 317.9 | 1.57 | 0.0025 | 0.018 |
| CKS1B | 150.55 | 286.61 | 1.90 | 0.0076 | 0.042 |
| CKS2 | 207.14 | 515.25 | 2.49 | 5.30E−09 | 2.90E−07 |
| CLASP1 | 444.4 | 669.3 | 1.51 | 9.30E−07 | 2.80E−05 |
| CLCF1 | 120.68 | 36.31 | 0.30 | 5.70E−05 | 0.00089 |
| CLDND1 | 2254.96 | 3825.53 | 1.70 | 1.20E−08 | 6.20E−07 |
| CLEC4E | 27.41 | 5.63 | 0.21 | 9.30E−05 | 0.0013 |
| CLEC7A | 146.1 | 47.56 | 0.33 | 1.40E−08 | 7.00E−07 |
| CLECL1 | 95.05 | 154.99 | 1.63 | 0.0078 | 0.043 |
| CLIC4 | 149.5 | 61.76 | 0.41 | 0.00056 | 0.0056 |
| CLIC5 | 220 | 485.08 | 2.20 | 4.90E−11 | 4.60E−09 |
| CLK4 | 350.91 | 563.72 | 1.61 | 1.40E−06 | 4.10E−05 |
| CLMN | 42.53 | 16.53 | 0.39 | 0.00024 | 0.0028 |
| CLNK | 44.75 | 157.15 | 3.51 | 1.20E−06 | 3.40E−05 |
| CLSPN | 54.96 | 113.26 | 2.06 | 0.0034 | 0.023 |
| CLU | 434.1 | 229.4 | 0.53 | 2.60E−06 | 6.70E−05 |
| CMKLR1 | 249.05 | 84.24 | 0.34 | 0.00017 | 0.0022 |
| CNN3 | 18.07 | 58.23 | 3.22 | 0.009 | 0.048 |
| COL6A2 | 196.74 | 55.93 | 0.28 | 3.60E−07 | 1.20E−05 |
| COL6A3 | 16.31 | 114.62 | 7.03 | 2.70E−06 | 6.90E−05 |
| COLEC12 | 57.76 | 33.08 | 0.57 | 0.0033 | 0.023 |
| CPNE7 | 114.75 | 286.15 | 2.49 | 6.90E−08 | 2.90E−06 |
| CPVL | 221.54 | 92.55 | 0.42 | 0.0016 | 0.013 |
| CRABP2 | 2.84 | 42.95 | 15.12 | 0.00055 | 0.0056 |
| CRIP1 | 5553.14 | 3364.55 | 0.61 | 1.80E−14 | 3.60E−12 |
| CRIP2 | 137.49 | 56.73 | 0.41 | 2.50E−05 | 0.00045 |
| CRTAM | 1134.13 | 1762.23 | 1.55 | 3.20E−05 | 0.00054 |
| CSDA | 261.42 | 138.86 | 0.53 | 9.10E−07 | 2.70E−05 |
| CSF1 | 679.12 | 1139.86 | 1.68 | 3.20E−06 | 7.90E−05 |
| CSF1R | 244.45 | 124.21 | 0.51 | 3.90E−07 | 1.30E−05 |
| CSF2RB | 59.01 | 24.99 | 0.42 | 0.0037 | 0.025 |
| CSPG4 | 16.65 | 1.29 | 0.08 | 0.0021 | 0.016 |
| CST3 | 1571.93 | 356.21 | 0.23 | 3.50E−09 | 2.10E−07 |
| CSTA | 72.89 | 29.59 | 0.41 | 4.30E−06 | 1.00E−04 |
| CSTF3 | 187.64 | 306.8 | 1.64 | 0.00044 | 0.0047 |
| CTBP2 | 143.44 | 40.02 | 0.28 | 4.10E−07 | 1.40E−05 |
| CTLA4 | 281.21 | 1077.82 | 3.83 | 1.40E−10 | 1.10E−08 |
| CTNNAL1 | 44.45 | 81.45 | 1.83 | 0.0079 | 0.044 |
| CTSD | 12888.26 | 6652.84 | 0.52 | 3.10E−07 | 1.10E−05 |
| CTSF | 184.49 | 77.98 | 0.42 | 0.0011 | 0.0099 |
| CTSS | 2886.11 | 1815.92 | 0.63 | 2.90E−09 | 1.70E−07 |
| CUL9 | 196.26 | 312.08 | 1.59 | 0.0027 | 0.019 |
| CX3CR1 | 3407.32 | 838.28 | 0.25 | 1.40E−16 | 4.60E−14 |
| CXCL13 | 172.01 | 3795.11 | 22.06 | 4.50E−23 | 8.50E−20 |
| CXCL16 | 837.33 | 173.81 | 0.21 | 9.20E−11 | 8.00E−09 |
| CXCL3 | 132.51 | 21.6 | 0.16 | 4.00E−10 | 2.90E−08 |
| CXCL5 | 111.31 | 9.35 | 0.08 | 5.90E−16 | 1.70E−13 |
| CXCR1 | 156.67 | 39.57 | 0.25 | 4.80E−05 | 0.00078 |
| CXCR2 | 160.53 | 31.15 | 0.19 | 1.40E−15 | 3.90E−13 |
| CXCR5 | 65.47 | 264.58 | 4.04 | 3.90E−09 | 2.20E−07 |
| CXCR6 | 3031.23 | 6111.37 | 2.02 | 6.20E−09 | 3.40E−07 |
| CXXC4 | 13.42 | 2.25 | 0.17 | 4.00E−04 | 0.0043 |
| CYB561 | 245.6 | 144.89 | 0.59 | 0.00047 | 0.0049 |
| CYB5R2 | 21.93 | 3.04 | 0.14 | 0.00032 | 0.0036 |
| CYB5R3 | 1375.95 | 869.38 | 0.63 | 1.40E−09 | 8.70E−08 |
| CYBA | 4565.56 | 2936.98 | 0.64 | 2.00E−09 | 1.30E−07 |
| CYBB | 827.95 | 309.88 | 0.37 | 1.80E−05 | 0.00035 |
| CYP27A1 | 682.79 | 102.76 | 0.15 | 2.90E−09 | 1.70E−07 |
| CYP2F1 | 19.03 | 8.02 | 0.42 | 0.0059 | 0.035 |
| CYSTM1 | 265.08 | 151.48 | 0.57 | 0.00092 | 0.0084 |
| CYTH1 | 1841.29 | 1109.35 | 0.60 | 4.30E−06 | 1.00E−04 |
| D4S234E | 298.55 | 95.47 | 0.32 | 4.80E−09 | 2.70E−07 |
| DAB2 | 262.46 | 101.11 | 0.39 | 3.10E−10 | 2.30E−08 |
| DAGLA | 12.16 | 3.02 | 0.25 | 0.0056 | 0.034 |
| DAPK1 | 144.08 | 49.84 | 0.35 | 0.00071 | 0.0068 |
| DAPK2 | 172.15 | 438.08 | 2.54 | 3.30E−11 | 3.20E−09 |
| DBH | 4.51 | 27.3 | 6.05 | 3.10E−05 | 0.00053 |
| DDIT3 | 176.63 | 290.74 | 1.65 | 0.0013 | 0.011 |
| DDX28 | 177.37 | 103.07 | 0.58 | 0.0049 | 0.031 |
| DDX60 | 848.42 | 1325.7 | 1.56 | 9.10E−07 | 2.80E−05 |
| DEFB1 | 16.76 | 1.29 | 0.08 | 1.00E−04 | 0.0014 |
| DEGS2 | 17.08 | 5.17 | 0.30 | 0.0081 | 0.044 |
| DENND5A | 184.1 | 83.27 | 0.45 | 0.00018 | 0.0022 |
| DEPDC1 | 5.57 | 51.58 | 9.26 | 3.00E−04 | 0.0034 |
| DGKD | 500.07 | 301.84 | 0.60 | 1.20E−05 | 0.00025 |
| DGKH | 77.19 | 141.48 | 1.83 | 0.0063 | 0.037 |
| DGKQ | 191.58 | 126.24 | 0.66 | 0.00037 | 0.004 |
| DHCR24 | 298.11 | 170.29 | 0.57 | 0.0011 | 0.0098 |
| DHFR | 69.52 | 156.69 | 2.25 | 8.00E−04 | 0.0075 |
| DHRS13 | 170.72 | 110.86 | 0.65 | 0.0013 | 0.011 |
| DIAPH3 | 4.83 | 26.3 | 5.45 | 0.00028 | 0.0033 |
| DKK3 | 113.56 | 361.06 | 3.18 | 2.70E−05 | 0.00047 |
| DLEC1 | 39.34 | 14.2 | 0.36 | 0.00021 | 0.0026 |
| DLEU2 | 46.86 | 80.74 | 1.72 | 0.0017 | 0.013 |
| DLG1 | 173.13 | 268.79 | 1.55 | 1.10E−05 | 0.00023 |
| DLG5 | 72.77 | 34.55 | 0.47 | 0.0021 | 0.016 |
| DLGAP5 | 17.83 | 102.7 | 5.76 | 2.20E−05 | 0.00039 |
| DMC1 | 5.14 | 13.57 | 2.64 | 0.0063 | 0.037 |
| DMKN | 24.67 | 7.8 | 0.32 | 1.00E−04 | 0.0015 |
| DNAH6 | 31.85 | 11.44 | 0.36 | 6.00E−04 | 0.0059 |
| DNAH7 | 32.93 | 6.8 | 0.21 | 9.80E−05 | 0.0014 |
| DNAI2 | 17.8 | 3.25 | 0.18 | 7.50E−06 | 0.00016 |
| DNAJA1 | 1407.56 | 2318.02 | 1.65 | 0.00072 | 0.0068 |
| DNAJA4 | 115.64 | 203.71 | 1.76 | 0.0012 | 0.01 |
| DNAJB1 | 5193.77 | 12981.84 | 2.50 | 2.00E−08 | 9.50E−07 |
| DNAJB4 | 88.29 | 402.67 | 4.56 | 5.40E−10 | 3.80E−08 |
| DNAJC28 | 24.37 | 12.33 | 0.51 | 0.0071 | 0.04 |
| DNAJC5 | 425.54 | 251.7 | 0.59 | 2.70E−05 | 0.00047 |
| DOK3 | 104.07 | 33.84 | 0.33 | 0.0018 | 0.014 |
| DPEP2 | 222.21 | 75.15 | 0.34 | 7.30E−08 | 3.00E−06 |
| DPYSL2 | 597.62 | 358.85 | 0.60 | 5.20E−08 | 2.20E−06 |
| DSC1 | 26.17 | 4 | 0.15 | 5.70E−06 | 0.00013 |
| DSEL | 66.84 | 28.67 | 0.43 | 0.00016 | 0.002 |
| DSG2 | 6.88 | 32.19 | 4.68 | 0.00036 | 0.0039 |
| DST | 113.35 | 39.23 | 0.35 | 0.0011 | 0.0094 |
| DSTN | 1337.48 | 853.58 | 0.64 | 1.30E−06 | 3.70E−05 |
| DTHD1 | 570.29 | 932.48 | 1.64 | 8.90E−06 | 0.00019 |
| DTL | 46.01 | 135.87 | 2.95 | 0.0031 | 0.022 |
| DTX4 | 35.6 | 6.48 | 0.18 | 0.00044 | 0.0047 |
| DUSP16 | 269.98 | 423.97 | 1.57 | 0.0088 | 0.047 |
| DUSP4 | 1013.21 | 2408.59 | 2.38 | 2.10E−12 | 2.60E−10 |
| DYNLRB2 | 19.49 | 9 | 0.46 | 0.0071 | 0.04 |
| DZIP3 | 405.74 | 760.99 | 1.88 | 3.30E−08 | 1.50E−06 |
| E2F7 | 6.14 | 34.62 | 5.64 | 0.00029 | 0.0033 |
| ECT2L | 25.43 | 8.71 | 0.34 | 0.00086 | 0.0079 |
| EDEM2 | 562.14 | 374.17 | 0.67 | 0.00016 | 0.0021 |
| EFHC2 | 22.06 | 4.32 | 0.20 | 0.0023 | 0.017 |
| EFHD2 | 1086.54 | 555.11 | 0.51 | 1.10E−16 | 3.90E−14 |
| EFNA5 | 111.74 | 46.67 | 0.42 | 0.0058 | 0.035 |
| EFNB1 | 60.33 | 34.73 | 0.58 | 0.0056 | 0.034 |
| EGR1 | 826.87 | 1401.52 | 1.69 | 0.0063 | 0.037 |
| EHD4 | 346.69 | 195.91 | 0.57 | 1.90E−06 | 5.10E−05 |
| EIF2AK2 | 575.62 | 912.79 | 1.59 | 0.00028 | 0.0033 |
| ELK2AP | 27.72 | 389.24 | 14.04 | 2.50E−16 | 8.10E−14 |
| ELL2 | 218.53 | 127.8 | 0.58 | 0.00063 | 0.0061 |
| EMILIN2 | 163.88 | 82.33 | 0.50 | 0.0019 | 0.015 |
| EMP3 | 3118 | 1779.13 | 0.57 | 1.90E−14 | 3.80E−12 |
| EMR1 | 71.3 | 6.77 | 0.09 | 8.10E−08 | 3.30E−06 |
| ENG | 425.13 | 237.99 | 0.56 | 9.80E−05 | 0.0014 |
| ENPP5 | 298.27 | 131.43 | 0.44 | 0.0017 | 0.013 |
| ENTPD1 | 333.35 | 1566.32 | 4.70 | 2.20E−12 | 2.70E−10 |
| EPB41L1 | 46.87 | 12.46 | 0.27 | 6.60E−05 | 0.001 |
| EPB41L4A | 136.55 | 63.29 | 0.46 | 0.0017 | 0.014 |
| EPCAM | 18.08 | 82.29 | 4.55 | 0.0011 | 0.0095 |
| EPG5 | 189.33 | 299.35 | 1.58 | 5.50E−05 | 0.00086 |
| EPHA1 | 245.57 | 471.69 | 1.92 | 3.70E−06 | 9.10E−05 |
| EPHX4 | 22.46 | 6.04 | 0.27 | 0.0038 | 0.025 |
| ERBB3 | 27.13 | 59.02 | 2.18 | 8.10E−05 | 0.0012 |
| ESCO2 | 17.58 | 60.51 | 3.44 | 0.0066 | 0.038 |
| ETAA1 | 165.36 | 254.98 | 1.54 | 0.00061 | 0.006 |
| ETV1 | 81.44 | 368.39 | 4.52 | 2.20E−08 | 1.00E−06 |
| EVC | 6.59 | 35.71 | 5.42 | 0.00011 | 0.0015 |
| EVC2 | 50.75 | 120.57 | 2.38 | 0.0033 | 0.023 |
| EZH2 | 149.67 | 333.68 | 2.23 | 0.00012 | 0.0016 |
| F8A1 | 49.51 | 27.42 | 0.55 | 0.00026 | 0.003 |
| FABP3 | 75.66 | 27.95 | 0.37 | 2.70E−07 | 9.90E−06 |
| FABP4 | 1434.18 | 64.61 | 0.05 | 2.80E−20 | 2.20E−17 |
| FADS1 | 133.9 | 78.05 | 0.58 | 0.0012 | 0.01 |
| FADS3 | 53.39 | 28.17 | 0.53 | 0.0031 | 0.021 |
| FAM105B | 442.43 | 701.97 | 1.59 | 1.70E−13 | 2.70E−11 |
| FAM109A | 23.01 | 7.14 | 0.31 | 0.0028 | 0.02 |
| FAM160B1 | 531.62 | 820.37 | 1.54 | 0.00083 | 0.0077 |
| FAM166B | 11.11 | 50.7 | 4.56 | 0.00097 | 0.0087 |
| FAM172BP | 14.69 | 39.65 | 2.70 | 0.00021 | 0.0025 |
| FAM174B | 6.83 | 38.99 | 5.71 | 0.0027 | 0.019 |
| FAM184A | 23.67 | 47.68 | 2.01 | 0.0083 | 0.045 |
| FAM18B2 | 45.58 | 70.54 | 1.55 | 0.0067 | 0.038 |
| FAM19A1 | 63.72 | 11.81 | 0.19 | 1.80E−07 | 6.80E−06 |
| FAM216B | 39.25 | 7.59 | 0.19 | 3.80E−08 | 1.70E−06 |
| FAM3B | 21.09 | 5.47 | 0.26 | 0.0054 | 0.033 |
| FAM40B | 9.12 | 16.26 | 1.78 | 0.0036 | 0.024 |
| FAM65A | 292.27 | 180.55 | 0.62 | 0.0024 | 0.017 |
| FAM65B | 2858.72 | 1347.03 | 0.47 | 3.10E−11 | 3.00E−09 |
| FAM82A2 | 551.93 | 321.09 | 0.58 | 1.10E−07 | 4.30E−06 |
| FAM83D | 38.6 | 133.48 | 3.46 | 0.00054 | 0.0055 |
| FAM89A | 75.74 | 32.97 | 0.44 | 0.0068 | 0.039 |
| FAM92B | 21.08 | 1.4 | 0.07 | 4.10E−07 | 1.40E−05 |
| FANCI | 112.84 | 245.02 | 2.17 | 0.0013 | 0.011 |
| FANCL | 101.37 | 180.82 | 1.78 | 0.003 | 0.021 |
| FANCM | 111.43 | 209.52 | 1.88 | 0.0019 | 0.014 |
| FASLG | 977.25 | 1653.28 | 1.69 | 3.20E−08 | 1.50E−06 |
| FBP1 | 1884.75 | 337.99 | 0.18 | 2.50E−15 | 6.10E−13 |
| FBXW5 | 1331.57 | 833.96 | 0.63 | 9.80E−09 | 5.20E−07 |
| FCER1G | 1503.38 | 476.39 | 0.32 | 2.40E−12 | 3.00E−10 |
| FCGR1A | 40.84 | 13.47 | 0.33 | 3.80E−05 | 0.00063 |
| FCGR2A | 430.23 | 149.82 | 0.35 | 8.10E−05 | 0.0012 |
| FCGR3A | 3198.42 | 731.82 | 0.23 | 9.30E−23 | 1.40E−19 |
| FCGR3B | 57.17 | 15.22 | 0.27 | 8.10E−06 | 0.00018 |
| FCGRT | 679.04 | 285.89 | 0.42 | 7.20E−06 | 0.00016 |
| FCN1 | 78.26 | 16.4 | 0.21 | 1.60E−07 | 6.40E−06 |
| FCRL6 | 2377.67 | 1451.72 | 0.61 | 0.00082 | 0.0076 |
| FCRLB | 35.86 | 11.74 | 0.33 | 0.0023 | 0.017 |
| FDPSL2A | 32.45 | 70.48 | 2.17 | 1.20E−06 | 3.60E−05 |
| FES | 54.81 | 24.46 | 0.45 | 0.0058 | 0.035 |
| FEZ1 | 117.65 | 44.95 | 0.38 | 1.30E−07 | 5.10E−06 |
| FGB | 5.92 | 19.58 | 3.31 | 0.002 | 0.015 |
| FGD2 | 127.19 | 76.48 | 0.60 | 0.0088 | 0.047 |
| FGD4 | 56.46 | 33.33 | 0.59 | 0.00096 | 0.0087 |
| FGD6 | 7.77 | 23.82 | 3.07 | 0.0012 | 0.01 |
| FGFBP2 | 1819.24 | 403.06 | 0.22 | 7.60E−22 | 1.00E−18 |
| FGFBP3 | 25.93 | 13.09 | 0.50 | 0.001 | 0.0091 |
| FGFR1 | 125.95 | 48.35 | 0.38 | 6.40E−06 | 0.00014 |
| FGR | 1278.09 | 407.19 | 0.32 | 2.70E−18 | 1.40E−15 |
| FHAD1 | 33.01 | 10.4 | 0.32 | 3.40E−05 | 0.00058 |
| FHL1 | 197.79 | 42.77 | 0.22 | 3.00E−09 | 1.70E−07 |
| FHL2 | 20.68 | 55.18 | 2.67 | 6.00E−04 | 0.0059 |
| FKBP14 | 33.49 | 51.84 | 1.55 | 0.0028 | 0.02 |
| FKBP1AP1 | 34.91 | 56.75 | 1.63 | 0.0025 | 0.018 |
| FKTN | 52.32 | 108.1 | 2.07 | 0.00044 | 0.0047 |
| FLJ14186 | 23.68 | 52.66 | 2.22 | 0.0088 | 0.047 |
| FLJ34690 | 7.17 | 12.13 | 1.69 | 0.0031 | 0.021 |
| FLNA | 5628.53 | 2950.61 | 0.52 | 2.60E−13 | 3.90E−11 |
| FLT1 | 17.99 | 46.96 | 2.61 | 0.00022 | 0.0026 |
| FLT3LG | 499.99 | 315.98 | 0.63 | 1.40E−08 | 6.90E−07 |
| FLT4 | 50.9 | 17.64 | 0.35 | 0.0066 | 0.038 |
| FLVCR2 | 130.87 | 54.02 | 0.41 | 0.0054 | 0.033 |
| FN1 | 3434.97 | 609.52 | 0.18 | 8.20E−10 | 5.50E−08 |
| FNDC3B | 594.31 | 337.14 | 0.57 | 1.00E−05 | 0.00021 |
| FOSL2 | 456.53 | 303.12 | 0.66 | 0.00093 | 0.0085 |
| FOXJ1 | 19.08 | 3.33 | 0.17 | 0.0034 | 0.023 |
| FPR1 | 264.22 | 33.41 | 0.13 | 1.10E−10 | 9.50E−09 |
| FPR2 | 82.24 | 11.26 | 0.14 | 0.00013 | 0.0017 |
| FRY | 50.44 | 20.74 | 0.41 | 0.0013 | 0.011 |
| FTH1 | 22475.03 | 9869.68 | 0.44 | 1.50E−18 | 8.90E−16 |
| FTL | 36289.33 | 16023.76 | 0.44 | 8.70E−16 | 2.50E−13 |
| FUT11 | 302.87 | 178.23 | 0.59 | 0.00035 | 0.0039 |
| FUT8 | 307.97 | 653.21 | 2.12 | 2.00E−09 | 1.30E−07 |
| FXYD7 | 35.11 | 12.98 | 0.37 | 0.00096 | 0.0087 |
| FZD4 | 54.49 | 35.62 | 0.65 | 0.0017 | 0.013 |
| FZD6 | 19.73 | 55.76 | 2.83 | 0.0027 | 0.019 |
| G6PC3 | 187.89 | 117.29 | 0.62 | 0.00084 | 0.0077 |
| G6PD | 766.07 | 430.23 | 0.56 | 7.90E−09 | 4.30E−07 |
| GAA | 661.85 | 233.43 | 0.35 | 9.00E−11 | 7.80E−09 |
| GALM | 426.56 | 891.22 | 2.09 | 6.90E−11 | 6.20E−09 |
| GALNT12 | 75.26 | 29.13 | 0.39 | 6.80E−05 | 0.001 |
| GALNT3 | 185.06 | 114.78 | 0.62 | 0.00025 | 0.003 |
| GAS2L1 | 33.59 | 6.13 | 0.18 | 5.10E−06 | 0.00012 |
| GAS7 | 187.89 | 83.74 | 0.45 | 1.10E−07 | 4.60E−06 |
| GBP1P1 | 9.05 | 30.96 | 3.42 | 0.0087 | 0.047 |
| GBP5 | 2472.04 | 4134.35 | 1.67 | 2.00E−04 | 0.0024 |
| GCHFR | 615.62 | 396.33 | 0.64 | 3.00E−04 | 0.0034 |
| GCNT1 | 263.21 | 446.71 | 1.70 | 0.0049 | 0.03 |
| GDPD5 | 178.72 | 74.03 | 0.41 | 4.00E−05 | 0.00066 |
| GEM | 10.37 | 300.91 | 29.02 | 1.00E−14 | 2.30E−12 |
| GGCT | 115.24 | 196.3 | 1.70 | 0.0046 | 0.029 |
| GGTA1P | 50.89 | 19.48 | 0.38 | 0.0058 | 0.035 |
| GINS1 | 13.35 | 37.92 | 2.84 | 0.009 | 0.048 |
| GLDC | 10.22 | 126.53 | 12.38 | 1.30E−06 | 3.80E−05 |
| GLDN | 157.83 | 12.66 | 0.08 | 2.70E−17 | 1.10E−14 |
| GLRX | 1011.42 | 648.92 | 0.64 | 3.80E−06 | 9.40E−05 |
| GLT25D1 | 725.84 | 394.44 | 0.54 | 3.20E−11 | 3.10E−09 |
| GLTPD1 | 178.6 | 118.82 | 0.67 | 0.0067 | 0.038 |
| GLUL | 4440.18 | 2366.67 | 0.53 | 8.50E−13 | 1.20E−10 |
| GNG4 | 14.32 | 49.9 | 3.48 | 2.30E−07 | 8.40E−06 |
| GNLY | 14788.96 | 7493.78 | 0.51 | 1.20E−05 | 0.00024 |
| GOLGA7B | 33.77 | 15.08 | 0.45 | 0.0065 | 0.037 |
| GOLIM4 | 140.92 | 278.08 | 1.97 | 0.0031 | 0.022 |
| GPA33 | 225.34 | 106.32 | 0.47 | 0.0059 | 0.035 |
| GPBAR1 | 33.85 | 4.94 | 0.15 | 0.0011 | 0.0096 |
| GPD1 | 215.95 | 18.01 | 0.08 | 7.00E−20 | 5.20E−17 |
| GPNMB | 1179.05 | 622.26 | 0.53 | 3.00E−04 | 0.0034 |
| GPR110 | 3.11 | 12.22 | 3.93 | 0.0082 | 0.045 |
| GPR113 | 26.47 | 57.48 | 2.17 | 1.80E−05 | 0.00034 |
| GPR141 | 26.15 | 10.17 | 0.39 | 0.0044 | 0.028 |
| GPR153 | 39.71 | 11.08 | 0.28 | 1.90E−06 | 5.20E−05 |
| GPR174 | 986.94 | 1655.89 | 1.68 | 1.80E−14 | 3.60E−12 |
| GPR25 | 179.82 | 403.48 | 2.24 | 5.70E−05 | 0.00089 |
| GPR34 | 74.66 | 214.15 | 2.87 | 0.00035 | 0.0039 |
| GPR56 | 3075.9 | 1323.58 | 0.43 | 2.70E−07 | 9.90E−06 |
| GPR82 | 54.58 | 116.76 | 2.14 | 0.00013 | 0.0017 |
| GPX1 | 1063.84 | 675.61 | 0.64 | 0.0019 | 0.015 |
| GPX3 | 222.15 | 61.53 | 0.28 | 3.40E−06 | 8.30E−05 |
| GRINA | 497.89 | 259.78 | 0.52 | 9.20E−07 | 2.80E−05 |
| GRK6 | 877.44 | 583.16 | 0.66 | 6.60E−07 | 2.10E−05 |
| GRN | 4617.98 | 1014.03 | 0.22 | 7.60E−14 | 1.30E−11 |
| GSG2 | 38.65 | 130.34 | 3.37 | 7.80E−10 | 5.30E−08 |
| GSN | 877.38 | 316 | 0.36 | 5.20E−08 | 2.20E−06 |
| GSTA1 | 26.16 | 10.25 | 0.39 | 0.0019 | 0.015 |
| GSTT1 | 85.59 | 40.09 | 0.47 | 0.0068 | 0.039 |
| GZMA | 6066.85 | 10287.1 | 1.70 | 4.60E−05 | 0.00074 |
| H2AFX | 138.63 | 264.94 | 1.91 | 8.60E−06 | 0.00018 |
| HAVCR1 | 4.86 | 23.42 | 4.82 | 0.00046 | 0.0049 |
| HAVCR2 | 448.29 | 1432.14 | 3.19 | 9.10E−08 | 3.70E−06 |
| HBEGF | 148.96 | 63.56 | 0.43 | 4.90E−07 | 1.60E−05 |
| HCAR2 | 73.49 | 16.45 | 0.22 | 0.00013 | 0.0017 |
| HCAR3 | 25.78 | 5.24 | 0.20 | 9.00E−05 | 0.0013 |
| HCK | 288.55 | 61.7 | 0.21 | 1.20E−08 | 6.20E−07 |
| HECTD2 | 63.8 | 162.49 | 2.55 | 2.90E−09 | 1.70E−07 |
| HELLS | 60.27 | 165.45 | 2.75 | 5.20E−10 | 3.80E−08 |
| HHEX | 23.72 | 7.16 | 0.30 | 0.00026 | 0.0031 |
| HIF1A | 1179.45 | 1781.47 | 1.51 | 1.10E−05 | 0.00022 |
| HIST1H1B | 40.41 | 100.55 | 2.49 | 0.0041 | 0.026 |
| HIST1H2AC | 110.89 | 176.63 | 1.59 | 0.00015 | 0.0019 |
| HIST1H2AG | 23.77 | 40.47 | 1.70 | 0.0091 | 0.048 |
| HIST1H2AH | 16.99 | 53.13 | 3.13 | 0.0039 | 0.026 |
| HIST1H2AJ | 11.71 | 32.43 | 2.77 | 5.90E−06 | 0.00014 |
| HIST1H2AL | 13.17 | 31.2 | 2.37 | 0.0048 | 0.03 |
| HIST1H2AM | 71.06 | 162.6 | 2.29 | 0.00024 | 0.0029 |
| HIST1H2BF | 11.43 | 30.26 | 2.65 | 0.009 | 0.048 |
| HIST1H2BH | 5.58 | 27.21 | 4.88 | 0.00069 | 0.0066 |
| HIST1H2BN | 24.98 | 37.96 | 1.52 | 0.0071 | 0.04 |
| HIST1H3D | 60 | 117.53 | 1.96 | 0.00068 | 0.0066 |
| HIST1H3G | 2.08 | 12.04 | 5.79 | 0.00049 | 0.0051 |
| HIST1H3I | 65.27 | 128.88 | 1.97 | 0.0015 | 0.012 |
| HIST2H2BE | 97.19 | 221.02 | 2.27 | 2.10E−08 | 1.00E−06 |
| HIST3H2A | 32.25 | 67.58 | 2.10 | 5.60E−05 | 0.00087 |
| HIVEP1 | 249.38 | 377.22 | 1.51 | 0.0044 | 0.028 |
| HJURP | 14.46 | 82.26 | 5.69 | 6.60E−07 | 2.10E−05 |
| HK3 | 163.76 | 34.3 | 0.21 | 5.90E−08 | 2.50E−06 |
| HLA-DQB1 | 1634.08 | 959.48 | 0.59 | 1.20E−05 | 0.00024 |
| HLA-DQB2 | 170.93 | 64.36 | 0.38 | 5.00E−06 | 0.00012 |
| HLA-DRA | 14618.44 | 7798.46 | 0.53 | 9.30E−05 | 0.0013 |
| HLA-DRB1 | 5400.93 | 3556.09 | 0.66 | 3.60E−05 | 6.00E−04 |
| HLA-DRB5 | 1693.01 | 1100.02 | 0.65 | 6.70E−05 | 0.001 |
| HMOX1 | 215.93 | 97.19 | 0.45 | 0.008 | 0.044 |
| HNMT | 106.27 | 43.01 | 0.40 | 0.0013 | 0.011 |
| HOXA1 | 21.52 | 8.01 | 0.37 | 0.0093 | 0.049 |
| HP | 102.01 | 11.19 | 0.11 | 4.50E−06 | 0.00011 |
| HPGDS | 26.03 | 6.32 | 0.24 | 0.0039 | 0.025 |
| HPS6 | 343.37 | 146.6 | 0.43 | 2.70E−05 | 0.00047 |
| HRH2 | 23.23 | 12.1 | 0.52 | 0.0064 | 0.037 |
| HS1BP3 | 214.99 | 112.68 | 0.52 | 0.0031 | 0.022 |
| HS6ST1 | 58.57 | 24.16 | 0.41 | 0.0023 | 0.017 |
| HSD3B7 | 104.77 | 33.15 | 0.32 | 3.50E−05 | 0.00058 |
| HSP90AA1 | 9901.12 | 17771.14 | 1.79 | 5.10E−07 | 1.70E−05 |
| HSPA1A | 577.27 | 2361.39 | 4.09 | 1.20E−10 | 9.80E−09 |
| HSPA1B | 502.34 | 2361.87 | 4.70 | 1.70E−14 | 3.40E−12 |
| HSPA6 | 225.3 | 545.74 | 2.42 | 5.40E−07 | 1.70E−05 |
| HSPA7 | 13.26 | 25.22 | 1.90 | 0.0016 | 0.013 |
| HSPD1 | 1196.28 | 1877.01 | 1.57 | 0.00024 | 0.0029 |
| HSPE1 | 267.3 | 413.26 | 1.55 | 0.00049 | 0.0051 |
| HSPH1 | 1075.8 | 2078.93 | 1.93 | 2.10E−05 | 0.00038 |
| HTRA1 | 62.69 | 141.7 | 2.26 | 0.0037 | 0.025 |
| HYDIN | 52.31 | 12.37 | 0.24 | 0.0015 | 0.012 |
| ICAM2 | 423.44 | 217.81 | 0.51 | 1.20E−08 | 6.40E−07 |
| ICOS | 770.04 | 1243.94 | 1.62 | 4.00E−04 | 0.0043 |
| ID1 | 12.31 | 44.16 | 3.59 | 0.0033 | 0.023 |
| ID3 | 34.62 | 107.4 | 3.10 | 0.00013 | 0.0017 |
| IFI30 | 4438.14 | 1285.27 | 0.29 | 1.50E−14 | 3.30E−12 |
| IFI44 | 499.41 | 814.89 | 1.63 | 1.70E−05 | 0.00032 |
| IFITM3 | 644.56 | 429.7 | 0.67 | 0.00019 | 0.0024 |
| IFNG | 1389.58 | 2911.74 | 2.10 | 1.70E−07 | 6.70E−06 |
| IFNGR2 | 190.69 | 92.78 | 0.49 | 0.00019 | 0.0023 |
| IGF1 | 29.22 | 17.6 | 0.60 | 0.00063 | 0.0061 |
| IGFBP2 | 198.22 | 68.92 | 0.35 | 0.0012 | 0.01 |
| IGFBP4 | 80.44 | 185.17 | 2.30 | 0.003 | 0.021 |
| IGFBP7 | 46.3 | 13.54 | 0.29 | 0.0016 | 0.013 |
| IGJ | 57.13 | 428.85 | 7.51 | 3.60E−07 | 1.20E−05 |
| IGLL5 | 40.97 | 991.9 | 24.21 | 2.40E−19 | 1.70E−16 |
| IGSF6 | 257.25 | 131.94 | 0.51 | 1.20E−05 | 0.00024 |
| IKZF3 | 2378.25 | 3620.27 | 1.52 | 8.10E−12 | 9.20E−10 |
| IKZF4 | 44.62 | 120.94 | 2.71 | 0.00013 | 0.0017 |
| IL10 | 19.45 | 46.46 | 2.39 | 0.0055 | 0.033 |
| IL17A | 10.19 | 236.83 | 23.24 | 2.00E−06 | 5.40E−05 |
| IL18 | 51.46 | 21.92 | 0.43 | 0.0017 | 0.013 |
| IL1B | 113.08 | 24.63 | 0.22 | 1.30E−08 | 6.60E−07 |
| IL26 | 8.33 | 39.58 | 4.75 | 1.60E−05 | 3.00E−04 |
| IL5RA | 87.57 | 48.32 | 0.55 | 0.0072 | 0.04 |
| IL6ST | 596.04 | 987.21 | 1.66 | 0.00076 | 0.0072 |
| IMPDH1 | 588.15 | 382.08 | 0.65 | 2.40E−06 | 6.20E−05 |
| INHBA | 297.79 | 51.56 | 0.17 | 1.20E−21 | 1.50E−18 |
| INPP5F | 79.63 | 211.64 | 2.66 | 0.00023 | 0.0027 |
| INTS7 | 231.86 | 368.25 | 1.59 | 0.0027 | 0.019 |
| IQSEC2 | 11.42 | 3.8 | 0.33 | 0.0011 | 0.0095 |
| IRAK3 | 62.83 | 15.13 | 0.24 | 1.80E−08 | 8.70E−07 |
| ITGA1 | 1022.28 | 2542.82 | 2.49 | 2.20E−18 | 1.20E−15 |
| ITGA2 | 44.29 | 133.12 | 3.01 | 7.60E−06 | 0.00017 |
| ITGA5 | 731.48 | 471.57 | 0.64 | 0.00037 | 0.0041 |
| ITGA6 | 351.02 | 188.53 | 0.54 | 1.80E−06 | 4.90E−05 |
| ITGAE | 1671.27 | 4241.95 | 2.54 | 1.20E−12 | 1.60E−10 |
| ITGAM | 595.99 | 173.99 | 0.29 | 2.90E−17 | 1.10E−14 |
| ITGAX | 459.16 | 190.42 | 0.41 | 4.50E−07 | 1.50E−05 |
| ITGB1 | 4571.31 | 2960.73 | 0.65 | 2.70E−06 | 6.90E−05 |
| ITGB2 | 9290.52 | 5935.14 | 0.64 | 7.90E−08 | 3.20E−06 |
| ITGB5 | 35.64 | 9.37 | 0.26 | 0.0014 | 0.012 |
| ITGB8 | 102.14 | 41.8 | 0.41 | 0.0029 | 0.021 |
| ITIH5 | 57.77 | 13.83 | 0.24 | 2.30E−05 | 0.00041 |
| ITM2A | 2325.9 | 4089.08 | 1.76 | 4.80E−07 | 1.60E−05 |
| ITM2C | 1373.78 | 2651.85 | 1.93 | 8.30E−10 | 5.50E−08 |
| JUN | 2921.26 | 5253.12 | 1.80 | 1.00E−09 | 6.80E−08 |
| KAL1 | 37.53 | 9.54 | 0.25 | 0.0092 | 0.048 |
| KCNE1 | 29.91 | 1.74 | 0.06 | 5.40E−06 | 0.00013 |
| KCNE3 | 50.92 | 19.17 | 0.38 | 1.00E−04 | 0.0014 |
| KCNK5 | 63.06 | 238.5 | 3.78 | 1.30E−07 | 5.00E−06 |
| KCNN2 | 4.79 | 9.83 | 2.05 | 0.0032 | 0.022 |
| KCNQ1 | 36.56 | 8.9 | 0.24 | 0.00022 | 0.0027 |
| KCNQ1OT1 | 188.81 | 339.16 | 1.80 | 0.00017 | 0.0022 |
| KCTD12 | 69.16 | 32.09 | 0.46 | 8.60E−05 | 0.0012 |
| KDELR3 | 6.67 | 28.34 | 4.25 | 0.0065 | 0.038 |
| KDM5B | 381.06 | 638.89 | 1.68 | 7.90E−07 | 2.50E−05 |
| KHDC1 | 20.46 | 56.99 | 2.79 | 0.0026 | 0.019 |
| KIAA0101 | 53.27 | 195.66 | 3.67 | 3.40E−05 | 0.00058 |
| KIAA0664 | 188.72 | 101.73 | 0.54 | 0.00035 | 0.0038 |
| KIAA0754 | 47.56 | 76.49 | 1.61 | 0.0093 | 0.049 |
| KIAA0825 | 278.49 | 434.26 | 1.56 | 3.50E−05 | 6.00E−04 |
| KIAA1467 | 118.5 | 70.3 | 0.59 | 0.0021 | 0.016 |
| KIAA1598 | 52.8 | 18.71 | 0.35 | 0.0041 | 0.027 |
| KIAA1671 | 376.01 | 599.31 | 1.59 | 0.00041 | 0.0045 |
| KIF11 | 91.31 | 297.89 | 3.26 | 5.80E−06 | 0.00013 |
| KIF14 | 20.7 | 62.17 | 3.00 | 1.40E−05 | 0.00027 |
| KIF15 | 22.12 | 84.15 | 3.80 | 0.0037 | 0.025 |
| KIF18A | 37.72 | 133.29 | 3.53 | 3.90E−06 | 9.50E−05 |
| KIF18B | 3.99 | 23.64 | 5.92 | 0.0017 | 0.014 |
| KIF19 | 31.35 | 5.65 | 0.18 | 0.00047 | 0.0049 |
| KIF20A | 16.98 | 74.45 | 4.38 | 0.00015 | 0.002 |
| KIF20B | 378.79 | 628.38 | 1.66 | 2.60E−05 | 0.00046 |
| KIF23 | 32.14 | 132.13 | 4.11 | 9.80E−06 | 0.00021 |
| KIF2C | 28.34 | 113.34 | 4.00 | 5.20E−07 | 1.70E−05 |
| KIF4A | 17.8 | 58.15 | 3.27 | 0.003 | 0.021 |
| KIR2DL1 | 132.84 | 44.07 | 0.33 | 0.0017 | 0.014 |
| KIR2DL3 | 180.89 | 74.43 | 0.41 | 0.0022 | 0.017 |
| KIR2DL4 | 173 | 474.44 | 2.74 | 0.0018 | 0.014 |
| KIR2DS4 | 188.83 | 45.79 | 0.24 | 2.00E−04 | 0.0025 |
| KIR3DL1 | 373.92 | 107.23 | 0.29 | 8.40E−06 | 0.00018 |
| KIR3DL2 | 397.54 | 146.99 | 0.37 | 3.20E−05 | 0.00054 |
| KIR3DX1 | 36.54 | 12.09 | 0.33 | 0.0029 | 0.021 |
| KLF11 | 205.3 | 72.52 | 0.35 | 2.40E−10 | 1.80E−08 |
| KLF2 | 1710.25 | 585.45 | 0.34 | 2.80E−17 | 1.10E−14 |
| KLF3 | 903.28 | 296.72 | 0.33 | 3.90E−14 | 7.10E−12 |
| KLF4 | 86.66 | 25.1 | 0.29 | 3.70E−10 | 2.70E−08 |
| KLF6 | 11771.81 | 7805.85 | 0.66 | 2.00E−06 | 5.30E−05 |
| KLHDC10 | 171.77 | 110.71 | 0.64 | 0.0026 | 0.019 |
| KLHL6 | 342.37 | 584.43 | 1.71 | 9.10E−06 | 0.00019 |
| KLK10 | 13.93 | 1.61 | 0.12 | 6.60E−05 | 0.001 |
| KLRAP1 | 267.68 | 157.39 | 0.59 | 0.00062 | 0.0061 |
| KLRC4- | 16.68 | 28.17 | 1.69 | 0.002 | 0.015 |
| KLRK1 | | | | | |
| KLRF1 | 914.94 | 182.19 | 0.20 | 1.70E−14 | 3.40E−12 |
| KLRG1 | 2846.29 | 1761.08 | 0.62 | 0.00029 | 0.0034 |
| KRT8 | 107.18 | 210.75 | 1.97 | 0.0031 | 0.022 |
| KRT81 | 12.62 | 51.35 | 4.07 | 3.50E−05 | 0.00059 |
| KRT86 | 147.55 | 499.34 | 3.38 | 5.80E−13 | 8.30E−11 |
| KYNU | 119.08 | 39.37 | 0.33 | 9.60E−05 | 0.0014 |
| LAIR1 | 921.72 | 453.09 | 0.49 | 2.00E−10 | 1.50E−08 |
| LAMB3 | 10.43 | 37.27 | 3.57 | 0.0041 | 0.027 |
| LAMP1 | 319.09 | 211.81 | 0.66 | 4.10E−05 | 0.00068 |
| LATS2 | 59.18 | 26.46 | 0.45 | 0.0021 | 0.016 |
| LAYN | 16.5 | 257.64 | 15.61 | 1.20E−14 | 2.60E−12 |
| LDLR | 553.42 | 179.27 | 0.32 | 6.10E−13 | 8.70E−11 |
| LEF1 | 579.69 | 262.33 | 0.45 | 1.70E−08 | 8.60E−07 |
| LGALS1 | 2315.43 | 1260.78 | 0.54 | 1.90E−12 | 2.40E−10 |
| LGALS3 | 2542.72 | 1607.56 | 0.63 | 0.00035 | 0.0039 |
| LGALS3BP | 1202.65 | 478 | 0.40 | 7.80E−06 | 0.00017 |
| LGR6 | 334.63 | 121.34 | 0.36 | 3.50E−07 | 1.20E−05 |
| LHPP | 138.68 | 92.34 | 0.67 | 0.0031 | 0.021 |
| LILRA2 | 27.25 | 10.5 | 0.39 | 1.70E−05 | 0.00032 |
| LILRA5 | 46.45 | 10.46 | 0.23 | 2.10E−08 | 1.00E−06 |
| LILRA6 | 68.15 | 18.66 | 0.27 | 4.60E−08 | 2.00E−06 |
| LILRB1 | 296.66 | 106.74 | 0.36 | 4.40E−08 | 2.00E−06 |
| LILRB3 | 81.28 | 26.05 | 0.32 | 1.30E−13 | 2.10E−11 |
| LILRP2 | 4.93 | 34.02 | 6.90 | 0.00016 | 0.0021 |
| LINC00152 | 332.66 | 625.25 | 1.88 | 6.20E−07 | 2.00E−05 |
| LINC00158 | 3.65 | 27.3 | 7.48 | 7.60E−05 | 0.0011 |
| LINC00299 | 8.91 | 50.43 | 5.66 | 2.50E−05 | 0.00044 |
| LINC00341 | 85.41 | 55.39 | 0.65 | 0.0015 | 0.012 |
| LINC00426 | 112.57 | 181.68 | 1.61 | 0.00077 | 0.0073 |
| LINC00574 | 6.24 | 12.99 | 2.08 | 0.0034 | 0.023 |
| LINGO3 | 23.11 | 4.67 | 0.20 | 3.00E−06 | 7.60E−05 |
| LIPE | 25.77 | 47.8 | 1.85 | 0.00035 | 0.0038 |
| LIPT2 | 40.92 | 16.66 | 0.41 | 0.0011 | 0.0097 |
| LITAF | 5968.91 | 3113.64 | 0.52 | 3.80E−14 | 7.00E−12 |
| LMCD1 | 23.71 | 107.81 | 4.55 | 0.00062 | 0.0061 |
| LMNA | 1908.21 | 396.77 | 0.21 | 2.90E−18 | 1.40E−15 |
| LMNB1 | 95.78 | 152.5 | 1.59 | 0.0045 | 0.029 |
| LMO7 | 88.82 | 136.8 | 1.54 | 0.0082 | 0.045 |
| LOC100129917 | 52.27 | 78.48 | 1.50 | 0.0063 | 0.037 |
| LOC100130298 | 4.56 | 26.69 | 5.85 | 8.00E−05 | 0.0012 |
| LOC100131176 | 94.23 | 44.05 | 0.47 | 0.00071 | 0.0068 |
| LOC100131234 | 6.9 | 20.69 | 3.00 | 0.0013 | 0.011 |
| LOC100131691 | 19.4 | 40.95 | 2.11 | 4.30E−05 | 7.00E−04 |
| LOC100132077 | 16.96 | 32.99 | 1.95 | 0.0049 | 0.031 |
| LOC100132247 | 53.81 | 87.28 | 1.62 | 0.00045 | 0.0047 |
| LOC100216479 | 12.6 | 3.87 | 0.31 | 0.0065 | 0.037 |
| LOC100216546 | 131.11 | 208.33 | 1.59 | 0.0066 | 0.038 |
| LOC100271836 | 39.6 | 59.54 | 1.50 | 0.00022 | 0.0026 |
| LOC100287616 | 119.93 | 66.5 | 0.55 | 1.20E−05 | 0.00025 |
| LOC100287722 | 33.16 | 52.12 | 1.57 | 0.0012 | 0.01 |
| LOC100302650 | 85.49 | 199.29 | 2.33 | 1.10E−06 | 3.20E−05 |
| LOC100306975 | 11.23 | 39.38 | 3.51 | 0.00049 | 0.0051 |
| LOC100335030 | 6.29 | 12.33 | 1.96 | 0.0021 | 0.016 |
| LOC100505576 | 14.4 | 39.54 | 2.75 | 0.0024 | 0.018 |
| LOC100505702 | 53.56 | 14.3 | 0.27 | 0.00058 | 0.0058 |
| LOC100506548 | 45.7 | 98.61 | 2.16 | 0.00025 | 0.003 |
| LOC100506585 | 37.51 | 5.84 | 0.16 | 2.80E−05 | 0.00049 |
| LOC100506660 | 15.48 | 38.12 | 2.46 | 0.00034 | 0.0037 |
| LOC202181 | 123.57 | 193.04 | 1.56 | 0.003 | 0.021 |
| LOC220729 | 65.82 | 103.46 | 1.57 | 0.0053 | 0.032 |
| LOC284276 | 31.33 | 16.25 | 0.52 | 0.00044 | 0.0047 |
| LOC284801 | 58.13 | 35.49 | 0.61 | 0.00052 | 0.0053 |
| LOC285965 | 47.23 | 135.33 | 2.87 | 4.90E−07 | 1.60E−05 |
| LOC439949 | 867.21 | 543.2 | 0.63 | 6.00E−04 | 0.0059 |
| LOC644656 | 18.9 | 51.27 | 2.71 | 0.0037 | 0.025 |
| LOC653061 | 36 | 18.55 | 0.52 | 0.0041 | 0.027 |
| LOC653075 | 22.17 | 7.11 | 0.32 | 0.0019 | 0.014 |
| LOC727896 | 14.85 | 31.94 | 2.15 | 0.0084 | 0.045 |
| LOC728558 | 22.3 | 41.8 | 1.87 | 3.30E−06 | 8.30E−05 |
| LOC728989 | 4.18 | 16.42 | 3.93 | 1.80E−08 | 8.90E−07 |
| LOC729513 | 31.15 | 47.87 | 1.54 | 0.0068 | 0.039 |
| LOC729603 | 47.92 | 79.05 | 1.65 | 0.0091 | 0.048 |
| LOC729678 | 229.09 | 362.32 | 1.58 | 0.00033 | 0.0037 |
| LOC731424 | 47.84 | 6.05 | 0.13 | 1.20E−09 | 7.80E−08 |
| LOC96610 | 65.23 | 105.06 | 1.61 | 0.0025 | 0.018 |
| LPAR6 | 727.78 | 417.78 | 0.57 | 0.00046 | 0.0048 |
| LPCAT1 | 911.65 | 494.62 | 0.54 | 0.00046 | 0.0048 |
| LPCAT2 | 74.25 | 35.15 | 0.47 | 1.20E−06 | 3.40E−05 |
| LPL | 514.46 | 98.6 | 0.19 | 3.30E−08 | 1.50E−06 |
| LRBA | 962.9 | 1448.48 | 1.50 | 6.80E−08 | 2.80E−06 |
| LRIG2 | 161.64 | 282.47 | 1.75 | 0.00098 | 0.0088 |
| LRP1 | 421.83 | 76.28 | 0.18 | 1.20E−17 | 5.50E−15 |
| LRP6 | 3.8 | 16.49 | 4.34 | 0.0032 | 0.022 |
| LRRC2 | 23.18 | 59.42 | 2.56 | 4.70E−05 | 0.00076 |
| LRRC25 | 79.43 | 38.97 | 0.49 | 0.00013 | 0.0017 |
| LRRC34 | 8.98 | 46.04 | 5.13 | 0.00032 | 0.0036 |
| LRRC8A | 270.45 | 136.32 | 0.50 | 1.20E−06 | 3.40E−05 |
| LRRIQ3 | 20.32 | 8.34 | 0.41 | 0.0084 | 0.045 |
| LRRN3 | 133.55 | 413.93 | 3.10 | 6.40E−05 | 0.00098 |
| LSAMP | 27.29 | 3.59 | 0.13 | 4.40E−07 | 1.50E−05 |
| LST1 | 164.48 | 65.47 | 0.40 | 4.10E−05 | 0.00068 |
| LTA4H | 1552.23 | 690.28 | 0.44 | 7.00E−10 | 4.90E−08 |
| LTB4R | 200.67 | 90.53 | 0.45 | 1.30E−06 | 3.80E−05 |
| LY86 | 139.78 | 36.68 | 0.26 | 2.00E−05 | 0.00037 |
| LYN | 407.98 | 188.1 | 0.46 | 0.00012 | 0.0017 |
| LYZ | 4979.4 | 1755.07 | 0.35 | 6.40E−14 | 1.10E−11 |
| MACC1 | 40.07 | 19.61 | 0.49 | 0.0055 | 0.033 |
| MAD2L1 | 174.34 | 324.4 | 1.86 | 0.0015 | 0.012 |
| MAFB | 147.98 | 64.24 | 0.43 | 0.00041 | 0.0044 |
| MAN1C1 | 41.34 | 81.44 | 1.97 | 0.00053 | 0.0054 |
| MAN2B1 | 912.47 | 574.73 | 0.63 | 0.00054 | 0.0055 |
| MAOB | 6.29 | 39.02 | 6.20 | 0.005 | 0.031 |
| MAP3K14 | 172.99 | 322.52 | 1.86 | 2.90E−09 | 1.70E−07 |
| MAP4K2 | 291.4 | 172.15 | 0.59 | 0.00018 | 0.0023 |
| MAP7D1 | 375.97 | 220.35 | 0.59 | 8.20E−06 | 0.00018 |
| MAPK12 | 2.04 | 14.38 | 7.05 | 0.0049 | 0.031 |
| MARCH3 | 18.68 | 50.54 | 2.71 | 0.00027 | 0.0031 |
| MARCO | 1798.95 | 240.91 | 0.13 | 3.30E−16 | 1.00E−13 |
| MARVELD1 | 27.75 | 5.71 | 0.21 | 8.30E−05 | 0.0012 |
| MAST4 | 119.6 | 243.64 | 2.04 | 2.60E−05 | 0.00045 |
| MATK | 1180.89 | 630.46 | 0.53 | 2.50E−11 | 2.60E−09 |
| MCAM | 14.98 | 31.52 | 2.10 | 0.0019 | 0.015 |
| MCM10 | 10.34 | 51.42 | 4.97 | 6.00E−05 | 0.00093 |
| MCM3AP- | 19.84 | 9.68 | 0.49 | 0.0033 | 0.023 |
| AS1 | | | | | |
| MCM4 | 178.41 | 402.78 | 2.26 | 0.00062 | 0.0061 |
| MCOLN1 | 213.58 | 88.26 | 0.41 | 1.70E−05 | 0.00033 |
| ME1 | 80.71 | 48.5 | 0.60 | 0.0069 | 0.039 |
| ME3 | 35.8 | 13.66 | 0.38 | 0.00094 | 0.0085 |
| MELK | 43.36 | 102.4 | 2.36 | 0.00067 | 0.0065 |
| MESDC1 | 113.78 | 61.89 | 0.54 | 3.70E−10 | 2.70E−08 |
| METTL8 | 139.37 | 229.7 | 1.65 | 0.0014 | 0.012 |
| MFSD7 | 45.4 | 14.25 | 0.31 | 9.30E−05 | 0.0013 |
| MGAT3 | 22.36 | 0 | 0.00 | 7.50E−05 | 0.0011 |
| MGC21881 | 64.49 | 119.7 | 1.86 | 0.0011 | 0.0094 |
| MIAT | 952.4 | 1552.35 | 1.63 | 5.00E−06 | 0.00012 |
| MICAL3 | 103.78 | 64.89 | 0.63 | 0.0039 | 0.025 |
| MIDN | 133.46 | 86.67 | 0.65 | 0.0011 | 0.0098 |
| MINA | 227.57 | 141.43 | 0.62 | 0.0014 | 0.011 |
| MIR155HG | 126.22 | 264.66 | 2.10 | 1.40E−05 | 0.00028 |
| MIR17HG | 38.33 | 127.04 | 3.31 | 2.00E−06 | 5.20E−05 |
| MIR21 | 10.16 | 24.93 | 2.45 | 9.50E−05 | 0.0013 |
| MIR210HG | 4.32 | 13.67 | 3.16 | 0.0056 | 0.034 |
| MIR600HG | 3.95 | 29.91 | 7.57 | 3.40E−06 | 8.30E−05 |
| MIRLET7BHG | 20.63 | 10.97 | 0.53 | 0.0067 | 0.038 |
| MITF | 80.84 | 15.4 | 0.19 | 3.30E−06 | 8.30E−05 |
| MKI67 | 141.66 | 532.16 | 3.76 | 1.90E−08 | 9.40E−07 |
| MKNK1 | 366.41 | 232.67 | 0.63 | 0.0047 | 0.03 |
| MLC1 | 88.87 | 25.29 | 0.28 | 0.0013 | 0.011 |
| MLLT3 | 363.1 | 595.43 | 1.64 | 4.50E−08 | 2.00E−06 |
| MLLT4 | 76 | 35.44 | 0.47 | 0.00018 | 0.0023 |
| MLPH | 123.79 | 31.38 | 0.25 | 5.40E−05 | 0.00086 |
| MMAB | 119.13 | 70.02 | 0.59 | 0.00025 | 0.0029 |
| MME | 193.23 | 17.49 | 0.09 | 2.50E−25 | 8.50E−22 |
| MMP12 | 5.1 | 166.03 | 32.55 | 0.0032 | 0.022 |
| MMP19 | 255.9 | 54.95 | 0.21 | 7.70E−10 | 5.20E−08 |
| MMS22L | 231.36 | 361.52 | 1.56 | 2.20E−05 | 4.00E−04 |
| MND1 | 4.96 | 9.71 | 1.96 | 0.0057 | 0.034 |
| MNDA | 220.93 | 69.49 | 0.31 | 2.50E−06 | 6.40E−05 |
| MNT | 44.97 | 26.97 | 0.60 | 0.0065 | 0.038 |
| MOB3B | 109.35 | 33.89 | 0.31 | 6.70E−05 | 0.001 |
| MPEG1 | 102.45 | 36.76 | 0.36 | 0.0042 | 0.027 |
| MPHOSPH9 | 292.27 | 587.69 | 2.01 | 5.70E−05 | 0.00089 |
| MRAS | 29.99 | 6.69 | 0.22 | 1.60E−06 | 4.50E−05 |
| MRC1 | 79.41 | 20.61 | 0.26 | 1.20E−12 | 1.60E−10 |
| MS4A4A | 328.31 | 117.94 | 0.36 | 0.0037 | 0.025 |
| MS4A7 | 869.66 | 180.97 | 0.21 | 7.10E−18 | 3.30E−15 |
| MSH2 | 299.42 | 482.79 | 1.61 | 0.0049 | 0.031 |
| MSR1 | 1410.66 | 314.49 | 0.22 | 7.00E−09 | 3.80E−07 |
| MSRA | 99.27 | 63.95 | 0.64 | 0.0015 | 0.012 |
| MSX2P1 | 43.78 | 17.87 | 0.41 | 0.00051 | 0.0053 |
| MTMR14 | 682.26 | 423.48 | 0.62 | 3.10E−06 | 7.80E−05 |
| MTSS1 | 527.19 | 269.25 | 0.51 | 0.00057 | 0.0058 |
| MTX3 | 192.2 | 342.63 | 1.78 | 1.00E−05 | 0.00022 |
| MYADM | 2827.6 | 1104.02 | 0.39 | 9.70E−14 | 1.60E−11 |
| MYBL1 | 498.26 | 251.88 | 0.51 | 9.00E−05 | 0.0013 |
| MYEF2 | 41.74 | 70.16 | 1.68 | 0.0065 | 0.038 |
| MYL6B | 18.92 | 68.45 | 3.62 | 8.70E−05 | 0.0013 |
| MYO1D | 156.63 | 78.63 | 0.50 | 0.0038 | 0.025 |
| MYO1E | 65.57 | 168.93 | 2.58 | 0.0015 | 0.012 |
| MYO1G | 2764.75 | 1742.78 | 0.63 | 6.60E−13 | 9.20E−11 |
| MYO5B | 34.66 | 122.9 | 3.55 | 0.001 | 0.0093 |
| MYO7A | 144.45 | 737.89 | 5.11 | 5.90E−11 | 5.30E−09 |
| MZB1 | 37.16 | 106.47 | 2.87 | 6.20E−06 | 0.00014 |
| N4BP2 | 358.23 | 580.14 | 1.62 | 9.50E−06 | 2.00E−04 |
| NAB1 | 312.04 | 611.28 | 1.96 | 5.80E−06 | 0.00013 |
| NACC2 | 94.72 | 50.07 | 0.53 | 0.0029 | 0.02 |
| NAPSB | 71.28 | 35.44 | 0.50 | 0.0023 | 0.017 |
| NBPF1 | 82.48 | 48.87 | 0.59 | 0.0042 | 0.027 |
| NBPF15 | 39.03 | 61.63 | 1.58 | 0.0026 | 0.019 |
| NCAM1 | 144.12 | 39.98 | 0.28 | 8.20E−05 | 0.0012 |
| NCAPG | 43.9 | 148.04 | 3.37 | 0.0027 | 0.019 |
| NCF1 | 64.52 | 18.83 | 0.29 | 4.90E−07 | 1.60E−05 |
| NCF1C | 35.28 | 16.87 | 0.48 | 0.0045 | 0.028 |
| NCF2 | 466.84 | 141.89 | 0.30 | 3.10E−08 | 1.40E−06 |
| NCKAP5L | 39.93 | 21.33 | 0.53 | 0.00066 | 0.0064 |
| NCR3 | 561.86 | 311.21 | 0.55 | 1.10E−08 | 5.80E−07 |
| NDFIP2 | 289.83 | 852.29 | 2.94 | 1.20E−08 | 6.30E−07 |
| NDST1 | 69.33 | 19.42 | 0.28 | 0.00085 | 0.0079 |
| NEBL | 9.28 | 51.82 | 5.58 | 0.00017 | 0.0022 |
| NEIL2 | 169.68 | 88.82 | 0.52 | 0.00011 | 0.0015 |
| NEK2 | 13.7 | 42.78 | 3.12 | 2.30E−05 | 0.00041 |
| NEK6 | 303.29 | 160.58 | 0.53 | 7.90E−05 | 0.0012 |
| NELF | 184.01 | 101.84 | 0.55 | 2.00E−06 | 5.30E−05 |
| NELL2 | 1098.57 | 1885.79 | 1.72 | 1.00E−06 | 3.10E−05 |
| NETO2 | 16.76 | 46.79 | 2.79 | 0.0057 | 0.034 |
| NFAM1 | 126.75 | 27.8 | 0.22 | 6.60E−14 | 1.10E−11 |
| NFKBIZ | 925.6 | 1802.78 | 1.95 | 2.40E−08 | 1.20E−06 |
| NHS | 20.61 | 154.09 | 7.48 | 5.00E−09 | 2.80E−07 |
| NHSL2 | 182.08 | 46.52 | 0.26 | 1.90E−07 | 7.20E−06 |
| NMUR1 | 408.84 | 239.41 | 0.59 | 0.0029 | 0.02 |
| NPHP3 | 31.42 | 52.59 | 1.67 | 0.0052 | 0.032 |
| NPHP3- | 10.38 | 17.62 | 1.70 | 0.007 | 0.04 |
| ACAD11 | | | | | |
| NR1H3 | 140.21 | 67.55 | 0.48 | 0.0071 | 0.04 |
| NR5A2 | 18.1 | 66.9 | 3.70 | 0.0049 | 0.031 |
| NTRK1 | 33.7 | 72.7 | 2.16 | 2.20E−06 | 5.80E−05 |
| NUP107 | 388.13 | 610.81 | 1.57 | 0.00013 | 0.0017 |
| NUPR1 | 131 | 71.17 | 0.54 | 0.0018 | 0.014 |
| NUSAP1 | 173.56 | 435.4 | 2.51 | 1.10E−05 | 0.00023 |
| O3FAR1 | 66.53 | 12.75 | 0.19 | 3.10E−09 | 1.80E−07 |
| OCIAD2 | 431.88 | 656.08 | 1.52 | 0.00011 | 0.0015 |
| ODF3L1 | 28.82 | 3.43 | 0.12 | 0.00095 | 0.0086 |
| OLFM2 | 76.33 | 125.28 | 1.64 | 0.0013 | 0.011 |
| OLR1 | 737.91 | 200.44 | 0.27 | 4.80E−10 | 3.50E−08 |
| OPN3 | 56.65 | 30.93 | 0.55 | 0.0091 | 0.048 |
| ORAI1 | 1069 | 660.58 | 0.62 | 2.20E−11 | 2.30E−09 |
| ORC6 | 21.97 | 48.67 | 2.22 | 0.00039 | 0.0043 |
| OSBPL5 | 379.47 | 77.03 | 0.20 | 2.60E−13 | 3.90E−11 |
| OSBPL7 | 205.2 | 125.87 | 0.61 | 0.0052 | 0.032 |
| OSCAR | 149.12 | 28.52 | 0.19 | 5.80E−11 | 5.30E−09 |
| OSMR | 12.11 | 51.66 | 4.27 | 5.60E−07 | 1.80E−05 |
| OTUB2 | 26.37 | 79.05 | 3.00 | 0.00059 | 0.0059 |
| OTUD1 | 137.32 | 81.03 | 0.59 | 0.0057 | 0.034 |
| OTUD7A | 15.17 | 6.61 | 0.44 | 0.0021 | 0.016 |
| PAG1 | 1744.56 | 2649.42 | 1.52 | 1.20E−06 | 3.40E−05 |
| PAIP2B | 55.88 | 25.71 | 0.46 | 0.004 | 0.026 |
| PALLD | 111.46 | 47.93 | 0.43 | 8.00E−04 | 0.0075 |
| PAQR5 | 33.94 | 9.84 | 0.29 | 8.00E−04 | 0.0074 |
| PARP14 | 1556.72 | 2335.17 | 1.50 | 4.10E−09 | 2.30E−07 |
| PATL2 | 640.15 | 340.96 | 0.53 | 0.00017 | 0.0022 |
| PCNA | 265.91 | 445.29 | 1.67 | 0.0047 | 0.029 |
| PCNXL2 | 231.13 | 365.61 | 1.58 | 1.50E−05 | 3.00E−04 |
| PCOLCE2 | 186.38 | 21.82 | 0.12 | 4.90E−14 | 8.80E−12 |
| PCSK1N | 37.78 | 13.79 | 0.37 | 0.0039 | 0.025 |
| PDCD1 | 775.88 | 1308.17 | 1.69 | 0.00059 | 0.0058 |
| PDE4A | 366.01 | 168.72 | 0.46 | 4.20E−12 | 5.00E−10 |
| PDE4DIP | 793.31 | 1726.58 | 2.18 | 8.00E−16 | 2.30E−13 |
| PDE6G | 15.06 | 1.25 | 0.08 | 0.00012 | 0.0016 |
| PDE7B | 10.18 | 82 | 8.06 | 3.80E−15 | 9.30E−13 |
| PDE8A | 133.4 | 71.23 | 0.53 | 0.0024 | 0.018 |
| PDGFD | 281.87 | 147.87 | 0.52 | 0.0026 | 0.019 |
| PDK1 | 169.29 | 263.6 | 1.56 | 0.00089 | 0.0082 |
| PDK4 | 183.59 | 34.42 | 0.19 | 1.30E−05 | 0.00026 |
| PDLIM1 | 435.66 | 149.48 | 0.34 | 0.00016 | 0.0021 |
| PDLIM4 | 23.81 | 95.56 | 4.01 | 6.30E−08 | 2.60E−06 |
| PDZD4 | 119.37 | 29.8 | 0.25 | 1.30E−06 | 3.70E−05 |
| PDZD8 | 222.52 | 147.83 | 0.66 | 0.0048 | 0.03 |
| PELI2 | 141 | 35.09 | 0.25 | 8.10E−06 | 0.00018 |
| PGD | 948.63 | 567.97 | 0.60 | 0.00096 | 0.0087 |
| PHEX | 2.39 | 69.34 | 29.01 | 2.60E−05 | 0.00046 |
| PHLDA3 | 80.14 | 23.52 | 0.29 | 0.00045 | 0.0048 |
| PIBF1 | 297.02 | 457.25 | 1.54 | 0.0016 | 0.013 |
| PIEZO1 | 523.85 | 337.71 | 0.64 | 2.50E−05 | 0.00044 |
| PIGV | 185.92 | 97.66 | 0.53 | 0.0056 | 0.034 |
| PIK3C2A | 237.98 | 379.31 | 1.59 | 0.0011 | 0.0094 |
| PIK3R2 | 110 | 32.19 | 0.29 | 1.10E−06 | 3.30E−05 |
| PIK3R5 | 1559.61 | 908.51 | 0.58 | 1.40E−11 | 1.50E−09 |
| PILRA | 191.48 | 37.86 | 0.20 | 8.30E−13 | 1.10E−10 |
| PION | 342.15 | 222.22 | 0.65 | 0.0092 | 0.049 |
| PIP5K1C | 222.38 | 146.43 | 0.66 | 0.005 | 0.031 |
| PIWIL2 | 7.32 | 14.37 | 1.96 | 0.0024 | 0.018 |
| PKP2 | 25.77 | 10.69 | 0.41 | 0.005 | 0.031 |
| PLAC8 | 2021.3 | 609.46 | 0.30 | 7.20E−19 | 4.60E−16 |
| PLAGL1 | 46.49 | 105.96 | 2.28 | 3.00E−04 | 0.0034 |
| PLAT | 6.43 | 45.58 | 7.09 | 0.00011 | 0.0015 |
| PLAUR | 317.9 | 155.43 | 0.49 | 0.00088 | 0.0081 |
| PLBD1 | 493.32 | 86.58 | 0.18 | 4.70E−12 | 5.50E−10 |
| PLBD2 | 446.23 | 285.82 | 0.64 | 0.00012 | 0.0016 |
| PLCD1 | 263.9 | 167.56 | 0.63 | 0.00037 | 0.004 |
| PLCXD2 | 111.9 | 221.12 | 1.98 | 1.70E−08 | 8.30E−07 |
| PLD3 | 1228.11 | 739.14 | 0.60 | 0.00018 | 0.0023 |
| PLEK | 2913.34 | 1286.68 | 0.44 | 9.80E−13 | 1.30E−10 |
| PLEKHG3 | 507.52 | 118.67 | 0.23 | 2.50E−11 | 2.60E−09 |
| PLIN2 | 1568.78 | 922.48 | 0.59 | 5.90E−06 | 0.00014 |
| PLOD1 | 383.21 | 232.5 | 0.61 | 0.0085 | 0.046 |
| PLS3 | 10.73 | 112.24 | 10.46 | 6.90E−06 | 0.00016 |
| PLXDC2 | 248.39 | 78.08 | 0.31 | 3.40E−07 | 1.20E−05 |
| PLXND1 | 300.12 | 174.54 | 0.58 | 6.20E−05 | 0.00095 |
| PMAIP1 | 359.94 | 663.39 | 1.84 | 6.10E−06 | 0.00014 |
| PMEPA1 | 42.7 | 114.59 | 2.68 | 8.50E−07 | 2.60E−05 |
| PNPLA6 | 846.6 | 387.35 | 0.46 | 1.30E−10 | 1.10E−08 |
| POLE2 | 16.68 | 59.23 | 3.55 | 0.0015 | 0.012 |
| POLQ | 25.63 | 69.52 | 2.71 | 0.0068 | 0.039 |
| POLR2J2 | 3.65 | 10.93 | 2.99 | 0.00011 | 0.0015 |
| PON2 | 121.1 | 212.74 | 1.76 | 0.00063 | 0.0062 |
| PON3 | 1.07 | 41.15 | 38.46 | 4.60E−05 | 0.00075 |
| POR | 718.24 | 440.4 | 0.61 | 0.00028 | 0.0032 |
| POU2AF1 | 7.97 | 32.48 | 4.08 | 5.00E−10 | 3.60E−08 |
| PPAP2A | 127.85 | 235.71 | 1.84 | 0.00059 | 0.0059 |
| PPARG | 246.37 | 69.28 | 0.28 | 5.20E−06 | 0.00012 |
| PPIC | 48.1 | 14.3 | 0.30 | 0.0012 | 0.011 |
| PPP1R14B | 86 | 221.48 | 2.58 | 3.90E−06 | 9.50E−05 |
| PPP1R9B | 72.86 | 44.29 | 0.61 | 0.0058 | 0.035 |
| PRAM1 | 61.16 | 15.78 | 0.26 | 2.80E−05 | 0.00048 |
| PRF1 | 16655.25 | 9812.88 | 0.59 | 8.40E−12 | 9.40E−10 |
| PRKAR1B | 76.38 | 131.57 | 1.72 | 0.0049 | 0.03 |
| PRKCD | 494.34 | 304.61 | 0.62 | 1.40E−05 | 0.00027 |
| PRKG2 | 14.31 | 2.36 | 0.16 | 0.0014 | 0.012 |
| PROCR | 97.29 | 43.39 | 0.45 | 0.004 | 0.026 |
| PROK2 | 63.46 | 23 | 0.36 | 0.00054 | 0.0055 |
| PROS1 | 93.81 | 38.02 | 0.41 | 0.0065 | 0.038 |
| PROX2 | 11.81 | 47.5 | 4.02 | 0.00033 | 0.0037 |
| PRR11 | 48.49 | 89.8 | 1.85 | 8.70E−05 | 0.0013 |
| PRR5 | 295.89 | 196.48 | 0.66 | 0.0014 | 0.012 |
| PRR7 | 84.47 | 48.24 | 0.57 | 7.00E−04 | 0.0067 |
| PRSS21 | 66.89 | 27.03 | 0.40 | 4.00E−04 | 0.0043 |
| PRSS23 | 672.84 | 176.9 | 0.26 | 5.30E−08 | 2.30E−06 |
| PRSS30P | 89.06 | 31.04 | 0.35 | 0.00022 | 0.0027 |
| PRSS8 | 11.15 | 53.88 | 4.83 | 0.0015 | 0.012 |
| PSAP | 10389.23 | 5346.87 | 0.51 | 1.90E−11 | 2.00E−09 |
| PSTPIP2 | 216.42 | 141.24 | 0.65 | 0.0027 | 0.019 |
| PTAFR | 216.72 | 81.54 | 0.38 | 6.10E−08 | 2.60E−06 |
| PTBP2 | 142.42 | 216.56 | 1.52 | 0.0071 | 0.04 |
| PTCH1 | 373.67 | 135.66 | 0.36 | 1.50E−10 | 1.20E−08 |
| PTGDR | 921.11 | 507.32 | 0.55 | 8.00E−04 | 0.0075 |
| PTGDR2 | 53.28 | 8.53 | 0.16 | 5.30E−05 | 0.00084 |
| PTGDS | 96.43 | 27.57 | 0.29 | 6.00E−06 | 0.00014 |
| PTGER2 | 1858.87 | 728.51 | 0.39 | 6.00E−12 | 7.00E−10 |
| PTGIS | 27.95 | 69.74 | 2.50 | 0.00015 | 0.002 |
| PTPN12 | 191.47 | 105.76 | 0.55 | 8.00E−04 | 0.0075 |
| PTPN13 | 8.27 | 37.28 | 4.51 | 0.0083 | 0.045 |
| PTPN18 | 1116.62 | 694.23 | 0.62 | 1.10E−07 | 4.50E−06 |
| PTPN22 | 1984.12 | 2976.77 | 1.50 | 3.40E−07 | 1.20E−05 |
| PTPN7 | 2573.74 | 3872.72 | 1.50 | 2.60E−05 | 0.00046 |
| PTPRF | 31.28 | 91.26 | 2.92 | 0.0024 | 0.018 |
| PTPRK | 42.9 | 120.01 | 2.80 | 7.00E−05 | 0.0011 |
| PTPRN2 | 69.51 | 137.01 | 1.97 | 0.0046 | 0.029 |
| PTPRO | 44.95 | 15.82 | 0.35 | 3.00E−04 | 0.0034 |
| PTTG1 | 293.69 | 625.45 | 2.13 | 7.10E−05 | 0.0011 |
| PVR | 63.72 | 24.95 | 0.39 | 0.0012 | 0.01 |
| PVT1 | 98.88 | 208.45 | 2.11 | 6.70E−06 | 0.00015 |
| PXN | 1867.53 | 583.78 | 0.31 | 1.30E−20 | 1.10E−17 |
| PYROXD2 | 63.5 | 26.42 | 0.42 | 7.00E−04 | 0.0067 |
| R3HDM1 | 353.44 | 569.53 | 1.61 | 0.00034 | 0.0037 |
| RAB11FIP5 | 152.27 | 38.35 | 0.25 | 1.90E−07 | 7.20E−06 |
| RAB13 | 64.36 | 28.8 | 0.45 | 0.0025 | 0.018 |
| RAB26 | 6.39 | 19.08 | 2.99 | 0.0025 | 0.019 |
| RAB27A | 1260.59 | 1894.13 | 1.50 | 8.90E−05 | 0.0013 |
| RAB31 | 329.33 | 107.84 | 0.33 | 1.70E−06 | 4.80E−05 |
| RAB3GAP1 | 271.3 | 900.81 | 3.32 | 4.80E−18 | 2.30E−15 |
| RAB9A | 483.87 | 287.71 | 0.59 | 9.60E−06 | 2.00E−04 |
| RAD54L | 12.82 | 33.75 | 2.63 | 0.0026 | 0.019 |
| RAP1GAP2 | 227.07 | 53.31 | 0.23 | 1.30E−08 | 6.60E−07 |
| RAP2A | 308.23 | 182.17 | 0.59 | 0.00091 | 0.0083 |
| RAP2B | 1152.96 | 694.31 | 0.60 | 1.50E−07 | 6.00E−06 |
| RARA | 461.29 | 230.91 | 0.50 | 2.80E−08 | 1.30E−06 |
| RASA3 | 805.78 | 408.32 | 0.51 | 1.10E−09 | 6.90E−08 |
| RASAL2 | 65.31 | 15.94 | 0.24 | 5.70E−09 | 3.20E−07 |
| RASGRP2 | 489.39 | 171.03 | 0.35 | 3.90E−16 | 1.20E−13 |
| RBM38 | 710.53 | 468.28 | 0.66 | 2.80E−05 | 0.00049 |
| RBP4 | 247.11 | 26.64 | 0.11 | 3.00E−11 | 3.00E−09 |
| RBPJ | 1944.93 | 3538.41 | 1.82 | 3.10E−07 | 1.10E−05 |
| RCBTB2 | 539.7 | 222.97 | 0.41 | 2.40E−05 | 0.00043 |
| REC8 | 105.61 | 169.33 | 1.60 | 0.003 | 0.021 |
| REG4 | 4.79 | 42.38 | 8.85 | 1.60E−05 | 3.00E−04 |
| REPS1 | 519.92 | 301.78 | 0.58 | 2.80E−06 | 7.10E−05 |
| RETN | 84.27 | 10.86 | 0.13 | 2.30E−24 | 6.30E−21 |
| RFC4 | 117.23 | 200.17 | 1.71 | 6.30E−05 | 0.00097 |
| RGL4 | 160.8 | 265.26 | 1.65 | 2.20E−06 | 5.80E−05 |
| RGMB | 27.51 | 7.32 | 0.27 | 0.0084 | 0.045 |
| RGNEF | 76.24 | 23.72 | 0.31 | 0.00021 | 0.0026 |
| RGS1 | 6864.23 | 20437.32 | 2.98 | 2.20E−12 | 2.70E−10 |
| RGS2 | 1467.53 | 4815.87 | 3.28 | 4.60E−12 | 5.50E−10 |
| RHBDD2 | 1444.91 | 935.25 | 0.65 | 2.20E−05 | 4.00E−04 |
| RHBDL2 | 7.84 | 16.2 | 2.07 | 0.0016 | 0.013 |
| RHOU | 47 | 25.72 | 0.55 | 0.005 | 0.031 |
| RMI1 | 79.17 | 160.44 | 2.03 | 0.00053 | 0.0054 |
| RNASEK | 35.49 | 20.76 | 0.58 | 0.0032 | 0.022 |
| RND3 | 86.47 | 16.56 | 0.19 | 0.00012 | 0.0016 |
| RNF122 | 34.85 | 57.48 | 1.65 | 0.0021 | 0.016 |
| RNF126 | 490.52 | 281.99 | 0.57 | 8.60E−07 | 2.60E−05 |
| RNF130 | 511.85 | 233.09 | 0.46 | 5.10E−11 | 4.70E−09 |
| RNF138P1 | 51.5 | 118.15 | 2.29 | 9.60E−06 | 2.00E−04 |
| RNF144A | 243.8 | 145.36 | 0.60 | 0.00031 | 0.0035 |
| RNPEPL1 | 1319.83 | 716.36 | 0.54 | 5.80E−06 | 0.00013 |
| ROPN1L | 26.66 | 9.96 | 0.37 | 0.0013 | 0.011 |
| RPP25 | 32.35 | 12.67 | 0.39 | 0.0075 | 0.042 |
| RPS16P5 | 40.42 | 126.65 | 3.13 | 3.40E−08 | 1.50E−06 |
| RPS6KA6 | 6.62 | 11.17 | 1.69 | 0.0016 | 0.013 |
| RRAGD | 90.64 | 29.4 | 0.32 | 5.80E−06 | 0.00013 |
| RRAS2 | 389.52 | 206.27 | 0.53 | 6.90E−06 | 0.00015 |
| RRBP1 | 205.69 | 122.91 | 0.60 | 0.00024 | 0.0029 |
| RRM2 | 143.12 | 447.47 | 3.13 | 1.50E−05 | 0.00029 |
| RSAD2 | 177.02 | 381.07 | 2.15 | 0.00011 | 0.0015 |
| RSPH1 | 15.64 | 3.77 | 0.24 | 0.0036 | 0.024 |
| RTN3 | 1113.78 | 731.88 | 0.66 | 2.50E−05 | 0.00044 |
| RTN4 | 964.33 | 580.12 | 0.60 | 6.10E−06 | 0.00014 |
| RUNX2 | 388.09 | 695.34 | 1.79 | 1.80E−07 | 6.90E−06 |
| RXRA | 149.61 | 78.07 | 0.52 | 1.30E−06 | 3.70E−05 |
| RYR2 | 20.01 | 113.01 | 5.65 | 5.40E−05 | 0.00085 |
| S100A10 | 5329.44 | 2435.31 | 0.46 | 1.10E−20 | 1.10E−17 |
| S100A11 | 3608.48 | 1834.68 | 0.51 | 1.60E−17 | 6.60E−15 |
| S100A4 | 11003.84 | 7224.12 | 0.66 | 4.00E−17 | 1.50E−14 |
| S100A8 | 311.42 | 89.66 | 0.29 | 6.00E−07 | 1.90E−05 |
| S100A9 | 713.64 | 278.7 | 0.39 | 0.00047 | 0.0049 |
| S100PBP | 322.1 | 551.19 | 1.71 | 6.10E−06 | 0.00014 |
| S1PR1 | 2333.29 | 773.74 | 0.33 | 1.10E−15 | 3.10E−13 |
| S1PR3 | 3.15 | 13.04 | 4.14 | 0.0035 | 0.024 |
| S1PR4 | 1586.58 | 827.19 | 0.52 | 2.30E−10 | 1.70E−08 |
| S1PR5 | 1167.22 | 286.79 | 0.25 | 1.10E−16 | 3.90E−14 |
| SAMD10 | 86.67 | 140.9 | 1.63 | 0.0023 | 0.017 |
| SAMHD1 | 1735.97 | 1132.92 | 0.65 | 9.50E−06 | 2.00E−04 |
| SAPCD2 | 4.6 | 20.37 | 4.43 | 0.0076 | 0.042 |
| SARDH | 93.95 | 409.61 | 4.36 | 5.70E−10 | 4.00E−08 |
| SASH1 | 19.2 | 10.65 | 0.55 | 0.0036 | 0.024 |
| SASS6 | 100.81 | 161.5 | 1.60 | 1.00E−05 | 0.00021 |
| SBK1 | 94.2 | 35.92 | 0.38 | 7.60E−07 | 2.40E−05 |
| SCD | 1000.29 | 323.16 | 0.32 | 0.00026 | 0.003 |
| SCGB1A1 | 1358.94 | 84.1 | 0.06 | 6.60E−13 | 9.20E−11 |
| SCGB3A1 | 693.6 | 115.64 | 0.17 | 3.70E−09 | 2.10E−07 |
| SCIMP | 25.42 | 12.05 | 0.47 | 0.0051 | 0.031 |
| SCPEP1 | 515.35 | 290.66 | 0.56 | 1.10E−06 | 3.20E−05 |
| SDC1 | 14.45 | 57.22 | 3.96 | 0.0011 | 0.0099 |
| SEC61A2 | 55.23 | 89.55 | 1.62 | 0.0093 | 0.049 |
| SECTM1 | 101.34 | 15.49 | 0.15 | 1.20E−09 | 7.80E−08 |
| SELL | 2056.97 | 1207.89 | 0.59 | 0.00041 | 0.0044 |
| SEMA7A | 70.17 | 169.74 | 2.42 | 0.00083 | 0.0077 |
| SENP7 | 541.78 | 845.4 | 1.56 | 8.80E−14 | 1.50E−11 |
| SEPT10 | 33.01 | 11.57 | 0.35 | 0.0038 | 0.025 |
| SEPT11 | 1174.64 | 658.32 | 0.56 | 1.10E−10 | 9.30E−09 |
| SEPT4 | 33.18 | 9.11 | 0.27 | 0.0019 | 0.015 |
| SERPINA1 | 3111.71 | 478.87 | 0.15 | 2.00E−13 | 3.20E−11 |
| SERPINB6 | 518.64 | 285.09 | 0.55 | 2.50E−05 | 0.00044 |
| SERPING1 | 1357.42 | 127.6 | 0.09 | 1.30E−20 | 1.10E−17 |
| SERTAD1 | 307.7 | 488.54 | 1.59 | 0.0021 | 0.016 |
| SESN3 | 23.88 | 53.99 | 2.26 | 0.0012 | 0.01 |
| SFMBT2 | 351.17 | 542.52 | 1.54 | 8.90E−05 | 0.0013 |
| SFTPA1 | 771.82 | 217.9 | 0.28 | 0.00094 | 0.0086 |
| SFTPA2 | 1100.91 | 321.95 | 0.29 | 0.00057 | 0.0058 |
| SFTPC | 1300.43 | 147.57 | 0.11 | 1.20E−13 | 1.90E−11 |
| SFXN2 | 63.14 | 130.46 | 2.07 | 0.0034 | 0.023 |
| SGMS1 | 548.79 | 857.93 | 1.56 | 1.90E−05 | 0.00036 |
| SGMS2 | 92.46 | 22.22 | 0.24 | 1.80E−05 | 0.00035 |
| SGOL1 | 38.82 | 65.62 | 1.69 | 0.008 | 0.044 |
| SGPP2 | 9.48 | 50.1 | 5.28 | 1.10E−05 | 0.00022 |
| SH3BP5 | 194.86 | 89.24 | 0.46 | 2.30E−07 | 8.40E−06 |
| SH3D21 | 5.46 | 21.13 | 3.87 | 0.0094 | 0.049 |
| SH3PXD2B | 21.9 | 5.85 | 0.27 | 0.0016 | 0.013 |
| SH3RF1 | 3.51 | 22.46 | 6.40 | 0.004 | 0.026 |
| SHCBP1 | 20.97 | 99.99 | 4.77 | 8.70E−05 | 0.0013 |
| SIDT2 | 185.04 | 102.02 | 0.55 | 0.0017 | 0.013 |
| SIGLEC1 | 91.18 | 33.28 | 0.36 | 0.0078 | 0.043 |
| SIGLEC11 | 21.1 | 5.83 | 0.28 | 6.60E−06 | 0.00015 |
| SIGLEC14 | 99.97 | 27.78 | 0.28 | 3.80E−07 | 1.30E−05 |
| SIGLEC7 | 93.94 | 20.47 | 0.22 | 1.10E−06 | 3.10E−05 |
| SIGLEC9 | 75.15 | 34.8 | 0.46 | 0.0016 | 0.013 |
| SIGLECP3 | 268.06 | 52.25 | 0.19 | 3.30E−11 | 3.20E−09 |
| SIPA1L1 | 334 | 522.28 | 1.56 | 2.00E−04 | 0.0025 |
| SIRPA | 190.71 | 56.83 | 0.30 | 2.40E−05 | 0.00043 |
| SIRPB1 | 211.46 | 59.18 | 0.28 | 2.20E−06 | 5.80E−05 |
| SIRPB2 | 34.56 | 12.91 | 0.37 | 0.00029 | 0.0033 |
| SIRPG | 577.55 | 1830.28 | 3.17 | 5.10E−15 | 1.20E−12 |
| SKA1 | 31.14 | 54.87 | 1.76 | 0.0022 | 0.017 |
| SKA2 | 328.42 | 512.26 | 1.56 | 0.00075 | 0.0071 |
| SKIL | 771.5 | 1201.15 | 1.56 | 2.30E−06 | 6.00E−05 |
| SLC10A1 | 10.2 | 34.33 | 3.37 | 8.60E−05 | 0.0013 |
| SLC10A3 | 669.13 | 422.44 | 0.63 | 8.10E−06 | 0.00018 |
| SLC11A1 | 507.86 | 119.17 | 0.23 | 7.50E−12 | 8.50E−10 |
| SLC12A7 | 205.86 | 87.52 | 0.43 | 2.50E−09 | 1.50E−07 |
| SLC15A3 | 83.81 | 21.93 | 0.26 | 5.80E−08 | 2.50E−06 |
| SLC19A3 | 54.83 | 11.35 | 0.21 | 4.70E−11 | 4.40E−09 |
| SLC22A15 | 18.09 | 10.27 | 0.57 | 0.0067 | 0.038 |
| SLC23A2 | 160.24 | 260.22 | 1.62 | 0.0047 | 0.03 |
| SLC23A3 | 6.55 | 15.32 | 2.34 | 0.0088 | 0.047 |
| SLC25A23 | 76.73 | 37.34 | 0.49 | 0.00064 | 0.0062 |
| SLC25A29 | 76.23 | 47.85 | 0.63 | 0.0085 | 0.046 |
| SLC25A33 | 122.14 | 64.13 | 0.53 | 0.0082 | 0.045 |
| SLC27A2 | 142.15 | 426.13 | 3.00 | 1.20E−05 | 0.00024 |
| SLC27A3 | 410.59 | 242.78 | 0.59 | 0.0017 | 0.013 |
| SLC29A2 | 41.73 | 21.04 | 0.50 | 0.0077 | 0.043 |
| SLC2A6 | 123.67 | 76.56 | 0.62 | 0.0035 | 0.024 |
| SLC31A1 | 277.98 | 177.38 | 0.64 | 0.0072 | 0.04 |
| SLC31A2 | 437.27 | 118.34 | 0.27 | 7.30E−09 | 4.00E−07 |
| SLC35C1 | 260.38 | 158.07 | 0.61 | 0.0059 | 0.035 |
| SLC35G1 | 8.71 | 22.28 | 2.56 | 0.0058 | 0.035 |
| SLC37A2 | 94.54 | 57.01 | 0.60 | 0.0071 | 0.04 |
| SLC44A2 | 2165.89 | 1339.68 | 0.62 | 3.60E−08 | 1.60E−06 |
| SLC44A4 | 72.74 | 33.69 | 0.46 | 0.0068 | 0.039 |
| SLC47A1 | 87.76 | 31.28 | 0.36 | 0.00078 | 0.0073 |
| SLC48A1 | 105 | 67.46 | 0.64 | 0.0017 | 0.013 |
| SLC4A5 | 43.19 | 74.03 | 1.71 | 0.0014 | 0.011 |
| SLC5A3 | 506.23 | 954.64 | 1.89 | 7.00E−07 | 2.20E−05 |
| SLC5A6 | 199.44 | 129.18 | 0.65 | 0.005 | 0.031 |
| SLC6A14 | 4.12 | 18.64 | 4.52 | 3.70E−05 | 0.00062 |
| SLC6A20 | 5.43 | 11.99 | 2.21 | 0.0017 | 0.013 |
| SLC7A5P2 | 157.49 | 288.89 | 1.83 | 1.00E−10 | 8.70E−09 |
| SLC7A7 | 307.99 | 67.11 | 0.22 | 2.80E−05 | 0.00048 |
| SLC7A8 | 222.73 | 45.13 | 0.20 | 1.20E−06 | 3.50E−05 |
| SLC8A1 | 48.47 | 16.37 | 0.34 | 3.80E−06 | 9.40E−05 |
| SLCO2B1 | 450.13 | 97.27 | 0.22 | 1.70E−06 | 4.60E−05 |
| SLCO3A1 | 208.5 | 65.48 | 0.31 | 9.80E−13 | 1.30E−10 |
| SLFN12L | 183.61 | 289.82 | 1.58 | 0.00015 | 0.002 |
| SLPI | 205.09 | 76.69 | 0.37 | 0.0027 | 0.019 |
| SMARCD3 | 32.35 | 95.53 | 2.95 | 6.60E−05 | 0.001 |
| SMC2 | 264.58 | 397.23 | 1.50 | 0.0022 | 0.017 |
| SMC4 | 631.28 | 1115.41 | 1.77 | 1.70E−10 | 1.40E−08 |
| SMURF2 | 261.22 | 507.59 | 1.94 | 1.40E−08 | 7.00E−07 |
| SNHG1 | 338.43 | 563.34 | 1.66 | 2.20E−06 | 5.80E−05 |
| SNHG11 | 71.98 | 39.83 | 0.55 | 0.0022 | 0.017 |
| SNORA29 | 3.27 | 13.35 | 4.08 | 2.20E−07 | 8.20E−06 |
| SNORA31 | 10.73 | 19.16 | 1.79 | 0.0077 | 0.043 |
| SNORA4 | 14.71 | 32.49 | 2.21 | 1.50E−08 | 7.50E−07 |
| SNORA63 | 19.17 | 38.43 | 2.00 | 1.20E−05 | 0.00024 |
| SNORD2 | 10.15 | 21.21 | 2.09 | 1.20E−05 | 0.00024 |
| SNORD22 | 17.65 | 31.26 | 1.77 | 0.0049 | 0.03 |
| SNORD25 | 4.73 | 12.19 | 2.58 | 0.00068 | 0.0066 |
| SNORD26 | 5.57 | 13.15 | 2.36 | 0.0033 | 0.023 |
| SNORD27 | 9.83 | 19.2 | 1.95 | 0.0026 | 0.019 |
| SNORD28 | 5.43 | 17.68 | 3.26 | 9.60E−06 | 2.00E−04 |
| SNORD29 | 5.56 | 14.92 | 2.68 | 4.10E−06 | 1.00E−04 |
| SNORD31 | 8.36 | 16.35 | 1.96 | 0.0026 | 0.019 |
| SNORD44 | 5.65 | 11.43 | 2.02 | 0.001 | 0.009 |
| SNORD45B | 3.52 | 11.19 | 3.18 | 0.00014 | 0.0019 |
| SNORD50A | 25.92 | 45.43 | 1.75 | 1.40E−05 | 0.00028 |
| SNORD50B | 42.54 | 85.19 | 2.00 | 6.70E−09 | 3.70E−07 |
| SNORD76 | 11.49 | 23.87 | 2.08 | 0.00097 | 0.0087 |
| SNORD79 | 9.87 | 20.72 | 2.10 | 7.20E−07 | 2.30E−05 |
| SNORD80 | 16.78 | 31.05 | 1.85 | 0.0014 | 0.011 |
| SNORD81 | 13.77 | 23.99 | 1.74 | 0.002 | 0.015 |
| SNTN | 27.41 | 11.09 | 0.40 | 0.0011 | 0.0098 |
| SOCS2 | 176.73 | 88.58 | 0.50 | 7.00E−04 | 0.0067 |
| SORBS3 | 268.33 | 158.24 | 0.59 | 6.10E−07 | 1.90E−05 |
| SORT1 | 240.83 | 59.83 | 0.25 | 3.30E−06 | 8.10E−05 |
| SOX4 | 33.1 | 135.89 | 4.11 | 6.30E−07 | 2.00E−05 |
| SPARC | 115.94 | 48.56 | 0.42 | 0.00053 | 0.0054 |
| SPATA6 | 22.63 | 6.37 | 0.28 | 0.008 | 0.044 |
| SPC25 | 7.46 | 34.66 | 4.65 | 7.60E−05 | 0.0011 |
| SPECC1 | 207.46 | 92.45 | 0.45 | 8.30E−05 | 0.0012 |
| SPI1 | 279.75 | 52.13 | 0.19 | 1.80E−15 | 4.70E−13 |
| SPIRE1 | 59.42 | 22.62 | 0.38 | 0.00073 | 0.0069 |
| SPN | 3765.6 | 2319.13 | 0.62 | 7.60E−10 | 5.20E−08 |
| SPON2 | 564.27 | 114.94 | 0.20 | 3.00E−14 | 5.60E−12 |
| SPP1 | 67.51 | 4220.13 | 62.51 | 7.40E−23 | 1.20E−19 |
| SPR | 42.37 | 14.45 | 0.34 | 0.0037 | 0.025 |
| SPSB1 | 52.34 | 142.63 | 2.73 | 0.00039 | 0.0042 |
| SPTB | 32.67 | 9.12 | 0.28 | 0.00037 | 0.0041 |
| SREBF2 | 376.04 | 250.57 | 0.67 | 0.0052 | 0.032 |
| SRGAP3 | 123.99 | 537 | 4.33 | 8.90E−19 | 5.40E−16 |
| SSBP3 | 49.99 | 22.13 | 0.44 | 9.40E−07 | 2.80E−05 |
| SSBP4 | 323.45 | 204.58 | 0.63 | 8.10E−07 | 2.50E−05 |
| ST6GALNAC2 | 56.6 | 14.94 | 0.26 | 0.00012 | 0.0016 |
| ST6GALNAC3 | 23.12 | 81.22 | 3.51 | 0.0014 | 0.012 |
| ST8SIA1 | 89.87 | 231.96 | 2.58 | 5.00E−08 | 2.20E−06 |
| STAC | 138.92 | 22.33 | 0.16 | 1.80E−05 | 0.00034 |
| STAT1 | 3572.42 | 6010.73 | 1.68 | 0.0019 | 0.015 |
| STON1 | 18.25 | 3.09 | 0.17 | 4.50E−05 | 0.00073 |
| STRBP | 161.32 | 358.23 | 2.22 | 2.20E−05 | 0.00039 |
| STX3 | 245.74 | 96.99 | 0.39 | 4.60E−08 | 2.00E−06 |
| STYXL1 | 174.92 | 287.84 | 1.65 | 0.005 | 0.031 |
| SUMO1P3 | 29.67 | 57.43 | 1.94 | 4.90E−05 | 0.00079 |
| SUN2 | 5714.36 | 3309.96 | 0.58 | 3.70E−09 | 2.10E−07 |
| SUPT3H | 187.47 | 313.87 | 1.67 | 1.50E−05 | 0.00029 |
| SUSD1 | 251.82 | 89.96 | 0.36 | 2.00E−06 | 5.30E−05 |
| SUSD3 | 426.27 | 764.9 | 1.79 | 5.00E−06 | 0.00012 |
| SUV39H2 | 53.45 | 81.87 | 1.53 | 0.0069 | 0.039 |
| SVIL | 193.06 | 76.8 | 0.40 | 4.80E−11 | 4.50E−09 |
| SYK | 219.29 | 101.28 | 0.46 | 0.00024 | 0.0028 |
| SYNJ1 | 144.05 | 225.25 | 1.56 | 2.00E−05 | 0.00037 |
| SYTL1 | 931.5 | 606.33 | 0.65 | 4.40E−07 | 1.50E−05 |
| TAGLN2 | 8378.09 | 4026.35 | 0.48 | 2.20E−26 | 1.00E−22 |
| TANC2 | 162.55 | 108.07 | 0.66 | 0.0018 | 0.014 |
| TAS2R19 | 2.43 | 18.1 | 7.45 | 0.0031 | 0.022 |
| TBC1D17 | 251.87 | 167.23 | 0.66 | 0.002 | 0.015 |
| TBC1D2 | 168.51 | 95.81 | 0.57 | 0.0054 | 0.033 |
| TBC1D4 | 193.08 | 444.84 | 2.30 | 0.00014 | 0.0019 |
| TBC1D9 | 26.68 | 14.33 | 0.54 | 0.0083 | 0.045 |
| TBCD | 1083.54 | 1764.92 | 1.63 | 5.60E−07 | 1.80E−05 |
| TBL1XR1 | 1724.49 | 2713.17 | 1.57 | 3.10E−08 | 1.40E−06 |
| TBX21 | 1423.31 | 602.11 | 0.42 | 7.30E−12 | 8.40E−10 |
| TCF7L2 | 172.44 | 48.34 | 0.28 | 1.10E−10 | 9.10E−09 |
| TFCP2L1 | 27.69 | 5.6 | 0.20 | 0.0051 | 0.032 |
| TFEB | 116.96 | 61.17 | 0.52 | 5.20E−05 | 0.00083 |
| TFEC | 69.56 | 20.77 | 0.30 | 2.10E−07 | 7.80E−06 |
| TGFBI | 837 | 402.27 | 0.48 | 0.0012 | 0.01 |
| TGFBR3 | 1808.02 | 786.77 | 0.44 | 3.70E−11 | 3.50E−09 |
| TGM2 | 722.79 | 206.62 | 0.29 | 2.60E−06 | 6.70E−05 |
| THAP6 | 209.72 | 316.93 | 1.51 | 0.0037 | 0.025 |
| THBD | 204.98 | 52.27 | 0.26 | 2.10E−07 | 8.00E−06 |
| THBS1 | 1075.72 | 361.65 | 0.34 | 1.90E−05 | 0.00035 |
| THEM4 | 371.48 | 246.99 | 0.66 | 0.0014 | 0.011 |
| THRA | 175.92 | 93.4 | 0.53 | 0.00029 | 0.0034 |
| TIAM1 | 183.24 | 431.88 | 2.36 | 1.70E−06 | 4.80E−05 |
| TIAM2 | 39.19 | 107.86 | 2.75 | 1.70E−06 | 4.60E−05 |
| TIGIT | 1790.85 | 2906.05 | 1.62 | 0.00027 | 0.0032 |
| TIMP1 | 889.13 | 347.74 | 0.39 | 1.00E−09 | 6.80E−08 |
| TIMP2 | 96.46 | 40.28 | 0.42 | 0.00031 | 0.0035 |
| TKTL1 | 94.13 | 23.29 | 0.25 | 0.0037 | 0.025 |
| TLR10 | 6.18 | 13.96 | 2.26 | 0.0055 | 0.033 |
| TLR4 | 129.76 | 52.24 | 0.40 | 0.0015 | 0.012 |
| TLR6 | 29.83 | 11.72 | 0.39 | 0.00055 | 0.0056 |
| TLR7 | 31.68 | 13.06 | 0.41 | 0.0036 | 0.024 |
| TLR8 | 61.03 | 17.26 | 0.28 | 0.0015 | 0.012 |
| TM7SF4 | 30.77 | 5.31 | 0.17 | 0.00019 | 0.0024 |
| TMBIM1 | 1822.98 | 1078.02 | 0.59 | 9.40E−10 | 6.30E−08 |
| TMCC3 | 210.98 | 52.28 | 0.25 | 1.60E−07 | 6.40E−06 |
| TMEM102 | 192.01 | 111.94 | 0.58 | 0.00062 | 0.0061 |
| TMEM104 | 230.44 | 139.71 | 0.61 | 0.0027 | 0.019 |
| TMEM14A | 171.93 | 260.46 | 1.51 | 0.0023 | 0.017 |
| TMEM155 | 9.25 | 79.37 | 8.58 | 0.00022 | 0.0026 |
| TMEM156 | 135.84 | 227.78 | 1.68 | 2.00E−04 | 0.0025 |
| TMEM173 | 1727.35 | 1136.57 | 0.66 | 1.60E−07 | 6.30E−06 |
| TMEM184B | 338.86 | 191.69 | 0.57 | 1.90E−05 | 0.00035 |
| TMEM185B | 266.93 | 151.58 | 0.57 | 0.0032 | 0.022 |
| TMEM220 | 28.09 | 11.39 | 0.41 | 5.00E−04 | 0.0052 |
| TMEM53 | 76.66 | 43.61 | 0.57 | 0.00014 | 0.0018 |
| TMEM63A | 545.2 | 362.65 | 0.67 | 0.00081 | 0.0075 |
| TMEM65 | 59.7 | 36.81 | 0.62 | 0.0045 | 0.029 |
| TMIGD2 | 60.6 | 151.67 | 2.50 | 0.002 | 0.016 |
| TMPO | 593.84 | 1079.45 | 1.82 | 2.80E−06 | 7.20E−05 |
| TMPRSS3 | 29.59 | 74.32 | 2.51 | 0.00021 | 0.0026 |
| TMPRSS4 | 3.24 | 24.16 | 7.46 | 0.0088 | 0.047 |
| TNFAIP2 | 72.59 | 23.46 | 0.32 | 1.10E−06 | 3.20E−05 |
| TNFAIP8L2 | 191.54 | 108.59 | 0.57 | 0.0011 | 0.0094 |
| TNFRSF9 | 272.09 | 1326.53 | 4.88 | 2.40E−21 | 2.60E−18 |
| TNFSF13 | 79.04 | 18.36 | 0.23 | 1.80E−06 | 4.80E−05 |
| TNFSF15 | 5.52 | 16.09 | 2.91 | 9.70E−05 | 0.0014 |
| TNFSF4 | 98.94 | 470.59 | 4.76 | 1.80E−10 | 1.40E−08 |
| TNFSF9 | 23.14 | 47.67 | 2.06 | 1.70E−08 | 8.40E−07 |
| TNIP3 | 364.47 | 717.57 | 1.97 | 4.70E−08 | 2.00E−06 |
| TNNI2 | 27.63 | 4.48 | 0.16 | 1.30E−05 | 0.00026 |
| TNS3 | 119.87 | 421.11 | 3.51 | 3.90E−05 | 0.00064 |
| TOM1L2 | 198.12 | 129.63 | 0.65 | 0.006 | 0.036 |
| TOP2A | 141.45 | 548.61 | 3.88 | 1.60E−09 | 1.00E−07 |
| TOR2A | 335.23 | 219.53 | 0.65 | 0.002 | 0.016 |
| TOX | 843.57 | 1329.31 | 1.58 | 2.20E−09 | 1.30E−07 |
| TOX2 | 72.98 | 184.99 | 2.53 | 0.00021 | 0.0025 |
| TP53BP1 | 257.92 | 468.89 | 1.82 | 9.70E−06 | 2.00E−04 |
| TP53INP1 | 255.18 | 414.51 | 1.62 | 3.90E−08 | 1.70E−06 |
| TP73 | 19.69 | 72.54 | 3.68 | 0.0031 | 0.021 |
| TPCN1 | 114.22 | 183.8 | 1.61 | 9.40E−05 | 0.0013 |
| TPGS1 | 201.93 | 130.28 | 0.65 | 5.60E−06 | 0.00013 |
| TPPP | 79.06 | 13.7 | 0.17 | 1.40E−12 | 1.80E−10 |
| TPPP3 | 112.15 | 19.81 | 0.18 | 8.70E−05 | 0.0013 |
| TPST2 | 1575.74 | 1042.48 | 0.66 | 1.10E−07 | 4.50E−06 |
| TPX2 | 58.26 | 220.32 | 3.78 | 0.00016 | 0.0021 |
| TRAF1 | 483.76 | 730.03 | 1.51 | 1.50E−05 | 0.00029 |
| TRAF5 | 812.26 | 1375.18 | 1.69 | 2.90E−09 | 1.70E−07 |
| TRAM2 | 181.62 | 86.95 | 0.48 | 4.20E−06 | 1.00E−04 |
| TREM1 | 763.65 | 112.14 | 0.15 | 1.40E−10 | 1.10E−08 |
| TREM2 | 164.98 | 106.94 | 0.65 | 0.0019 | 0.014 |
| TRIM13 | 347.46 | 546.62 | 1.57 | 1.50E−06 | 4.10E−05 |
| TRIM44 | 517.41 | 329.06 | 0.64 | 6.80E−05 | 0.001 |
| TRIM59 | 509.51 | 805.85 | 1.58 | 3.60E−07 | 1.20E−05 |
| TRIM69 | 32.9 | 86.18 | 2.62 | 0.004 | 0.026 |
| TROAP | 18.48 | 54.75 | 2.96 | 0.0013 | 0.011 |
| TRPA1 | 8.77 | 25.91 | 2.95 | 0.0017 | 0.014 |
| TRPC1 | 18.06 | 9.62 | 0.53 | 0.0068 | 0.039 |
| TRPC6 | 29.52 | 5.14 | 0.17 | 0.00028 | 0.0032 |
| TRPS1 | 417.36 | 751.46 | 1.80 | 0.00043 | 0.0046 |
| TSC22D3 | 18891.64 | 11302.78 | 0.60 | 5.90E−08 | 2.50E−06 |
| TSHZ2 | 13.57 | 47.73 | 3.52 | 2.50E−06 | 6.50E−05 |
| TSHZ3 | 55.04 | 16.54 | 0.30 | 0.00033 | 0.0037 |
| TSPAN15 | 97.67 | 33.1 | 0.34 | 0.0019 | 0.015 |
| TSPAN2 | 370.47 | 123.68 | 0.33 | 4.80E−09 | 2.70E−07 |
| TSPAN5 | 387.68 | 618.17 | 1.59 | 0.00028 | 0.0032 |
| TSPYL4 | 557.87 | 903.74 | 1.62 | 1.20E−09 | 7.80E−08 |
| TTC14 | 652.51 | 1014.03 | 1.55 | 2.20E−07 | 8.10E−06 |
| TTC16 | 276.77 | 168.35 | 0.61 | 0.0029 | 0.021 |
| TTC24 | 90.99 | 226 | 2.48 | 6.80E−05 | 0.001 |
| TTC38 | 796.85 | 342.2 | 0.43 | 5.90E−09 | 3.30E−07 |
| TTK | 16.7 | 82.08 | 4.91 | 0.0023 | 0.017 |
| TTN | 924.63 | 2137.03 | 2.31 | 4.30E−15 | 1.00E−12 |
| TTYH2 | 99.44 | 22.6 | 0.23 | 2.60E−08 | 1.20E−06 |
| TUBB4A | 85.06 | 43.27 | 0.51 | 0.00079 | 0.0074 |
| TUBB6 | 119.37 | 26.51 | 0.22 | 2.20E−05 | 0.00039 |
| TXK | 440.47 | 212.44 | 0.48 | 0.00041 | 0.0044 |
| TXNDC3 | 42.04 | 5.41 | 0.13 | 7.30E−10 | 5.10E−08 |
| TYMS | 28.21 | 80.14 | 2.84 | 0.0023 | 0.017 |
| TYROBP | 1664.05 | 496.36 | 0.30 | 9.60E−15 | 2.20E−12 |
| UBE2C | 38.87 | 179.14 | 4.61 | 7.70E−05 | 0.0011 |
| UBE2E2 | 70.91 | 25.98 | 0.37 | 0.00019 | 0.0023 |
| UBXN10 | 59.35 | 13.72 | 0.23 | 0.00021 | 0.0025 |
| UCP2 | 8236.16 | 4949.76 | 0.60 | 2.80E−12 | 3.40E−10 |
| UHRF1 | 30.49 | 104.92 | 3.44 | 0.0017 | 0.013 |
| ULK2 | 161.01 | 90.83 | 0.56 | 0.00048 | 0.005 |
| UNC13B | 30.62 | 5.88 | 0.19 | 0.0029 | 0.02 |
| UNC5B | 36.36 | 2.13 | 0.06 | 2.30E−18 | 1.20E−15 |
| UNC93B1 | 247.43 | 128.37 | 0.52 | 0.00024 | 0.0029 |
| UPP1 | 835.94 | 474.87 | 0.57 | 9.40E−11 | 8.10E−09 |
| VAMP2 | 1141.43 | 758.44 | 0.66 | 7.60E−08 | 3.10E−06 |
| VARS | 438.94 | 288.23 | 0.66 | 6.90E−05 | 0.001 |
| VASH1 | 121.05 | 62.36 | 0.52 | 0.0041 | 0.027 |
| VAT1 | 980.21 | 360.71 | 0.37 | 2.10E−10 | 1.60E−08 |
| VCAM1 | 194.14 | 635.43 | 3.27 | 3.40E−05 | 0.00058 |
| VCAN | 226.28 | 135.6 | 0.60 | 0.001 | 0.0091 |
| VCL | 616.26 | 220.57 | 0.36 | 2.50E−14 | 4.80E−12 |
| VDR | 49.82 | 156.49 | 3.14 | 0.00013 | 0.0018 |
| VGLL3 | 28.23 | 1.98 | 0.07 | 1.50E−07 | 6.00E−06 |
| VIM | 10359.24 | 6551.63 | 0.63 | 1.60E−10 | 1.20E−08 |
| VMO1 | 43.05 | 5.68 | 0.13 | 3.10E−11 | 3.00E−09 |
| VPS18 | 359.33 | 232.63 | 0.65 | 0.0036 | 0.024 |
| VPS54 | 108.35 | 163.46 | 1.51 | 0.00058 | 0.0058 |
| VSIG4 | 1532.41 | 251.18 | 0.16 | 5.30E−12 | 6.20E−10 |
| VSTM4 | 17.6 | 30.26 | 1.72 | 0.007 | 0.04 |
| WDFY4 | 43.98 | 13.46 | 0.31 | 0.0061 | 0.036 |
| WDR13 | 347.89 | 206.36 | 0.59 | 0.00023 | 0.0028 |
| WDR45 | 499.73 | 328.49 | 0.66 | 0.00046 | 0.0049 |
| WDR81 | 177.73 | 108.84 | 0.61 | 0.0089 | 0.047 |
| WDR96 | 22.84 | 3.81 | 0.17 | 2.10E−05 | 0.00039 |
| WIPF3 | 11.47 | 108.36 | 9.45 | 1.50E−14 | 3.30E−12 |
| WNT10A | 68.19 | 111.13 | 1.63 | 0.00091 | 0.0083 |
| WWC2 | 43.71 | 15.15 | 0.35 | 0.0023 | 0.017 |
| XBP1 | 3890.25 | 2024.85 | 0.52 | 8.80E−21 | 9.10E−18 |
| XIST | 1638.7 | 2573.51 | 1.57 | 6.90E−05 | 0.001 |
| XPNPEP2 | 48.75 | 12.58 | 0.26 | 0.0028 | 0.02 |
| YPEL1 | 449.2 | 274.75 | 0.61 | 5.50E−05 | 0.00087 |
| YPEL2 | 151.79 | 253.18 | 1.67 | 1.00E−05 | 0.00021 |
| ZBED2 | 101.64 | 394.29 | 3.88 | 9.10E−14 | 1.50E−11 |
| ZBTB16 | 247.24 | 103.68 | 0.42 | 0.001 | 0.009 |
| ZBTB3 | 107.93 | 70.94 | 0.66 | 0.0059 | 0.035 |
| ZBTB7C | 21.33 | 7.47 | 0.35 | 0.0085 | 0.046 |
| ZDBF2 | 69.43 | 128.44 | 1.85 | 0.0074 | 0.041 |
| ZDHHC11 | 20.44 | 8.25 | 0.40 | 0.00046 | 0.0049 |
| ZFP14 | 103.17 | 161.8 | 1.57 | 0.00033 | 0.0037 |
| ZMAT1 | 106.55 | 174.08 | 1.63 | 0.0024 | 0.018 |
| ZMYM2 | 640.4 | 989.71 | 1.55 | 3.10E−06 | 7.90E−05 |
| ZMYND10 | 34.15 | 12.6 | 0.37 | 0.0086 | 0.046 |
| ZNF248 | 88.34 | 147.94 | 1.67 | 0.0089 | 0.048 |
| ZNF267 | 516.61 | 792.4 | 1.53 | 5.50E−10 | 3.90E−08 |
| ZNF280C | 68.83 | 108.39 | 1.57 | 0.0035 | 0.024 |
| ZNF362 | 310.97 | 190.12 | 0.61 | 0.00064 | 0.0062 |
| ZNF365 | 117.51 | 21.31 | 0.18 | 5.60E−09 | 3.10E−07 |
| ZNF385A | 33.47 | 11.8 | 0.35 | 0.00061 | 0.006 |
| ZNF408 | 134.28 | 66.78 | 0.50 | 0.0039 | 0.026 |
| ZNF414 | 41.09 | 24.42 | 0.59 | 0.00028 | 0.0032 |
| ZNF460 | 430.04 | 707.06 | 1.64 | 0.00033 | 0.0037 |
| ZNF518B | 219.31 | 344.03 | 1.57 | 7.00E−04 | 0.0067 |
| ZNF783 | 105.33 | 59.99 | 0.57 | 6.40E−05 | 0.00098 |
| ZNF827 | 168.82 | 258.55 | 1.53 | 0.00024 | 0.0028 |
| ZNF837 | 27.76 | 15.44 | 0.56 | 0.0052 | 0.032 |
| ZNF841 | 63.19 | 108.56 | 1.72 | 0.0016 | 0.013 |
| ZNRF1 | 86.03 | 272.46 | 3.17 | 1.00E−05 | 0.00021 |

TABLE 5
Pathway analysis of differentially expressed genes (DEGs) in CD8+ TILs from NSCLC.

| | | | Percent- | | | |
|---|---|---|---|---|---|---|
| | | Number | age of | Fraction | | |
| Ingenuity | −log | of genes | DEGs | of down- | Fraction | |
| canonical | (P- | in | in | regulated | of up- | |
| pathways | value) | pathway | pathway | genes | regulated | DEGS in the pathway |
| ATM Signaling | 9.18 | 59 | 22% | 0/59 | 13/59 | CDC25C, TP73, CCNB2, CBX5, MAPK12, CDK1, |
| | | | | (0%) | (22%) | CHEK1, CCNB1, JUN, SMC2, H2AFX, TP53BP1, BLM |
| Hereditary Breast | 5.21 | 144 | 10% | 5/144 | 14/144 | FANCM, POLR2J2/POLR2J3, CDC25C, PIK3C2A, |
| Cancer Signaling | | | | (3%) | (10%) | FGFR1, BARD1, PIK3R5, FANCL, CDK1, SMARCD3, |
| (HBCS) | | | | | | CHEK1, CCNB1, RRAS2, MSH2, RFC4, H2AFX, MRAS, |
| | | | | | | PIK3R2, BLM |
| Role of Osteoblasts, | 4.96 | 238 | 8% | 16/238 | 18/238 | CAMK4, AXIN1, LRP6, PIK3R5, JUN, IGF1, DKK3, |
| Osteoclasts and | | | | (7%) | (8%) | RUNX2, PIK3R2, TRAF5, ITGB1, IFNG, MAP3K14, SPP1, |
| Chondrocytes in | | | | | | PIK3C2A, IL10, BMP8A, FGFR1, BMP8B, ITGA2, ITGA5, |
| Rheumatoid | | | | | | GSN, MAPK12, CSF1R, IL17A, IL18, FZD4, WNT10A, |
| Arthritis | | | | | | CSF1, FZD6, IL1B, LEF1, LRP1, TCF7L2 |
| Role of BRCA1 in | 4.93 | 78 | 13% | 0/78 | 10/78 | FANCM, IFNG, MSH2, RFC4, BARD1, STAT1, |
| DNA Damage | | | | (0%) | (13%) | BLM, SMARCD3, FANCL, CHEK1 |
| Response | | | | | | |
| Cell Cycle: G2/M | 4.84 | 49 | 16% | 0/49 | 8/49 | CDC25C, CKS2, CKS1B, TOP2A, CCNB2, CDK1, |
| DNA Damage | | | | (0%) | (16%) | CHEK1, CCNB1 |
| Checkpoint | | | | | | |
| Regulation | | | | | | |
| Mitotic Roles of | 4.71 | 66 | 14% | 0/66 | 9/66 | KIF23, CDC25C, CDC20, PTTG1, CCNB2, HSP90AA1, |
| Polo-Like Kinase | | | | (0%) | (14%) | CDK1, KIF11, CCNB1 |
| Altered T Cell and | 4.47 | 88 | 11% | 12/88 | 10/88 | IFNG, MAP3K14, SPP1, IL10, TLR8, CD79A, |
| B Cell Signaling in | | | | (14%) | (11%) | HLA-DQB1, IL17A, TLR4, IL18, TLR10, HLA- |
| Rheumatoid | | | | | | DRB1, CXCL13, CSF1, TNFSF13, HLA-DRA, |
| Arthritis | | | | | | TLR6, TLR7, FCER1G, IL1B, HLA-DRB5, FASLG |
| 4-1BB Signaling in | 4.19 | 31 | 19% | 0/31 | 6/31 | MAP3K14, TNFRSF9, JUN, TNFSF9, MAPK12, TRAF1 |
| T Lymphocytes | | | | (0%) | (19%) | |
| 3-phosphoinositide | 3.65 | 153 | 8% | 4/153 | 12/153 | CDC25C, PTPN7, PTPN13, MTMR14, STYXL1, |
| Degradation | | | | (3%) | (8%) | PTPN12, PPP1R14B, PTPRF, TNS3, INPP5F, |
| | | | | | | SYNJ1, PDCD1, PTPRO, PTPN22, SIRPA, DUSP16 |
| D-myo-inositol | 3.53 | 135 | 8% | 4/135 | 11/135 | CDC25C, PTPN7, ATP, PTPN13, STYXL1, PTPN12, |
| (1,4,5,6)- | | | | (3%) | (8%) | PPP1R14B, PTPRF, TNS3, SYNJ1, PTPRO, PDCD1, |
| Tetrakisphosphate | | | | | | PTPN22, SIRPA, DUSP16 |
| Biosynthesis | | | | | | |
| D-myo-inositol | 3.53 | 135 | 8% | 4/135 | 11/135 | CDC25C, PTPN7, ATP, PTPN13, STYXL1, PTPN12, |
| (3,4,5,6)- | | | | (3%) | (8%) | PPP1R14B, PTPRF, TNS3, SYNJ1, PTPRO, |
| tetrakisphosphate | | | | | | PDCD1, PTPN22, SIRPA, DUSP16 |
| Biosynthesis | | | | | | |
| Cell Cycle Control | 3.48 | 27 | 19% | 0/27 | 5/27 | CDC45, CDT1, CDC6, ORC6, MCM4 |
| of Chromosomal | | | | (0%) | (19%) | |
| Replication | | | | | | |
| Protein Kinase A | 3.35 | 402 | 5% | 16/402 | 21/402 | PDE6G, CAMK4, ATP, TNNI2, PTPN13, PDE4A, |
| Signaling | | | | (4%) | (5%) | LIPE, MYL6B, PPP1R14B, PTPN12, PTPRF, CDKN3, |
| | | | | | | PLCD1, PDE7B, PTPRO, FLNA, PRKAR1B, GNG4, |
| | | | | | | AKAP5, PXN, CDC25C, PTPN7, PTPRK, RYR2, PTCH1, |
| | | | | | | PTPN18, TTN, PDE8A, HIST1H1B, PRKCD, KDELR3, |
| | | | | | | DUSP4, LEF1, PTPN22, TCF7L2, SIRPA, DUSP16 |
| 3-phosphoinositide | 3.18 | 197 | 7% | 8/197 | 13/197 | CDC25C, PTPN7, ATP, PIK3C2A, PTPN13, FGFR1, |
| Biosynthesis | | | | (4%) | (7%) | PIK3R5, ERBB3, STYXL1, PTPN12, PPP1R14B, PTPRF, |
| | | | | | | TNS3, SYNJ1, PDCD1, PIP5K1C, PTPRO, PIK3R2, |
| | | | | | | PTPN22, SIRPA, DUSP16 |
| D-myo-inositol-5- | 3.05 | 154 | 7% | 5/154 | 11/154 | CDC25C, PTPN7, ATP, PTPN13, STYXL1, PTPN12, |
| phosphate | | | | (3%) | (7%) | PPP1R14B, PTPRF, PLCD1, TNS3, SYNJ1, PDCD1, |
| Metabolism | | | | | | PTPRO, PTPN22, SIRPA, DUSP16 |
| p53 Signaling | 2.97 | 111 | 8% | 5/111 | 9/111 | PMAIP1, TP53INP1, PIK3C2A, TP73, PLAGL1, FGFR1, |
| | | | | (5%) | (8%) | PIK3R5, HIF1A, CHEK1, PCNA, JUN, BBC3, THBS1, |
| | | | | | | PIK3R2 |
| T Helper Cell | 2.88 | 72 | 10% | 8/72 | 7/72 | IL6ST, IFNG, IL10, IFNGR2, HLA-DQB1, TBX21, |
| Differentiation | | | | (11%) | (10%) | IL17A, IL18, HLA-DRB1, HLA-DRA, ICOS, FCER1G, |
| | | | | | | CXCR5, STAT1, HLA-DRB5 |
| Role of CHK | 2.8 | 55 | 11% | 0/55 | 6/55 | PCNA, CDC25C, RFC4, CLSPN, CDK1, CHEK1 |
| Proteins in Cell | | | | (0%) | (11%) | |
| Cycle Checkpoint | | | | | | |
| Control | | | | | | |
| April Mediated | 2.77 | 38 | 13% | 1/38 | 5/38 | MAP3K14, JUN, TNFSF13, TRAF5, MAPK12, |
| Signaling | | | | (3%) | (13%) | TRAF1 |
| Superpathway of | 2.74 | 247 | 6% | 9/247 | 14/247 | CDC25C, PTPN7, ATP, PIK3C2A, PTPN13, FGFR1, |
| Inositol Phosphate | | | | (4%) | (6%) | PIK3R5, ERBB3, STYXL1, PTPN12, PPP1R14B, PTPRF, |
| Compounds | | | | | | PLCD1, TNS3, SYNJ1, INPP5F, PDCD1, PIP5K1C, |
| | | | | | | PTPRO, PIK3R2, PTPN22, SIRPA, DUSP16 |
| B Cell Activating | 2.67 | 40 | 13% | 0/40 | 5/40 | MAP3K14, JUN, TRAF5, MAPK12, TRAF1 |
| Factor Signaling | | | | (0%) | (13%) | |
| Colorectal Cancer | 2.66 | 252 | 6% | 20/252 | 14/252 | IL6ST, ATP, AXIN1, LRP6, PIK3R5, TLR8, |
| Metastasis | | | | (8%) | (6%) | TLR10, ARRB1, JUN, TLR7, MRAS, RHOU, PRKAR1B, |
| Signaling | | | | | | PIK3R2, MMP12, STAT1, MMP19, GNG4, IFNG, |
| | | | | | | PIK3C2A, FGFR1, MAPK12, TLR4, RRAS2, FZD4, |
| | | | | | | WNT10A, MSH2, RND3, TLR6, FZD6, LEF1, PTGER2, |
| | | | | | | LRP1, TCF7L2 |
| Role of JAK family | 2.62 | 25 | 16% | 0/25 | 4/25 | IL6ST, OSMR, STAT1, MAPK12 |
| kinases in IL-6-type | | | | (0%) | (16%) | |
| Cytokine Signaling | | | | | | |
| Role of | 2.56 | 315 | 5% | 26/315 | 16/315 | IL6ST, CAMK4, FN1, AXIN1, LRP6, TLR8, PIK3R5, |
| Macrophages, | | | | (8%) | (5%) | FCGR1A, PLCD1, TLR10, JUN, DKK3, TLR7, CEBPA, |
| Fibroblasts and | | | | | | MRAS, TRAF5, PIK3R2, FCGR3A/FCGR3B, TRAF1, |
| Endothelial Cells in | | | | | | MAP3K14, VCAM1, C5AR1, PIK3C2A, IL10, FGFR1, |
| Rheumatoid | | | | | | CEBPB, IRAK3, IL17A, TLR4, IL18, RRAS2, FZD4, |
| Arthritis | | | | | | WNT10A, CSF1, PRKCD, TLR6, FZD6, IL1B, LEF1, |
| | | | | | | PDGFD, LRP1, TCF7L2 |
| dTMP De Novo | 2.44 | 14 | 21% | 0/14 | 3/14 | TYMS, NADPH, DHFR |
| Biosynthesis | | | | (0%) | (21%) | |
| NF-κB Activation | 2.41 | 87 | 8% | 12/87 | 7/87 | ITGB1, MAP3K14, CCR5, PIK3C2A, FGFR1, |
| by Viruses | | | | (14%) | (8%) | CD4, ITGA2, PIK3R5, ITGA6, ITGA5, ITGB2, |
| | | | | | | RRAS2, PRKCD, MRAS, ITGA1, PIK3R2, CXCR5, |
| | | | | | | EIF2AK2, ITGB5 |
| Hepatic Fibrosis/ | 2.39 | 187 | 6% | 13/187 | 11/187 | IFNG, IGFBP4, CCR5, VCAM1, FN1, FLT1, COL6A2, |
| Hepatic Stellate | | | | (7%) | (6%) | IL10, FGFR1, KLF6, FLT4, IFNGR2, MYL6B, TLR4, |
| Cell Activation | | | | | | CXCL3, COL6A3, IGF1, CSF1, TIMP1, IL1B, STAT1, |
| | | | | | | PDGFD, FASLG, TIMP2 |
| iNOS Signaling | 2.36 | 47 | 11% | 3/47 | 5/47 | TLR4, IFNG, CAMK4, JUN, IFNGR2, IRAK3, STAT1, |
| | | | | (6%) | (11%) | MAPK12 |
| Agrin Interactions | 2.27 | 70 | 9% | 7/70 | 6/70 | ITGB1, ITGB2, PXN, JUN, RRAS2, ITGA2, MRAS, |
| at Neuromuscular | | | | (10%) | (9%) | ITGA6, ITGA5, ITGA1, ERBB3, ACTG2, MAPK12 |
| Junction | | | | | | |
| CD27 Signaling in | 2.17 | 52 | 10% | 0/52 | 5/52 | MAP3K14, JUN, TRAF5, CD27, MAPK12 |
| Lymphocytes | | | | (0%) | (10%) | |
| CCR5 Signaling in | 2.15 | 74 | 8% | 4/74 | 6/74 | GNG4, CCR5, CAMK4, JUN, PRKCD, CD4, MRAS, |
| Macrophages | | | | (5%) | (8%) | FCER1G, MAPK12, FASLG |
| Toll-like Receptor | 2.15 | 74 | 8% | 7/74 | 6/74 | TLR4, MAP3K14, TLR10, IL18, JUN, TLR6, TLR8, |
| Signaling | | | | (9%) | (8%) | TLR7, IL1B, IRAK3, EIF2AK2, MAPK12, TRAF1 |
| Protein | 2.14 | 259 | 5% | 4/259 | 13/259 | IFNG, ATP, CDC20, DNAJB4, HSPH1, HSPA1A/HSPA1B, |
| Ubiquitination | | | | (2%) | (5%) | HSPA6, HSPD1, DNAJA1, DNAJC28, DNAJC5, HSPE1, |
| Pathway | | | | | | UBE2E2, HSP90AA1, SMURF2, DNAJB1, UBE2C |
| Mismatch Repair in | 2.12 | 18 | 17% | 1/18 | 3/18 | PCNA, ATP, MSH2, RFC4 |
| Eukaryotes | | | | (6%) | (17%) | |
| Aldosterone | 2.11 | 176 | 6% | 8/176 | 10/176 | PIK3C2A, DNAJB4, HSPH1, FGFR1, HSPA1A/HSPA1B, |
| Signaling in | | | | (5%) | (6%) | HSPA6, PIK3R5, HSPD1, DNAJA1, PLCD1, DNAJC28, |
| Epithelial Cells | | | | | | DNAJC5, PIP5K1C, PRKCD, HSPE1, HSP90AA1, |
| | | | | | | PIK3R2, DNAJB1 |
| Unfolded protein | 2.1 | 54 | 9% | 5/54 | 5/54 | PPARG, DDIT3, SREBF2, HSPH1, HSPA1A/HSPA1B, |
| response | | | | (9%) | (9%) | HSPA6, CEBPA, XBP1, CD82, CEBPB |
| IL-17A Signaling in | 2.09 | 35 | 11% | 2/35 | 4/35 | JUN, CXCL5, CEBPB, MAPK12, NFKBIZ, IL17A |
| Fibroblasts | | | | (6%) | (11%) | |
| BMP signaling | 2.07 | 77 | 8% | 3/77 | 6/77 | CAMK4, JUN, RRAS2, RUNX2, BMP8A, BMP8B, MRAS, |
| pathway | | | | (4%) | (8%) | PRKAR1B, MAPK12 |
| GADD45 Signaling | 2.05 | 19 | 16% | 1/19 | 3/19 | PCNA, CCND3, CDK1, CCNB1 |
| | | | | (5%) | (16%) | |
| DNA damage- | 2.05 | 19 | 16% | 0/19 | 3/19 | CCNB2, CDK1, CCNB1 |
| induced 14-3-3σ | | | | (0%) | (16%) | |
| Signaling | | | | | | |
| CD40 Signaling | 2.04 | 78 | 8% | 3/78 | 6/78 | MAP3K14, JUN, PIK3C2A, FGFR1, PIK3R5, TRAF5, |
| | | | | (4%) | (8%) | PIK3R2, MAPK12, TRAF1 |
| B Cell Receptor | 1.89 | 190 | 5% | 11/190 | 10/190 | RAP2B, RAP2A, MAP3K14, CAMK4, PIK3C2A, FCGR2A, |
| Signaling | | | | (6%) | (5%) | EGR1, FGFR1, PIK3R5, CD79A, MAPK12, BTK, JUN, |
| | | | | | | RRAS2, SYNJ1, INPP5F, SYK, PAG1, MRAS, LYN, |
| | | | | | | PIK3R2 |
| Role of PKR in | 1.88 | 40 | 10% | 1/40 | 4/40 | IFNG, TRAF5, EIF2AK2, STAT1, FCGR1A |
| Interferon Induction | | | | (3%) | (10%) | |
| and Antiviral | | | | | | |
| Response | | | | | | |
| PCP pathway | 1.83 | 63 | 8% | 2/63 | 5/63 | JUN, SDC1, FZD4, WNT10A, EFNB1, FZD6, MAPK12 |
| | | | | (3%) | (8%) | |
| TGF-β Signaling | 1.82 | 87 | 7% | 4/87 | 6/87 | JUN, RRAS2, RUNX2, MRAS, SMURF2, ACVR2B, VDR, |
| | | | | (5%) | (7%) | MAPK12, INHBA, PMEPA1 |
| Differential | 1.82 | 23 | 13% | 2/23 | 3/23 | IFNG, IL10, IL1B, DEFB1, IL17A |
| Regulation of | | | | (9%) | (13%) | |
| Cytokine | | | | | | |
| Production in | | | | | | |
| Intestinal Epithelial | | | | | | |
| Cells by IL-17A | | | | | | |
| and IL-17F | | | | | | |
| Androgen Signaling | 1.79 | 114 | 6% | 2/114 | 7/114 | POLR2J2/POLR2J3, GNG4, CAMK4, JUN, PRKCD, |
| | | | | (2%) | (6%) | MRAS, PRKAR1B, HSP90AA1, DNAJB1 |
| Glucocorticoid | 1.73 | 293 | 4% | 16/293 | 13/293 | HSPA1A/HSPA1B, PIK3R5, HSPA6, SLPI, CD163, |
| Receptor Signaling | | | | (5%) | (4%) | FCGR1A, TSC22D3, CXCL3, JUN, ANXA1, CEBPA, |
| | | | | | | MRAS, PIK3R2, STAT1, ADRB2, POLR2J2/POLR2J3, |
| | | | | | | MAP3K14, IFNG, VCAM1, PIK3C2A, IL10, FGFR1, |
| | | | | | | CEBPB, MAPK12, SMARCD3, SCGB1A1, RRAS2, |
| | | | | | | HSP90AA1, IL1B |
| IL-17A Signaling in | 1.72 | 25 | 12% | 0/25 | 3/25 | JUN, MAPK12, IL17A |
| Gastric Cells | | | | (0%) | (12%) | |
| Lymphotoxin β | 1.67 | 69 | 7% | 3/69 | 5/69 | MAP3K14, VCAM1, PIK3C2A, FGFR1, PIK3R5, TRAF5, |
| Receptor Signaling | | | | (4%) | (7%) | PIK3R2, TRAF1 |

TABLE 6
Analysis of TCR beta chain sequences from RNA-Seq
data of CD8+ N-TIL versus NSCLC CD8+ TIL.
Table lists the number of clonotypes based on their
frequencies in CD8+ TILs and N-TILs from each patient.

| | Sample type | | | | | | | |
|---|---|---|---|---|---|---|---|---|
| | CD8+ | CD8+ | CD8+ | CD8+ | CD8+ | CD8+ | CD8+ | CD8+ |
| | N- | | N- | | N- | | N- | |
| | TILs | TILs | TILs | TILs | TILs | TILs | TILs | TILs |
| | Frequency of clonotypes | | | | | | | |
| Patient ID | >1 | >1 | >2 | >2 | >3 | >3 | >4 | >4 |
| NSCLC_30 | 16 | 7 | 8 | 2 | 7 | 1 | 4 | 1 |
| NSCLC_35 | 10 | 11 | 2 | 7 | 0 | 3 | 0 | 2 |
| NSCLC_33 | 26 | 31 | 15 | 16 | 12 | 11 | 9 | 8 |
| NSCLC_26 | 8 | 6 | 3 | 5 | 3 | 3 | 3 | 2 |
| NSCLC_27 | 21 | 18 | 12 | 14 | 10 | 10 | 8 | 8 |
| NSCLC_12 | 17 | 22 | 13 | 8 | 8 | 5 | 6 | 4 |
| NSCLC_17 | 6 | 27 | 3 | 11 | 1 | 6 | 0 | 5 |
| NSCLC_11 | 9 | 10 | 7 | 9 | 4 | 7 | 2 | 5 |
| NSCLC_22 | 21 | 22 | 15 | 14 | 10 | 10 | 9 | 8 |
| NSCLC_25 | 9 | 21 | 6 | 12 | 4 | 9 | 4 | 5 |
| NSCLC_05 | 9 | 12 | 3 | 8 | 3 | 7 | 2 | 7 |
| NSCLC_08 | 24 | 23 | 8 | 9 | 7 | 4 | 7 | 3 |
| NSCLC_32 | 20 | 24 | 11 | 11 | 8 | 7 | 6 | 6 |
| NSCLC_36 | 16 | 11 | 6 | 7 | 4 | 2 | 3 | 0 |
| NSCLC_23 | 28 | 16 | 16 | 10 | 12 | 5 | 7 | 1 |
| NSCLC_34 | 8 | 6 | 5 | 3 | 3 | 1 | 3 | 1 |
| NSCLC_29 | 6 | 9 | 3 | 4 | 3 | 2 | 2 | 1 |
| NSCLC_28 | 15 | 16 | 6 | 8 | 5 | 6 | 3 | 5 |
| NSCLC_03 | 14 | 15 | 7 | 8 | 4 | 3 | 3 | 2 |
| NSCLC_14 | 10 | 28 | 8 | 15 | 5 | 12 | 4 | 9 |
| NSCLC_01 | 10 | 25 | 4 | 18 | 4 | 10 | 2 | 8 |
| NSCLC_16 | 13 | 3 | 4 | 0 | 2 | 0 | 1 | 0 |
| NSCLC_10 | 10 | 10 | 6 | 8 | 2 | 5 | 0 | 4 |
| NSCLC_02 | 15 | 23 | 9 | 13 | 5 | 8 | 4 | 5 |
| NSCLC_19 | 17 | 13 | 8 | 9 | 6 | 6 | 6 | 5 |
| NSCLC_39 | 11 | NA | 8 | NA | 7 | NA | 6 | NA |
| NSCLC_40 | 9 | NA | 3 | NA | 2 | NA | 2 | NA |
| NSCLC_37 | 15 | NA | 11 | NA | 5 | NA | 2 | NA |
| NSCLC_38 | 8 | NA | 4 | NA | 2 | NA | 2 | NA |
| NSCLC_41 | 12 | NA | 8 | NA | 4 | NA | 4 | NA |
| NSCLC_42 | 8 | NA | 3 | NA | 2 | NA | 1 | NA |
| NSCLC_43 | 12 | NA | 6 | NA | 2 | NA | 1 | NA |
| NSCLC_07 | NA | 20 | NA | 13 | NA | 11 | NA | 10 |
| NSCLC_31 | NA | 15 | NA | 9 | NA | 5 | NA | 4 |
| NSCLC_15 | NA | 32 | NA | 19 | NA | 14 | NA | 11 |
| NSCLC_20 | NA | 8 | NA | 6 | NA | 4 | NA | 3 |
| NSCLC_06 | NA | 20 | NA | 13 | NA | 11 | NA | 8 |
| NSCLC_04 | NA | 26 | NA | 15 | NA | 12 | NA | 9 |
| NSCLC_21 | NA | 20 | NA | 12 | NA | 9 | NA | 9 |
| NSCLC_18 | NA | 22 | NA | 7 | NA | 3 | NA | 3 |
| NSCLC_24 | NA | 8 | NA | 5 | NA | 5 | NA | 4 |
| NSCLC_13 | NA | 14 | NA | 10 | NA | 7 | NA | 6 |
| NSCLC_09 | NA | 19 | NA | 13 | NA | 12 | NA | 10 |

Data not available is indicated by ‘NA’

TABLE 7
List of differentially expressed genes in NSCLC CD8+
TILs from TIL high versus TIL low tumors.

| | Normalized | | DE-Seq statistics | | |
|---|---|---|---|---|---|
| Gene | mean counts | | Fold | | |
| Symbol | TIL low | TIL high | Change | P value | P adj |
| ACTN4 | 2519.83 | 3813.73 | 1.51 | 0.00073 | 0.043 |
| ADD3 | 1672.21 | 1047.43 | 0.63 | 1.00E−05 | 4.20E−03 |
| ADRB2 | 1461.63 | 777.29 | 0.53 | 0.00036 | 0.029 |
| AHCTF1 | 714.98 | 431.4 | 0.60 | 0.00017 | 0.019 |
| AKAP5 | 116.13 | 513.11 | 4.42 | 0.000000055 | 0.000062 |
| ANP32E | 1455.41 | 1890.54 | 1.30 | 0.00082 | 0.047 |
| ANTXR2 | 772.57 | 303.87 | 0.39 | 0.000024 | 0.0065 |
| ARL6IP6 | 460.97 | 651.77 | 1.41 | 0.00088 | 0.048 |
| ASB2 | 292.48 | 670.05 | 2.29 | 0.00077 | 0.045 |
| ATP1B1 | 414.23 | 149.85 | 0.36 | 0.00025 | 0.024 |
| ATP5G2 | 2054.29 | 2736.15 | 1.33 | 8.20E−04 | 4.70E−02 |
| BCAS4 | 176.16 | 405.54 | 2.30 | 0.000028 | 0.0068 |
| BST2 | 684.27 | 1148.36 | 1.68 | 2.90E−04 | 2.60E−02 |
| C6orf108 | 194.74 | 442.24 | 2.27 | 0.000021 | 0.0063 |
| CA5B | 408.77 | 226.19 | 0.55 | 7.10E−04 | 4.20E−02 |
| CAST | 1340.86 | 995.56 | 0.74 | 1.70E−04 | 0.019 |
| CCL3 | 1284.07 | 2684.22 | 2.09 | 8.40E−04 | 4.70E−02 |
| CCL5 | 21219.07 | 30156.58 | 1.42 | 9.20E−04 | 0.048 |
| CD200R1 | 405.14 | 782.31 | 1.93 | 0.00038 | 0.031 |
| CD38 | 107.41 | 585.58 | 5.45 | 0.000000021 | 0.00004 |
| CD8A | 16973.02 | 22695.83 | 1.34 | 9.40E−05 | 0.013 |
| COTL1 | 5140.46 | 9857.62 | 1.92 | 4.00E−05 | 8.20E−03 |
| CX3CR1 | 1495.32 | 262.14 | 0.18 | 0.000000088 | 0.000082 |
| CXCR6 | 3780.01 | 8082.91 | 2.14 | 3.10E−09 | 8.60E−06 |
| DSTN | 1160.97 | 739.82 | 0.64 | 0.000000053 | 0.000062 |
| DUSP6 | 938.68 | 411.48 | 0.44 | 0.000038 | 0.0081 |
| EPSTI1 | 176.68 | 482.2 | 2.73 | 9.10E−06 | 4.20E−03 |
| FAM113B | 630.98 | 978.4 | 1.55 | 0.00023 | 0.023 |
| FCGR3A | 1160.83 | 304.78 | 0.26 | 0.0005 | 0.033 |
| FGFBP2 | 683.23 | 201.09 | 0.29 | 0.00045 | 0.032 |
| FUT8 | 433.47 | 828.78 | 1.91 | 5.90E−04 | 0.036 |
| GBP1 | 1075.11 | 2449.9 | 2.28 | 6.00E−05 | 1.10E−02 |
| GBP2 | 1716.46 | 3149.91 | 1.84 | 9.70E−06 | 4.20E−03 |
| GBP4 | 1111.12 | 2230.95 | 2.01 | 0.000057 | 0.011 |
| GBP5 | 2587.64 | 5517.27 | 2.13 | 3.00E−06 | 1.90E−03 |
| GMPS | 498.06 | 787.05 | 1.58 | 0.00057 | 0.035 |
| GNL3L | 527.99 | 362.95 | 0.69 | 0.000021 | 0.0063 |
| GPI | 2960.44 | 4398.01 | 1.49 | 4.80E−04 | 0.032 |
| GZMA | 7225.21 | 13673.22 | 1.89 | 2.30E−05 | 6.50E−03 |
| HAVCR2 | 515.37 | 2154.62 | 4.18 | 4.80E−06 | 2.70E−03 |
| HNRNPK | 5826.88 | 7527.09 | 1.29 | 0.00023 | 0.023 |
| HNRPLL | 930.78 | 1413.62 | 1.52 | 0.00057 | 0.035 |
| IGFLR1 | 451.21 | 927.91 | 2.06 | 0.00028 | 0.026 |
| IL21R | 433.09 | 695.55 | 1.61 | 2.50E−04 | 2.40E−02 |
| ITGAE | 2740.41 | 5777.05 | 2.11 | 9.80E−05 | 1.30E−02 |
| KLF2 | 1098.98 | 351.43 | 0.32 | 0.00092 | 0.048 |
| LDHB | 3256.94 | 4900.2 | 1.50 | 0.0000011 | 0.00077 |
| LPAR6 | 601.89 | 265.48 | 0.44 | 0.00048 | 0.032 |
| MCM4 | 247.48 | 583.68 | 2.36 | 0.00054 | 0.034 |
| MLLT10 | 447.64 | 244.31 | 0.55 | 7.80E−05 | 1.20E−02 |
| MRPL37 | 417.14 | 613.5 | 1.47 | 0.00033 | 0.028 |
| NAB1 | 411.07 | 921.51 | 2.24 | 0.000081 | 0.012 |
| NDUFS8 | 556.25 | 952.93 | 1.71 | 2.90E−05 | 0.0068 |
| NECAP1 | 486.82 | 336.44 | 0.69 | 0.000096 | 0.013 |
| NOTCH1 | 365.49 | 672.33 | 1.84 | 8.50E−04 | 4.70E−02 |
| NPC2 | 855.28 | 248.27 | 0.29 | 0.00025 | 0.024 |
| OAS3 | 526.48 | 929.18 | 1.76 | 4.10E−04 | 3.20E−02 |
| PAG1 | 1962.17 | 3135.86 | 1.60 | 6.90E−05 | 1.10E−02 |
| PARP9 | 1032.74 | 1764.92 | 1.71 | 0.00044 | 0.032 |
| PCMTD2 | 486.25 | 273.91 | 0.56 | 0.00051 | 0.033 |
| PCNT | 597.4 | 399.92 | 0.67 | 9.20E−04 | 4.80E−02 |
| PDCD1 | 902.98 | 1791.4 | 1.98 | 0.00028 | 0.026 |
| PLAC8 | 959.85 | 316.57 | 0.33 | 5.40E−04 | 3.40E−02 |
| POLR1D | 648.33 | 882.86 | 1.36 | 5.10E−04 | 3.30E−02 |
| PPM1M | 729.91 | 1108.49 | 1.52 | 1.60E−04 | 1.90E−02 |
| PPP2R4 | 372.32 | 651.97 | 1.75 | 0.00019 | 0.02 |
| PRDM2 | 1117.78 | 754.48 | 0.67 | 8.30E−04 | 4.70E−02 |
| PRKAG1 | 407.63 | 672.7 | 1.65 | 4.40E−04 | 0.032 |
| PRKAR1A | 2441.24 | 3577.97 | 1.47 | 0.000016 | 0.0056 |
| PSMB8 | 2196.61 | 3613.86 | 1.65 | 3.40E−05 | 7.50E−03 |
| PSMB9 | 2542.74 | 4211.37 | 1.66 | 0.00019 | 0.02 |
| PSMD8 | 925.56 | 1493.93 | 1.61 | 0.0002 | 0.021 |
| PSME2 | 1697.08 | 3083.82 | 1.82 | 3.00E−04 | 2.60E−02 |
| PTTG1 | 375.15 | 848.43 | 2.26 | 0.00011 | 0.015 |
| PURA | 373.61 | 222.01 | 0.59 | 0.00011 | 0.014 |
| R3HDM1 | 404.06 | 680.97 | 1.69 | 0.0006 | 0.036 |
| RAB3GAP1 | 575.58 | 1058.87 | 1.84 | 0.00043 | 0.032 |
| RABAC1 | 937.27 | 1296.02 | 1.38 | 3.10E−04 | 2.70E−02 |
| RARRES3 | 2370.82 | 4399.49 | 1.86 | 6.70E−05 | 1.10E−02 |
| RBBP4 | 1347 | 1852.53 | 1.38 | 0.00091 | 0.048 |
| S100A10 | 3109.64 | 1927.52 | 0.62 | 3.90E−04 | 3.10E−02 |
| S1PR1 | 1184.39 | 390.45 | 0.33 | 0.000019 | 0.0063 |
| SEC11A | 678.62 | 1069.04 | 1.58 | 9.40E−04 | 4.80E−02 |
| SF3B3 | 1227.63 | 1738.82 | 1.42 | 0.00067 | 0.04 |
| SIRPG | 1052.21 | 2594.02 | 2.47 | 2.50E−09 | 8.60E−06 |
| SLC27A2 | 209.02 | 621.75 | 2.97 | 4.20E−04 | 3.20E−02 |
| SNX17 | 1090.28 | 1562.14 | 1.43 | 9.00E−04 | 4.80E−02 |
| SRA1 | 229.62 | 398.37 | 1.73 | 5.00E−05 | 0.01 |
| STAT1 | 3308.41 | 8166.42 | 2.47 | 2.80E−07 | 0.00022 |
| STAT2 | 518.14 | 772.49 | 1.49 | 9.40E−04 | 0.048 |
| STK38 | 1077.55 | 607.04 | 0.56 | 0.000067 | 0.011 |
| STMN1 | 715.35 | 2001.3 | 2.80 | 0.000082 | 0.012 |
| SYT11 | 877.99 | 1467.19 | 1.67 | 0.000089 | 0.013 |
| TAZ | 383.69 | 223 | 0.58 | 0.00047 | 0.032 |
| TGFBR3 | 1078.72 | 546.16 | 0.51 | 4.60E−04 | 3.20E−02 |
| TIAM1 | 248.36 | 545.18 | 2.20 | 5.70E−05 | 1.10E−02 |
| TIMP1 | 508.71 | 187.64 | 0.37 | 1.20E−05 | 4.60E−03 |
| TMEM140 | 412.13 | 670.89 | 1.63 | 9.80E−05 | 1.30E−02 |
| TNF | 2071 | 806.83 | 0.39 | 0.00017 | 0.019 |
| TNFRSF9 | 614.89 | 1886.64 | 3.07 | 0.000026 | 0.0066 |
| TNFSF4 | 202.36 | 627.96 | 3.10 | 3.60E−04 | 2.90E−02 |
| TNRC6C | 489.35 | 238.97 | 0.49 | 1.60E−04 | 0.019 |
| TOP2A | 243.52 | 763.8 | 3.14 | 0.00043 | 0.032 |
| TP53BP2 | 363.83 | 241.13 | 0.66 | 0.00016 | 0.019 |
| TRAPPC10 | 948.07 | 653.94 | 0.69 | 0.00048 | 0.032 |
| TUG1 | 742.03 | 517.6 | 0.70 | 3.10E−04 | 2.70E−02 |
| UBE2L6 | 1777.58 | 3418.7 | 1.92 | 0.00001 | 0.0042 |
| UBE2Q2 | 466.85 | 276.57 | 0.59 | 0.00014 | 0.017 |
| ZFYVE26 | 379.61 | 199.16 | 0.52 | 0.00031 | 0.027 |

TABLE 8
List of differentially expressed genes in NSCLC CD8+
TILs from CD103 high versus CD103 low tumors.

| Gene | Normalized mean counts | | DE-Seq statistics | | |
|---|---|---|---|---|---|
| Symbol | CD103 low | CD103 high | Fold change | P value | P adj |
| A2M | 1017.35 | 378.05 | 0.37 | 0.00046 | 0.013 |
| ABCB1 | 546.19 | 932.05 | 1.71 | 3.20E−04 | 9.80E−03 |
| ABI3 | 1006.55 | 1783.19 | 1.77 | 0.00023 | 0.0079 |
| ABL2 | 150.24 | 50.27 | 0.33 | 0.00036 | 0.011 |
| ACOT7 | 187.02 | 539.27 | 2.88 | 0.0000097 | 0.00087 |
| ACP5 | 872.59 | 1998.81 | 2.29 | 0.000045 | 0.0025 |
| ACSL6 | 304.68 | 124.78 | 0.41 | 0.0022 | 0.038 |
| ACTN4 | 2367.3 | 4148.19 | 1.75 | 0.0011 | 0.024 |
| ACTR3 | 4711.26 | 6568.16 | 1.39 | 0.0006 | 0.016 |
| ADAMTSL4 | 92.02 | 25.54 | 0.28 | 0.002 | 0.036 |
| ADD3 | 1433.55 | 1181.2 | 0.82 | 1.70E−03 | 3.30E−02 |
| ADRB2 | 1340.27 | 712.13 | 0.53 | 0.000071 | 0.0034 |
| AFAP1L2 | 129.16 | 424.05 | 3.28 | 2.90E−03 | 4.50E−02 |
| AGXT2L2 | 445.92 | 665.22 | 1.49 | 0.0011 | 0.024 |
| AHNAK | 11994.26 | 7483.26 | 0.62 | 9.50E−06 | 8.60E−04 |
| AIM1 | 1727.22 | 942.42 | 0.55 | 1.20E−03 | 0.025 |
| AKAP5 | 145.76 | 618.83 | 4.25 | 2.90E−06 | 3.30E−04 |
| AKAP9 | 2156.87 | 1407.34 | 0.65 | 1.60E−03 | 0.031 |
| ALDOC | 300.81 | 698.27 | 2.32 | 0.00015 | 0.006 |
| ALOX5AP | 4048.19 | 6542.77 | 1.62 | 0.000015 | 0.0012 |
| ANAPC11 | 470.21 | 640.32 | 1.36 | 1.90E−04 | 0.0069 |
| ANK3 | 364.63 | 180.84 | 0.50 | 2.20E−03 | 3.90E−02 |
| ANKRD12 | 2859.79 | 2152.45 | 0.75 | 0.0015 | 0.029 |
| ANKRD20A9P | 232.88 | 115.92 | 0.50 | 2.00E−03 | 3.60E−02 |
| ANKRD44 | 4481.24 | 3278.35 | 0.73 | 0.00025 | 0.0083 |
| ANKS1B | 9.17 | 75.29 | 8.21 | 0.0015 | 0.03 |
| ANKS6 | 86.7 | 12.25 | 0.14 | 3.00E−03 | 4.60E−02 |
| ANP32B | 513.13 | 779 | 1.52 | 0.0002 | 0.0072 |
| ANP32E | 1435.03 | 2009.06 | 1.40 | 9.3E−09 | 0.0000041 |
| ANTXR2 | 604.98 | 317.17 | 0.52 | 0.000036 | 0.0021 |
| ANXA5 | 2037.69 | 3630 | 1.78 | 7.10E−04 | 0.018 |
| AP4S1 | 135.21 | 55.48 | 0.41 | 3.70E−05 | 2.10E−03 |
| ARHGAP26 | 919.69 | 495.07 | 0.54 | 1.70E−03 | 3.20E−02 |
| ARHGAP27 | 572.59 | 372.87 | 0.65 | 0.0025 | 0.041 |
| ARL3 | 148.9 | 322.73 | 2.17 | 3.60E−07 | 6.90E−05 |
| ARL4C | 3049.03 | 1913.58 | 0.63 | 0.00092 | 0.021 |
| ARL5A | 1109.47 | 893.07 | 0.80 | 0.0022 | 0.038 |
| ARL6IP1 | 2832.03 | 4268.71 | 1.51 | 5.10E−04 | 0.014 |
| ARL6IP6 | 470.8 | 635.1 | 1.35 | 1.30E−03 | 2.70E−02 |
| ARPC2 | 8776.4 | 12575.65 | 1.43 | 6.10E−05 | 3.00E−03 |
| ARPC3 | 2749.26 | 3605.55 | 1.31 | 0.000059 | 0.003 |
| ASB2 | 298.95 | 763.66 | 2.55 | 0.0000058 | 0.00059 |
| ASF1B | 50.63 | 307.62 | 6.08 | 3.9E−10 | 0.00000031 |
| ASPM | 109.39 | 564.19 | 5.16 | 2.50E−05 | 1.70E−03 |
| ATG14 | 201.58 | 124.82 | 0.62 | 2.10E−03 | 3.70E−02 |
| ATM | 1928.43 | 1083.2 | 0.56 | 0.000035 | 0.0021 |
| ATP10D | 371.86 | 571.3 | 1.54 | 0.00091 | 0.021 |
| ATP2B1 | 950.31 | 593.36 | 0.62 | 0.0017 | 0.032 |
| ATP5B | 5179.06 | 7348.78 | 1.42 | 0.00096 | 0.022 |
| ATP5C1 | 1231.67 | 1869.3 | 1.52 | 5.60E−05 | 2.90E−03 |
| ATP5E | 318.51 | 330.37 | 1.04 | 0.00094 | 0.022 |
| ATP5EP2 | 41.3 | 51.87 | 1.26 | 0.00062 | 0.016 |
| ATP5G3 | 1706.19 | 2281.79 | 1.34 | 5.90E−04 | 0.015 |
| ATP5J2 | 309.84 | 381.83 | 1.23 | 0.0012 | 0.025 |
| ATP5L | 2481.62 | 2782.12 | 1.12 | 1.60E−03 | 3.10E−02 |
| ATP8B4 | 82.69 | 309.33 | 3.74 | 0.00017 | 0.0065 |
| ATXN7 | 1423.54 | 887.92 | 0.62 | 9.60E−04 | 2.20E−02 |
| ATXN7L1 | 375.3 | 200.54 | 0.53 | 3.20E−04 | 9.90E−03 |
| AUH | 131.99 | 286.99 | 2.17 | 0.00000061 | 0.000099 |
| AURKA | 58.84 | 155.92 | 2.65 | 0.00000048 | 0.000085 |
| AURKB | 10.18 | 186.53 | 18.32 | 1.90E−05 | 1.40E−03 |
| BARD1 | 160.96 | 356.77 | 2.22 | 0.00019 | 0.0068 |
| BATF | 479.45 | 1033.1 | 2.15 | 8.70E−08 | 2.30E−05 |
| BAZ2B | 940.01 | 525.04 | 0.56 | 3.60E−05 | 2.10E−03 |
| BBX | 1583.19 | 1370.97 | 0.87 | 3.00E−03 | 4.60E−02 |
| BCCIP | 457.64 | 600.6 | 1.31 | 0.0026 | 0.043 |
| BCL11B | 1244.44 | 798.79 | 0.64 | 1.20E−03 | 2.50E−02 |
| BEX2 | 108.9 | 25.44 | 0.23 | 5.30E−04 | 0.014 |
| BIRC5 | 25.85 | 247.76 | 9.58 | 0.000035 | 0.0021 |
| BLOC1S1 | 168.34 | 266.7 | 1.58 | 6.90E−05 | 3.30E−03 |
| BRIP1 | 47.47 | 137.24 | 2.89 | 0.0031 | 0.047 |
| BST2 | 826.52 | 1230.81 | 1.49 | 0.00014 | 0.0058 |
| BUB1 | 107.58 | 468.39 | 4.35 | 8.50E−05 | 3.90E−03 |
| C10orf54 | 1717.68 | 1352.74 | 0.79 | 0.000016 | 0.0012 |
| C14orf166 | 715.22 | 1046.02 | 1.46 | 0.00002 | 0.0014 |
| C15orf17 | 669.03 | 303.32 | 0.45 | 0.00024 | 0.0081 |
| C16orf54 | 1888.59 | 1219.16 | 0.65 | 0.00041 | 0.012 |
| C1orf21 | 358.82 | 118.57 | 0.33 | 1.00E−05 | 8.90E−04 |
| C20orf112 | 597.98 | 261.09 | 0.44 | 2.50E−05 | 1.70E−03 |
| C4orf34 | 224.92 | 80.06 | 0.36 | 0.000025 | 0.0017 |
| C4orf48 | 119.44 | 152.55 | 1.28 | 1.00E−04 | 4.60E−03 |
| C6orf108 | 245.44 | 473.36 | 1.93 | 0.00000016 | 0.000037 |
| C9orf16 | 379.58 | 577.16 | 1.52 | 2.30E−03 | 3.90E−02 |
| CA5B | 383.93 | 207.63 | 0.54 | 0.00002 | 0.0014 |
| CACYBP | 933.15 | 1610.97 | 1.73 | 2.70E−03 | 4.30E−02 |
| CALCOCO2 | 1506.95 | 2160.6 | 1.43 | 6.10E−05 | 3.00E−03 |
| CALM3 | 838.76 | 1412.41 | 1.68 | 2.10E−06 | 2.50E−04 |
| CAMK1D | 205.81 | 134.02 | 0.65 | 6.70E−04 | 0.017 |
| CAPZA1 | 4668.15 | 5824.83 | 1.25 | 2.90E−03 | 0.045 |
| CAPZB | 2419.92 | 3951.14 | 1.63 | 1.70E−03 | 0.032 |
| CASC5 | 58.04 | 276.8 | 4.77 | 0.00023 | 0.008 |
| CAST | 1311.85 | 1053.78 | 0.80 | 0.001 | 0.023 |
| CCDC109B | 900.87 | 541.67 | 0.60 | 0.0002 | 0.0072 |
| CCDC12 | 516.79 | 629.66 | 1.22 | 0.00037 | 0.011 |
| CCL3 | 1395.15 | 2885.68 | 2.07 | 3.40E−05 | 2.10E−03 |
| CCL5 | 21385.86 | 30378.31 | 1.42 | 3.30E−04 | 1.00E−02 |
| CCNA2 | 58.08 | 370.2 | 6.37 | 4.60E−07 | 8.30E−05 |
| CCNB2 | 18.58 | 260.7 | 14.03 | 8.30E−07 | 1.30E−04 |
| CCND3 | 3636.55 | 2760.86 | 0.76 | 0.003 | 0.046 |
| CCNE2 | 39.44 | 176.73 | 4.48 | 0.00046 | 0.013 |
| CD2 | 7410.34 | 9346.67 | 1.26 | 1.70E−03 | 3.20E−02 |
| CD200R1 | 473.16 | 861.38 | 1.82 | 1.80E−05 | 0.0013 |
| CD300A | 373.4 | 154.91 | 0.41 | 0.00047 | 0.013 |
| CD38 | 116.46 | 705.81 | 6.06 | 6.4E−12 | 9.5E−09 |
| CD3D | 8389.86 | 11155.32 | 1.33 | 0.00000053 | 0.000091 |
| CD3G | 2804.98 | 3963.96 | 1.41 | 3.80E−05 | 2.20E−03 |
| CD40LG | 230.49 | 42.94 | 0.19 | 0.00017 | 0.0065 |
| CD63 | 2297.87 | 3275.04 | 1.43 | 0.0022 | 0.038 |
| CD7 | 573.54 | 1078.82 | 1.88 | 0.0011 | 0.024 |
| CD82 | 1093.16 | 2136.68 | 1.95 | 0.0000071 | 0.00069 |
| CD96 | 6296.83 | 8704.35 | 1.38 | 0.000048 | 0.0026 |
| CDC123 | 542.1 | 803.54 | 1.48 | 2.60E−04 | 8.50E−03 |
| CDC20 | 28.79 | 255.55 | 8.88 | 0.0007 | 0.017 |
| CDC45 | 11.11 | 144.32 | 12.99 | 0.0012 | 0.025 |
| CDC6 | 46.27 | 174.91 | 3.78 | 1.10E−03 | 2.50E−02 |
| CDCA2 | 21.44 | 162 | 7.56 | 0.00049 | 0.014 |
| CDCA7 | 181.8 | 571.82 | 3.15 | 0.00000046 | 0.000083 |
| CDCA8 | 22.6 | 168.17 | 7.44 | 0.0006 | 0.016 |
| CDK1 | 121.79 | 407.66 | 3.35 | 0.00087 | 0.021 |
| CDKN3 | 56.86 | 232.47 | 4.09 | 2.80E−04 | 0.009 |
| CENPF | 164.5 | 573.54 | 3.49 | 2.50E−09 | 0.0000015 |
| CENPM | 93.69 | 267.2 | 2.85 | 0.00000055 | 0.000092 |
| CEP350 | 1927.15 | 1331.77 | 0.69 | 0.002 | 0.036 |
| CEP55 | 22.44 | 121.06 | 5.39 | 0.00049 | 0.014 |
| CERK | 817.88 | 391.05 | 0.48 | 0.00083 | 0.02 |
| CERKL | 101.36 | 40.25 | 0.40 | 5.20E−05 | 2.70E−03 |
| CFL1 | 10823.85 | 15883.78 | 1.47 | 2.10E−03 | 3.60E−02 |
| CHEK1 | 79.04 | 180.89 | 2.29 | 0.0009 | 0.021 |
| CHMP4A | 510.59 | 738.53 | 1.45 | 0.0018 | 0.034 |
| CHORDC1 | 327.33 | 482.24 | 1.47 | 2.00E−03 | 3.60E−02 |
| CIRBP | 1564.81 | 1006.92 | 0.64 | 0.0014 | 0.028 |
| CISD1 | 145.5 | 249.37 | 1.71 | 0.00071 | 0.018 |
| CKAP2 | 463.97 | 778.02 | 1.68 | 2.80E−03 | 4.50E−02 |
| CKAP2L | 36.1 | 198.24 | 5.49 | 0.0018 | 0.034 |
| CKLF | 291.13 | 366.7 | 1.26 | 0.00025 | 0.0082 |
| CKS1B | 189.82 | 465.66 | 2.45 | 1.00E−04 | 4.60E−03 |
| CKS2 | 330.79 | 803 | 2.43 | 2.50E−05 | 1.60E−03 |
| CLEC2B | 1539.43 | 2004.43 | 1.30 | 4.40E−06 | 4.80E−04 |
| CLIC1 | 4981.72 | 6647.21 | 1.33 | 0.00081 | 0.019 |
| CLNK | 78.26 | 269.75 | 3.45 | 2.00E−04 | 7.20E−03 |
| CLSPN | 64.58 | 200.76 | 3.11 | 0.0000064 | 0.00064 |
| CMC2 | 340 | 560.12 | 1.65 | 0.0000013 | 0.00017 |
| CNNM3 | 389.86 | 179.98 | 0.46 | 0.00016 | 0.0061 |
| COL6A2 | 107.42 | 21.43 | 0.20 | 0.0019 | 0.034 |
| COMMD7 | 651.6 | 1012.88 | 1.55 | 1.90E−04 | 6.80E−03 |
| COPB1 | 1462.34 | 2213.04 | 1.51 | 1.50E−03 | 2.90E−02 |
| COPE | 1441.03 | 1927.3 | 1.34 | 1.70E−03 | 3.30E−02 |
| COPS6 | 959.42 | 1130.08 | 1.18 | 1.90E−03 | 3.50E−02 |
| COPZ1 | 1057.45 | 1459.53 | 1.38 | 1.10E−03 | 2.30E−02 |
| COTL1 | 5161.28 | 10851.46 | 2.10 | 1.00E−06 | 1.50E−04 |
| COX5A | 1147.18 | 1663.06 | 1.45 | 0.0021 | 0.037 |
| COX6A1 | 1577.67 | 1722.18 | 1.09 | 0.00074 | 0.018 |
| COX7B | 1030.5 | 1181.44 | 1.15 | 0.00079 | 0.019 |
| COX8A | 1222.22 | 1528.76 | 1.25 | 2.50E−03 | 4.10E−02 |
| CROCC | 124.18 | 42.06 | 0.34 | 0.0021 | 0.037 |
| CRTAP | 906.89 | 578.84 | 0.64 | 1.30E−03 | 0.028 |
| CSF1 | 674.49 | 1812.46 | 2.69 | 3.50E−05 | 2.10E−03 |
| CUEDC2 | 298.95 | 493.7 | 1.65 | 6.30E−04 | 0.016 |
| CUX1 | 342.34 | 159.04 | 0.46 | 0.002 | 0.036 |
| CX3CR1 | 1321.47 | 282.23 | 0.21 | 5.20E−04 | 0.014 |
| CXCL13 | 950.75 | 6139.74 | 6.46 | 0.00038 | 0.011 |
| CXCR6 | 4485.82 | 7662.29 | 1.71 | 1.10E−04 | 4.90E−03 |
| CYTH1 | 1256.96 | 880.39 | 0.70 | 0.0011 | 0.023 |
| DAPK2 | 293.53 | 663.18 | 2.26 | 1.30E−05 | 0.001 |
| DBN1 | 128.26 | 328.38 | 2.56 | 0.00058 | 0.015 |
| DDB2 | 309.8 | 475.04 | 1.53 | 2.30E−03 | 0.039 |
| DENND1B | 860.15 | 1250.41 | 1.45 | 0.00073 | 0.018 |
| DENND5A | 142.24 | 21.2 | 0.15 | 0.00065 | 0.017 |
| DGKD | 403.47 | 262.05 | 0.65 | 3.10E−03 | 0.047 |
| DHFR | 87.68 | 233.09 | 2.66 | 0.00028 | 0.0089 |
| DHRS3 | 924.4 | 300.14 | 0.32 | 1.70E−11 | 0.000000022 |
| DHX36 | 805.7 | 562.79 | 0.70 | 0.0018 | 0.034 |
| DIXDC1 | 36.39 | 111.8 | 3.07 | 0.0022 | 0.037 |
| DLG1 | 317.63 | 204.1 | 0.64 | 2.30E−03 | 0.039 |
| DLGAP5 | 6.87 | 195.6 | 28.47 | 2.50E−13 | 8.2E−10 |
| DNAJA1 | 1702.01 | 2923.76 | 1.72 | 1.50E−03 | 3.00E−02 |
| DNAJB11 | 454.26 | 710.37 | 1.56 | 0.00021 | 0.0074 |
| DNAJB6 | 936.51 | 1381.01 | 1.47 | 2.00E−03 | 0.036 |
| DPEP2 | 130.93 | 27.43 | 0.21 | 1.00E−04 | 0.0044 |
| DPP3 | 351.88 | 579.53 | 1.65 | 1.60E−03 | 0.032 |
| DPY30 | 474.86 | 615.62 | 1.30 | 0.0011 | 0.025 |
| DRAP1 | 441.94 | 634.53 | 1.44 | 0.00063 | 0.016 |
| DTL | 39.22 | 232.62 | 5.93 | 2.80E−04 | 0.009 |
| DTX2 | 258.98 | 443.74 | 1.71 | 0.003 | 0.046 |
| DUT | 601.93 | 868.19 | 1.44 | 0.0024 | 0.039 |
| DYNLRB1 | 661.35 | 897.3 | 1.36 | 0.00044 | 0.012 |
| DYNLT3 | 763.81 | 575.71 | 0.75 | 1.50E−03 | 0.03 |
| ECH1 | 1310.37 | 1857.74 | 1.42 | 7.00E−04 | 0.017 |
| EEA1 | 468.29 | 297.47 | 0.64 | 3.30E−05 | 2.00E−03 |
| EIF3I | 929.81 | 1346.23 | 1.45 | 0.0021 | 0.037 |
| ELL2 | 212.89 | 68.81 | 0.32 | 0.00045 | 0.013 |
| EMB | 3368.41 | 2914.16 | 0.87 | 2.20E−03 | 3.80E−02 |
| EMP3 | 2523.16 | 1343.08 | 0.53 | 0.00094 | 0.022 |
| ENC1 | 359.95 | 142.58 | 0.40 | 1.70E−03 | 0.033 |
| ENO1 | 5231.48 | 8258.6 | 1.58 | 0.002 | 0.036 |
| ENSA | 654.71 | 970.33 | 1.48 | 0.0028 | 0.044 |
| ENTPD1 | 658.82 | 2651.92 | 4.03 | 2.00E−07 | 4.30E−05 |
| EPB41 | 942.58 | 706.74 | 0.75 | 1.60E−04 | 6.30E−03 |
| EPB41L5 | 112.71 | 21.94 | 0.19 | 0.00017 | 0.0064 |
| EPSTI1 | 175.58 | 555.14 | 3.16 | 1.8E−09 | 0.0000013 |
| ERBB2 | 114.75 | 28.73 | 0.25 | 1.60E−03 | 3.10E−02 |
| ERC1 | 393.09 | 207.81 | 0.53 | 2.90E−03 | 4.50E−02 |
| ERMP1 | 376.92 | 192.9 | 0.51 | 1.80E−03 | 0.034 |
| ERP27 | 140.76 | 33.15 | 0.24 | 0.00000086 | 0.00013 |
| ETFA | 619.88 | 851.63 | 1.37 | 2.30E−03 | 3.90E−02 |
| ETFB | 303.05 | 463.27 | 1.53 | 1.60E−03 | 3.10E−02 |
| ETV3 | 138.64 | 65.07 | 0.47 | 2.60E−03 | 4.20E−02 |
| ETV7 | 24.54 | 224.6 | 9.15 | 0.0023 | 0.039 |
| EXOSC10 | 791.23 | 1051.15 | 1.33 | 3.40E−04 | 0.01 |
| EXOSC6 | 249.88 | 143.49 | 0.57 | 0.0029 | 0.045 |
| EZH2 | 146.14 | 475.89 | 3.26 | 6.90E−06 | 6.80E−04 |
| F11R | 398.06 | 215.16 | 0.54 | 4.20E−04 | 0.012 |
| FABP5 | 462.37 | 1141.17 | 2.47 | 3.40E−05 | 0.0021 |
| FAM105B | 654.22 | 783.17 | 1.20 | 2.60E−03 | 0.042 |
| FAM111B | 32.11 | 126.69 | 3.95 | 1.90E−03 | 3.40E−02 |
| FAM113B | 703.07 | 1075.84 | 1.53 | 0.0018 | 0.034 |
| FAM117A | 247.53 | 135.73 | 0.55 | 5.30E−04 | 1.40E−02 |
| FAM179A | 98.34 | 250.94 | 2.55 | 4.10E−05 | 2.30E−03 |
| FAM65B | 1850.24 | 777.82 | 0.42 | 7.10E−09 | 3.40E−06 |
| FAM84B | 181.79 | 73.71 | 0.41 | 0.0008 | 0.019 |
| FANCI | 111.59 | 426.66 | 3.82 | 5.9E−14 | 3.1E−10 |
| FANCL | 94.85 | 230.08 | 2.43 | 8.70E−04 | 2.10E−02 |
| FARSA | 455.79 | 697.21 | 1.53 | 0.0014 | 0.028 |
| FBXO5 | 84.93 | 254.49 | 3.00 | 2.00E−05 | 1.50E−03 |
| FBXW4 | 233.07 | 96.44 | 0.41 | 0.00048 | 0.013 |
| FDPS | 380.77 | 611.53 | 1.61 | 0.00000014 | 0.000033 |
| FEN1 | 123.47 | 388.89 | 3.15 | 0.00023 | 0.008 |
| FGFBP2 | 715.14 | 133.73 | 0.19 | 0.00000041 | 0.000077 |
| FIBP | 431.19 | 720.96 | 1.67 | 0.000084 | 0.0039 |
| FIS1 | 547.61 | 718.67 | 1.31 | 8.90E−05 | 0.0041 |
| FKBP1A | 1227.39 | 2296.82 | 1.87 | 2.30E−08 | 0.0000082 |
| FOXK1 | 238.18 | 121.61 | 0.51 | 0.0011 | 0.024 |
| FOXP1 | 2150.08 | 1586.19 | 0.74 | 0.00012 | 0.0052 |
| FRYL | 1237.4 | 915.93 | 0.74 | 2.70E−03 | 4.30E−02 |
| FUT11 | 260.1 | 128.89 | 0.50 | 0.000023 | 0.0016 |
| FXC1 | 653.67 | 440.68 | 0.67 | 2.10E−03 | 0.037 |
| FZD3 | 167.76 | 66.62 | 0.40 | 1.70E−03 | 3.20E−02 |
| FZD4 | 88.11 | 4.52 | 0.05 | 0.000000019 | 0.0000072 |
| GALM | 610.26 | 1136.46 | 1.86 | 0.0000012 | 0.00016 |
| GALNT1 | 593.26 | 1036.87 | 1.75 | 1.40E−03 | 0.029 |
| GALNT2 | 493.65 | 1089.43 | 2.21 | 8.80E−07 | 0.00013 |
| GAPDH | 15822.78 | 32559.55 | 2.06 | 4.2E−09 | 0.0000023 |
| GBP1 | 1024.16 | 2782.3 | 2.72 | 0.00000022 | 0.000045 |
| GBP2 | 1556.13 | 3641.25 | 2.34 | 7.30E−09 | 3.40E−06 |
| GBP4 | 1237.21 | 2570.17 | 2.08 | 0.0003 | 0.0093 |
| GBP5 | 2971.45 | 5985.91 | 2.01 | 1.90E−06 | 2.30E−04 |
| GDPD1 | 65.51 | 25.51 | 0.39 | 9.60E−04 | 2.20E−02 |
| GIMAP1 | 731.4 | 439.41 | 0.60 | 0.00019 | 0.0069 |
| GLDC | 22.23 | 245.95 | 11.06 | 0.000012 | 0.001 |
| GNAO1 | 63.5 | 19.24 | 0.30 | 0.00000016 | 0.000037 |
| GOLGA1 | 280.31 | 120.02 | 0.43 | 0.0023 | 0.039 |
| GPI | 2901.6 | 4930.81 | 1.70 | 2.60E−04 | 8.40E−03 |
| GPR25 | 233.78 | 681.78 | 2.92 | 0.00013 | 0.0054 |
| GRAMD1A | 667.81 | 404.52 | 0.61 | 0.0013 | 0.027 |
| GRK6 | 820.48 | 439.36 | 0.54 | 2.20E−03 | 3.80E−02 |
| GSTM3 | 146.28 | 55.13 | 0.38 | 0.00021 | 0.0074 |
| GTSE1 | 16.76 | 105.62 | 6.30 | 0.000014 | 0.0011 |
| GZMA | 7962.1 | 13460.35 | 1.69 | 0.0000034 | 0.00038 |
| GZMB | 4247.1 | 17025.77 | 4.01 | 5.00E−11 | 5.20E−08 |
| GZMK | 7553.09 | 3894.68 | 0.52 | 2.30E−03 | 3.90E−02 |
| H2AFX | 183.6 | 392.57 | 2.14 | 3.10E−05 | 0.002 |
| H2AFZ | 1728.79 | 3027.42 | 1.75 | 8.60E−06 | 8.10E−04 |
| HAPLN3 | 107.51 | 331.04 | 3.08 | 1.20E−05 | 0.00099 |
| HAVCR2 | 543.14 | 2467.99 | 4.54 | 3.10E−13 | 8.20E−10 |
| HCLS1 | 3022.85 | 3853.64 | 1.27 | 0.00055 | 0.015 |
| HDLBP | 798.65 | 1477.04 | 1.85 | 0.0000052 | 0.00055 |
| HIST1H1B | 34.75 | 185.79 | 5.35 | 3.10E−04 | 9.70E−03 |
| HIST1H1C | 347.93 | 541.33 | 1.56 | 3.00E−05 | 1.90E−03 |
| HIST1H1E | 381.19 | 482.67 | 1.27 | 0.0015 | 0.03 |
| HIST1H2AC | 123.56 | 230.84 | 1.87 | 9.70E−04 | 2.20E−02 |
| HIST1H2AH | 13.17 | 96.1 | 7.30 | 0.0000059 | 0.00059 |
| HIST1H2AM | 91.24 | 281.58 | 3.09 | 8.00E−08 | 2.30E−05 |
| HIST1H2BK | 284.5 | 499.61 | 1.76 | 0.000049 | 0.0026 |
| HIST1H4C | 2652.14 | 4340 | 1.64 | 0.000000012 | 0.0000047 |
| HIST1H4I | 18.82 | 59.32 | 3.15 | 1.80E−04 | 6.70E−03 |
| HIST3H2A | 52.78 | 95.05 | 1.80 | 1.30E−04 | 5.40E−03 |
| HLA-DRA | 7126.75 | 10478.03 | 1.47 | 0.00001 | 0.00091 |
| HLA-DRB1 | 3009.24 | 5091.8 | 1.69 | 2.40E−05 | 1.60E−03 |
| HLTF | 402.3 | 726.42 | 1.81 | 0.0028 | 0.044 |
| HMGB1 | 5473.99 | 7820.28 | 1.43 | 0.0014 | 0.028 |
| HMGB2 | 1480.91 | 2918.89 | 1.97 | 9.00E−05 | 4.10E−03 |
| HMGN1 | 1686.19 | 2523.22 | 1.50 | 1.20E−06 | 0.00016 |
| HMGN2 | 3367 | 5966.64 | 1.77 | 6.50E−07 | 0.0001 |
| HMMR | 6.44 | 83.13 | 12.91 | 1.80E−04 | 6.80E−03 |
| HNRNPK | 6106.43 | 7502.18 | 1.23 | 2.20E−03 | 3.70E−02 |
| HPRT1 | 449.79 | 880.26 | 1.96 | 7.50E−05 | 3.50E−03 |
| HSD17B10 | 458.18 | 620.32 | 1.35 | 0.0000081 | 0.00077 |
| HSPA8 | 13805.53 | 21838.93 | 1.58 | 0.00023 | 0.0079 |
| HSPA9 | 1683.28 | 2223.56 | 1.32 | 1.50E−04 | 6.00E−03 |
| HSPD1 | 1255.23 | 2432.67 | 1.94 | 2.50E−03 | 4.10E−02 |
| HSPE1 | 311.24 | 544.17 | 1.75 | 0.000097 | 0.0043 |
| ICAM2 | 319.08 | 118.43 | 0.37 | 9.00E−04 | 2.10E−02 |
| ID2 | 5169.39 | 7345.85 | 1.42 | 2.90E−04 | 9.30E−03 |
| IDI1 | 955.38 | 1287.13 | 1.35 | 0.0018 | 0.034 |
| IFI16 | 2590.54 | 4075.78 | 1.57 | 1.20E−03 | 2.60E−02 |
| IFI27L2 | 423.49 | 543.14 | 1.28 | 0.0015 | 0.031 |
| IFI35 | 389.91 | 856.45 | 2.20 | 2.20E−05 | 1.50E−03 |
| IFNG | 1819 | 4097.04 | 2.25 | 0.000016 | 0.0012 |
| IGFLR1 | 518.19 | 1075.99 | 2.08 | 5.80E−05 | 3.00E−03 |
| IL10RA | 3655.02 | 2375.04 | 0.65 | 7.70E−04 | 1.90E−02 |
| IL17RA | 689.13 | 430.92 | 0.63 | 1.90E−03 | 3.50E−02 |
| IL1RAP | 240.48 | 53.51 | 0.22 | 9.40E−06 | 8.50E−04 |
| IL21R | 442.5 | 739.71 | 1.67 | 1.40E−03 | 2.90E−02 |
| IL5RA | 77.29 | 6.62 | 0.09 | 1.00E−04 | 0.0046 |
| IL7R | 7126.48 | 2448.61 | 0.34 | 2.70E−05 | 1.80E−03 |
| INADL | 865.78 | 434.98 | 0.50 | 1.30E−04 | 5.40E−03 |
| IQGAP2 | 1632.7 | 873.83 | 0.54 | 6.80E−05 | 3.30E−03 |
| IQSEC1 | 401.75 | 251.08 | 0.62 | 4.50E−04 | 0.013 |
| IRF2BPL | 124.49 | 69.08 | 0.55 | 0.000029 | 0.0019 |
| IRF9 | 873.2 | 1401.67 | 1.61 | 0.000051 | 0.0027 |
| ITGA4 | 3339.43 | 2390.58 | 0.72 | 3.40E−05 | 2.10E−03 |
| ITGA5 | 664.55 | 317.12 | 0.48 | 1.10E−06 | 1.50E−04 |
| ITGA6 | 319.59 | 72.84 | 0.23 | 7.30E−06 | 0.0007 |
| ITGAE | 1970.92 | 6923.08 | 3.51 | 5.70E−28 | 6.00E−24 |
| ITGAM | 289.34 | 61.05 | 0.21 | 0.00092 | 0.021 |
| ITM2A | 2984.64 | 4642.52 | 1.56 | 0.0011 | 0.025 |
| JAK3 | 1817.89 | 2694.73 | 1.48 | 2.80E−03 | 4.40E−02 |
| JAKMIP1 | 357.63 | 672.99 | 1.88 | 1.00E−03 | 2.30E−02 |
| JHDM1D | 718.14 | 423.83 | 0.59 | 5.60E−04 | 1.50E−02 |
| KCNA3 | 1451.86 | 907.13 | 0.62 | 0.0008 | 0.019 |
| KIAA0100 | 609.29 | 380.25 | 0.62 | 0.0018 | 0.034 |
| KIAA0101 | 61.35 | 369.26 | 6.02 | 4.40E−08 | 1.40E−05 |
| KIAA1147 | 436.49 | 107.02 | 0.25 | 2.70E−07 | 0.000053 |
| KIAA1671 | 426.96 | 849.71 | 1.99 | 0.000037 | 0.0021 |
| KIF11 | 117.04 | 452.31 | 3.86 | 0.000018 | 0.0013 |
| KIF15 | 5.98 | 176.05 | 29.44 | 1.00E−06 | 1.50E−04 |
| KIF1B | 354.49 | 249.09 | 0.70 | 1.60E−03 | 0.031 |
| KIF2C | 40.18 | 234.86 | 5.85 | 0.0026 | 0.043 |
| KIF4A | 25.35 | 95.13 | 3.75 | 0.0027 | 0.043 |
| KIR2DL4 | 111.06 | 944.03 | 8.50 | 1.20E−06 | 0.00016 |
| KLF12 | 2256.22 | 1213.07 | 0.54 | 1.10E−03 | 0.025 |
| KLF13 | 409.4 | 220.24 | 0.54 | 0.00012 | 0.005 |
| KLF2 | 1151.11 | 258.17 | 0.22 | 0.000033 | 0.0021 |
| KLF3 | 444.94 | 172.84 | 0.39 | 0.00012 | 0.0053 |
| KLRB1 | 1379.8 | 1927.19 | 1.40 | 9.00E−04 | 0.021 |
| KLRG1 | 2562.52 | 794.41 | 0.31 | 0.00000082 | 0.00013 |
| KPNA2 | 419.39 | 1361.29 | 3.25 | 0.000026 | 0.0017 |
| LAG3 | 958.2 | 2583.31 | 2.70 | 2.70E−06 | 0.00031 |
| LAGE3 | 126.79 | 184.2 | 1.45 | 0.0024 | 0.039 |
| LAP3 | 552.73 | 1092.21 | 1.98 | 0.00066 | 0.017 |
| LAYN | 76.14 | 477.08 | 6.27 | 2.60E−05 | 0.0017 |
| LDHA | 6266.88 | 11706.93 | 1.87 | 0.000043 | 0.0024 |
| LDHB | 3591.38 | 4684.25 | 1.30 | 9.70E−04 | 0.022 |
| LDLRAP1 | 510.68 | 248.65 | 0.49 | 0.000095 | 0.0042 |
| LEF1 | 343.64 | 152.3 | 0.44 | 4.00E−04 | 0.012 |
| LIMK1 | 247.49 | 557.97 | 2.25 | 1.10E−04 | 0.0048 |
| LINC00152 | 558.54 | 832.44 | 1.49 | 4.50E−05 | 0.0025 |
| LINC00299 | 17.26 | 82.56 | 4.78 | 2.20E−03 | 0.038 |
| LIX1L | 242.32 | 181.63 | 0.75 | 0.0026 | 0.042 |
| LMAN2 | 1451.23 | 1875.51 | 1.29 | 0.0013 | 0.027 |
| LOC100132356 | 126.92 | 66.73 | 0.53 | 3.60E−04 | 1.10E−02 |
| LOC144571 | 283.07 | 72.61 | 0.26 | 1.40E−04 | 5.80E−03 |
| LOC541471 | 215.33 | 427.15 | 1.98 | 0.000042 | 0.0024 |
| LOC648987 | 150.68 | 82.6 | 0.55 | 5.50E−04 | 0.015 |
| LPP | 891.93 | 575.74 | 0.65 | 0.0021 | 0.037 |
| LPXN | 2938.89 | 3782.86 | 1.29 | 1.90E−03 | 3.50E−02 |
| LSM2 | 307.04 | 512.33 | 1.67 | 4.60E−04 | 1.30E−02 |
| LSP1 | 4879.17 | 6802.88 | 1.39 | 1.10E−03 | 0.025 |
| LYAR | 697.1 | 344.73 | 0.49 | 0.00019 | 0.0069 |
| MAD2L1 | 168.62 | 503.57 | 2.99 | 0.000000084 | 0.000023 |
| MAD2L2 | 427.75 | 713.04 | 1.67 | 0.00031 | 0.0097 |
| MAN2C1 | 397.72 | 247 | 0.62 | 7.20E−04 | 1.80E−02 |
| MAP4K1 | 1104.65 | 1626.19 | 1.47 | 5.30E−04 | 0.014 |
| MAP4K4 | 389.98 | 179.52 | 0.46 | 0.0000033 | 0.00038 |
| MAST4 | 154.9 | 367.2 | 2.37 | 0.00023 | 0.008 |
| MATK | 856.92 | 476.07 | 0.56 | 0.002 | 0.036 |
| MCM2 | 157.36 | 577.07 | 3.67 | 4.30E−04 | 1.20E−02 |
| MCM4 | 162.75 | 737.54 | 4.53 | 4.8E−13 | 9.9E−10 |
| MCM5 | 405.73 | 1182.59 | 2.91 | 9.10E−06 | 8.50E−04 |
| MCM6 | 482.96 | 1319.35 | 2.73 | 0.00000058 | 0.000095 |
| MCM7 | 388.24 | 928.54 | 2.39 | 0.00031 | 0.0097 |
| MEA1 | 366.69 | 540.54 | 1.47 | 0.00066 | 0.017 |
| MECP2 | 456.18 | 301.89 | 0.66 | 0.0013 | 0.027 |
| MELK | 30.59 | 192 | 6.28 | 9.90E−07 | 1.50E−04 |
| METTL5 | 374.5 | 483.97 | 1.29 | 0.00045 | 0.013 |
| MFN1 | 261.64 | 140.15 | 0.54 | 0.0018 | 0.034 |
| MIR155HG | 153.97 | 276.09 | 1.79 | 0.00043 | 0.012 |
| MKI67 | 232.03 | 991.22 | 4.27 | 2.30E−05 | 1.60E−03 |
| MLF1IP | 60.26 | 198.4 | 3.29 | 0.0027 | 0.043 |
| MLLT10 | 418.64 | 251.91 | 0.60 | 2.00E−03 | 3.60E−02 |
| MNF1 | 114.18 | 208.67 | 1.83 | 0.0000001 | 0.000026 |
| MOB1A | 786.12 | 1396.97 | 1.78 | 0.00082 | 0.02 |
| MPHOSPH8 | 1163.08 | 687.44 | 0.59 | 3.90E−04 | 1.20E−02 |
| MRPL51 | 443.68 | 673.09 | 1.52 | 1.50E−04 | 5.90E−03 |
| MSRB2 | 95.02 | 24 | 0.25 | 4.70E−04 | 0.013 |
| MT2A | 1218.49 | 1825.36 | 1.50 | 0.00014 | 0.0058 |
| MTHFD1 | 342.89 | 799.12 | 2.33 | 3.50E−06 | 3.90E−04 |
| MTHFD2 | 396.11 | 1033.04 | 2.61 | 1.10E−06 | 0.00015 |
| MYBL1 | 458.44 | 94.12 | 0.21 | 0.00001 | 0.0009 |
| MYBL2 | 13.24 | 108.17 | 8.17 | 0.00026 | 0.0085 |
| MYL6 | 8143.79 | 8799.36 | 1.08 | 3.10E−03 | 0.047 |
| MYO7A | 284.04 | 1385.72 | 4.88 | 1.80E−07 | 4.00E−05 |
| NAB1 | 459.14 | 960.08 | 2.09 | 0.000092 | 0.0041 |
| NACC2 | 80.3 | 24.67 | 0.31 | 1.10E−04 | 0.0047 |
| NCAPD2 | 338.64 | 662.17 | 1.96 | 0.0019 | 0.035 |
| NCAPG | 22.16 | 260.99 | 11.78 | 0.000000058 | 0.000017 |
| NCF1B | 93.35 | 36.06 | 0.39 | 1.70E−04 | 6.50E−03 |
| NDFIP2 | 550.06 | 1228.28 | 2.23 | 0.0000041 | 0.00045 |
| NDRG1 | 502.6 | 394.48 | 0.78 | 0.00062 | 0.016 |
| NDUFA6 | 717.75 | 831.62 | 1.16 | 0.0025 | 0.041 |
| NDUFB11 | 583.45 | 746.14 | 1.28 | 0.000036 | 0.0021 |
| NDUFB6 | 386.28 | 513.97 | 1.33 | 3.00E−03 | 4.60E−02 |
| NDUFB8 | 1124.76 | 1310.84 | 1.17 | 1.10E−03 | 2.40E−02 |
| NDUFS4 | 264.78 | 358.16 | 1.35 | 0.0029 | 0.045 |
| NDUFS6 | 394.45 | 576.11 | 1.46 | 0.00075 | 0.018 |
| NDUFS8 | 711.2 | 999.56 | 1.41 | 3.60E−04 | 1.10E−02 |
| NEB | 72.35 | 14.88 | 0.21 | 0.00067 | 0.017 |
| NEIL3 | 13.64 | 99.07 | 7.26 | 0.0012 | 0.025 |
| NEK2 | 16.76 | 79.94 | 4.77 | 0.000076 | 0.0035 |
| NFYC | 305.25 | 550.67 | 1.80 | 0.0011 | 0.024 |
| NONO | 2202.06 | 2710.13 | 1.23 | 0.002 | 0.036 |
| NOTCH1 | 410.74 | 714.39 | 1.74 | 0.000089 | 0.0041 |
| NR2C2 | 388.7 | 215.71 | 0.55 | 0.00097 | 0.022 |
| NSMCE2 | 217.56 | 360.38 | 1.66 | 2.00E−05 | 1.40E−03 |
| NUAK2 | 117.92 | 46 | 0.39 | 2.30E−03 | 3.90E−02 |
| NUDT5 | 493.42 | 744.8 | 1.51 | 0.0011 | 0.024 |
| NUSAP1 | 214.38 | 756.67 | 3.53 | 0.000005 | 0.00053 |
| OAS2 | 1085.85 | 1982.3 | 1.83 | 0.0001 | 0.0046 |
| OASL | 1204.86 | 2315.98 | 1.92 | 1.30E−04 | 5.40E−03 |
| ODF2 | 202.56 | 380.47 | 1.88 | 3.60E−04 | 1.10E−02 |
| ODZ1 | 383.31 | 170.26 | 0.44 | 0.0022 | 0.038 |
| OFD1 | 1125.95 | 731.52 | 0.65 | 0.00049 | 0.014 |
| ORC1 | 8.75 | 83.03 | 9.49 | 2.90E−04 | 9.20E−03 |
| ORC6 | 24.85 | 77.63 | 3.12 | 0.00032 | 0.0098 |
| PAG1 | 1851.17 | 3347.22 | 1.81 | 4.9E−09 | 0.0000025 |
| PARK7 | 1821.25 | 2917.33 | 1.60 | 0.000059 | 0.003 |
| PARP8 | 4114.98 | 2838.74 | 0.69 | 1.90E−04 | 6.80E−03 |
| PARP9 | 1006.89 | 1962.18 | 1.95 | 2.10E−06 | 2.60E−04 |
| PATL2 | 501.85 | 202.04 | 0.40 | 1.30E−05 | 1.10E−03 |
| PBK | 3.31 | 97.83 | 29.56 | 5.30E−05 | 2.80E−03 |
| PCK2 | 201.05 | 373.6 | 1.86 | 3.30E−03 | 0.049 |
| PCMT1 | 1002.67 | 1421.64 | 1.42 | 2.70E−03 | 0.044 |
| PCMTD2 | 468.17 | 274.21 | 0.59 | 3.20E−03 | 4.80E−02 |
| PCNXL3 | 423.15 | 207.77 | 0.49 | 1.30E−03 | 0.027 |
| PDE8A | 95.63 | 28.09 | 0.29 | 6.50E−04 | 0.017 |
| PDIA6 | 1136.83 | 1816.68 | 1.60 | 9.50E−05 | 4.20E−03 |
| PGAM1 | 1588.46 | 2950.9 | 1.86 | 3E−10 | 0.00000026 |
| PGK1 | 5481.98 | 8759.84 | 1.60 | 0.00018 | 0.0068 |
| PHF1 | 550.76 | 360.53 | 0.65 | 9.00E−04 | 2.10E−02 |
| PHF12 | 397.36 | 234.65 | 0.59 | 0.0025 | 0.041 |
| PIAS2 | 284.69 | 92.79 | 0.33 | 5.20E−08 | 1.60E−05 |
| PIH1D1 | 294.58 | 486.16 | 1.65 | 0.0028 | 0.044 |
| PIK3R5 | 1103.75 | 774.82 | 0.70 | 0.0029 | 0.045 |
| PIN1 | 369.65 | 515.71 | 1.40 | 0.00074 | 0.018 |
| PIP4K2A | 4592.08 | 3658.94 | 0.80 | 0.0015 | 0.03 |
| PITPNC1 | 757.99 | 472.99 | 0.62 | 2.30E−03 | 3.90E−02 |
| PKI55 | 91.09 | 12.46 | 0.14 | 0.0012 | 0.025 |
| PKM2 | 2422.68 | 4984.49 | 2.06 | 1.10E−05 | 0.00097 |
| PKMYT1 | 12.52 | 121.61 | 9.71 | 7.30E−04 | 1.80E−02 |
| PLA2G16 | 369.14 | 537.42 | 1.46 | 2.50E−03 | 0.041 |
| PLAC8 | 922.22 | 286.56 | 0.31 | 2.10E−04 | 7.40E−03 |
| PLEK | 1739.86 | 897.36 | 0.52 | 1.70E−03 | 0.032 |
| PLEKHA5 | 210.13 | 122.87 | 0.58 | 0.0018 | 0.034 |
| PLEKHG3 | 199.42 | 30.41 | 0.15 | 0.00032 | 0.0098 |
| PLK1 | 30 | 188.28 | 6.28 | 0.00027 | 0.0086 |
| PLXND1 | 271.96 | 104.62 | 0.38 | 0.000022 | 0.0015 |
| PMF1 | 167.12 | 283.48 | 1.70 | 0.00015 | 0.006 |
| POLR2G | 1101.8 | 1409.59 | 1.28 | 8.70E−04 | 2.10E−02 |
| PPA1 | 841.25 | 1809.07 | 2.15 | 0.000023 | 0.0016 |
| PPAP2A | 175.05 | 333.13 | 1.90 | 2.80E−03 | 4.50E−02 |
| PPIB | 913.69 | 1129.62 | 1.24 | 0.0027 | 0.043 |
| PPM1M | 762.76 | 1206.19 | 1.58 | 0.000021 | 0.0015 |
| PPP1R13B | 104.74 | 49.38 | 0.47 | 2.80E−03 | 4.40E−02 |
| PPP1R7 | 372.57 | 536.43 | 1.44 | 4.20E−04 | 0.012 |
| PPP2R4 | 409.91 | 686.97 | 1.68 | 0.0021 | 0.037 |
| PPP2R5D | 592.29 | 1010.23 | 1.71 | 0.00024 | 0.0082 |
| PPP5C | 398.93 | 625.73 | 1.57 | 3.00E−03 | 4.60E−02 |
| PRC1 | 55.96 | 230.73 | 4.12 | 0.0013 | 0.027 |
| PRDM2 | 1151.46 | 736.69 | 0.64 | 0.0006 | 0.016 |
| PRDX5 | 867.79 | 1168.84 | 1.35 | 1.80E−04 | 6.70E−03 |
| PRDX6 | 971.03 | 1665.03 | 1.71 | 9.40E−08 | 2.40E−05 |
| PRKAG1 | 411.81 | 693.23 | 1.68 | 0.00087 | 0.021 |
| PRKAR1A | 2397.56 | 3587.37 | 1.50 | 4.60E−05 | 2.50E−03 |
| PSMA2 | 1271.36 | 1890.5 | 1.49 | 0.00029 | 0.0092 |
| PSMA5 | 1481.87 | 1996.42 | 1.35 | 0.0021 | 0.037 |
| PSMA6 | 1196.06 | 1908.5 | 1.60 | 0.0023 | 0.039 |
| PSMB6 | 777.79 | 1003.83 | 1.29 | 0.00088 | 0.021 |
| PSMB8 | 2339.04 | 3802.45 | 1.63 | 8.80E−08 | 2.30E−05 |
| PSMB9 | 2886.73 | 4217.66 | 1.46 | 7.70E−04 | 1.90E−02 |
| PSMC1 | 744.94 | 1036.16 | 1.39 | 4.70E−04 | 1.30E−02 |
| PSMC3 | 695.56 | 1144.2 | 1.65 | 1.30E−04 | 0.0055 |
| PSMD8 | 936.35 | 1551.03 | 1.66 | 0.000000031 | 0.00001 |
| PSME1 | 4634.03 | 6465.06 | 1.40 | 4.20E−04 | 0.012 |
| PSME2 | 1876.32 | 3417.48 | 1.82 | 2.90E−08 | 9.90E−06 |
| PTAR1 | 674.12 | 408.5 | 0.61 | 0.0021 | 0.037 |
| PTCH1 | 233.54 | 70.41 | 0.30 | 2.50E−03 | 0.041 |
| PTGDR | 729.27 | 265.66 | 0.36 | 2.50E−03 | 0.041 |
| PTGER2 | 1044.16 | 377.31 | 0.36 | 0.00000023 | 0.000047 |
| PTMA | 4907.9 | 6500.19 | 1.32 | 4.80E−05 | 0.0026 |
| PTMS | 105.43 | 244.99 | 2.32 | 1.60E−05 | 1.20E−03 |
| PTPN22 | 2231.66 | 3526.12 | 1.58 | 0.00029 | 0.0091 |
| PTPN7 | 2890.98 | 5136.25 | 1.78 | 0.000000086 | 0.000023 |
| PTTG1 | 401.03 | 922.08 | 2.30 | 1.7E−12 | 2.9E−09 |
| PXN | 785.4 | 359.13 | 0.46 | 2.60E−06 | 3.00E−04 |
| PZP | 352.53 | 110.35 | 0.31 | 7.90E−04 | 1.90E−02 |
| RAB27A | 1364.45 | 2439.95 | 1.79 | 7.90E−05 | 3.70E−03 |
| RAB3GAP1 | 573.3 | 1232.27 | 2.15 | 1.70E−06 | 2.10E−04 |
| RACGAP1 | 58.09 | 341.87 | 5.89 | 0.00053 | 0.014 |
| RAN | 2554 | 3494.86 | 1.37 | 5.80E−04 | 1.50E−02 |
| RANBP1 | 292.4 | 521 | 1.78 | 5.60E−06 | 5.90E−04 |
| RAP2B | 945.19 | 545.54 | 0.58 | 9.40E−06 | 0.00085 |
| RARRES3 | 2822.7 | 4449.56 | 1.58 | 0.00019 | 0.0069 |
| RASA3 | 642.85 | 224.91 | 0.35 | 0.000037 | 0.0021 |
| RASGRP2 | 260.76 | 91.61 | 0.35 | 0.000051 | 0.0027 |
| RASSF3 | 439.25 | 217.17 | 0.49 | 0.0000098 | 0.00087 |
| RBBP4 | 1287.12 | 1849.22 | 1.44 | 3.10E−03 | 4.70E−02 |
| RBBP8 | 76.55 | 272.07 | 3.55 | 3.00E−05 | 0.0019 |
| RBCK1 | 687.92 | 1033.84 | 1.50 | 0.0024 | 0.04 |
| RBL2 | 3510.25 | 2900.91 | 0.83 | 2.40E−03 | 0.039 |
| RBPJ | 2046.58 | 4981.33 | 2.43 | 1.1E−10 | 0.0000001 |
| RBX1 | 729.98 | 862.9 | 1.18 | 5.00E−04 | 1.40E−02 |
| RERE | 175.46 | 102 | 0.58 | 1.20E−03 | 2.60E−02 |
| RFC2 | 150.24 | 331.86 | 2.21 | 7.30E−04 | 0.018 |
| RFX5 | 569.8 | 908.03 | 1.59 | 0.00055 | 0.015 |
| RFX7 | 371.29 | 580.63 | 1.56 | 2.20E−03 | 3.70E−02 |
| RG9MTD3 | 186.25 | 91.24 | 0.49 | 0.00081 | 0.019 |
| RHOA | 5313.23 | 7571.11 | 1.42 | 0.00024 | 0.008 |
| RIC3 | 81.14 | 14.71 | 0.18 | 3.00E−03 | 4.60E−02 |
| RMI2 | 15.81 | 110.96 | 7.02 | 2.00E−03 | 3.60E−02 |
| RNASEH2B | 434.21 | 636.56 | 1.47 | 1.60E−03 | 3.10E−02 |
| RNF144A | 237.31 | 54.88 | 0.23 | 1.40E−04 | 5.80E−03 |
| RNF149 | 2402.73 | 1907.45 | 0.79 | 2.60E−03 | 4.20E−02 |
| RNF26 | 430.8 | 313.33 | 0.73 | 2.30E−03 | 3.90E−02 |
| ROMO1 | 537.59 | 643.14 | 1.20 | 0.0000015 | 0.00018 |
| RPS26 | 1759.55 | 2182.35 | 1.24 | 0.00015 | 0.006 |
| RREB1 | 365.55 | 136.97 | 0.37 | 5.60E−05 | 0.0028 |
| RRM1 | 398.16 | 946.36 | 2.38 | 6.50E−05 | 0.0032 |
| RRM2 | 147.32 | 890.79 | 6.05 | 0.000000011 | 0.0000047 |
| S100A10 | 2989.32 | 1990.42 | 0.67 | 1.20E−03 | 2.50E−02 |
| S1PR1 | 1197.42 | 322.99 | 0.27 | 2.10E−09 | 1.40E−06 |
| S1PR5 | 447.46 | 113.55 | 0.25 | 0.00065 | 0.017 |
| SACS | 830.41 | 435.62 | 0.52 | 1.00E−04 | 0.0046 |
| SAMD3 | 1387.57 | 678.58 | 0.49 | 5.00E−06 | 5.30E−04 |
| SAMSN1 | 1788.24 | 3033.55 | 1.70 | 2.50E−03 | 4.10E−02 |
| SCARNA17 | 372.71 | 154.29 | 0.41 | 1.20E−03 | 2.50E−02 |
| SCCPDH | 254.04 | 487.04 | 1.92 | 0.0019 | 0.035 |
| SCUBE1 | 32.17 | 183.17 | 5.69 | 2.10E−04 | 0.0074 |
| SEC11A | 703.82 | 1027.6 | 1.46 | 0.0016 | 0.031 |
| SEC61B | 760.13 | 792.55 | 1.04 | 0.0019 | 0.035 |
| SEC62 | 1067.72 | 844.62 | 0.79 | 0.00049 | 0.014 |
| SEL1L3 | 1148.83 | 1692.06 | 1.47 | 0.003 | 0.046 |
| SELL | 1766.39 | 811.51 | 0.46 | 2.70E−04 | 0.0089 |
| SEMA4C | 89.64 | 18.59 | 0.21 | 0.000013 | 0.0011 |
| SEMA7A | 65.36 | 245.67 | 3.76 | 0.00011 | 0.0049 |
| SF3B14 | 572.01 | 775.61 | 1.36 | 0.000042 | 0.0024 |
| SFXN1 | 775.31 | 1137.9 | 1.47 | 0.0028 | 0.044 |
| SFXN2 | 75.71 | 195.71 | 2.58 | 0.00035 | 0.011 |
| SGMS1 | 633.46 | 1106.9 | 1.75 | 0.0021 | 0.037 |
| SGOL1 | 30.8 | 95.21 | 3.09 | 0.000021 | 0.0015 |
| SGOL2 | 50.63 | 172.61 | 3.41 | 0.0012 | 0.025 |
| SH2B3 | 365.85 | 144.99 | 0.40 | 6.10E−05 | 3.00E−03 |
| SH3BP5 | 106.02 | 62.13 | 0.59 | 1.70E−05 | 0.0013 |
| SHFM1 | 717.24 | 973 | 1.36 | 0.000014 | 0.0011 |
| SIDT1 | 439.77 | 306.45 | 0.70 | 1.30E−03 | 2.70E−02 |
| SIRPG | 1076.24 | 2764.48 | 2.57 | 1.80E−06 | 2.20E−04 |
| SLC25A5 | 1963.73 | 2852.39 | 1.45 | 5.00E−05 | 0.0026 |
| SLC27A2 | 147.29 | 720.72 | 4.89 | 2.60E−07 | 0.000053 |
| SLC30A7 | 1003.52 | 758.88 | 0.76 | 1.70E−03 | 0.032 |
| SLC39A10 | 579.85 | 359.61 | 0.62 | 1.70E−03 | 3.20E−02 |
| SLC44A1 | 284.81 | 164.79 | 0.58 | 1.60E−03 | 0.031 |
| SMAD5 | 482.74 | 277.17 | 0.57 | 0.00043 | 0.012 |
| SMC2 | 279.36 | 582.55 | 2.09 | 0.00095 | 0.022 |
| SMC4 | 766.42 | 1409.18 | 1.84 | 0.00023 | 0.008 |
| SNAP47 | 387.08 | 709.91 | 1.83 | 2.30E−03 | 0.039 |
| SNHG9 | 124.25 | 54.06 | 0.44 | 0.003 | 0.046 |
| SNRPB | 1574.18 | 2223.3 | 1.41 | 2.60E−05 | 0.0017 |
| SNRPE | 638.08 | 882.67 | 1.38 | 0.00099 | 0.022 |
| SNRPG | 1039.23 | 1302.29 | 1.25 | 0.0015 | 0.029 |
| SOCS5 | 382.45 | 140.36 | 0.37 | 0.000045 | 0.0025 |
| SOD1 | 2102.07 | 2741.64 | 1.30 | 7.50E−04 | 0.018 |
| SORBS3 | 165.73 | 123.56 | 0.75 | 1.60E−03 | 3.20E−02 |
| SORL1 | 2103.87 | 885.33 | 0.42 | 2.10E−07 | 4.50E−05 |
| SP140 | 1068.93 | 1523.34 | 1.43 | 2.70E−03 | 4.30E−02 |
| SPAG5 | 29.26 | 183.8 | 6.28 | 7.60E−04 | 1.90E−02 |
| SPDYE1 | 66.5 | 14.96 | 0.22 | 0.00005 | 0.0026 |
| SPON2 | 169.84 | 63.15 | 0.37 | 0.00083 | 0.02 |
| SQLE | 92.27 | 340.14 | 3.69 | 2.7E−09 | 0.0000016 |
| SRGAP3 | 353.52 | 802.28 | 2.27 | 2.50E−03 | 0.041 |
| SRP14 | 3250.7 | 4175.9 | 1.28 | 0.0028 | 0.044 |
| SRSF4 | 823.62 | 1118.39 | 1.36 | 0.00035 | 0.011 |
| SSBP2 | 276.52 | 79.96 | 0.29 | 1.40E−04 | 5.70E−03 |
| SSH2 | 1388.31 | 810.75 | 0.58 | 1.20E−03 | 0.025 |
| STAT1 | 3437.52 | 9109.41 | 2.65 | 0.00000049 | 0.000086 |
| STAT3 | 1597.28 | 2752.8 | 1.72 | 0.00017 | 0.0065 |
| STAT5A | 679.07 | 1001.22 | 1.47 | 1.70E−03 | 3.20E−02 |
| STIL | 25.33 | 105.04 | 4.15 | 1.60E−03 | 0.031 |
| STK38 | 1099.61 | 596.48 | 0.54 | 0.0000013 | 0.00017 |
| STMN1 | 590.71 | 2186.64 | 3.70 | 4.00E−11 | 4.60E−08 |
| STX3 | 126.37 | 29.28 | 0.23 | 0.000007 | 0.00068 |
| SUB1 | 1939.81 | 2734.93 | 1.41 | 0.0024 | 0.04 |
| SUMO3 | 653.71 | 940.91 | 1.44 | 0.00019 | 0.0068 |
| SYPL1 | 870.6 | 564.93 | 0.65 | 0.0013 | 0.027 |
| SYT11 | 839.77 | 1558.28 | 1.86 | 0.0000011 | 0.00015 |
| TALDO1 | 645.73 | 979.31 | 1.52 | 2.30E−04 | 7.90E−03 |
| TBC1D4 | 271.96 | 635.29 | 2.34 | 7.20E−05 | 3.40E−03 |
| TBL1XR1 | 2180.12 | 3095.97 | 1.42 | 1.70E−04 | 0.0065 |
| TC2N | 2434.21 | 1514.2 | 0.62 | 1.60E−05 | 1.20E−03 |
| TCF7 | 929.6 | 348.42 | 0.37 | 0.00025 | 0.0084 |
| TCP1 | 1136.95 | 1555.5 | 1.37 | 0.0012 | 0.026 |
| TFDP1 | 257.02 | 506.17 | 1.97 | 1.80E−03 | 3.30E−02 |
| TGFBR3 | 1043.07 | 598.43 | 0.57 | 0.0015 | 0.03 |
| THEM4 | 345.7 | 150.11 | 0.43 | 1.80E−07 | 4.00E−05 |
| THRA | 136.85 | 60.02 | 0.44 | 1.70E−03 | 3.30E−02 |
| TIGIT | 2349.71 | 3822.88 | 1.63 | 0.0006 | 0.016 |
| TLE4 | 451.6 | 223.59 | 0.50 | 0.00025 | 0.0083 |
| TMEM14C | 484.01 | 650.25 | 1.34 | 1.20E−03 | 2.60E−02 |
| TMEM181 | 571.69 | 373.72 | 0.65 | 3.00E−03 | 4.60E−02 |
| TMEM63A | 535.36 | 212.68 | 0.40 | 0.00025 | 0.0083 |
| TMPO | 765.25 | 1272.26 | 1.66 | 0.00054 | 0.015 |
| TMSB4X | 61515.24 | 70294.98 | 1.14 | 0.001 | 0.023 |
| TNFRSF10A | 659.13 | 287.2 | 0.44 | 6.80E−05 | 3.30E−03 |
| TNFRSF10B | 363.08 | 171.64 | 0.47 | 1.70E−03 | 3.20E−02 |
| TNFRSF9 | 642.02 | 2048.09 | 3.19 | 1.50E−05 | 0.0012 |
| TNFSF4 | 182.3 | 741.26 | 4.07 | 9.5E−09 | 0.0000041 |
| TNIK | 601.58 | 424.55 | 0.71 | 1.00E−03 | 2.30E−02 |
| TNRC6B | 2131.91 | 1512.37 | 0.71 | 1.80E−04 | 6.70E−03 |
| TNRC6C | 534.28 | 214 | 0.40 | 1.80E−05 | 1.30E−03 |
| TNS3 | 186.99 | 886.05 | 4.74 | 2.60E−03 | 4.20E−02 |
| TOMM5 | 702.41 | 824.16 | 1.17 | 1.50E−04 | 6.00E−03 |
| TOP2A | 178.96 | 964.34 | 5.39 | 4.90E−07 | 8.60E−05 |
| TOX2 | 107.4 | 344.69 | 3.21 | 0.00018 | 0.0067 |
| TP53BP2 | 380.46 | 244.48 | 0.64 | 0.0029 | 0.045 |
| TP73 | 20.9 | 145.52 | 6.96 | 2.90E−03 | 0.045 |
| TPI1 | 3283.17 | 6630.63 | 2.02 | 5.50E−08 | 1.70E−05 |
| TPM3 | 6051.12 | 8502.4 | 1.41 | 2.50E−03 | 4.10E−02 |
| TPX2 | 55.89 | 430.95 | 7.71 | 1.30E−08 | 5.00E−06 |
| TRAFD1 | 644.7 | 1117.26 | 1.73 | 0.00021 | 0.0074 |
| TRIM44 | 515.13 | 232.49 | 0.45 | 8.90E−06 | 0.00084 |
| TRIP12 | 875.83 | 1220.13 | 1.39 | 1.30E−04 | 0.0054 |
| TRIP13 | 11.31 | 71.96 | 6.36 | 3.50E−05 | 2.10E−03 |
| TRMT2B | 363.84 | 175.58 | 0.48 | 0.00042 | 0.012 |
| TROAP | 17.87 | 116.81 | 6.54 | 0.00024 | 0.008 |
| TSC22D2 | 426.31 | 252.23 | 0.59 | 0.0019 | 0.035 |
| TSHZ2 | 13.66 | 81.1 | 5.94 | 2.30E−06 | 0.00027 |
| TSPAN17 | 303.16 | 591.26 | 1.95 | 0.00019 | 0.0069 |
| TSPAN32 | 357.88 | 115.79 | 0.32 | 2.00E−09 | 1.40E−06 |
| TTC16 | 276.63 | 73.08 | 0.26 | 0.000051 | 0.0027 |
| TTC24 | 136.13 | 310.33 | 2.28 | 1.30E−03 | 0.027 |
| TTC3 | 1935.99 | 1542.03 | 0.80 | 0.00099 | 0.022 |
| TTC9 | 65.14 | 18.91 | 0.29 | 0.0019 | 0.035 |
| TTYH3 | 142.48 | 368.28 | 2.58 | 1.00E−03 | 2.30E−02 |
| TUBA1B | 1146.26 | 2248.41 | 1.96 | 0.000073 | 0.0035 |
| TUBB | 3094.93 | 6024.03 | 1.95 | 0.000044 | 0.0024 |
| TXNL4A | 355.5 | 387.27 | 1.09 | 0.0003 | 0.0093 |
| TYMS | 27.02 | 160.27 | 5.93 | 7.60E−05 | 0.0035 |
| UBB | 7122.51 | 10855.69 | 1.52 | 1.40E−05 | 0.0011 |
| UBC | 6474.06 | 10239.8 | 1.58 | 0.00025 | 0.0082 |
| UBE2A | 600.05 | 1029.69 | 1.72 | 3.60E−04 | 1.10E−02 |
| UBE2C | 59.86 | 357.7 | 5.98 | 0.00058 | 0.015 |
| UBE2L6 | 1931.55 | 3639.96 | 1.88 | 0.0000011 | 0.00015 |
| UBE2N | 1101.27 | 1455.97 | 1.32 | 0.000013 | 0.0011 |
| UBE2Q2 | 379.1 | 282.93 | 0.75 | 1.60E−03 | 0.032 |
| UBL5 | 1243.23 | 1305.32 | 1.05 | 1.70E−03 | 0.032 |
| UCP2 | 3767.53 | 6929.37 | 1.84 | 2.00E−03 | 3.60E−02 |
| UHRF1 | 46.05 | 227.23 | 4.93 | 0.00071 | 0.018 |
| UQCR10 | 979.75 | 1046.33 | 1.07 | 0.0028 | 0.044 |
| UQCRH | 704.34 | 871.49 | 1.24 | 0.000091 | 0.0041 |
| UROD | 296.45 | 412.88 | 1.39 | 0.0013 | 0.027 |
| USP1 | 508.66 | 928.84 | 1.83 | 5.80E−06 | 0.00059 |
| USP3 | 1219.84 | 769.72 | 0.63 | 6.90E−05 | 0.0033 |
| USP53 | 160.26 | 97.43 | 0.61 | 2.10E−03 | 0.037 |
| UTRN | 4399.63 | 2870.85 | 0.65 | 0.000063 | 0.0031 |
| VCAM1 | 283.21 | 1010.31 | 3.57 | 5.40E−05 | 2.80E−03 |
| VCL | 357.23 | 106.8 | 0.30 | 2.60E−04 | 8.60E−03 |
| WARS | 779.76 | 1874.98 | 2.40 | 2.30E−03 | 3.90E−02 |
| WDR1 | 2698.42 | 3744.26 | 1.39 | 4.30E−04 | 1.20E−02 |
| WDR34 | 99.4 | 263.39 | 2.65 | 7.50E−04 | 1.80E−02 |
| WDR37 | 380.8 | 198.7 | 0.52 | 0.0014 | 0.029 |
| WHAMMP3 | 54.36 | 31.03 | 0.57 | 5.90E−04 | 0.015 |
| WHSC1 | 227.72 | 341.87 | 1.50 | 3.00E−03 | 0.046 |
| XPO6 | 1042.48 | 660.02 | 0.63 | 0.0011 | 0.024 |
| XRCC6 | 3037.04 | 3964.44 | 1.31 | 0.00017 | 0.0066 |
| YWHAE | 856.84 | 1154.67 | 1.35 | 5.40E−04 | 1.50E−02 |
| YWHAQ | 3336.67 | 4797.11 | 1.44 | 0.00015 | 0.0059 |
| ZBTB16 | 143.23 | 54.08 | 0.38 | 0.00062 | 0.016 |
| ZBTB4 | 422.39 | 272.46 | 0.65 | 1.50E−03 | 0.031 |
| ZCRB1 | 498.37 | 756.91 | 1.52 | 2.70E−04 | 8.70E−03 |
| ZHX3 | 151.2 | 60.64 | 0.40 | 1.00E−03 | 0.023 |
| ZMAT1 | 242.9 | 65.86 | 0.27 | 4.40E−04 | 1.20E−02 |
| ZMIZ1 | 206.73 | 421.03 | 2.04 | 0.0033 | 0.049 |
| ZNF286A | 190.25 | 105.37 | 0.55 | 3.10E−03 | 0.047 |
| ZNF33A | 917.13 | 548.2 | 0.60 | 0.00021 | 0.0074 |
| ZNF394 | 773.63 | 454.31 | 0.59 | 2.40E−03 | 3.90E−02 |
| ZNF43 | 287.04 | 193.03 | 0.67 | 2.10E−03 | 3.70E−02 |
| ZNF528 | 171.05 | 56.59 | 0.33 | 2.90E−03 | 4.50E−02 |
| ZNF83 | 477.6 | 235.19 | 0.49 | 1.80E−04 | 6.80E−03 |
| ZNRF1 | 152.52 | 473.53 | 3.10 | 0.000022 | 0.0015 |
| ZWINT | 70.8 | 318.92 | 4.50 | 5.80E−04 | 0.015 |
| ZXDC | 482.74 | 262.21 | 0.54 | 2.40E−03 | 3.90E−02 |
| ZZEF1 | 615.13 | 433.28 | 0.70 | 0.00037 | 0.011 |

TABLE 9
Pathway analysis of differentially expressed genes in CD103 high TILs from NSCLC.

| | Disease or | | Predicted | | | Number |
|---|---|---|---|---|---|---|
| | functions | | activation | Activation | | of mol- |
| Categories | annotation | P value | state | z-score | Molecules | ecules |
| Cellular | proliferation | 1.68E−25 | Increased | 7.486 | ABCB1, ACTN4, AFAP1L2, ALOX5AP, ARL3, ASB2, ASPM, | 211 |
| Growth | of cells | | | | ATP5B, AURKA, AURKB, BARD1, BATF, BCCIP, | |
| and | | | | | BIRC5, BRIP1, BST2, BUB1, CACYBP, CALCOCO2, CALM1 | |
| Proliferation | | | | | (includes others), CAPZA1, CCL3, CCL5, CCNA2, CCNB2, CCNE2, | |
| | | | | | CD2, CD38, CD3G, CD63, CD82, CDC123, CDC20, CDC45, | |
| | | | | | CDC6, CDCA2, CDCA7, CDCA8, CDK1, CDKN3, CENPF, | |
| | | | | | CFL1, CHEK1, CISD1, CKLF, CKS1B, CKS2, CLIC1, | |
| | | | | | CLSPN, CMC2, COPE, COPS6, COPZ1, CSF1, DAPK2, | |
| | | | | | DBN1, DDB2, DHFR, DIXDC1, DLGAP5, DNAJA1, DNAJB6, | |
| | | | | | DNPH1, DPY30, DTL, EIF3I, ENO1, ENTPD1, ETFB, | |
| | | | | | ETV7, EZH2, FABP5, FANCL, FEN1, FIS1, FKBP1A, GALNT2, | |
| | | | | | GAPDH, GBP1, GBP2, GLDC, GPI, GZMB, H2AFX, | |
| | | | | | H2AFZ, HAVCR2, HCLS1, HIST1H1B, HIST1H2AC, | |
| | | | | | HLA-DRB1, HLTF, HMGB1, HMGB2, HMGN1, HMMR, HNRNPK, | |
| | | | | | HPRT1, HSPA8, HSPA9, HSPD1, ID2, IFI16, IFNG, IL21R, | |
| | | | | | JAK3, KIAA0101, KIF11, KIF15, KIF2C, KLRB1, | |
| | | | | | KPNA2, LAG3, LAP3, LDHA, LIMK1, MAD2L1, MAD2L2, | |
| | | | | | MAP4K1, MCM2, MCM4, MCM5, MCM7, MELK, MKI67, | |
| | | | | | MOB1A, MT2A, MTHFD1, MYBL2, NAB1, NCAPG, | |
| | | | | | NDUFS4, NEIL3, NEK2, NOTCH1, ORC1, PAG1, PARK7, | |
| | | | | | PBK, PCK2, PGK1, PIN1, PKM, PLA2G16, PLK1, PPIB, | |
| | | | | | PPP5C, PRC1, PRKAR1A, PSMA5, PSMC1, PSMC3, PSME2, | |
| | | | | | PTMA, PTPN22, PTTG1, RAB27A, RACGAP1, RAN, | |
| | | | | | RANBP1, RARRES3, RBBP4, RBCK1, RBPJ, RBX1, | |
| | | | | | RHOA, RNASEH2B, ROMO1, RRM1, RRM2, SAMSN1, | |
| | | | | | SEMA7A, SGMS1, SHFM1, SIRPG, SLC25A5, SOD1, SRGAP3, | |
| | | | | | STAT1, STAT3, STAT5A, STIL, STMN1, SUMO3, | |
| | | | | | TCP1, TFDP1, TIGIT, TMPO, TMSB10/TMSB4X, TNFRSF9, | |
| | | | | | TNFSF4, TNS3, TOP2A, TP73, TPM3, TPX2, TUBB, | |
| | | | | | TYMS, UBC, UBE2A, UBE2C, UBE2L6, UBE2N, UCP2, | |
| | | | | | UHRF1, USP1, VCAM1, WARS, WHSC1, XRCC6, YWHAQ, | |
| | | | | | ZMIZ1 | |
| Cell Cycle | cell cycle | 2.01E−25 | Increased | 2.021 | AFAP1L2, ANAPC11, AURKA, AURKB, BCCIP, BIRC5, | 97 |
| | progression | | | | BUB1, CASC5, CCL3, CCNA2, CCNE2, CDC123, CDC20, | |
| | | | | | CDC6, CDCA8, CDK1, CDKN3, CENPF, CHEK1, CKAP2, | |
| | | | | | CKS1B, CKS2, CLSPN, COPZ1, CSF1, DHFR, DIXDC1, | |
| | | | | | DLGAP5, DTL, EZH2, FBXO5, FKBP1A, GBP2, GPI, GTSE1, | |
| | | | | | HMGB1, HSPA8, HSPA9, ID2, IFI16, IFNG, IRF9, JAK3, | |
| | | | | | KIAA0101, KIF11, KIF15, KIF2C, KIF4A, LAG3, MAD2L1, | |
| | | | | | MAD2L2, MCM2, MCM7, MELK, MKI67, MYBL2, | |
| | | | | | NEK2, NOTCH1, NUSAP1, ORC6, PIN1, PKMYT1, PLA2G16, | |
| | | | | | PLK1, PPP5C, PRKAR1A, PTMA, PTTG1, RACGAP1, | |
| | | | | | RAN, RBBP8, RBX1, RHOA, RMI2, RRM1, SGO1, | |
| | | | | | SPAG5, STAT1, STAT3, STAT5A, STIL, STMN1, TBL1XR1, | |
| | | | | | TCP1, TFDP1, TMPO, TOP2A, TP73, TPX2, TUBB, | |
| | | | | | TYMS, UBC, UBE2C, USP1, XRCC6, YWHAE, ZWINT | |
| Cell Cycle, | segregation | 5.6E−21 | | | AURKA, AURKB, BUB1, CCNA2, CCNB2, CDC6, CDCA2, | 29 |
| Cellular | of | | | | CENPF, KIF11, KIF2C, MAD2L1, NCAPD2, NCAPG, | |
| Assembly and | chromosomes | | | | NEK2, NUSAP1, ODF2, PLK1, PMF1/PMF1- | |
| Organization, | | | | | BGLAP, PPP1R7, PTTG1, RAN, RHOA, SGO1, SMC2, SMC4, | |
| DNA | | | | | SPAG5, TOP2A, TPX2, ZWINT | |
| Replication, | | | | | | |
| Recombination, | | | | | | |
| and Repair | | | | | | |
| Cell Cycle | mitosis | 3.97E−19 | | | AFAP1L2, ANAPC11, AURKA, AURKB, BIRC5, BUB1, | 55 |
| | | | | | CASC5, CCNA2, CDC123, CDC20, CDCA8, CDK1, CDKN3, | |
| | | | | | CENPF, CHEK1, CKAP2, CSF1, DLGAP5, FBXO5, JAK3, | |
| | | | | | KIF11, KIF15, KIF2C, KIF4A, MAD2L1, MAD2L2, | |
| | | | | | MYBL2, NEK2, NUSAP1, ORC6, PIN1, PKMYT1, PLK1, | |
| | | | | | PPP5C, PTMA, PTTG1, RACGAP1, RAN, RBBP8, RMI2, | |
| | | | | | SGO1, SPAG5, STAT1, STAT3, STIL, STMN1, TBL1XR1, | |
| | | | | | TCP1, TOP2A, TP73, TPX2, TUBB, UBE2C, YWHAE, ZWINT | |
| Cell Cycle | mitosis of | 2.87E−18 | | | AURKA, BIRC5, BUB1, CASC5, CDC20, CDCA8, CDK1, | 27 |
| | tumor cell | | | | CHEK1, DLGAP5, FBXO5, KIF11, KIF4A, MAD2L1, MAD2L2, | |
| | lines | | | | PKMYT1, PLK1, PTTG1, RACGAP1, RBBP8, RMI2, | |
| | | | | | SPAG5, STIL, TBL1XR1, TCP1, TOP2A, TPX2, YWHAE | |
| Cell Cycle | mitosis of | 1.14E−17 | | | AURKA, BIRC5, BUB1, CASC5, CDC20, CDCA8, CDK1, | 23 |
| | cervical | | | | DLGAP5, FBXO5, KIF11, KIF4A, MAD2L1, PKMYT1, PLK1, | |
| | cancer cell | | | | PTTG1, RACGAP1, RMI2, SPAG5, STIL, TBL1XR1, | |
| | lines | | | | TCP1, TOP2A, TPX2 | |
| Cell Cycle | arrest in | 1.21E−17 | | | AURKB, BIRC5, BUB1, CASC5, CDC20, CDK1, CHEK1, | 22 |
| | mitosis | | | | CKAP2, FBXO5, KIF11, KIF4A, MAD2L1, MYBL2, PLK1, | |
| | | | | | RACGAP1, RMI2, SGO1, SPAG5, TBL1XR1, TCP1, TPX2, | |
| | | | | | ZWINT | |
| DNA | metabolism | 6.38E−16 | Increased | 3.131 | ABCB1, BARD1, BIRC5, BRIP1, CACYBP, CCNA2, CCNE2, | 44 |
| Replication, | of DNA | | | | CD2, CDC6, CDK1, CHEK1, CKS2, CSF1, DUT, ENO1, | |
| Recombination, | | | | | FEN1, GZMA, GZMB, HMGB1, HMGB2, HMGN1, HMGN2, | |
| and Repair | | | | | HSD17B10, IFNG, KIAA0101, KPNA2, MCM2, | |
| | | | | | MCM7, ORC1, ORC6, PIN1, PLK1, PPIB, PRDX6, PTMS, | |
| | | | | | RAN, RBPJ, RHOA, SOD1, STAT1, TFDP1, TMPO, TOP2A, | |
| | | | | | XRCC6 | |
| Cell Cycle | interphase | 2.39E−15 | Increased | 3.373 | ABCB1, AURKA, BARD1, BIRC5, BUB1, CCL3, CCNA2, | 59 |
| | | | | | CCNE2, CDC20, CDC6, CDCA2, CDK1, CDKN3, CHEK1, | |
| | | | | | CKS1B, CKS2, CLSPN, CSF1, DTL, EZH2, FBXO5, FEN1, | |
| | | | | | FKBP1A, GPI, HMGN1, ID2, IFI16, IFNG, JAK3, KIAA0101, | |
| | | | | | KIF11, LIMK1, MAD2L1, MAD2L2, MCM7, MELK, | |
| | | | | | MYBL2, NOTCH1, PIN1, PKM, PLK1, PPP5C, PTPN22, | |
| | | | | | PTTG1, RBBP8, RBCK1, RBX1, RHOA, RNASEH2B, | |
| | | | | | STAT1, STAT3, TCP1, TFDP1, TMPO, TP73, TYMS, UBL5, | |
| | | | | | UHRF1, YWHAQ | |
| Cell Death and | cell death | 3.13E−15 | | | ABCB1, ACTN4, AFAP1L2, ALDOC, ANXA5, ARL6IP1, | 174 |
| Survival | | | | | ASB2, AURKA, AURKB, BARD1, BIRC5, BUB1, CACYBP, | |
| | | | | | CALCOCO2, CALM1 (includes others), CASC5, CCL3, | |
| | | | | | CCL5, CCNA2, CD2, CD38, CD3G, CD7, CD82, CD96, | |
| | | | | | CDC20, CDC45, CDC6, CDCA2, CDK1, CDKN3, CENPF, | |
| | | | | | CFL1, CHEK1, CKAP2, CLNK, CLSPN, COPZ1, COX5A, | |
| | | | | | COX8A, CSF1, DAPK2, DDB2, DHFR, DNAJA1, | |
| | | | | | DNAJB6, DTL, DUT, EIF3I, ENO1, ENTPD1, EZH2, FANCL, | |
| | | | | | FBXO5, FDPS, FEN1, FIS1, FKBP1A, GAPDH, GPI, GZMA, | |
| | | | | | GZMB, H2AFX, HAVCR2, HCLS1, HIST1H1C, HLA- | |
| | | | | | DRB1, HMGB1, HMGB2, HMMR, HNRNPK, HPRT1, HSD17B10, | |
| | | | | | HSPA8, HSPA9, HSPD1, HSPE1, ID2, IFI16, IFNG, | |
| | | | | | IL21R, IRF9, JAK3, KIAA0101, KIF11, KIR2DL4, KLRB1, | |
| | | | | | KPNA2, LAG3, LDHA, LSP1, MAD2L1, MAD2L2, MAP4K1, | |
| | | | | | MCM2, MCM7, MELK, MKI67, MOB1A, MT2A, | |
| | | | | | MYBL2, MYO7A, NDUFS4, NEK2, NOTCH1, NUSAP1, | |
| | | | | | PARK7, PBK, PCK2, PCMT1, PIN1, PKM, PKMYT1, PLA2G16, | |
| | | | | | PLK1, PPIB, PPP2R4, PPP5C, PRDX5, PRDX6, PRKAG1, | |
| | | | | | PRKAR1A, PSMA5, PSMA6, PSMB8, PSMC1, PSME2, | |
| | | | | | PTMA, PTPN22, PTTG1, RAB27A, RACGAP1, RAN, | |
| | | | | | RANBP1, RBBP4, RBCK1, RBPJ, RBX1, RHOA, RRM1, | |
| | | | | | RRM2, SEMA7A, SGMS1, SLC25A5, SNRPB, SOD1, SPAG5, | |
| | | | | | STAT1, STAT3, STAT5A, STIL, STMN1, SUB1, TBL1XR1, | |
| | | | | | TCP1, TFDP1, TMSB10/TMSB4X, TNFRSF9, | |
| | | | | | TOP2A, TP73, TPM3, TPX2, TUBB, TYMS, UBB, UBC, UBE2C, | |
| | | | | | UBE2N, UCP2, VCAM1, WHSC1, XRCC6, YWHAE, | |
| | | | | | YWHAQ | |
| Dermatological | psoriasis | 1.13E−14 | | | CD2, CD63, CD7, CFL1, DBN1, DHFR, DTX2, EPSTI1, FABP5, | 53 |
| Diseases and | | | | | FEN1, FKBP1A, GAPDH, GBP1, GBP2, GZMB, H2AFX, | |
| Conditions | | | | | H2AFZ, HSPA8, HSPE1, IFI16, IFI35, IFNG, ITGAE, | |
| | | | | | JAK3, KIAA0101, KPNA2, MAD2L1, MKI67, NAB1, OAS2, | |
| | | | | | OASL, PARP9, PCMT1, PGAM1, PKM, PLA2G16, PSMA6, | |
| | | | | | PSMB6, PSME2, RAB27A, RAN, RANBP1, SEC61B, | |
| | | | | | SLC25A5, SQLE, STAT1, SUB1, TUBB, UBE2L6, UBE2N, | |
| | | | | | VCAM1, YWHAE, YWHAQ | |
| Cell Cycle | M phase | 1.3E−14 | | | ACTN4, ARL3, AURKA, AURKB, BIRC5, CALM1 (includes | 32 |
| | | | | | others), CDC20, CDC6, CDK1, CEP55, CFL1, CKAP2, CKS2, | |
| | | | | | FBXO5, KIF4A, LIMK1, MAD2L1, MCM4, MCM7, MOB1A, | |
| | | | | | NCAPD2, NEK2, NUSAP1, PIN1, PLK1, PRC1, PTTG1, | |
| | | | | | RACGAP1, RHOA, TOP2A, TRIP13, UBE2C | |
| Cell | morphology | 1.55E−13 | | | AURKA, BIRC5, BUB1, CDK1, FBXO5, KIF11, NUSAP1, | 13 |
| Morphology, | of mitotic | | | | ORC6, PLK1, PTTG1, RACGAP1, RAN, TPX2 | |
| Cellular | spindle | | | | | |
| Assembly and | | | | | | |
| Organization, | | | | | | |
| DNA | | | | | | |
| Replication, | | | | | | |
| Recombination, | | | | | | |
| and Repair | | | | | | |
| Cellular | cell | 1.56E−13 | Increased | 5.794 | ABCB1, ACTN4, AURKA, AURKB, BARD1, BCCIP, BIRC5, | 100 |
| Development, | proliferation | | | | BUB1, CACYBP, CCL3, CCNA2, CCNB2, CCNE2, | |
| Cellular | of tumor cell | | | | CD38, CDCA2, CDCA8, CDK1, CDKN3, CHEK1, CISD1, | |
| Growth | lines | | | | CKS1B, CMC2, COPS6, COPZ1, CSF1, DAPK2, DDB2, DLGAP5, | |
| and | | | | | DNAJB6, DTL, ENTPD1, ETV7, EZH2, FABP5, FEN1, | |
| Proliferation | | | | | GALNT2, GAPDH, GLDC, GZMB, H2AFZ, HAVCR2, | |
| | | | | | HMGB1, HMMR, HNRNPK, ID2, IFI16, IFNG, JAK3, | |
| | | | | | KIAA0101, KIF2C, KPNA2, LDHA, LIMK1, MCM2, MCM7, | |
| | | | | | MELK, MKI67, MT2A, NCAPG, NEK2, NOTCH1, PBK, | |
| | | | | | PCK2, PIN1, PKM, PLK1, PPP5C, PRKAR1A, PSMA5, | |
| | | | | | PTMA, PTPN22, PTTG1, RAN, RARRES3, RBCK1, RBX1, | |
| | | | | | RHOA, RRM1, RRM2, SGMS1, SOD1, STAT1, STAT3, | |
| | | | | | STAT5A, STMN1, SUMO3, TCP1, TMPO, TMSB10/TMSB4X, | |
| | | | | | TP73, TPX2, TUBB, TYMS, UBE2C, UHRF1, VCAM1, | |
| | | | | | WHSC1, XRCC6, YWHAQ, ZMIZ1 | |
| Cancer, | mammary | 2.02E−13 | | | ABCB1, ACP5, ACTN4, ALDOC, ANKS1B, ASF1B, ATP10D, | 119 |
| Organismal | tumor | | | | ATP5C1, ATP5J2, AURKB, BARD1, BCCIP, BIRC5, | |
| Injury and | | | | | BRIP1, CALM1 (includes others), | |
| Abnormalities, | | | | | CCL3, CCL5, CCNA2, CCNB2, CCNE2, CD3G, CDC20, | |
| Reproductive | | | | | CDC6, CDCA7, CDK1, CHEK1, CISD1, COMMD7, | |
| System Disease | | | | | CSF1, DDB2, DHFR, DIXDC1, DNAJB6, DPP3, ENO1, | |
| | | | | | ENSA, ETFA, ETFB, EZH2, FABP5, FAM179A, FARSA, | |
| | | | | | FBXO5, FDPS, FEN1, FIS1, FKBP1A, H2AFX, H2AFZ, HAPLN3, | |
| | | | | | HDLBP, HIST1H1C, HMMR, IFNG, IRF9, ITGAE, | |
| | | | | | JAK3, KIF11, KIF2C, KPNA2, LAGE3, LIMK1, LSP1, | |
| | | | | | MAST4, MCM2, MCM4, MCM6, MELK, MKI67, MT2A, | |
| | | | | | MYBL2, MYO7A, NCAPD2, NEK2, NFYC, NOTCH1, ODF2, | |
| | | | | | PGK1, PIN1, PKM, PRC1, PRDX5, PSMA5, PTPN22, | |
| | | | | | PTTG1, RANBP1, RBBP8, RBX1, RFX5, RHOA, RRM1, | |
| | | | | | RRM2, SCCPDH, SCUBE1, SGO1, SLC25A5, SOD1, SQLE, | |
| | | | | | SRGAP3, STAT1, STAT3, STMN1, TBL1XR1, TCP1, | |
| | | | | | TNFRSF9, TOP2A, TP73, TPI1, TSHZ2, TUBA1B, TUBB, | |
| | | | | | TYMS, UBB, UBE2C, UHRF1, WDR1, WHSC1, YWHAQ, | |
| | | | | | ZMIZ1 | |
| Cell Cycle | arrest in | 3.54E−13 | | | BIRC5, BUB1, CASC5, CDK1, CHEK1, FBXO5, KIF11, KIF4A, | 16 |
| | mitosis of | | | | MAD2L1, PLK1, RACGAP1, RMI2, SPAG5, TBL1XR1, | |
| | tumor cell | | | | TCP1, TPX2 | |
| | lines | | | | | |
| Cell Cycle | M phase of | 5.04E−12 | | | AURKB, BIRC5, CEP55, FBXO5, KIF4A, LIMK1, MAD2L1, | 18 |
| | tumor cell | | | | MCM7, MOB1A, NCAPD2, NEK2, PIN1, PLK1, PTTG1, | |
| | lines | | | | RACGAP1, RHOA, TOP2A, TRIP13 | |
| Cell Death and | necrosis | 5.37E−12 | | | ABCB1, AFAP1L2, ARL6IP1, AURKA, AURKB, BARD1, | 137 |
| Survival | | | | | BIRC5, BUB1, CASC5, CCL3, CCL5, CD2, CD38, CD7, | |
| | | | | | CD82, CDC20, CDC45, CDC6, CDCA2, CDK1, CHEK1, CKAP2, | |
| | | | | | CLSPN, COPZ1, COX5A, COX8A, CSF1, DAPK2, | |
| | | | | | DDB2, DHFR, DTL, DUT, EIF3I, ENO1, EZH2, FANCL, FBXO5, | |
| | | | | | FDPS, FEN1, FIS1, FKBP1A, GAPDH, GPI, GZMA, | |
| | | | | | GZMB, H2AFX, HAVCR2, HCLS1, HIST1H1C, HMGB1, | |
| | | | | | HMGB2, HMMR, HNRNPK, HPRT1, HSD17B10, HSPA8, | |
| | | | | | HSPA9, HSPD1, HSPE1, ID2, IFI16, IFNG, IL21R, IRF9, | |
| | | | | | JAK3, KIAA0101, KIF11, KPNA2, LAG3, LDHA, LSP1, | |
| | | | | | MAD2L1, MAD2L2, MAP4K1, MCM2, MCM7, MELK, | |
| | | | | | MKI67, MT2A, MYBL2, NEK2, NOTCH1, PARK7, PBK, | |
| | | | | | PCK2, PIN1, PKM, PKMYT1, PLA2G16, PLK1, PPP5C, PRDX6, | |
| | | | | | PRKAR1A, PSMA5, PSMA6, PSMB8, PSMC1, PTMA, | |
| | | | | | PTPN22, PTTG1, RACGAP1, RAN, RBBP4, RBCK1, | |
| | | | | | RBPJ, RBX1, RHOA, RRM1, RRM2, SEMA7A, SGMS1, | |
| | | | | | SLC25A5, SNRPB, SOD1, SPAG5, STAT1, STAT3, STAT5A, | |
| | | | | | STMN1, TBL1XR1, TCP1, TFDP1, TMSB10/TMSB4X, | |
| | | | | | TNFRSF9, TOP2A, TP73, TPX2, TUBB, TYMS, UBB, | |
| | | | | | UBE2C, UCP2, VCAM1, WHSC1, XRCC6, YWHAE, YWHAQ | |
| Cell Death and | apoptosis | 9.84E−12 | | | ABCB1, ACTN4, ALDOC, ANXA5, ARL6IP1, ASB2, AURKA, | 136 |
| Survival | | | | | AURKB, BARD1, BIRC5, BUB1, CASC5, CCL3, CCL5, | |
| | | | | | CCNA2, CD2, CD38, CD7, CD82, CDC20, CDC45, CDC6, | |
| | | | | | CDCA2, CDK1, CDKN3, CENPF, CFL1, CHEK1, CKAP2, | |
| | | | | | COPZ1, COX5A, COX8A, CSF1, DAPK2, DDB2, | |
| | | | | | DHFR, DNAJA1, DUT, EIF3I, ENO1, ENTPD1, EZH2, FBXO5, | |
| | | | | | FEN1, FIS1, FKBP1A, GAPDH, GPI, GZMA, GZMB, | |
| | | | | | H2AFX, HAVCR2, HCLS1, HIST1H1C, HMGB1, HMMR, | |
| | | | | | HNRNPK, HSD17B10, HSPA8, HSPA9, HSPD1, HSPE1, | |
| | | | | | ID2, IFI16, IFNG, JAK3, KIAA0101, KIF11, KPNA2, LAG3, | |
| | | | | | LDHA, LSP1, MAD2L1, MAD2L2, MAP4K1, MCM2, | |
| | | | | | MELK, MKI67, MOB1A, MT2A, MYBL2, NOTCH1, NUSAP1, | |
| | | | | | PARK7, PBK, PCMT1, PIN1, PKM, PKMYT1, PLA2G16, | |
| | | | | | PLK1, PPP2R4, PPP5C, PRDX5, PRDX6, PRKAR1A, | |
| | | | | | PSMB8, PTMA, PTPN22, PTTG1, RAB27A, RACGAP1, | |
| | | | | | RANBP1, RBBP4, RBCK1, RBPJ, RBX1, RHOA, RRM1, | |
| | | | | | RRM2, SEMA7A, SGMS1, SLC25A5, SOD1, SPAG5, STAT1, | |
| | | | | | STAT3, STAT5A, STIL, STMN1, SUB1, TBL1XR1, | |
| | | | | | TCP1, TFDP1, TMSB10/TMSB4X, TNFRSF9, TOP2A, | |
| | | | | | TP73, TPX2, TYMS, UBB, UCP2, WHSC1, XRCC6, YWHAE, | |
| | | | | | YWHAQ | |
| Cell Death and | cell death of | 1.23E−11 | | | ABCB1, ARL6IP1, AURKA, AURKB, BARD1, BIRC5, BUB1, | 93 |
| Survival | tumor cell | | | | CASC5, CD7, CD82, CDC20, CDC6, CDCA2, CDK1, | |
| | lines | | | | CHEK1, CKAP2, CLSPN, COPZ1, COX5A, COX8A, DAPK2, | |
| | | | | | DHFR, DTL, DUT, ENO1, EZH2, FANCL, FBXO5, FEN1, | |
| | | | | | FIS1, FKBP1A, GAPDH, GPI, GZMB, H2AFX, HCLS1, | |
| | | | | | HMMR, HNRNPK, HSPA8, HSPA9, HSPD1, IFI16, IFNG, | |
| | | | | | JAK3, KIF11, KPNA2, LAG3, LSP1, MAD2L1, MAP4K1, | |
| | | | | | MCM7, MELK, MT2A, MYBL2, NEK2, NOTCH1, PARK7, | |
| | | | | | PBK, PCK2, PKM, PKMYT1, PLA2G16, PLK1, PPP5C, | |
| | | | | | PRKAR1A, PTMA, PTPN22, PTTG1, RACGAP1, RBX1, | |
| | | | | | RHOA, RRM1, RRM2, SGMS1, SLC25A5, SOD1, SPAG5, | |
| | | | | | STAT1, STAT3, STAT5A, STMN1, TBL1XR1, TCP1, | |
| | | | | | TMSB10/TMSB4X, TOP2A, TP73, TPX2, TYMS, UBE2C, | |
| | | | | | UCP2, WHSC1, XRCC6, YWHAE | |
| Cancer, | female | 3.17E−11 | | | ABCB1, ACP5, AURKA, BUB1, CALCOCO2, CCNA2, CCNB2, | 36 |
| Organismal | genital tract | | | | CNB2, CDC20, CDC6, CKS1B, ENTPD1, FEN1, GZMA, | |
| Injury and | serous cancer | | | | H2AFX, HIST1H1C, HMMR, ITM2A, KIF11, KIF2C, KPNA2, | |
| Abnormalities, | | | | | MYBL2, NOTCH1, PKM, PLK1, PLPP1, PTTG1, RACGAP1, | |
| Reproductive | | | | | SEL1L3, SMC4, STAT1, TBL1XR1, TOP2A, TPX2, | |
| System Disease | | | | | TRIP13, TSHZ2, UBE2C | |
| Cell Cycle | delay in | 4.82E−11 | | | AURKA, CDC20, CDK1, DLGAP5, MAD2L1, PKMYT1, | 9 |
| | mitosis of | | | | PLK1, PTTG1, TOP2A | |
| | tumor cell | | | | | |
| | lines | | | | | |
| Cell Cycle | arrest in | 7.39E−11 | | | AURKA, BARD1, BIRC5, CCNA2, CDC6, CDCA2, CDK1, | 38 |
| | interphase | | | | CHEK1, CKS1B, CKS2, CSF1, EZH2, FBXO5, FEN1, FKBP1A, | |
| | | | | | IFNG, JAK3, KIF11, LIMK1, MAD2L1, MAD2L2, | |
| | | | | | MCM7, MELK, MYBL2, NOTCH1, PKM, PLK1, PTPN22, | |
| | | | | | RBCK1, RBX1, RHOA, STAT1, TCP1, TFDP1, TMPO, | |
| | | | | | TP73, TYMS, UBL5 | |
| Cancer, | uterine | 7.71E−11 | | | ABCB1, AURKA, BUB1, CCNA2, CCNB2, CDC20, CDC6, | 26 |
| Organismal | serous | | | | CKS1B, ENTPD1, FEN1, GZMA, HIST1H1C, HMMR, ITM2A, | |
| Injury and | papillary | | | | KIF11, KIF2C, KPNA2, MYBL2, PLK1, PTTG1, SEL1L3, | |
| Abnormalities, | cancer | | | | STAT1, TOP2A, TPX2, TRIP13, UBE2C | |
| Reproductive | | | | | | |
| System Disease | | | | | | |
| Cell Cycle | arrest in cell | 8.92E−11 | | | AURKA, AURKB, BIRC5, CCNA2, CCNE2, CDC123, CDKN3, | 33 |
| | cycle | | | | CHEK1, CKAP2, CLSPN, EZH2, FKBP1A, ID2, IFI16, | |
| | progression | | | | IFNG, IRF9, LAG3, MAD2L1, MELK, NOTCH1, PLK1, | |
| | | | | | RBX1, RHOA, RRM1, STAT1, STAT3, TCP1, TFDP1, TP73, | |
| | | | | | TYMS, USP1, XRCC6, YWHAE | |
| Cellular | alignment of | 1.19E−10 | | | AURKA, BIRC5, CCNA2, DLGAP5, KIF2C, NCAPD2, NCAPG, | 10 |
| Assembly and | chromosomes | | | | PLK1, SGO1, SMC4 | |
| Organization, | | | | | | |
| DNA | | | | | | |
| Replication, | | | | | | |
| Recombination, | | | | | | |
| and Repair | | | | | | |
| Cell Death and | apoptosis of | 1.2E−10 | | | ABCB1, AURKA, BARD1, BIRC5, BUB1, CASC5, CD7, | 77 |
| Survival | tumor cell | | | | CD82, CDC20, CDC6, CDCA2, CDK1, CHEK1, CKAP2, COPZ1, | |
| | lines | | | | COX5A, COX8A, DAPK2, DUT, ENO1, EZH2, FBXO5, | |
| | | | | | FIS1, GAPDH, GZMB, H2AFX, HCLS1, HMMR, HNRNPK, | |
| | | | | | HSPA8, HSPA9, HSPD1, IFI16, IFNG, JAK3, KIF11, | |
| | | | | | KPNA2, LAG3, LSP1, MAD2L1, MAP4K1, MELK, | |
| | | | | | MT2A, NOTCH1, PARK7, PBK, PKM, PKMYT1, PLA2G16, | |
| | | | | | PLK1, PPP5C, PRKAR1A, PTMA, PTPN22, PTTG1, RACGAP1, | |
| | | | | | RBX1, RHOA, RRM1, RRM2, SGMS1, SLC25A5, | |
| | | | | | SOD1, SPAG5, STAT1, STAT3, STAT5A, STMN1, TBL1XR1, | |
| | | | | | TCP1, TMSB10/TMSB4X, TOP2A, TP73, TPX2, | |
| | | | | | TYMS, WHSC1, YWHAE | |
| Infectious | Viral | 1.31E−10 | Increased | 3.525 | ABCB1, ACTR3, ANXA5, ARPC3, ATP5B, BST2, C14orf166, | 92 |
| Diseases | Infection | | | | CCL3, CCL5, CCNA2, CD200R1, CD38, CD63, CHMP4A, | |
| | | | | | CHORDC1, COPB1, COPZ1, COX6A1, CSF1, CXCR6, | |
| | | | | | DAPK2, DHFR, DNAJA1, DTX2, EIF3I, EXOSC10, EZH2, | |
| | | | | | FDPS, FKBP1A, GAPDH, GBP1, GPI, GZMA, HAVCR2, | |
| | | | | | HIST1H1C, HIST1H2AC, HIST1H2BK, HMGB1, HMGN2, | |
| | | | | | HNRNPK, HSPA9, HSPD1, IFI35, IFNG, IRF9, KIF11, | |
| | | | | | LIMK1, LMAN2, LSP1, MT2A, NDUFA6, NDUFS6, | |
| | | | | | NEIL3, OASL, PARP9, PBK, PDIA6, PIN1, PKMYT1, PLK1, | |
| | | | | | PPIB, PPP5C, PSMA2, PSMA5, PSMB6, PSMB9, PSMC3, | |
| | | | | | PSME2, PTTG1, RACGAP1, RAN, RANBP1, RARRES3, | |
| | | | | | RBPJ, RHOA, RRM2, SAMSN1, STAT1, STAT3, STAT5A, | |
| | | | | | SUB1, TALDO1, TMPO, TNFRSF9, TOP2A, TP73, | |
| | | | | | TRAFD1, TUBB, TXNL4A, TYMS, UBE2C, UBE2L6 | |
| DNA | DNA | 1.34E−10 | | | BATF, BIRC5, BRIP1, CDC6, CHEK1, GAPDH, GZMA, | 24 |
| Replication, | damage | | | | H2AFX, HLA- | |
| Recombination, | | | | | DRB1, HLTF, IFNG, MAD2L2, PARK7, PBK, PLK1, RBX1, | |
| and Repair | | | | | RHOA, RNASEH2B, RRM2, SOD1, STAT5A, TOP2A, | |
| | | | | | TP73, YWHAE | |
| Cell Cycle | arrest in | 1.4E−10 | | | BIRC5, BUB1, CASC5, FBXO5, KIF11, KIF4A, PLK1, RACGAP1, | 13 |
| | mitosis of | | | | RMI2, SPAG5, TBL1XR1, TCP1, TPX2 | |
| | cervical | | | | | |
| | cancer cell | | | | | |
| | lines | | | | | |
| Cell Death and | cytotoxicity | 1.6E−10 | Increased | 2.208 | CALM1 (includes others), | 26 |
| Survival | | | | | CCL5, CD2, CD38, CD96, CHEK1, DHFR, FKBP1A, | |
| | | | | | GZMA, GZMB, HAVCR2, HLA-DRB1, | |
| | | | | | HPRT1, HSPA8, IFNG, IL21R, KIR2DL4, KLRB1, MYBL2, | |
| | | | | | NDUFS4, NOTCH1, PPIB, RAB27A, SOD1, STAT1, TOP2A | |
| Cell Cycle | delay in | 3.44E−10 | | | AURKA, CDC20, DLGAP5, MAD2L1, PKMYT1, PLK1, | 8 |
| | mitosis of | | | | PTTG1, TOP2A | |
| | cervical | | | | | |
| | cancer cell | | | | | |
| | lines | | | | | |
| Immunological | systemic | 3.83E−10 | | | ABCB1, ALOX5AP, BIRC5, CCL3, CCL5, CD38, CD3D, | 68 |
| Disease | autoimmune | | | | CD3G, CD7, CD96, CLEC2B, CSF1, CXCL13, CXCR6, DHFR, | |
| | syndrome | | | | ENO1, FDPS, FKBP1A, GALNT2, GBP2, GBP4, GZMA, | |
| | | | | | GZMB, HAVCR2, HCLS1, HIST1H2AC, HLA- | |
| | | | | | DRA, HLA-DRB1, HMGB1, HPRT1, HSPA8, HSPD1, IFI16, IFNG, | |
| | | | | | IL21R, ITGAE, JAK3, KLRB1, LAP3, LDHB, LSM2, MT2A, | |
| | | | | | NONO, OASL, PGK1, PPP1R7, PRDX5, PSMB8, PSMB9, | |
| | | | | | PTMA, PTPN22, RAB27A, SCUBE1, SQLE, SRP14, STAT1, | |
| | | | | | STAT3, STAT5A, TNFRSF9, TNFSF4, TRAFD1, TUBB, | |
| | | | | | TYMS, UBB, UBE2L6, UCP2, UQCC2, VCAM1 | |
| Neurological | neuromuscular | 5.99E−10 | | | ABCB1, ARL3, ATP5C1, ATP5L, CAPZB, CCL5, CD38, | 59 |
| Disease, | disease | | | | COPE, COX7B, DHFR, DNAJA1, DNAJB11, DNAJB6, EPSTI1, | |
| Skeletal | | | | | PSTI1, ETFB, FKBP1A, GAPDH, GBP1, GPI, HAVCR2, HLA- | |
| and Muscular | | | | | DRA, HLA-DRB1, HMGB2, HMGN1, HSPA8, IFNG, LDHA, LDHB, | |
| Disorders | | | | | LIMK1, MT2A, NDUFB6, PARK7, PCMT1, PGK1, PIN1, | |
| | | | | | PKM, PRDX6, PSMA2, PSMB6, PSMB8, PSMB9, PSMC1, | |
| | | | | | PSME1, RAN, RANBP1, RARRES3, RRM1, RRM2, SOD1, | |
| | | | | | STMN1, SUB1, SUMO3, TOP2A, TPI1, TPM3, TUBA1B, | |
| | | | | | UBB, UQCR10, XRCC6 | |
| Cell Cycle, | segregation | 6.23E−10 | | | CCNA2, MAD2L1, NCAPD2, NEK2, NUSAP1, PLK1, SMC4, | 9 |
| Cellular | of sister | | | | SPAG5, ZWINT | |
| Assembly and | chromatids | | | | | |
| Organization, | | | | | | |
| DNA | | | | | | |
| Replication, | | | | | | |
| Recombination, | | | | | | |
| and Repair | | | | | | |
| Cell Cycle | anaphase | 6.39E−10 | | | CDC20, CKAP2, LIMK1, MAD2L1, MCM4, NCAPD2, TOP2A, | 8 |
| | | | | | TRIP13 | |
| Nucleic Acid | metabolism | 6.6E−10 | Increased | 3.396 | ATP5B, ATP5C1, ATP5J2, ATP5L, CDK1, COX8A, ENTPD1, | 19 |
| Metabolism, | of nucleoside | | | | HMGB1, HSPA8, HSPD1, NDUFS6, PKM, SGMS1, | |
| Small Molecule | triphosphate | | | | SLC25A5, SOD1, STAT3, TYMS, UCP2, UQCC2 | |
| Biochemistry | | | | | | |
| Cell Cycle, | check | 8.51E−10 | | | BUB1, CCNB2, CCNE2, CDC20, CDC6, CHEK1, CKS1B, | 16 |
| DNA | point control | | | | CKS2, HMGN1, MAD2L1, MCM7, PIN1, PLK1, PTTG1, | |
| Replication, | | | | | RBBP8, ZWINT | |
| Recombination, | | | | | | |
| and Repair | | | | | | |
| Cancer, | breast cancer | 1.04E−09 | | | ABCB1, ACP5, ACTN4, ALDOC, ANKS1B, ASF1B, ATP10D, | 104 |
| Organismal | | | | | ATP5C1, ATP5J2, AURKB, BARD1, BCCIP, BIRC5, | |
| Injury and | | | | | BRIP1, CALM1 (includes others), | |
| Abnormalities, | | | | | CCL3, CD3G, CDC20, CDCA7, CDK1, CHEK1, COMMD7, | |
| Reproductive | | | | | DHFR, DIXDC1, DNAJB6, DPP3, ENO1, ENSA, | |
| System Disease | | | | | ETFA, ETFB, EZH2, FABP5, FAM179A, FARSA, FDPS, | |
| | | | | | FIS1, FKBP1A, H2AFX, H2AFZ, HAPLN3, HIST1H1C, | |
| | | | | | HMMR, IRF9, ITGAE, JAK3, KIF11, KPNA2, LAGE3, LIMK1, | |
| | | | | | LSP1, MAST4, MCM6, MELK, MKI67, MT2A, MYBL2, | |
| | | | | | MYO7A, NCAPD2, NEK2, NFYC, NOTCH1, ODF2, | |
| | | | | | PGK1, PIN1, PKM, PRC1, PRDX5, PSMA5, PTPN22, PTTG1, | |
| | | | | | RANBP1, RBBP8, RBX1, RFX5, RHOA, RRM1, RRM2, | |
| | | | | | SCCPDH, SCUBE1, SGO1, SLC25A5, SOD1, SQLE, SRGAP3, | |
| | | | | | STAT1, STAT3, STMN1, TBL1XR1, TCP1, TNFRSF9, | |
| | | | | | TOP2A, TP73, TPI1, TSHZ2, TUBA1B, TUBB, TYMS, | |
| | | | | | UBB, UBE2C, UHRF1, WDR1, WHSC1, YWHAQ, ZMIZ1 | |
| Cancer, | lymphocytic | 1.85E−09 | | | AFAP1L2, AURKA, AURKB, BIRC5, BST2, CCNA2, | 54 |
| Hematological | cancer | | | | CDC123, CDCA2, CXCL13, DDB2, DHFR, EZH2, FEN1, | |
| Disease, | | | | | FKBP1A, H2AFX, HIST1H1C, HIST1H2BK, HLA- | |
| Immunological | | | | | DRB1, HMGB1, ID2, IFNG, JAK3, KIF11, LIMK1, LSM2, | |
| Disease, | | | | | MAP4K1, MCM5, MKI67, NOTCH1, PAG1, PSMB9, RAN, | |
| Organismal | | | | | RANBP1, RBBP8, RHOA, RRM1, RRM2, SFXN1, SNRPB, | |
| Injury and | | | | | SP140, STAT1, STAT3, STAT5A, STMN1, TNFRSF9, | |
| Abnormalities | | | | | TOP2A, TP73, TPX2, TRIP12, TSHZ2, TYMS, UBE2A, | |
| | | | | | WHSC1, XRCC6 | |
| Cell Cycle | ploidy | 2.11E−09 | | | AURKA, AURKB, BIRC5, BUB1, CCNA2, CDC20, CKAP2, | 20 |
| | | | | | CKS1B, CKS2, FBXO5, KIF11, MAD2L1, NEK2, PIN1, | |
| | | | | | PLK1, PTTG1, STAT1, STMN1, TOP2A, TPX2 | |
| Connective | Rheumatic | 2.46E−09 | | | ABCB1, ALOX5AP, BARD1, BIRC5, CCL3, CCL5, CD200R1, | 67 |
| Tissue | Disease | | | | CD3D, CLEC2B, CSF1, CXCL13, DHFR, ENO1, FDPS, | |
| Disorders, | | | | | FKBP1A, GALNT2, GBP2, GPI, GZMA, GZMB, HAVCR2, | |
| Inflammatory | | | | | HCLS1, HIST1H2AC, HLA-DRA, HLA- | |
| Disease, | | | | | DRB1, HLTF, HMGB1, HMMR, HPRT1, HSPA8, HSPD1, | |
| Skeletal | | | | | HSPE1, IFNG, IL21R, JAK3, KLRB1, LAP3, LDHB, MAP4K1, | |
| and Muscular | | | | | MCM5, MT2A, NONO, OASL, PGK1, PPP1R7, PRDX5, | |
| Disorders | | | | | PSMB8, PSMB9, PTMA, PTPN22, RAB27A, RBPJ, RHOA, | |
| | | | | | SCUBE1, SQLE, SRP14, STAT1, STAT3, STAT5A, | |
| | | | | | TNFRSF9, TNFSF4, TP73, TYMS, UBB, UBE2L6, UQCC2, | |
| | | | | | VCAM1 | |
| Cancer, | plasma cell | 2.55E−09 | | | ACP5, AKAP5, ANXA5, BIRC5, CCL3, CCL5, CD38, CDC20, | 31 |
| Hematological | dyscrasia | | | | CDK1, DHFR, EPSTI1, FDPS, FEN1, FKBP1A, IFNG, | |
| Disease, | | | | | KIF2C, KPNA2, MKI67, NONO, PDIA6, PMF1/PMF1- | |
| Immunological | | | | | BGLAP, PSMA2, PSMB8, PSMB9, RRM1, RRM2, STAT3, | |
| Disease, | | | | | TFDP1, TOP2A, XRCC6, YWHAE | |
| Organismal | | | | | | |
| Injury and | | | | | | |
| Abnormalities | | | | | | |
| Cell Cycle | formation of | 3.45E−09 | | | AURKA, BIRC5, CKAP2, FBXO5, KIF11, KIF2C, KIF4A, | 13 |
| | mitotic | | | | NEK2, PLK1, RAN, STMN1, TPX2, TUBB | |
| | spindle | | | | | |
| Cell | morphology | 3.58E−09 | | | AURKA, BIRC5, BUB1, CDK1, FBXO5, KIF11, KIF2C, KIF4A, | 17 |
| Morphology, | of | | | | LIMK1, NUSAP1, ORC6, PLK1, PRKAR1A, PTTG1, | |
| Cellular | cytoskeleton | | | | RACGAP1, RAN, TPX2 | |
| Assembly and | | | | | | |
| Organization | | | | | | |
| Gene | expression of | 4.52E−09 | Increased | 2.11 | ACTR3, AFAP1L2, AKAP5, ASPM, AURKB, BATF, BIRC5, | 110 |
| Expression | RNA | | | | BRIP1, BST2, C14orf166, CCL3, CCL5, CCNA2, CD38, | |
| | | | | | CD3D, CD82, CDK1, CENPF, CKAP2, CKS1B, CKS2, COMMD7, | |
| | | | | | COPZ1, CSF1, DDB2, DIXDC1, DNAJB6, DRAP1, | |
| | | | | | EIF3I, ENO1, ETV7, EZH2, FKBP1A, GAPDH, GBP2, | |
| | | | | | H2AFX, H2AFZ, HAVCR2, HCLS1, HIST1H1B, HIST1H1C, | |
| | | | | | HLTF, HMGB1, HMGB2, HMGN1, HMGN2, HNRNPK, | |
| | | | | | HSPA8, ID2, IFI16, IFNG, IRF9, JAK3, KPNA2, MAD2L2, | |
| | | | | | MCM7, MELK, MRPL51, MYBL2, NAB1, NDFIP2, NFYC, | |
| | | | | | NONO, NOTCH1, PARK7, PIN1, PLK1, PMF1/PMF1- | |
| | | | | | BGLAP, POLR2G, PPP2R5D, PPP5C, PRKAG1, PRKAR1A, | |
| | | | | | PSMC3, PTMA, PTMS, PTPN22, PTPN7, PTTG1, RAB27A, | |
| | | | | | RAB3GAP1, RARRES3, RBBP8, RBCK1, RBPJ, RBX1, | |
| | | | | | RFX5, RHOA, RNASEH2B, SGMS1, SP140, STAT1, | |
| | | | | | STAT3, STAT5A, SUB1, TBL1XR1, TFDP1, TMPO, TNFSF4, | |
| | | | | | TOP2A, TOX2, TP73, TRIP13, UHRF1, VCAM1, WARS, | |
| | | | | | WHSC1, XRCC6, YWHAQ, ZMIZ1 | |
| Connective | rheumatoid | 4.71E−09 | | | ABCB1, BIRC5, CCL3, CCL5, CD3D, CLEC2B, CSF1, CXCL13, | 48 |
| Tissue | arthritis | | | | DHFR, ENO1, FDPS, FKBP1A, GALNT2, GBP2, GZMA, | |
| Disorders, | | | | | GZMB, HAVCR2, HCLS1, HIST1H2AC, HLA- | |
| Immunological | | | | | DRA, HLA-DRB1, HMGB1, HPRT1, HSPA8, HSPD1, IFNG, JAK3, | |
| Disease, | | | | | LAP3, LDHB, NONO, PGK1, PPP1R7, PRDX5, PSMB8, PSMB9, | |
| Inflammatory | | | | | PTMA, PTPN22, SQLE, SRP14, STAT1, STAT3, STAT5A, | |
| Disease, | | | | | TNFSF4, TYMS, UBB , UBE2L6, UQCC2, VCAM1 | |
| Inflammatory | | | | | | |
| Response, | | | | | | |
| Skeletal and | | | | | | |
| Muscular | | | | | | |
| Disorders | | | | | | |
| Cell Death and | cell survival | 5.63E−09 | Increased | 5.356 | ABCB1, AFAP1L2, AKAP5, ANKS1B, ANXA5, AURKA, | 79 |
| Survival | | | | | AURKB, BARD1, BIRC5, BRIP1, CCL3, CCL5, CCNA2, | |
| | | | | | CD38, CD7, CD82, CDK1, CHEK1, CSF1, DNAJB6, DPP3, | |
| | | | | | ENO1, FBXO5, GZMB, H2AFX, HMGB1, HMGB2, HMGN1, | |
| | | | | | HNRNPK, HSD17B10, HSPD1, ID2, IFNG, IL21R, IRF9, | |
| | | | | | JAK3, KIF11, LDHA, MAD2L1, MAD2L2, MYBL2, | |
| | | | | | NOTCH1, PARK7, PBK, PIN1, PKM, PLK1, PLPP1, PPIB, | |
| | | | | | PPM1M, PRDX6, PRKAR1A, PSMA6, PSMC3, PTPN22, | |
| | | | | | PTPN7, RARRES3, RBBP4, RHOA, RRM1, RRM2, SHFM1, | |
| | | | | | SNRPE, SOD1, STAT1, STAT3, STAT5A, STMN1, TCP1, | |
| | | | | | TNFRSF9, TOP2A, TP73, TUBB, TYMS, UCP2, UHRF1, | |
| | | | | | VCAM1, WHSC1, XRCC6 | |
| Cell Cycle | delay in | 6.1E−09 | | | BIRC5, CDC20, CKAP2, LIMK1, MAD2L1, PLK1, RACGAP1, | 9 |
| | initiation of | | | | TOP2A, TRIP13 | |
| | M phase | | | | | |
| Cancer, | benign | 6.11E−09 | | | ABCB1, ACP5, ALDOC, ANXA5, AURKA, AURKB, BIRC5, | 65 |
| Organismal | neoplasia | | | | BRIP1, BUB1, C9orf16, CALCOCO2, CASC5, CCNB2, | |
| Injury and | | | | | CCNE2, CDK1, CSF1, DBN1, DDB2, DUT, EZH2, FABP5, | |
| Abnormalities | | | | | FANCI, FKBP1A, H2AFX, HIST1H1C, HMGB1, HMGB2, | |
| | | | | | HMGN1, HMMR, HSD17B10, IFNG, JAK3 , KIF11, | |
| | | | | | LSP1, MKI67, MTHFD2, MYBL2, MYL6, NOTCH1, NUSAP1, | |
| | | | | | PAG1, PARK7, PKM, PLPP1, PPA1, PRDX6, PRKAR1A, | |
| | | | | | PSMA6, PTTG1, RACGAP1, RBBP8, RRM2, SMC4, | |
| | | | | | SRGAP3, STAT1, STAT3, STIL, TBL1XR1, TFDP1, TNFRSF9, | |
| | | | | | TOP2A, TP73, TSHZ2, UQCRH, VCAM1 | |

TABLE 10
Disease-specific survival in NSCLC patients based on CD8a and CD103
density in tumors.

| | | |
|---|---|---|
| 1 = Adenocarcinoma | 1 = male | 1 = dead |
| 2 = Squamous carcinoma | 2 = female | 2 = alive |

| | |
|---|---|
| 1 = low | 1 = low |
| 2 = intermediate | 2 = intermediate |
| 3 = high | 3 = high |

| Patient | Tumor | | | Disease-free | Tumor | | |
|---|---|---|---|---|---|---|---|
| ID | histology | Gender | Dead/Alive | survival | Stage | CD8a | CD103 |
| 1 | 1 | 1 | 1 | 31 | IV | NA | 1 |
| 2 | 1 | 2 | 2 | 32 | IA | 2 | 1 |
| 3 | 1 | 1 | 2 | 38 | IIB | 1 | 1 |
| 4 | 1 | 1 | 1 | 39 | IA | 1 | 1 |
| 5 | 1 | 2 | 2 | 42 | IB | 1 | 1 |
| 6 | 1 | 1 | 1 | 48 | IB | 1 | 1 |
| 7 | 1 | 2 | 1 | 50 | IIIB | 1 | 1 |
| 8 | 1 | 1 | 1 | 32 | IIB | 2 | 2 |
| 9 | 1 | 1 | 1 | 34 | IIIA | 1 | 2 |
| 10 | 1 | 2 | 2 | 35 | IIB | 2 | 2 |
| 11 | 1 | 2 | 1 | 35 | NA | 2 | 2 |
| 12 | 1 | 1 | 1 | 81 | IA | 1 | 1 |
| 13 | 1 | 2 | 2 | 38 | IB | 2 | 2 |
| 14 | 1 | 1 | 1 | 89 | IIIB | 1 | 1 |
| 15 | 1 | 1 | 1 | 89 | IIB | 1 | 1 |
| 16 | 1 | 1 | 1 | 44 | NA | 2 | 2 |
| 17 | 1 | 2 | 1 | 93 | IIA | 1 | 1 |
| 18 | 1 | 2 | 1 | 115 | IV | 1 | 1 |
| 19 | 1 | 1 | 1 | 51 | IIB | 2 | 2 |
| 20 | 1 | 1 | 1 | 52 | NA | 2 | 2 |
| 21 | 1 | 1 | 2 | 56 | IIB | 2 | 2 |
| 22 | 1 | 2 | 1 | 62 | IB | 2 | 2 |
| 23 | 1 | 1 | 1 | 128 | IA | 1 | 1 |
| 24 | 1 | 2 | 2 | 71 | IIA | 3 | 2 |
| 25 | 1 | 2 | 1 | 139 | IIA | 2 | 1 |
| 26 | 1 | 2 | 1 | 153 | IIIA | 2 | 1 |
| 27 | 1 | 2 | 1 | 174 | IIB | 2 | 1 |
| 28 | 1 | 2 | 1 | 206 | IIA | 2 | 1 |
| 29 | 1 | 1 | 1 | 96 | IB | 1 | NA |
| 30 | 1 | 2 | 1 | 114 | IIIA | 2 | 2 |
| 31 | 2 | 1 | 1 | 145 | NA | 1 | 1 |
| 32 | 1 | 1 | 1 | 209 | IIIB | 1 | 1 |
| 33 | 1 | 2 | 1 | 248 | IV | 1 | 1 |
| 34 | 1 | 2 | 1 | 130 | IIA | NA | NA |
| 35 | 1 | 1 | 1 | 248 | IB | 1 | 1 |
| 36 | 1 | 2 | 1 | 140 | IA | 1 | 2 |
| 37 | 1 | 1 | 1 | 144 | NA | 1 | NA |
| 38 | 1 | 1 | 1 | 253 | IB | 3 | 1 |
| 39 | 2 | 2 | 2 | 2443 | NA | 1 | NA |
| 40 | 1 | 2 | 1 | 257 | IV | 1 | 1 |
| 41 | 1 | 2 | 1 | 287 | IIIA | 1 | 1 |
| 42 | 1 | 1 | 1 | 151 | IIB | 1 | 2 |
| 43 | 1 | 2 | 1 | 290 | IIA | 1 | 1 |
| 44 | 1 | 2 | 1 | 299 | IB | 1 | 1 |
| 45 | 1 | 1 | 1 | 154 | IB | 2 | 2 |
| 46 | 1 | 2 | 1 | 161 | IA | 2 | 2 |
| 47 | 1 | 2 | 1 | 299 | IIA | 1 | 1 |
| 48 | 1 | 2 | 1 | 327 | IIIA | 1 | 1 |
| 49 | 1 | 1 | 1 | 337 | NA | 1 | 1 |
| 50 | 1 | 1 | 1 | 342 | IV | 1 | 1 |
| 51 | 1 | 1 | 1 | 345 | IIIA | 1 | 1 |
| 52 | 1 | 2 | 1 | 222 | IIA | 1 | 2 |
| 53 | 1 | 2 | 1 | 226 | IIIB | 2 | 2 |
| 54 | 1 | 2 | 1 | 237 | IA | 3 | 2 |
| 55 | 1 | 1 | 1 | 237 | IIB | 2 | 2 |
| 56 | 1 | 1 | 1 | 357 | IV | 1 | 1 |
| 57 | 1 | 2 | 1 | 368 | IIIA | 1 | 1 |
| 58 | 1 | 1 | 1 | 371 | IA | 1 | 1 |
| 59 | 1 | 2 | 1 | 393 | IIB | 1 | 1 |
| 60 | 1 | 1 | 1 | 394 | IIB | 1 | 1 |
| 61 | 1 | 1 | 1 | 275 | NA | 2 | 2 |
| 62 | 1 | 2 | 1 | 411 | IB | 1 | 1 |
| 63 | 1 | 2 | 1 | 423 | IA | 2 | 1 |
| 64 | 1 | 1 | 1 | 431 | NA | 1 | 1 |
| 65 | 1 | 1 | 1 | 460 | IV | 2 | 1 |
| 66 | 1 | 2 | 1 | 289 | IV | 3 | 2 |
| 67 | 1 | 1 | 1 | 467 | NA | 1 | 1 |
| 68 | 1 | 1 | 1 | 290 | IV | 2 | 2 |
| 69 | 1 | 1 | 1 | 292 | IV | 2 | 2 |
| 70 | 1 | 1 | 1 | 470 | IV | NA | 1 |
| 71 | 1 | 2 | 1 | 473 | IB | 1 | 1 |
| 72 | 1 | 2 | 1 | 307 | NA | NA | NA |
| 73 | 1 | 1 | 1 | 315 | NA | 2 | 2 |
| 74 | 1 | 1 | 1 | 324 | IIIB | 2 | 2 |
| 75 | 1 | 1 | 1 | 473 | IIIB | 1 | 1 |
| 76 | 1 | 1 | 1 | 480 | IB | 1 | 1 |
| 77 | 1 | 2 | 1 | 483 | IB | 1 | 1 |
| 78 | 1 | 1 | 1 | 490 | NA | 1 | 1 |
| 79 | 1 | 1 | 1 | 359 | IIIB/IV | 1 | 2 |
| 80 | 1 | 1 | 1 | 493 | IIB | 1 | 1 |
| 81 | 1 | 2 | 1 | 494 | IIB | 1 | 1 |
| 82 | 1 | 2 | 1 | 538 | IIIA | 1 | 1 |
| 83 | 1 | 2 | 1 | 543 | IV | 2 | 1 |
| 84 | 1 | 2 | 1 | 553 | IIIB | 1 | 1 |
| 85 | 1 | 1 | 1 | 569 | IIIA | 1 | 1 |
| 86 | 1 | 2 | 1 | 593 | IIIA | 2 | 1 |
| 87 | 1 | 1 | 1 | 610 | IV | 1 | 1 |
| 88 | 1 | 2 | 1 | 402 | IA | 2 | 2 |
| 89 | 1 | 1 | 1 | 614 | IV | 1 | 1 |
| 90 | 1 | 1 | 1 | 615 | IB | 1 | 1 |
| 91 | 1 | 1 | 2 | 416 | IA | 1 | 2 |
| 92 | 1 | 2 | 1 | 417 | IV | 2 | 2 |
| 93 | 1 | 1 | 1 | 638 | IIIA | 1 | 1 |
| 94 | 1 | 1 | 2 | 652 | IIB | NA | 1 |
| 95 | 1 | 2 | 1 | 663 | IIIA | 1 | 1 |
| 96 | 1 | 2 | 1 | 664 | IB | 1 | 1 |
| 97 | 1 | 1 | 1 | 696 | IV | 1 | 1 |
| 98 | 1 | 2 | 1 | 708 | IV | 2 | 1 |
| 99 | 1 | 1 | 1 | 709 | IV | 2 | 1 |
| 100 | 1 | 1 | 1 | 472 | IIIA | 3 | 2 |
| 101 | 1 | 2 | 1 | 721 | IA | 1 | 1 |
| 102 | 1 | 2 | 1 | 774 | IIIA | 1 | 1 |
| 103 | 1 | 2 | 1 | 808 | IV | 2 | 1 |
| 104 | 1 | 2 | 1 | 814 | IIIA | 1 | 1 |
| 105 | 2 | 2 | 2 | 1874 | NA | 2 | 2 |
| 106 | 1 | 1 | 1 | 814 | IIIA | 2 | 1 |
| 107 | 1 | 1 | 1 | 489 | IA | 2 | 2 |
| 108 | 1 | 2 | 1 | 834 | IIB | 2 | 1 |
| 109 | 1 | 2 | 1 | 862 | NA | 3 | 1 |
| 110 | 1 | 2 | 1 | 863 | IB | 1 | 1 |
| 111 | 1 | 2 | 2 | 926 | IA | 1 | 1 |
| 112 | 1 | 1 | 1 | 496 | IB | 1 | 2 |
| 113 | 1 | 2 | 1 | 497 | IIB | 2 | 2 |
| 114 | 1 | 1 | 1 | 511 | IA | 2 | 2 |
| 115 | 2 | 1 | 2 | 1680 | NA | 2 | 1 |
| 116 | 1 | 1 | 1 | 931 | IIIA | 1 | 1 |
| 117 | 1 | 1 | 1 | 540 | IIIA | 1 | 2 |
| 118 | 1 | 1 | 2 | 933 | IA | 2 | 1 |
| 119 | 1 | 1 | 1 | 544 | IB | 2 | 2 |
| 120 | 1 | 2 | 1 | 948 | IA | 1 | 1 |
| 121 | 1 | 1 | 2 | 950 | IIB | 1 | 1 |
| 122 | 1 | 2 | 2 | 951 | IIB | 2 | 1 |
| 123 | 1 | 2 | 1 | 969 | IA | 1 | 1 |
| 124 | 1 | 2 | 2 | 971 | IA | 1 | 1 |
| 125 | 1 | 1 | 2 | 973 | IIIA | 2 | 1 |
| 126 | 1 | 2 | 2 | 973 | IB | 2 | 1 |
| 127 | 1 | 1 | 1 | 589 | IIA | 2 | 2 |
| 128 | 1 | 2 | 2 | 973 | IA | 2 | 1 |
| 129 | 1 | 2 | 1 | 975 | IIA | 1 | 1 |
| 130 | 1 | 2 | 2 | 979 | IA | 1 | 1 |
| 131 | 1 | 2 | 2 | 986 | IIIA | 1 | 1 |
| 132 | 1 | 1 | 1 | 611 | IA | 1 | 2 |
| 133 | 1 | 2 | 1 | 996 | IV | 1 | 1 |
| 134 | 2 | 1 | 2 | 1554 | NA | 1 | 1 |
| 135 | 1 | 1 | 2 | 1006 | IIB | 1 | 1 |
| 136 | 1 | 2 | 2 | 1016 | IIB | 1 | 1 |
| 137 | 1 | 1 | 1 | 618 | IIB | 2 | 2 |
| 138 | 1 | 1 | 1 | 1019 | IIA | 2 | 1 |
| 139 | 1 | 1 | 1 | 1020 | IIIA | 1 | 1 |
| 140 | 1 | 1 | 1 | 639 | IB | 1 | 2 |
| 141 | 1 | 2 | 2 | 1027 | IIIA | 1 | 1 |
| 142 | 1 | 2 | 2 | 1028 | IIB | 1 | 1 |
| 143 | 1 | 2 | 2 | 1049 | IIA | 1 | 1 |
| 144 | 1 | 1 | 2 | 1058 | IA | 1 | 1 |
| 145 | 1 | 2 | 2 | 1065 | IV | 1 | 1 |
| 146 | 1 | 2 | 1 | 1068 | IIB | 2 | 1 |
| 147 | 2 | 1 | 1 | 930 | IIIA | 2 | 2 |
| 148 | 1 | 1 | 2 | 1070 | IIB | 2 | 1 |
| 149 | 1 | 1 | 1 | 695 | IIB | NA | NA |
| 150 | 1 | 2 | 2 | 1071 | IA | 1 | 1 |
| 151 | 1 | 1 | 1 | 1073 | IIIB | 1 | 1 |
| 152 | 1 | 1 | 1 | 709 | IB | 2 | NA |
| 153 | 1 | 1 | 2 | 1077 | IA | 2 | 1 |
| 154 | 1 | 1 | 2 | 1078 | NA | 3 | 1 |
| 155 | 1 | 2 | 1 | 719 | IB | 2 | 2 |
| 156 | 1 | 1 | 1 | 1080 | IIIA | 1 | 1 |
| 157 | 1 | 1 | 1 | 730 | IV | 1 | 2 |
| 158 | 1 | 1 | 2 | 750 | IB | 2 | 2 |
| 159 | 1 | 1 | 1 | 752 | IIB | 2 | 2 |
| 160 | 1 | 2 | 2 | 1104 | IA | 1 | 1 |
| 161 | 1 | 1 | 2 | 1107 | IA | 1 | 1 |
| 162 | 1 | 2 | 2 | 1114 | IIB | 1 | 1 |
| 163 | 1 | 2 | 2 | 1128 | IB | 3 | 1 |
| 164 | 1 | 2 | 2 | 1139 | IA | 1 | 1 |
| 165 | 1 | 1 | 1 | 1161 | IB | 1 | 1 |
| 166 | 1 | 2 | 2 | 1171 | IIB | 2 | 1 |
| 167 | 1 | 2 | 2 | 1209 | IV | 2 | 1 |
| 168 | 1 | 2 | 1 | 847 | IIIA | 1 | 2 |
| 169 | 1 | 2 | 2 | 1210 | IIB | 1 | 1 |
| 170 | 1 | 1 | 2 | 1213 | IA | 1 | 1 |
| 171 | 1 | 1 | 2 | 1218 | IB | 1 | 1 |
| 172 | 1 | 1 | 1 | 895 | IIIB | 2 | 2 |
| 173 | 1 | 1 | 2 | 1219 | IIA | 1 | 1 |
| 174 | 1 | 1 | 1 | 908 | IB | 2 | 2 |
| 175 | 1 | 2 | 1 | 911 | IIIA | 3 | 2 |
| 176 | 1 | 2 | 2 | 1220 | IIA | 1 | 1 |
| 177 | 1 | 1 | 2 | 1223 | IIIA | 1 | 1 |
| 178 | 1 | 1 | 2 | 1227 | IIB | 1 | 1 |
| 179 | 1 | 1 | 2 | 1240 | IIA | 2 | 1 |
| 180 | 1 | 2 | 2 | 943 | IA | 2 | 2 |
| 181 | 1 | 1 | 2 | 944 | IA | 3 | 2 |
| 182 | 1 | 1 | 2 | 947 | IB | 2 | 2 |
| 183 | 1 | 1 | 2 | 1240 | NA | NA | 1 |
| 184 | 1 | 1 | 2 | 1248 | IB | 1 | 1 |
| 185 | 1 | 2 | 2 | 1254 | IA | 1 | 1 |
| 186 | 1 | 1 | 1 | 1261 | IB | NA | 1 |
| 187 | 1 | 2 | 2 | 953 | IIA | 2 | 2 |
| 188 | 1 | 2 | 2 | 1273 | IA | 1 | 1 |
| 189 | 1 | 1 | 1 | 1282 | IIA | 1 | 1 |
| 190 | 1 | 2 | 2 | 1300 | IB | 2 | 1 |
| 191 | 1 | 2 | 2 | 1324 | IIIA | 2 | 1 |
| 192 | 1 | 2 | 1 | 1325 | IB | 1 | 1 |
| 193 | 1 | 1 | 2 | 1325 | IIIA | 1 | 1 |
| 194 | 1 | 2 | 2 | 978 | IIA | 2 | 2 |
| 195 | 1 | 2 | 1 | 979 | IV | 3 | 2 |
| 196 | 1 | 2 | 1 | 1326 | IIIB | 1 | 1 |
| 197 | 1 | 1 | 2 | 1332 | IV | 1 | 1 |
| 198 | 1 | 2 | 2 | 1342 | IIB | 2 | 1 |
| 199 | 1 | 1 | 1 | 996 | IIA | 2 | 2 |
| 200 | 1 | 2 | 2 | 1002 | IA | 2 | 2 |
| 201 | 1 | 1 | 2 | 1345 | NA | 1 | 1 |
| 202 | 1 | 2 | 2 | 1013 | IA | 2 | 2 |
| 203 | 1 | 2 | 1 | 1016 | IIA | 2 | 2 |
| 204 | 1 | 2 | 1 | 1360 | IB | 1 | 1 |
| 205 | 1 | 2 | 1 | 1386 | IIB | 1 | 1 |
| 206 | 1 | 1 | 2 | 1387 | IB | 1 | 1 |
| 207 | 1 | 2 | 2 | 1399 | IA | 1 | 1 |
| 208 | 1 | 2 | 2 | 1436 | IIIA | 1 | 1 |
| 209 | 1 | 2 | 1 | 1028 | IV | 1 | 2 |
| 210 | 1 | 1 | 2 | 1443 | IIB | 2 | 1 |
| 211 | 1 | 1 | 2 | 1475 | IA | 1 | 1 |
| 212 | 1 | 1 | 2 | 1034 | IA | 2 | 2 |
| 213 | 1 | 1 | 2 | 1476 | IB | 1 | 1 |
| 214 | 1 | 2 | 1 | 1482 | IIIB | 1 | 1 |
| 215 | 1 | 1 | 2 | 1527 | IB | 2 | 1 |
| 216 | 1 | 2 | 2 | 1539 | IIB | 2 | 1 |
| 217 | 1 | 2 | 2 | 1568 | IIB | 1 | 1 |
| 218 | 1 | 1 | 1 | 1598 | IIA | 3 | 1 |
| 219 | 2 | 1 | 2 | 1108 | NA | NA | NA |
| 220 | 1 | 1 | 2 | 1626 | IIB | 2 | 1 |
| 221 | 1 | 1 | 1 | 1656 | IA | 2 | 1 |
| 222 | 1 | 1 | 2 | 1660 | IIB | 1 | 1 |
| 223 | 1 | 1 | 2 | 1661 | IIIA | 1 | 1 |
| 224 | 1 | 1 | 2 | 1720 | IA | 2 | 1 |
| 225 | 2 | 1 | 1 | 469 | NA | NA | NA |
| 226 | 1 | 2 | 2 | 1079 | IB | 1 | NA |
| 227 | 1 | 2 | 2 | 1723 | NA | 1 | 1 |
| 228 | 1 | 2 | 1 | 1763 | IIIA | 1 | 1 |
| 229 | 1 | 2 | 2 | 1084 | NA | 1 | 2 |
| 230 | 1 | 1 | 2 | 1093 | IB | 1 | 2 |
| 231 | 1 | 1 | 2 | 1777 | IB | 3 | 1 |
| 232 | 2 | 2 | 2 | 1022 | NA | 2 | NA |
| 233 | 1 | 1 | 1 | 1818 | IA | 1 | 1 |
| 234 | 1 | 2 | 2 | 1833 | IIIA | 2 | 1 |
| 235 | 2 | 1 | 1 | 30 | NA | 1 | NA |
| 236 | 1 | 2 | 2 | 1836 | IA | 1 | 1 |
| 237 | 1 | 2 | 2 | 1850 | IIA | 1 | 1 |
| 238 | 1 | 1 | 2 | 1874 | IA | 2 | 1 |
| 239 | 1 | 2 | 2 | 1896 | IA | 1 | 1 |
| 240 | 1 | 2 | 2 | 1905 | IIIA | 1 | 1 |
| 241 | 1 | 2 | 1 | 1134 | IIA | 2 | 2 |
| 242 | 2 | 1 | 1 | 555 | IA | 2 | NA |
| 243 | 2 | 2 | 2 | 945 | NA | 3 | NA |
| 244 | 2 | 2 | 1 | 409 | NA | NA | NA |
| 245 | 1 | 1 | 2 | 1139 | IB | 3 | 2 |
| 246 | 1 | 1 | 2 | 1987 | IB | 2 | 1 |
| 247 | 1 | 2 | 2 | 1140 | IIA | 1 | 2 |
| 248 | 1 | 2 | 1 | 1153 | IB | 2 | 2 |
| 249 | 1 | 1 | 2 | 1990 | IV | 1 | 1 |
| 250 | 1 | 2 | 2 | 2035 | IA | 2 | 1 |
| 251 | 1 | 1 | 1 | 1172 | IA | 1 | 2 |
| 252 | 1 | 1 | 1 | 1184 | NA | 1 | 2 |
| 253 | 1 | 2 | 1 | 1187 | IIIA | 2 | 2 |
| 254 | 1 | 2 | 2 | 1197 | IIA | 3 | 2 |
| 255 | 1 | 1 | 2 | 1198 | IB | 2 | 2 |
| 256 | 1 | 1 | 1 | 1209 | IA | 3 | 2 |
| 257 | 1 | 2 | 2 | 1213 | IIA | 2 | 2 |
| 258 | 1 | 1 | 2 | 2121 | IIB | 1 | 1 |
| 259 | 1 | 2 | 2 | 2128 | IIIA | 1 | 1 |
| 260 | 1 | 1 | 1 | 2132 | IB | 1 | 1 |
| 261 | 1 | 1 | 2 | 1219 | IA | 2 | 2 |
| 262 | 1 | 2 | 2 | 2137 | IA | 1 | 1 |
| 263 | 1 | 2 | 2 | 2149 | IA | 1 | 1 |
| 264 | 1 | 2 | 2 | 2154 | IA | 1 | 1 |
| 265 | 1 | 2 | 1 | 2164 | IV | 1 | 1 |
| 266 | 1 | 1 | 2 | 1231 | IB | 3 | 2 |
| 267 | 1 | 1 | 2 | 2210 | IIB | 2 | 1 |
| 268 | 1 | 1 | 2 | 2225 | IIA | 1 | 1 |
| 269 | 1 | 1 | 2 | 2226 | IA | 1 | 1 |
| 270 | 1 | 2 | 2 | 2234 | IIIB | 1 | 1 |
| 271 | 1 | 1 | 2 | 2284 | IB | 2 | 1 |
| 272 | 1 | 2 | 2 | 2288 | IB | 1 | 1 |
| 273 | 1 | 2 | 2 | 2294 | IB | 1 | 1 |
| 274 | 1 | 1 | 2 | 1254 | IB | 1 | 2 |
| 275 | 1 | 2 | 2 | 2295 | IA | 1 | 1 |
| 276 | 1 | 1 | 2 | 2296 | IIA | 1 | 1 |
| 277 | 1 | 2 | 2 | 2304 | IIIB | 1 | 1 |
| 278 | 1 | 2 | 2 | 1269 | IB | 2 | 2 |
| 279 | 1 | 1 | 2 | 2364 | IB | 1 | 1 |
| 280 | 1 | 2 | 1 | 2393 | IB | 1 | 1 |
| 281 | 1 | 2 | 2 | 1280 | IB | 2 | 2 |
| 282 | 1 | 1 | 2 | 2401 | IA | 1 | 1 |
| 283 | 1 | 2 | 2 | 1293 | IA | 2 | 2 |
| 284 | 1 | 2 | 2 | 2420 | IB | 2 | 1 |
| 285 | 1 | 2 | 2 | 2449 | IB | 1 | 1 |
| 286 | 1 | 1 | 2 | 1317 | IB | 2 | 2 |
| 287 | 1 | 2 | 1 | 1320 | IB | 3 | 2 |
| 288 | 1 | 2 | 2 | 2470 | IV | 1 | 1 |
| 289 | 1 | 2 | 2 | 2500 | IIB | 2 | 1 |
| 290 | 1 | 1 | 2 | 2522 | IA | 1 | 1 |
| 291 | 1 | 2 | 2 | 2536 | IB | 2 | 1 |
| 292 | 1 | 2 | 2 | 2571 | IA | 2 | 1 |
| 293 | 1 | 2 | 2 | 2669 | IA | 1 | 1 |
| 294 | 1 | 2 | 2 | 2703 | IA | 2 | 1 |
| 295 | 1 | 1 | 2 | 1349 | IV | 2 | 2 |
| 296 | 1 | 2 | 2 | 1350 | IV | 2 | 2 |
| 297 | 1 | 1 | 2 | 1353 | NA | 2 | 2 |
| 298 | 1 | 2 | 2 | 2715 | IB | 2 | 1 |
| 299 | 1 | 2 | 2 | 1377 | IV | 1 | NA |
| 300 | 1 | 2 | 2 | 2900 | IIIB | 1 | 1 |
| 301 | 1 | 2 | 2 | 1387 | IB | 3 | 2 |
| 302 | 1 | 1 | 2 | 3060 | IB | 1 | 1 |
| 303 | 2 | 1 | 2 | 3072 | IB | 1 | 1 |
| 304 | 2 | 1 | 1 | 1017 | IIIA | 2 | 1 |
| 305 | 2 | 1 | 2 | 1105 | IIIA | 1 | 1 |
| 306 | 2 | 1 | 2 | 1142 | IIA | 1 | 1 |
| 307 | 1 | 1 | 2 | 1401 | IIB | 2 | 2 |
| 308 | 2 | 1 | 2 | 1149 | IIA | 2 | 1 |
| 309 | 1 | 2 | 2 | 1409 | IA | 2 | 2 |
| 310 | 2 | 1 | 1 | 1201 | IIIA | 1 | 1 |
| 311 | 2 | 2 | 2 | 1233 | IV | 2 | 1 |
| 312 | 2 | 1 | 1 | 1269 | IB | 2 | 1 |
| 313 | 2 | 1 | 2 | 1286 | IIIA | 1 | 1 |
| 314 | 1 | 1 | 2 | 1429 | IB | 2 | 2 |
| 315 | 2 | 1 | 2 | 1289 | IIB | 1 | 1 |
| 316 | 2 | 1 | 2 | 1309 | IA | 1 | 1 |
| 317 | 2 | 1 | 1 | 131 | IIIA | 2 | 1 |
| 318 | 2 | 1 | 2 | 134 | IIB | 1 | 1 |
| 319 | 1 | 2 | 2 | 1462 | IB | 2 | 2 |
| 320 | 2 | 1 | 2 | 1374 | IIB | 1 | 1 |
| 321 | 1 | 2 | 2 | 1470 | IA | 2 | 2 |
| 322 | 1 | 1 | 2 | 1475 | IIIA | 2 | 2 |
| 323 | 2 | 2 | 1 | 1432 | IIA | 1 | 1 |
| 324 | 2 | 2 | 2 | 1441 | IV | 2 | 1 |
| 325 | 1 | 2 | 2 | 1478 | NA | 2 | 2 |
| 326 | 2 | 1 | 2 | 1441 | IA | 1 | 1 |
| 327 | 2 | 1 | 2 | 1465 | IB | 1 | 1 |
| 328 | 2 | 1 | 2 | 1490 | IIB | 1 | 1 |
| 329 | 2 | 1 | 2 | 1552 | IB | 2 | 1 |
| 330 | 1 | 2 | 1 | 1496 | NA | 1 | NA |
| 331 | 2 | 2 | 2 | 1560 | IIB | 1 | 1 |
| 332 | 1 | 1 | 2 | 1511 | IA | 1 | 2 |
| 333 | 1 | 1 | 1 | 1514 | IIB | 1 | 2 |
| 334 | 1 | 1 | 2 | 1518 | IA | 1 | 2 |
| 335 | 2 | 2 | 2 | 1660 | IB | 1 | 1 |
| 336 | 2 | 1 | 1 | 168 | IV | 1 | 1 |
| 337 | 2 | 2 | 2 | 1764 | IIIA | 2 | 1 |
| 338 | 1 | 1 | 2 | 1540 | IA | 1 | 2 |
| 339 | 2 | 1 | 2 | 1822 | IIIA | 1 | 1 |
| 340 | 1 | 2 | 1 | 1565 | NA | 1 | 2 |
| 341 | 2 | 1 | 2 | 1860 | IIIA | 2 | 1 |
| 342 | 2 | 1 | 2 | 1932 | IIA | 2 | 1 |
| 343 | 2 | 1 | 2 | 1993 | IIIA | 1 | 1 |
| 344 | 2 | 2 | 2 | 2016 | IIB | 1 | 1 |
| 345 | 1 | 1 | 2 | 1570 | IB | 2 | 2 |
| 346 | 2 | 2 | 2 | 2017 | IB | 2 | 1 |
| 347 | 1 | 1 | 2 | 1580 | IIB | 3 | 2 |
| 348 | 1 | 1 | 1 | 1587 | IIIB | 1 | 2 |
| 349 | 1 | 2 | 2 | 1589 | IB | 1 | 2 |
| 350 | 1 | 2 | 1 | 1592 | IA | 3 | 2 |
| 351 | 2 | 2 | 2 | 2017 | IB | 1 | 1 |
| 352 | 2 | 2 | 2 | 2022 | IA | 1 | 1 |
| 353 | 2 | 2 | 2 | 2044 | IB | 1 | 1 |
| 354 | 2 | 2 | 2 | 2081 | IB | 2 | 1 |
| 355 | 1 | 2 | 2 | 1619 | NA | 2 | 2 |
| 356 | 2 | 2 | 2 | 2109 | IB | 1 | 1 |
| 357 | 1 | 1 | 1 | 1650 | IIB | 2 | 2 |
| 358 | 2 | 1 | 2 | 2234 | IA | 1 | 1 |
| 359 | 2 | 1 | 2 | 2269 | IIB | 1 | 1 |
| 360 | 2 | 1 | 2 | 2294 | IIIA | 2 | 1 |
| 361 | 2 | 1 | 2 | 2309 | IB | 2 | 1 |
| 362 | 2 | 1 | 2 | 2318 | IIB | 1 | 1 |
| 363 | 2 | 1 | 2 | 2326 | IA | 1 | 1 |
| 364 | 2 | 1 | 2 | 2338 | IIIA | 1 | 1 |
| 365 | 2 | 1 | 1 | 241 | IIB | 3 | 1 |
| 366 | 1 | 2 | 2 | 1678 | IA | 1 | 2 |
| 367 | 1 | 2 | 1 | 1713 | IIB | 2 | 2 |
| 368 | 2 | 1 | 2 | 2452 | IIB | 2 | 1 |
| 369 | 2 | 1 | 1 | 252 | IIB | 2 | 1 |
| 370 | 2 | 2 | 1 | 252 | IIB | 1 | 1 |
| 371 | 2 | 2 | 1 | 270 | IIB | 1 | 1 |
| 372 | 1 | 2 | 2 | 1737 | IA | 3 | 2 |
| 373 | 2 | 1 | 2 | 2774 | IIIB | 2 | 1 |
| 374 | 2 | 1 | 2 | 2966 | IA | 1 | 1 |
| 375 | 2 | 1 | 2 | 2992 | IIB | 1 | 1 |
| 376 | 1 | 2 | 2 | 1780 | IB | 2 | 2 |
| 377 | 1 | 1 | 2 | 1800 | IIB | 2 | 2 |
| 378 | 1 | 2 | 2 | 1801 | IB | 2 | 2 |
| 379 | 1 | 2 | 2 | 1804 | IB | 3 | 2 |
| 380 | 2 | 1 | 1 | 313 | IB | 1 | 1 |
| 381 | 1 | 2 | 2 | 1814 | IA | 2 | 2 |
| 382 | 2 | 1 | 1 | 314 | IIIA | 1 | 1 |
| 383 | 2 | 2 | 1 | 358 | IIIA | 2 | 1 |
| 384 | 2 | 1 | 1 | 368 | IIIA | 2 | 1 |
| 385 | 2 | 1 | 2 | 37 | IIB | 1 | 1 |
| 386 | 2 | 1 | 1 | 381 | IIB | 3 | 1 |
| 387 | 2 | 1 | 1 | 413 | IA | 1 | 1 |
| 388 | 2 | 2 | 1 | 428 | IIA | 1 | 1 |
| 389 | 2 | 1 | 1 | 436 | NA | 1 | 1 |
| 390 | 2 | 1 | 1 | 44 | IB | 1 | 1 |
| 391 | 2 | 1 | 1 | 500 | IB | 2 | 1 |
| 392 | 2 | 2 | 1 | 504 | NA | 1 | 1 |
| 393 | 1 | 2 | 2 | 1909 | IIIA | 1 | 2 |
| 394 | 1 | 2 | 1 | 1916 | IIIB | 1 | NA |
| 395 | 2 | 2 | 1 | 532 | IB | 3 | 1 |
| 396 | 1 | 2 | 2 | 1926 | IIB | 3 | 2 |
| 397 | 2 | 1 | 1 | 55 | IIA | 2 | 1 |
| 398 | 1 | 1 | 2 | 1961 | IA | 2 | 2 |
| 399 | 2 | 1 | 1 | 617 | IIIB | 1 | 1 |
| 400 | 1 | 1 | 2 | 1982 | IB | 2 | 2 |
| 401 | 2 | 1 | 1 | 638 | IIB | 2 | 1 |
| 402 | 2 | 1 | 1 | 651 | IB | 1 | 1 |
| 403 | 2 | 1 | 2 | 733 | IA | 2 | 1 |
| 404 | 2 | 1 | 1 | 746 | IIB | 2 | 1 |
| 405 | 2 | 1 | 1 | 765 | IIA | 1 | 1 |
| 406 | 1 | 1 | 1 | 2026 | IA | 2 | 2 |
| 407 | 2 | 1 | 1 | 931 | IB | 1 | 1 |
| 408 | 1 | 2 | 2 | 2051 | IA | NA | 2 |
| 409 | 2 | 2 | 2 | 958 | IIB | 2 | 1 |
| 410 | 1 | 2 | 2 | 2074 | IIIA | 2 | 2 |
| 411 | 2 | 2 | 2 | 985 | IA | 2 | 1 |
| 412 | 1 | 2 | 2 | 2086 | IB | 2 | NA |
| 413 | 1 | 1 | 2 | 2121 | IA | 1 | 2 |
| 414 | 1 | 1 | 2 | 2143 | IIIA | 1 | 2 |
| 415 | 1 | 1 | 2 | 2247 | IA | 2 | 2 |
| 416 | 1 | 2 | 2 | 2253 | IB | 2 | 2 |
| 417 | 1 | 2 | 2 | 2322 | IB | 1 | 2 |
| 418 | 1 | 2 | 2 | 2346 | IIB | 1 | 2 |
| 419 | 1 | 2 | 2 | 2360 | IIIA | 3 | 2 |
| 420 | 1 | 2 | 2 | 2445 | IA | 3 | 2 |
| 421 | 1 | 2 | 2 | 2450 | IB | 3 | 2 |
| 422 | 1 | 2 | 2 | 2469 | IA | 2 | 2 |
| 423 | 1 | 1 | 2 | 2492 | IIA | 2 | 2 |
| 424 | 1 | 2 | 2 | 2533 | IIIA | 3 | 2 |
| 425 | 2 | 1 | 2 | 2535 | NA | 2 | 1 |
| 426 | 1 | 2 | 2 | 2676 | IA/IIA | 2 | 2 |
| 427 | 1 | 2 | 2 | 2681 | IB | 2 | 2 |
| 428 | 1 | 2 | 2 | 2711 | IB | 1 | 2 |
| 429 | 1 | 1 | 1 | 146 | IB | 1 | 1 |
| 430 | 1 | 1 | 1 | 149 | IB | 2 | 1 |
| 431 | 1 | 2 | 2 | 2764 | IB | 1 | 2 |
| 432 | 1 | 1 | 2 | 2829 | IB | 3 | 2 |
| 433 | 1 | 2 | 1 | 171 | IA | 1 | 1 |
| 434 | 1 | 2 | 1 | 215 | IV | 2 | 1 |
| 435 | 1 | 1 | 2 | 2897 | IB | 2 | 2 |
| 436 | 1 | 1 | 1 | 279 | IIIA | 2 | 1 |
| 437 | 1 | 2 | 2 | 2934 | IA | 2 | 2 |
| 438 | 1 | 2 | 1 | 390 | IV | 2 | 1 |
| 439 | 1 | 1 | 1 | 464 | NA | 2 | 1 |
| 440 | 1 | 1 | 2 | 2960 | IIB | 1 | 2 |
| 441 | 1 | 2 | 2 | 2963 | IIIA | 1 | 2 |
| 442 | 1 | 1 | 1 | 522 | IB | 2 | 1 |
| 443 | 1 | 2 | 2 | 3032 | IB | 2 | 2 |
| 444 | 1 | 2 | 1 | 577 | IA | 1 | 1 |
| 445 | 1 | 1 | 1 | 598 | IIIA | 2 | 1 |
| 446 | 1 | 2 | 1 | 605 | IA | 2 | 1 |
| 447 | 1 | 1 | 2 | 617 | IA | 2 | 1 |
| 448 | 1 | 1 | 1 | 620 | IIIA | 3 | 1 |
| 449 | 2 | 1 | 1 | 1008 | IIA | 1 | NA |
| 450 | 1 | 1 | 1 | 710 | IV | 1 | 1 |
| 451 | 2 | 1 | 2 | 101 | IA | 1 | NA |
| 452 | 2 | 2 | 2 | 1021 | IA | 2 | NA |
| 453 | 1 | 1 | 1 | 866 | IA | 2 | 1 |
| 454 | 1 | 2 | 1 | 908 | IB | 1 | 1 |
| 455 | 1 | 2 | 1 | 1110 | IA | 1 | 1 |
| 456 | 1 | 1 | 2 | 1322 | IB | 2 | 1 |
| 457 | 1 | 1 | 2 | 1388 | IIA | 1 | 1 |
| 458 | 1 | 2 | 2 | 1423 | IIB | 2 | 1 |
| 459 | 2 | 1 | 2 | 1056 | IIB | NA | NA |
| 460 | 1 | 2 | 1 | 1441 | IIIB | 1 | 1 |
| 461 | 1 | 1 | 1 | 1441 | IA | 1 | 1 |
| 462 | 1 | 1 | 2 | 1468 | IA | 2 | 1 |
| 463 | 2 | 2 | 2 | 1084 | IB | 2 | 2 |
| 464 | 2 | 1 | 2 | 1087 | IB | 3 | 2 |
| 465 | 2 | 2 | 2 | 1094 | IB | NA | NA |
| 466 | 1 | 1 | 1 | 1485 | IB | 2 | 1 |
| 467 | 2 | 2 | 2 | 1107 | IIIA | 1 | NA |
| 468 | 1 | 2 | 2 | 1534 | IV | 2 | 1 |
| 469 | 1 | 2 | 2 | 1713 | IB | 2 | 1 |
| 470 | 1 | 2 | 2 | 1764 | IA | 2 | 1 |
| 471 | 1 | 1 | 2 | 1771 | IIA | 2 | 1 |
| 472 | 1 | 2 | 2 | 1807 | IB | 2 | 1 |
| 473 | 2 | 1 | 2 | 1160 | IIIA | 2 | 2 |
| 474 | 1 | 2 | 2 | 1898 | IIIA | 2 | 1 |
| 475 | 1 | 1 | 2 | 1946 | IB | 2 | 1 |
| 476 | 2 | 1 | 2 | 118 | IIIA/IIIB | 2 | 2 |
| 477 | 1 | 2 | 1 | 1970 | IB | 2 | 1 |
| 478 | 1 | 2 | 2 | 1986 | IA | 2 | 1 |
| 479 | 2 | 1 | 2 | 1188 | IA | 2 | 2 |
| 480 | 2 | 1 | 2 | 1192 | IIIA | 2 | 2 |
| 481 | 1 | 2 | 2 | 2067 | IIIA | 2 | 1 |
| 482 | 2 | 2 | 2 | 1216 | IA | NA | NA |
| 483 | 1 | 2 | 2 | 2184 | IA | 1 | 1 |
| 484 | 1 | 1 | 2 | 2185 | IIIB | 1 | 1 |
| 485 | 1 | 2 | 2 | 2408 | IB | 2 | 1 |
| 486 | 2 | 2 | 2 | 1223 | IB | 2 | 2 |
| 487 | 1 | 2 | 2 | 2535 | IB | 2 | 1 |
| 488 | 1 | 2 | 2 | 2667 | IB | 2 | 1 |
| 489 | 1 | 2 | 2 | 2857 | IIB | 1 | 1 |
| 490 | 2 | 1 | 2 | 1238 | IIIA | 2 | 2 |
| 491 | 2 | 2 | 2 | 1239 | IB | 2 | NA |
| 492 | 2 | 1 | 1 | 1241 | IA | 2 | 2 |
| 493 | 1 | 2 | 2 | 2975 | IA | 2 | 1 |
| 494 | 2 | 1 | 2 | 1248 | IIIA | 2 | 2 |
| 495 | 2 | 1 | 1 | 1258 | IA | 2 | 2 |
| 496 | 2 | 2 | 2 | 1258 | IIIA | 2 | NA |
| 497 | 1 | 1 | 2 | 3065 | IA | 1 | 1 |
| 498 | 2 | 1 | 2 | 1030 | IIIA | 2 | 1 |
| 499 | 2 | 2 | 2 | 1031 | IIIA | 2 | 1 |
| 500 | 2 | 2 | 1 | 1053 | IIB | 2 | 1 |
| 501 | 2 | 2 | 1 | 1080 | IIA | 2 | 1 |
| 502 | 2 | 1 | 1 | 1122 | IIA | 1 | 1 |
| 503 | 2 | 1 | 2 | 1296 | IB | 2 | 1 |
| 504 | 2 | 2 | 2 | 1309 | IB | 1 | 2 |
| 505 | 2 | 1 | 1 | 133 | IIB | 1 | 1 |
| 506 | 2 | 1 | 1 | 1348 | IA | 1 | 1 |
| 507 | 2 | 2 | 1 | 131 | IV | 1 | NA |
| 508 | 2 | 2 | 2 | 1318 | IA | 2 | 2 |
| 509 | 2 | 1 | 1 | 1323 | IB | 2 | 2 |
| 510 | 2 | 2 | 1 | 1367 | IB | 1 | 1 |
| 511 | 2 | 1 | 2 | 1482 | IB | 2 | 1 |
| 512 | 2 | 1 | 1 | 1495 | IIIA | 2 | 1 |
| 513 | 2 | 1 | 2 | 1563 | IA | 2 | 1 |
| 514 | 2 | 1 | 2 | 1591 | IIB | 2 | 1 |
| 515 | 2 | 1 | 1 | 1371 | IIB | 2 | 2 |
| 516 | 2 | 1 | 2 | 1702 | IB | 2 | 1 |
| 517 | 2 | 1 | 1 | 171 | IIB | 2 | 1 |
| 518 | 2 | 1 | 2 | 1387 | IA | 2 | 2 |
| 519 | 2 | 1 | 2 | 1758 | IIIA | 2 | 1 |
| 520 | 2 | 2 | 1 | 1848 | IB | 1 | 1 |
| 521 | 2 | 1 | 2 | 1855 | IA | 2 | 1 |
| 522 | 2 | 2 | 2 | 1940 | IB | 2 | 1 |
| 523 | 2 | 1 | 2 | 1454 | IB | 2 | 2 |
| 524 | 2 | 1 | 1 | 1944 | IA | 1 | 1 |
| 525 | 2 | 1 | 2 | 2395 | IB | 2 | 1 |
| 526 | 2 | 2 | 2 | 1478 | IB | 2 | 2 |
| 527 | 2 | 1 | 2 | 2590 | IIIA | 2 | 1 |
| 528 | 2 | 1 | 1 | 2607 | IIB | 2 | 1 |
| 529 | 2 | 1 | 2 | 1485 | IB | 3 | 2 |
| 530 | 2 | 1 | 2 | 279 | IB | 2 | 1 |
| 531 | 2 | 1 | 2 | 2827 | IB | 1 | 1 |
| 532 | 2 | 1 | 1 | 297 | IB | 1 | 1 |
| 533 | 2 | 1 | 2 | 3023 | IB | 1 | 1 |
| 534 | 2 | 1 | 2 | 3024 | IIIA | 1 | 1 |
| 535 | 2 | 1 | 2 | 3054 | IIB | 1 | 1 |
| 536 | 2 | 2 | 2 | 1499 | IB | 2 | 2 |
| 537 | 2 | 1 | 1 | 455 | IIIA | 1 | 1 |
| 538 | 2 | 2 | 2 | 841 | IIA | 2 | 1 |
| 539 | 2 | 1 | 1 | 1537 | IB | 1 | 2 |
| 540 | 2 | 1 | 2 | 1540 | IIIB | 3 | 2 |
| 541 | 1 | 2 | 1 | 386 | IIIA | 3 | 3 |
| 542 | 2 | 1 | 2 | 1559 | IIA | 2 | 2 |
| 543 | 2 | 2 | 2 | 1357 | IB | 2 | 3 |
| 544 | 2 | 1 | 1 | 1374 | IA | 2 | 3 |
| 545 | 2 | 1 | 2 | 1577 | IB | 2 | 3 |
| 546 | 2 | 1 | 1 | 158 | IIIA | 2 | 2 |
| 547 | 2 | 2 | 2 | 1615 | IIA | 3 | 2 |
| 548 | 2 | 2 | 2 | 1668 | IA | 3 | 2 |
| 549 | 2 | 2 | 2 | 1554 | NA | 2 | 3 |
| 550 | 2 | 2 | 1 | 186 | NA | 2 | 3 |
| 551 | 1 | 2 | 1 | 482 | IIB | 3 | 3 |
| 552 | 1 | 1 | 1 | 688 | IB | 3 | 3 |
| 553 | 2 | 1 | 2 | 178 | IIA | 1 | 2 |
| 554 | 2 | 2 | 2 | 1793 | IIIA | 2 | 2 |
| 555 | 1 | 2 | 1 | 1409 | IB | 3 | 3 |
| 556 | 2 | 1 | 2 | 1843 | IIIA | 2 | 2 |
| 557 | 1 | 1 | 2 | 1496 | IB | 3 | 3 |
| 558 | 1 | 2 | 2 | 1506 | NA | 2 | 3 |
| 559 | 1 | 2 | 2 | 1533 | IIA | 2 | 3 |
| 560 | 1 | 1 | 2 | 1555 | NA | 2 | 3 |
| 561 | 2 | 1 | 2 | 189 | IIIA | 2 | 2 |
| 562 | 2 | 2 | 2 | 1898 | IB | 1 | 2 |
| 563 | 2 | 2 | 2 | 1902 | IIB | 2 | 2 |
| 564 | 1 | 2 | 1 | 1611 | IIIB | 3 | 3 |
| 565 | 1 | 1 | 2 | 1715 | IA | 3 | 3 |
| 566 | 2 | 2 | 1 | 192 | IA | NA | NA |
| 567 | 1 | 1 | 2 | 1815 | NA | 1 | 3 |
| 568 | 2 | 2 | 2 | 1937 | IIB | 1 | 2 |
| 569 | 1 | 2 | 2 | 2170 | IA | 1 | 3 |
| 570 | 1 | 2 | 2 | 2466 | IB | 3 | 3 |
| 571 | 2 | 1 | 2 | 1973 | IIB | 3 | 2 |
| 572 | 1 | 1 | 2 | 2955 | IIB | 2 | 3 |
| 573 | 2 | 1 | 2 | 1069 | IB | 3 | 3 |
| 574 | 2 | 2 | 2 | 1511 | IA | 3 | 3 |
| 575 | 2 | 2 | 2 | 152 | IIA | 3 | 3 |
| 576 | 2 | 1 | 2 | 1736 | IA | 1 | 3 |
| 577 | 2 | 1 | 1 | 192 | IIIA | 3 | 3 |
| 578 | 2 | 1 | 1 | 2047 | IA | 2 | 2 |
| 579 | 2 | 2 | 1 | 206 | IIIB | 3 | 3 |
| 580 | 2 | 2 | 2 | 2591 | IB | 3 | 3 |
| 581 | 2 | 1 | 1 | 2079 | IB | 2 | 2 |
| 582 | 2 | 1 | 2 | 2778 | IB | 2 | 3 |
| 583 | 2 | 1 | 1 | 324 | IIA | 2 | 3 |
| 584 | 2 | 1 | 2 | 2115 | IA | 3 | 2 |
| 585 | 2 | 1 | 2 | 2116 | IB | 2 | 2 |
| 586 | 2 | 2 | 2 | 2156 | IB | 2 | 2 |
| 587 | 2 | 2 | 2 | 2157 | IIIA | 2 | 2 |
| 588 | 2 | 1 | 2 | 2178 | IIB | 2 | 2 |
| 589 | 2 | 2 | 1 | 2188 | IB | 2 | 2 |
| 590 | 2 | 1 | 2 | 41 | IIA | 3 | 3 |
| 591 | 2 | 1 | 2 | 2261 | IIIA | 2 | 2 |
| 592 | 2 | 1 | 2 | 2269 | IA | 2 | 2 |
| 593 | 2 | 1 | 2 | 2290 | IIB | 1 | 2 |
| 594 | 2 | 1 | 2 | 2291 | IB | 1 | 2 |
| 595 | 1 | 2 | 2 | 1253 | IA | 3 | 3 |
| 596 | 1 | 2 | 1 | 66 | IIA | 3 | 3 |
| 597 | 1 | 2 | 1 | 152 | IIIA | 3 | 3 |
| 598 | 1 | 2 | 1 | 263 | IIIA | 2 | 3 |
| 599 | 2 | 1 | 1 | 238 | IV | 1 | 2 |
| 600 | 1 | 1 | 1 | 274 | IA | 2 | 3 |
| 601 | 1 | 2 | 1 | 288 | IIA | 3 | 3 |
| 602 | 2 | 2 | 2 | 2430 | IB | 1 | 2 |
| 603 | 1 | 1 | 1 | 398 | IA | 2 | 3 |
| 604 | 1 | 2 | 1 | 402 | IV | 3 | 3 |
| 605 | 1 | 2 | 1 | 494 | IIIB | 3 | 3 |
| 606 | 2 | 1 | 2 | 2557 | IB | 2 | 2 |
| 607 | 1 | 1 | 1 | 551 | IB | 2 | 3 |
| 608 | 1 | 1 | 1 | 564 | IIB | 2 | 3 |
| 609 | 1 | 1 | 1 | 663 | IA | 2 | 3 |
| 610 | 1 | 1 | 1 | 841 | IIB | 3 | 3 |
| 611 | 1 | 1 | 2 | 938 | IIIA | 2 | 3 |
| 612 | 1 | 1 | 2 | 1023 | IA | 2 | 3 |
| 613 | 1 | 2 | 1 | 1030 | IB | 3 | 3 |
| 614 | 2 | 2 | 2 | 2780 | IIA | 2 | 2 |
| 615 | 1 | 2 | 2 | 1045 | IIIA | 2 | 3 |
| 616 | 1 | 1 | 1 | 1050 | IB | 2 | 3 |
| 617 | 2 | 2 | 2 | 2807 | IB | 1 | 2 |
| 618 | 1 | 2 | 2 | 1057 | IA | 3 | 3 |
| 619 | 1 | 1 | 2 | 1083 | IA | 2 | 3 |
| 620 | 2 | 1 | 1 | 291 | IIIA | NA | NA |
| 621 | 1 | 1 | 1 | 1108 | IIB | 2 | 3 |
| 622 | 1 | 1 | 2 | 1112 | IIA | 2 | 3 |
| 623 | 1 | 2 | 2 | 1118 | IA | 2 | 3 |
| 624 | 2 | 1 | 2 | 2998 | IB | 1 | 2 |
| 625 | 1 | 1 | 2 | 1195 | IIB | 2 | 3 |
| 626 | 1 | 1 | 2 | 1265 | IIIA | 3 | 3 |
| 627 | 2 | 2 | 2 | 3054 | IA | 1 | 2 |
| 628 | 1 | 1 | 2 | 1297 | IIIA | 2 | 3 |
| 629 | 1 | 1 | 2 | 1310 | IB | 3 | 3 |
| 630 | 1 | 2 | 2 | 1331 | IIA | 3 | 3 |
| 631 | 1 | 1 | 2 | 1393 | IIA | 3 | 3 |
| 632 | 2 | 2 | 1 | 331 | IIB | 2 | NA |
| 633 | 2 | 2 | 1 | 347 | IIIB | 1 | 2 |
| 634 | 2 | 1 | 2 | 35 | IA | 2 | 2 |
| 635 | 1 | 1 | 2 | 1394 | IA | 3 | 3 |
| 636 | 1 | 2 | 2 | 1409 | NA | 3 | 3 |
| 637 | 1 | 1 | 2 | 1414 | IA | 2 | 3 |
| 638 | 2 | 1 | 1 | 373 | IA | 3 | 2 |
| 639 | 2 | 1 | 1 | 381 | IIIB | 1 | NA |
| 640 | 1 | 2 | 2 | 1476 | NA | 3 | 3 |
| 641 | 2 | 2 | 1 | 396 | IIB | 2 | 2 |
| 642 | 1 | 1 | 2 | 1567 | IB | 2 | 3 |
| 643 | 1 | 2 | 2 | 1612 | IIB | 2 | 3 |
| 644 | 1 | 1 | 2 | 1657 | IIB | 3 | 3 |
| 645 | 2 | 1 | 2 | 42 | IIA | 2 | 2 |
| 646 | 1 | 2 | 2 | 1660 | IV | 2 | 3 |
| 647 | 1 | 2 | 2 | 1819 | IV | 3 | 3 |
| 648 | 1 | 2 | 2 | 1848 | IIA | 3 | 3 |
| 649 | 1 | 1 | 2 | 1871 | IA | 3 | 3 |
| 650 | 2 | 2 | 1 | 468 | IIIA | 2 | NA |
| 651 | 2 | 1 | 1 | 471 | IB | 2 | 2 |
| 652 | 1 | 1 | 2 | 1897 | IIB | 2 | 3 |
| 653 | 2 | 2 | 1 | 49 | IIB | 3 | 2 |
| 654 | 1 | 2 | 2 | 1919 | IIB | 3 | 3 |
| 655 | 1 | 2 | 2 | 2119 | IA | 2 | 3 |
| 656 | 1 | 1 | 2 | 2260 | IA | 3 | 3 |
| 657 | 2 | 2 | 1 | 533 | IB | 2 | 2 |
| 658 | 2 | 2 | 1 | 534 | IIIA | 2 | 2 |
| 659 | 1 | 1 | 2 | 2302 | IB | 3 | 3 |
| 660 | 2 | 1 | 1 | 554 | IIIA | 2 | NA |
| 661 | 2 | 1 | 1 | 577 | IB | 1 | 2 |
| 662 | 2 | 1 | 1 | 580 | IB | 2 | 2 |
| 663 | 2 | 2 | 1 | 587 | IIB | 2 | 2 |
| 664 | 1 | 1 | 2 | 3018 | IA | 3 | 3 |
| 665 | 2 | 2 | 2 | 1035 | IIIA | 2 | 3 |
| 666 | 2 | 2 | 2 | 1156 | IIIA | 2 | 3 |
| 667 | 2 | 2 | 2 | 1171 | IA | 1 | 3 |
| 668 | 2 | 1 | 1 | 630 | IIIA | 1 | 2 |
| 669 | 2 | 1 | 1 | 636 | IA | 2 | 2 |
| 670 | 2 | 2 | 2 | 1185 | IB | 3 | 3 |
| 671 | 2 | 2 | 2 | 1217 | IA | 3 | 3 |
| 672 | 2 | 2 | 1 | 696 | IIIA | 2 | 2 |
| 673 | 2 | 1 | 1 | 707 | IA | 2 | 2 |
| 674 | 2 | 1 | 2 | 1316 | IB | 2 | 3 |
| 675 | 2 | 1 | 2 | 1902 | IB | 3 | 3 |
| 676 | 2 | 1 | 1 | 760 | IIB | 2 | 2 |
| 677 | 2 | 1 | 1 | 765 | IIIA | 2 | 2 |
| 678 | 2 | 1 | 2 | 2059 | IIIA | 3 | 3 |
| 679 | 2 | 1 | 1 | 800 | IIB | 2 | 2 |
| 680 | 2 | 1 | 1 | 825 | IB | 1 | 2 |
| 681 | 2 | 2 | 2 | 2371 | IA | 3 | 3 |
| 682 | 2 | 2 | 1 | 861 | IB | 1 | NA |
| 683 | 2 | 2 | 2 | 2753 | IA | 3 | 3 |
| 684 | 2 | 2 | 1 | 912 | IA | 1 | 2 |
| 685 | 2 | 1 | 2 | 923 | IA | NA | NA |
| 686 | 2 | 2 | 1 | 282 | IB | 3 | 3 |
| 687 | 2 | 1 | 2 | 485 | IIIA | 3 | 3 |
| 688 | 2 | 1 | 1 | 591 | IA | 3 | 3 |
| 689 | 2 | 1 | 2 | 996 | IIIA | 2 | 2 |

Data not available is indicated by ′NA′

TABLE 11
Gene lists utilized for GSEA analysis.
Genes upregulated in exhaustion; obtained from Wherry et al,
Immunity 2007; 27(4): 670-84

1110006I15Rik
1110067D22Rik
1810035L17Rik
1810054D07Rik
2010100O12Rik
2510004L0Rik
2700084L22Rik
5730469M10Rik
9130009C22Rik
9130410M22Rik
A430109M19Rik
Adfp
Ai181996
Art3
Atf1
BC024955
Bub1
C330007P06Rik
C79248
Capzb
Car2
Casp3
Casp4
Ccl3
Ccl4
Ccrl2
Cd160
Cd244
Cd7
Cd9
Chl1
Cit
Coch
Cpa3
Cpsf2
Cpt2
Crygb
Ctla4
Cxcl10
D15Ertd781e
D8Ertd531e
Dock7
Entpd1
Eomes
Eomes
Etf1
G1p2
Gein
Gdf3
Gp49b
Gpd2
Gpr56
Gpr65
Icsbp1
Ier5
Isg20
Itgav
Jak3
Kdt1
Klk6
Lag3
Lman2
MKi67
Mox2
Mtv43
Myh4
NDfip1
Nftac1
Nptxr
Nr4a2
Nr4a2
Pawr
Pbx3
Pdcd1
Penk1
Plscr1
Pon2
Prkwnk1
Ptger2
Ptger4
Ptpn13
Rcn
Rgs16
Rnf11
S100a13
Sept4
Serpina3g
Sh2d2a
Shkbp1
Snrpb2
Spock2
Spp1
Sybl1
Tank
Tcea2
Tcrb-V13
Tcrg-V4
Tcrg-V4
Tnfrsf1a
TnfrsP9
Tnfsf6
Tor3a
Trim25
Trim47
Tubb2
Wbp5
Wbscr5
Zfp91

Genes downregulated in exhaustion; obtained from
Wherry et al, Immunity 2007; 27(4): 670-84

1110020B03Rik
1110038D17Rik
1810009A16Rik
1810045K07Rik
2010315L10Rik
2810024B22Rik
2810404F18Rik
2810407C02Rik
4833420G17Rik
6330406L22Rik
A630038C17Rik
Abce1
Ablim1
Acadm
Acas2l
Acp5
Adcy7
Add1
Adh5
Akap8
Al325941
Al447904
Anapc5
Arbp
Arhgap1
Arhgef1
Ascc1
Atp6v0a2
Atp6v0b
B230114J08Rik
Bat1a
Bnip31
Bzw1
C79468
Cct3
Cct4
Cct5
Cct8
Cd1d1
Clk2
Cmah
Crlf3
D10Wsu52e
D14Wsu123e
Dgka
Dtx1
Eef2
Eif2s1
Ephb4
Erdr1
Ets1
Fkbp4
Gm2a
Gnb2-rs1
Gtf2i
Hcph
Hexa
Hmgcs1
Hspa8
Iap
Iap(II-3)
Iap11-1
Icam2
Ifnar1
Il17r
Il17r
Itgb7
Jmjd1a
Kcnn4
Kctd10
Klf13
Klf2
Klf3
Klf3
Lbr
Lef1
LOC280487
LOC381438
Macf1
Map4k4
Mapk8
Mat2a
Nfe2l2
Nme2
Numb
Osbpl11
Pak2
Pdha1
Pdlim1
Pik3cd
Pld3
Ppp2r5a
Prps1
Prss19
Pscdbp
Ptk9l
Rap1gds1
Rnpc1
Rpl10
Rpl10a
Rpl13
Rpl22
Rpl28
Rpl3
Rp18
Rpn2
Rps16
Rps3
Rps3a
Rps4x
Rps7
Rps8
Satb1
Sema4a
Sgk
Shda
Siah1a
Skp1a
Snrpd3
Snx4
Srpk1
Ss18
Stk38
Supt5h
Tex292
Tmc6
Tubb5
Tubb5
Ubp1
Znrf2

Lung cancer-associated T cell signature genes; obtained
from Johnston et al, Cancer Cell 2014; 26(6): 9723-37

ARHGAP15
CCL5
CCR5
CD2
CD247
CD48
CD6
CD96
CRTAM
CST7
CTLA4
CXCR3
CXCR6
GIMAP1
GIMAP4
GIMAP5
GIMAP7
GPR171
GPR174
GVINP1
GZMH
IL10RA
IL12RB1
IL2RG
ITGAL
ITK
LCK
LINC00426
LOC100506776
LOC100652927
NKG7
P2RY10
PCED1B-AS1
PDCD1
PTPN22
PTPRC
PTPRCAP
PYHIN1
SASH3
SCML4
SH2D1A
SIRPG
SIT1
SLA2
SLAMF1
SLAMF6
TBC1D10C
TESPA1
TIGIT
UBASH3A

Genes upregulated in senescence; obtained from
Safford et al, Nature Immunology 2005; 6(5): 472-80

ACTN4
ADORA2A
ADORA2B
AGT
ANGPTL2
ANKRD28
ANP32A
ARFIP1
BNIP3
CASP4
CCL1
CCL3
CCRN4L
CD40LG
CD97
CDC14A
CLEC4E
CSF1
CTSE
DDR1
DLG2
DTNA
DUSP6
EGR2
ETV6
F2R
FBXO34
FOXP1
FURIN
FYN
GABRA4
GADD45B
GCH1
GGA2
HEBP2
HIF1AN
HLF
HSD17B6
HSPA4L
IER3
ING4
IRF4
ISYNA1
JAK3
JARID2
JUP
KCNJ11
KCNK5
KCNQ5
KIF15
KIFC3
LAG3
LDHB
LPAR4
LRRC3
MARCH2
MMD
MPZL2
MYH14
MYL7
MYO1C
MYO1E
NDRG1
NFATC1
NOTCH1
NR4A2
NR4A3
OAZ3
PFKP
PLA2G10
RCBTB1
RNF19A
S100A5
SFRP4
SLC29A3
SOCS4
SRGN
STX11
TEKT2
TINAG
TNFRSF19
TNFRSF4
TNFSF11
TNFSF9
TP53RK
ZFP36L1
ZNF629

Genes upregulated in senescence; obtained from
Fridman et al, Oncogene 2008; 27(46): 5975-87

ALDH1A3
C9orf3
CCND1
CD44
CDKN1A
CDKN1C
CDKN2A
CDKN2B
CDKN2D
CITED2
CLTB
COL1A2
CREG1
CRYAB
CTGF
CXCL14
CYP1B1
EIF2S2
ESM1
F3
FILIP1L
FN1
GSN
GUK1
HBS1L
HPS5
HSPA2
HTATIP2
IFI16
IFNG
IGFBP1
IGFBP2
IGFBP3
IGFBP4
IGFBP5
IGFBP6
IGFBP7
IGSF3
ING1
IRF5
IRF7
ISG15
MAP1LC3B
MAP2K3
MDM2
MMP1
NDN
NME2
NRG1
OPTN
PEA15
RAB13
RAB31
RAB5B
RABGGTA
RAC1
RBL2
RGL2
RHOB
RRAS
S100A11
SERPINB2
SERPINE1
SMPD1
SMURF2
SOD1
SPARC
STAT1
TES
TFAP2A
TGFB1I1
THBS1
TNFAIP2
TNFAIP3
TP53
TSPYL5
VIM

Genes upregulated in TRM cells; obtained from
Mackay et al, Science 2016; 352(6284): 459-63

8430419L09RIK
ABI3
AMICA1
ARRDC3
ATF3
B4GALNT4
BTG2
CD244
CD69
CDH1
CISH
CSRNP1
CTNNA1
CXCR6
DDX3X
DGAT1
DHCR24
DUSP1
DUSP5
DUSP6
EGR1
EHD1
EYA2
FOS
FOSB
FOSL2
FRMD4B
GADD45B
GLRX
GPR171
GPR34
GPR55
GPR56
GSG2
HILPDA
HMGCS1
HOBIT
HPGDS
HSPA5
HSPD1
IFNG
INPP4B
INSIG1
IRF4
ISG20
ITGAE
JUN
JUNB
KLF6
LAD1
LDLRAD4
LITAF
LY6G5B
MAPKAPK3
NEDD4
NEURL3
NFKBID
NR4A1
NR4A2
ODC1
OSGIN1
P2RY10
P4HB
PER1
PLK3
PNRC1
PPP1R15A
PPP1R16B
PYGL
QPCT
RGS2
RHOB
RNF149
SC4MOL
SIK1
SKIL
SMIM3
SPSB1
STARD4
TNFAIP3
TRAF4
VDAC1
XCL1
ZFP36

Genes downregulated in TRM cells; obtained from
Mackay et al, Science 2016; 352(6284): 459-63

2010016I18RIK
A430078G23
AAED1
AB124611
ABHD8
ABTB2
ACP5
ACPL2
AI413582
ARHGAP26
ARHGEF18
ASRGL1
ATP10D
ATP1B3
AVEN
B3GAT3
BC094916
BC147527
BCL9L
BE692007
CCL5
CD84
CD97
CDC25B
CMAH
CXCR4
D1ERTD622E
DOCK2
EHD3
ELMO1
EMB KBTBD11
EML3
EOMES
FAM117A
FAM49A
FAM65B
FAM89B
FGF13
GAB3
GLIPR2
GM11346
GM1966
GM20140
GM8369
GM9835
GMFG
GNPDA2
GRAMD4
HAAO
HBA-A2
HEXB
ICAM2
IL10RA
ITGA4
ITGB1
ITGB2
KCNAB2
KCNN4
KLF2
KLF3
KLHL6
LCN4
LEF1
LFNG
LPIN1
LY6C2
LYRM2
LYST
MPND
MS4A4B
MS4A4C
NCLN
PAQR7
PCED1B
PDE2A
PHF11B
PIK3R5
PODNL1
POGK
PRKCQ
PYHIN1
RACGAP1
RASA3
RASGRP2
RBM43
RIK
S1PR1
S1PR4
S1PR5
SAMHD1
SBK1
SETX
SH2D1A
SIDT1
SMPDL3B
SNX10
ST3GAL1
STK38
TBXA2R
TCF7
THAP7
TMEM71
TSR3
TTC7B
TXK
TXNDC5
VOPP1
XRN2
*

TABLE 12
Genes Upregulated in T_{RM} cells.
UPREGULATED GENES

| | | GENECARDS |
|---|---|---|
| GENE | GENE DETAILS | ID |
| MYO7A | myosin VIIA(MYO7A) | GC11P077128 |
| GPR25 | G protein-coupled receptor 25(GPR25) | GC01P200872 |
| CLNK | cytokine dependent hematopoietic cell linker(CLNK) | GC04M010491 |
| SRGAP3 | SLIT-ROBO Rho GTPase activating protein 3(SRGAP3) | GC03M008998 |
| ATP8B4 | ATPase phospholipid transporting 8B4 | GC15M049858 |
| | (putative)(ATP8B4) | |
| AFAP1L2 | actin filament associated protein 1 like 2(AFAP1L2) | GC10M114281 |
| DAPK2 | death associated protein kinase 2(DAPK2) | GC15M063907 |
| PTMS | parathymosin(PTMS) | GC12P006765 |
| ATP10D | ATPase phospholipid transporting 10D | GC04P047487 |
| | (putative)(ATP10D) | |
| SLC27A2 | solute carrier family 27 member 2(SLC27A2) | GC15P050182 |
| LAYN | layilin(LAYN) | GC11P111541 |
| TNS3 | tensin 3(TNS3) | GC07M047281 |
| KIR2DL4 | killer cell immunoglobulin like receptor, two Ig domains | GC19P054994 |
| | and long cytoplasmic tail 4(KIR2DL4) | |
| ENTPD1 | ectonucleoside triphosphate diphosphohydrolase | GC10P095711 |
| | 1(ENTPD1) | |
| AKAP5 | A-kinase anchoring protein 5(AKAP5) | GC14P064465 |
| TTYH3 | tweety family member 3(TTYH3) | GC07P002638 |
| ASB2 | ankyrin repeat and SOCS box containing 2(ASB2) | GC14M093934 |
| DBN1 | drebrin 1(DBN1) | GC05M177456 |
| ACP5 | acid phosphatase 5, tartrate resistant (ACP5) | GC19M011574 |
| ABCB1 | ATP binding cassette subfamily B member 1(ABCB1) | GC07M087504 |
| KLRB1 | killer cell lectin like receptor B1(KLRB1) | GC12M011717 |
| ALOX5AP | arachidonate 5-lipoxygenase activating | GC13P030713 |
| | protein(ALOX5AP) | |
| GALNT2 | polypeptide N-acetylgalactosaminyltransferase | GC01P230057 |
| | 2(GALNT2) | |
| SIRPG | signal regulatory protein gamma(SIRPG) | GC20M001628 |
| NDFIP2 | Nedd4 family interacting protein 2(NDFIP2) | GC13P079481 |
| SNAP47 | synaptosome associated protein 47(SNAP47) | GC01P227730 |
| CD200R1 | CD200 receptor 1(CD200R1) | GC03M112921 |

TABLE 13
Genes Downregulated in T_{RM} cells.
DOWNREGULATED GENES

| | | GENECARDS |
|---|---|---|
| GENE | GENE DETAILS | ID |
| PATL2 | PAT1 homolog 2(PATL2) | GC15M044665 |
| FAM65B | family with sequence similarity 65 | GC06M024805 |
| | member B(FAM65B) | |
| ADRB2 | adrenoceptor beta 2(ADRB2) | GC05P148825 |
| SORL1 | sortilin related receptor 1(SORL1) | GC11P121452 |
| CD300A | CD300a molecule (CD300A) | GC17P074466 |
| C1orf21 | chromosome 1 open reading | GC01P184356 |
| | frame 21(C1orf21) | |
| PLEK | pleckstrin (PLEK) | GC02P068365 |
| PLAC8 | placenta specific 8(PLAC8) | GC04M083090 |
| ATM | ATM serine/threonine kinase(ATM) | GC11P108127 |
| PTGDR | prostaglandin D2 receptor(PTGDR) | GC14P052267 |
| PXN | paxillin(PXN) | GC12M120210 |
| DHRS3 | dehydrogenase/reductase 3(DHRS3) | GC01M012567 |

### REFERENCES

1. Pardoll D M. The blockade of immune checkpoints in cancer immunotherapy. Nature reviews Cancer 2012, 12(4): 252-264.
2. Drake C G, Lipson E J, Brahmer J R. Breathing new life into immunotherapy: review of melanoma, lung and kidney cancer. Nature reviews Clinical oncology 2014, 11(1): 24-37.
3. Garon E B, Rizvi N A, Hui R, Leighl N, Balmanoukian A S, Eder J P, et al. Pembrolizumab for the treatment of non-small-cell lung cancer. The New England journal of medicine 2015, 372(21): 2018-2028.
4. Sharma P, Allison J P. Immune checkpoint targeting in cancer therapy: toward combination strategies with curative potential. Cell 2015, 161(2): 205-214.
5. Tumeh P C, Harview C L, Yearley J H, Shintaku I P, Taylor E J, Robert L, et al. PD-1 blockade induces responses by inhibiting adaptive immune resistance. Nature 2014, 515(7528): 568-571.
6. Galon J, Costes A, Sanchez-Cabo F, Kirilovsky A, Mlecnik B, Lagorce-Pages C, et al. Type, density, and location of immune cells within human colorectal tumors predict clinical outcome. Science 2006, 313(5795): 1960-1964.
7. Yang Y. Cancer immunotherapy: harnessing the immune system to battle cancer. J Clin Invest 2015, 125(9): 3335-3337.
8. Baitsch L, Baumgaertner P, Devevre E, Raghav S K, Legat A, Barba L, et al. Exhaustion of tumor-specific CD8(+) T cells in metastases from melanoma patients. The Journal of clinical investigation 2011, 121(6): 2350-2360.
9. Bindea G, Mlecnik B, Tosolini M, Kirilovsky A, Waldner M, Obenauf A C, et al. Spatiotemporal dynamics of intratumoral immune cells reveal the immune landscape in human cancer. Immunity 2013, 39(4): 782-795.
10. Collins N, Godec J, Zou L H, Mihm M C, Getz G, Haining W N. Transcriptional Hallmarks Of Tumor Infiltrating Lymphocyte Responses To Melanoma. Blood 2013, 122(21).
11. Tirosh I, Izar B, Prakadan S M, Wadsworth M H, 2nd, Treacy D, Trombetta J J, et al. Dissecting the multicellular ecosystem of metastatic melanoma by single-cell RNA-seq. Science 2016, 352(6282): 189-196.
12. Pauken K E, Wherry E J. Overcoming T cell exhaustion in infection and cancer. Trends in immunology 2015, 36(4): 265-276.
13. Brown I E, Blank C, Kline J, Kacha A K, Gajewski T F. Homeostatic proliferation as an isolated variable reverses CD8+ T cell anergy and promotes tumor rejection. Journal of immunology 2006, 177(7): 4521-4529.
14. Montes C L, Chapoval A I, Nelson J, Orhue V, Zhang X, Schulze D H, et al. Tumor-induced senescent T cells with suppressor function: a potential form of tumor immune evasion. Cancer research 2008, 68(3): 870-879.
15. Johnston R J, Comps-Agrar L, Hackney J, Yu X, Huseni M, Yang Y, et al. The immunoreceptor TIGIT regulates antitumor and antiviral CD8(+) T cell effector function. Cancer cell 2014, 26(6): 923-937.
16. Curran M A, Geiger T L, Montalvo W, Kim M, Reiner S L, Al-Shamkhani A, et al. Systemic 4-1BB activation induces a novel T cell phenotype driven by high expression of Eomesodermin. J Exp Med 2013, 210(4): 743-755.
17. Willoughby J E, Kerr J P, Rogel A, Taraban V Y, Buchan S L, Johnson P W, et al. Differential impact of CD27 and 4-1BB costimulation on effector and memory CD8 T cell generation following peptide immunization. J Immunol 2014, 193(1): 244-251.
18. Topalian S L, Drake C G, Pardoll D M. Immune checkpoint blockade: a common denominator approach to cancer therapy. Cancer Cell 2015, 27(4): 450-461.
19. Gros A, Robbins P F, Yao X, Li YF, Turcotte S, Tran E, et al. PD-1 identifies the patient-specific CD8(+) tumor-reactive repertoire infiltrating human tumors. The Journal of clinical investigation 2014, 124(5): 2246-2259.
20. Fourcade J, Sun Z, Benallaoua M, Guillaume P, Luescher I F, Sander C, et al. Upregulation of Tim-3 and PD-1 expression is associated with tumor antigen-specific CD8+ T cell dysfunction in melanoma patients. The Journal of experimental medicine 2010, 207(10): 2175-2186.
21. Wolfi M, Kuball J, Ho W Y, Nguyen H, Manley T J, Bleakley M, et al. Activation-induced expression of CD137 permits detection, isolation, and expansion of the full repertoire of CD8+ T cells responding to antigen without requiring knowledge of epitope specificities. Blood 2007, 110(1): 201-210.
22. Mackay L K, Rahimpour A, Ma J Z, Collins N, Stock A T, Hafon M L, et al. The developmental pathway for CD103(+)CD8+ tissue-resident memory T cells of skin. Nature immunology 2013, 14(12): 1294-1301.
23. Mueller S N, Gebhardt T, Carbone F R, Heath W R. Memory T cell subsets, migration patterns, and tissue residence. Annual review of immunology 2013, 31: 137-161.
24. Mackay L K, Minnich M, Kragten N A, Liao Y, Nota B, Seillet C, et al. Hobit and Blimp1 instruct a universal transcriptional program of tissue residency in lymphocytes. Science 2016, 352(6284): 459-463.
25. Lee L N, Ronan E O, de Lara C, Franken K L, Ottenhoff T H, Tchilian E Z, et al. CXCR6 is a marker for protective antigen-specific cells in the lungs after intranasal immunization against Mycobacterium tuberculosis. Infection and immunity 2011, 79(8): 3328-3337.
26. Tse S W, Radtke A J, Espinosa D A, Cockburn I A, Zavala F. The chemokine receptor CXCR6 is required for the maintenance of liver memory CD8(+) T cells specific for infectious pathogens. The Journal of infectious diseases 2014, 210(9): 1508-1516.
27. Skon C N, Lee J Y, Anderson K G, Masopust D, Hogquist K A, Jameson S C.

Transcriptional downregulation of Slprl is required for the establishment of resident memory CD8+ T cells. Nature immunology 2013, 14(12): 1285-1293.
28. Ariotti S, Hogenbirk M A, Dijkgraaf F E, Visser L L, Hoekstra M E, Song J Y, et al. T cell memory. Skin-resident memory CD8(+) T cells trigger a state of tissue-wide pathogen alert. Science 2014, 346(6205): 101-105.
29. Schenkel J M, Fraser K A, Beura L K, Pauken K E, Vezys V, Masopust D. T cell memory. Resident memory CD8 T cells trigger protective innate and adaptive immune responses. Science 2014, 346(6205): 98-101.
30. Best J A, Blair D A, Knell J, Yang E, Mayya V, Doedens A, et al. Transcriptional insights into the CD8(+) T cell response to infection and memory T cell formation. Nature immunology 2013, 14(4): 404-412.
31. Faure M, Long E O. KIR2DL4 (CD158d), an NK cell-activating receptor with inhibitory potential. Journal of immunology 2002, 168(12): 6208-6214.
32. Rajagopalan S, Long E O. KIR2DL4 (CD158d): An activation receptor for HLA-G. Frontiers in immunology 2012, 3: 258.
33. Wisniewski A, Kowal A, Wyrodek E, Nowak I, Majorczyk E, Wagner M, et al. Genetic polymorphisms and expression of HLA-G and its receptors, KIR2DL4 and LILRB1, in non-small cell lung cancer. Tissue antigens 2015, 85(6): 466-475.
34. Brooke G, Holbrook J D, Brown M H, Barclay A N. Human lymphocytes interact directly with CD47 through a novel member of the signal regulatory protein (SIRP) family. Journal of immunology 2004, 173(4): 2562-2570.
35. Piccio L, Vermi W, Boles K S, Fuchs A, Strader C A, Facchetti F, et al. Adhesion of human T cells to antigen-presenting cells through SIRPbeta2-CD47 interaction costimulates T-cell proliferation. Blood 2005, 105(6): 2421-2427.

1.-16. (canceled)

17. A method of eliciting an anti-tumor response comprising contacting a tumor or tumor cell with an effective amount of a population of T-cells that exhibit higher than baseline expression of one or more genes set forth in Table 11.

18. (canceled)

19. The method of claim 17, wherein the T-cells are tissue-resident memory cells (T_{RM}).

20. The method of any one of claim 17, wherein baseline expression is normalized mean gene expression.

21. The method of claim 20, wherein higher than baseline expression is at least about a 2-fold increase in expression relative to baseline expression and/or lower than baseline expression is at least about a 2-fold decrease in expression relative to baseline expression.

22.-44. (canceled)

45. A method of diagnosing a subject having cancer, comprising contacting the same with an agent that detects the presence of one or more genes set forth in Table 11 in the cancer or a sample thereof, wherein the presence of the one or more genes at higher than baseline levels is diagnostic of cancer.

46. The method of claim 45, wherein the presence of the one or more genes set forth in Table 11 at higher than baseline levels is further indicative of a higher probability and/or duration of survival.

47. (canceled)

48. The method of claim 45, wherein the presence of the one or more genes set forth in Table 11 at lower than baseline levels is further indicative of a higher probability and/or duration of survival.

49.-63. (canceled)

64. The method of claim 45, wherein the cancer is an epithelial cancer or tumor.

65. The method of claim 45, wherein the cancer or tumor is in head, neck, lung, lung, prostate, colon, pancreas, esophagus, liver, skin, kidney, adrenal gland, brain, or comprises a lymphoma, breast, endometrium, uterus, ovary, testes, lung, prostate, colon, pancreas, esophagus, liver, skin, kidney, adrenal gland, or brain of the subject.

66. The method of claim 45, wherein the cancer comprises a metastasis or recurring tumor, cancer or neoplasia.

67. The method of claim 45, wherein the cancer comprises a non-small cell lung cancer (NSCLC) or head and neck squamous cell cancer (HNSCC).

68.-137. (canceled)

138. A method of determining prognosis of a subject having cancer comprising measuring the density of tumor infiltrating lymphocytes (TILs) in the cancer or a sample thereof, wherein a high density of TILs indicates an increased probability and/or duration of survival.

139. The method of claim 138, wherein the TILs are enriched for tissue-resident memory cells (T_{RM}).

140. The method of claim 139, wherein the TILs are enriched for T_{RM} by contacting the TILs with an effective amount of an active agent that induces higher than baseline expression of one or more genes set forth in Table 11.

141. The method of claim 140, wherein the active agent is an antibody, a small molecule, or a nucleic acid.

142. The method of claim 139, wherein the TILs enriched for T_{RM} have enhanced cytotoxicity and proliferation.

143-178. (canceled)
